(19) United States
(12) Patent Application Publication
(10) Pub. No.: US 2024/0260553 A1
(43) Pub. Date: Aug. 8, 2024

(54) CHIMERIC TRANSGENIC IMMUNOGLOBULIN MICE WITH AN ALTERED HEAVY CHAIN LOCUS AND METHODS OF MAKING AND USING SAME

(71) Applicant: Gilead Sciences, Inc., Foster City, CA (US)

(72) Inventors: Daniel Rohrer, Los Gatos, CA (US); Peter Brams, Sacramento, CA (US)

(21) Appl. No.: 18/413,972

(22) Filed: Jan. 16, 2024

Related U.S. Application Data
(60) Provisional application No. 63/439,801, filed on Jan. 18, 2023.

(51) Int. Cl. A01K 67/0278; C07K 16/10; C12N 9/22; C12N 15/11

(52) U.S. Cl. CPC A01K 67/0278; C07K 16/1003; C12N 9/22; C12N 15/111; A01K 2217/072; A01K 2227/105; A01K 2267/01; C07K 2317/56; C12N 2310/20

(57) ABSTRACT

Chimeric transgenic immunoglobulin (Ig) mice are provided that comprise an altered heavy chain locus in which endogenous mouse D and J segments have been deleted and a human heavy chain Ig transgene is inserted at that location such that the transgenic Ig mouse expresses an antibody repertoire that utilizes human VH and mouse VH in heavy chains, each linked to human DH and JH segments, leading to enhanced diversity. Methods of preparing and using the transgenic animals (e.g., to raise antibodies) are also provided.

### CROSS-REFERENCE TO RELATED APPLICATIONS

This application claims the benefit under 35 U.S.C. § 119(c) of U.S. Provisional Application No. 63/439,801, filed on Jan. 18, 2023, which is hereby incorporated herein by reference in its entirety for all purposes.

### SEQUENCE LISTING

The instant application contains a Sequence Listing which has been submitted electronically in .XML file format and is hereby incorporated by reference in its entirety. Said .XML copy, created on created on Apr. 7, 2024, is named ZL8018-WO-PCT_SL.xml and is 153,978 bytes in size.

### BACKGROUND OF THE DISCLOSURE

Immunotherapy has revolutionized the treatment of a wide variety of diseases, including cancer and autoimmune disorders. A key component in immunotherapy is the ability to create a therapeutic antibody with desired binding characteristics against an antigen of interest. A wide variety of approaches have been established in the art for generating such antibodies, which were developed subsequent to the discovery of the original B cell hybridoma technology for making monoclonal antibodies (mAbs) (for a review, see e.g., Lu et al. (2018) J. Biomed. Sci. 27:1). Initially, mouse mAbs were made more human-like by recombinantly swapping in a human constant region for the mouse constant region, thereby creating a chimeric antibody. Alternatively, mouse (or other non-human) mAb-derived CDR sequences were grafted into a human antibody framework to thereby create a humanized antibody with more human-derived sequences than chimeric antibodies. Subsequently, fully human therapeutic antibodies were able to be generated using either of two different general approaches that are now established in the art: screening diverse human antibody libraries in vitro, for example libraries expressed on display packages such as bacteriophage, and generating antibodies in animals carrying transgenic Ig sequences (e.g., human Ig sequences).

The initial human antibody transgenic mice created in the art integrated a fully human transgene (human variable and constant regions) into the mouse genome while disabling the endogenous mouse Ig locus such that mouse antibodies were not produced in the animals (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; and U.S. Pat. Nos. 5,939,598; 6,075,181; 6,114,598; 6,150,584 and 6,162,963, all to Kucherlapati et al.).

Transgenic mice that only carry human variable region transgene sequences operatively linked to the endogenous mouse constant region, thus generating chimeric antibodies in the animals, also have been described (see e.g., U.S. Pat. Nos. 6,596,541; 10,584,364; and US Patent Publication No. 2016/0316731). The use of the mouse constant region, which preserves more of the native endogenous Ig locus architecture and signaling function when compared to fully human transgenes, may generate antibodies that would not be formed using a human constant region, thereby providing a different antibody repertoire for testing responsiveness to antigens of interest. The chimeric antibodies produced in the mice then can be reverse engineered to be fully human.

Additional approaches for achieving different antibody repertoire diversity in Ig transgenic mice have been described in the art. For example, one approach involves use of long heavy chain CDR3 sequences in the heavy chain transgene, which can generate additional HCDR3 diversity (sec e.g., U.S. Pat. Nos. 10,640,574; 10,562,980; 10,259,863; 10,259,863; 9,504,236; and US Patent Publication No. 2020/0181241). Another approach involves use of a fixed light chain transgene sequence, which may allow for the identification of different heavy chain pairings than obtained using a full repertoire of light chains (see e.g., U.S. Pat. No. 10,412,940; and US Patent Publication No. 2020/0024368).

While some advances have been made, additional approaches and compositions are needed for preparing animals that express transgenic Ig sequences for the generation of therapeutic antibodies, particularly ones that generate hosts with increased diversity in the antibody repertoire.

### SUMMARY OF THE DISCLOSURE

The disclosure pertains to transgenic mice with a chimeric immunoglobulin (Ig) heavy chain locus, and methods of making same. The disclosure provides methods of preparing a chimeric Ig heavy chain locus in a mouse cell, as well as transgenic mice produced by the methods, in which the endogenous mouse heavy chain locus has the D and J segments downstream of the Adam6b gene and upstream of IgM segments deleted and has a human heavy chain variable region transgene, comprising a plurality of human V, D and J segments, inserted at that location. In some embodiments, the disclosed methods produce a transgenic mouse that expresses an antibody repertoire that utilizes human VH segments and mouse VH segments in the resultant heavy chains, through either hVH-D-J recombination or mVH-D-J recombination at the altered heavy chain locus. Thus, in some embodiments, the transgenic mouse of the disclosure, referred to herein as a Supra-diversity mouse that carries a Supra allele, provides a tool for generation of a greater diversity of VH sequences than is generated by either an unaltered endogenous mouse HC locus or a humanized VDJ locus alone (i.e., a locus with active human VH segments and inactivated mouse VH segments).

Accordingly, in one aspect, the disclosure pertains to a method of preparing a chimeric immunoglobulin heavy chain locus in a mouse cell, the method comprising:
(a) deleting mouse D segments and J segments downstream of Adam6b and upstream of IgM in the endogenous mouse immunoglobulin (Ig) heavy chain locus; and
(b) introducing a human heavy chain variable region transgene into the endogenous mouse Ig heavy chain locus downstream of Adam6b and upstream of IgM, wherein the transgene comprises a plurality of unrearranged human variable segments (VHs), a plurality of human D segments (DH) and a plurality of human J segments (JH) to thereby prepare a chimeric Ig heavy chain locus in the mouse cell, wherein a mouse comprising the chimeric Ig heavy chain locus expresses an antibody repertoire comprising mouse VH segments and human VH segments, each linked to human DH and JH segments.

In an embodiment, steps (a) and (b) are conducted simultaneously using CRISPR-Cas9-mediated knock-out/knock-in technology. In another embodiment, the mouse D segments and mouse J segments are deleted by CRISPR-Cas9-mediated gene editing (i.e., step (a) of the method) and the human heavy chain variable region transgene is introduced into the mouse Ig heavy chain locus by Cre-Lox-mediated recombination (i.e., step (b) of the method).

In an embodiment, the human heavy chain variable region transgene is carried on a bacterial artificial chromosome (BAC).

In embodiments, a mouse comprising the chimeric Ig heavy chain locus expresses an antibody repertoire comprising at least twenty, at least thirty or at least forty different human VH segments. In an embodiment, a mouse comprising the chimeric Ig heavy chain locus expresses an antibody repertoire comprising human VH segments 3-74, 3-73, 3-72, 2-70, 1-69, 3-66, 3-64, 4-61, 4-59, 1-58, 3-53, 5-51, 3-49, 3-48, 1-46, 1-45, 3-43, 4-39, 4-34, 3-33, 4-31, 3-30, 4-28, 2-26, 1-24, 3-23, 3-21, 3-20, 1-18, 3-15, 3-13, 3-11, 3-9, 1-8, 3-7, 2-5, 7-4-1, 4-4, 1-3, 1-2 and 6-1.

In an embodiment, a mouse comprising the chimeric Ig heavy chain locus expresses an antibody repertoire comprising at least 100 different mouse VH segments.

In an embodiment, a mouse comprising the chimeric Ig heavy chain locus expresses an antibody repertoire comprising at least fifteen different human DH segments. In an embodiment, a mouse comprising the chimeric Ig heavy chain locus expresses an antibody repertoire comprising at least twenty-six different human DH segments.

In an embodiment, a mouse comprising the chimeric Ig heavy chain locus expresses an antibody repertoire comprising six different human JH segments.

In another aspect, the disclosure pertains to a transgenic mouse cell, e.g., prepared according to a method of the disclosure. In one embodiment, the disclosure provides a transgenic mouse cell comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHs), a plurality of human D segments (DH) and a plurality of human J segments (JH),
   wherein the transgenic mouse cell comprising the chimeric Ig heavy chain locus expresses an antibody repertoire comprising mouse VH segments and human VH segments, each linked to human DH and JH segments.

In another aspect, the disclosure pertains to a transgenic mouse, e.g., prepared from a transgenic mouse cell of the disclosure. In one embodiment, the disclosure provides a transgenic mouse comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHs), a plurality of human D segments (DH) and a plurality of human J segments (JH),
   wherein the transgenic mouse comprising the chimeric Ig heavy chain locus expresses an antibody repertoire comprising mouse VH segments and human VH segments, each linked to human DH and JH segments.

In an embodiment, the transgenic mouse further comprises a transgene construct encoding a human immunoglobulin light chain such that the mouse expresses antibodies comprising human immunoglobulin light chain variable domains operably linked to either a mouse or human C-kappa constant region.

In yet another embodiment, a chimeric immunoglobulin heavy chain locus of the disclosure can serve as the starting point for creating a full-diversity human VH segment locus through deletion of the endogenous mouse VH segments, as described herein.

In another aspect, the disclosure pertains to a method of generating antibodies to an antigen of interest, the method comprising administering the antigen of interest to the transgenic mouse of the disclosure, such that antibodies that bind to the antigen of interest are generated. In an embodiment, the method further comprises isolating an antibody of interest from the mouse. In an embodiment, the method further comprises isolating nucleic acid encoding an antibody of interest from the mouse and replacing mouse constant region sequences within the nucleic acid with human constant region sequences.

### BRIEF DESCRIPTION OF THE DRAWINGS

FIG. 1 is a schematic diagram of a one-step process for inserting a human heavy chain transgene construct into the endogenous mouse Ig locus.

FIG. 2A-D shows a schematic diagram illustrating how the Supra allele supports both fully human VDJ and chimeric VDJ recombination (FIG. 2A) and graphs showing the usage of mouse VH vs. human VH, as determined by Next Generation Sequencing (NGS), in naïve spleen cells (FIG. 2B), Covid spike protein-immunized spleen cells (FIG. 2C) and single B cells from Target X-immunized lymph nodes (FIG. 2D) of transgenic mice carrying the Supra allele.

FIG. 3 is a schematic diagram summarizing the hVH, D and J segment usage in the Supra allele transgenic mice.

FIG. 4 is a schematic diagram summarizing the mVH, hVH, D and J segment usage in the Supra allele transgenic mice.

FIG. 5 is a schematic diagram of a representative vector construct of a human Ig heavy chain transgene of the disclosure.

FIG. 6 is a schematic diagram of a representative approach for converting a Supra allele to a full-diversity human VH segment transgene.

### DETAILED DESCRIPTION

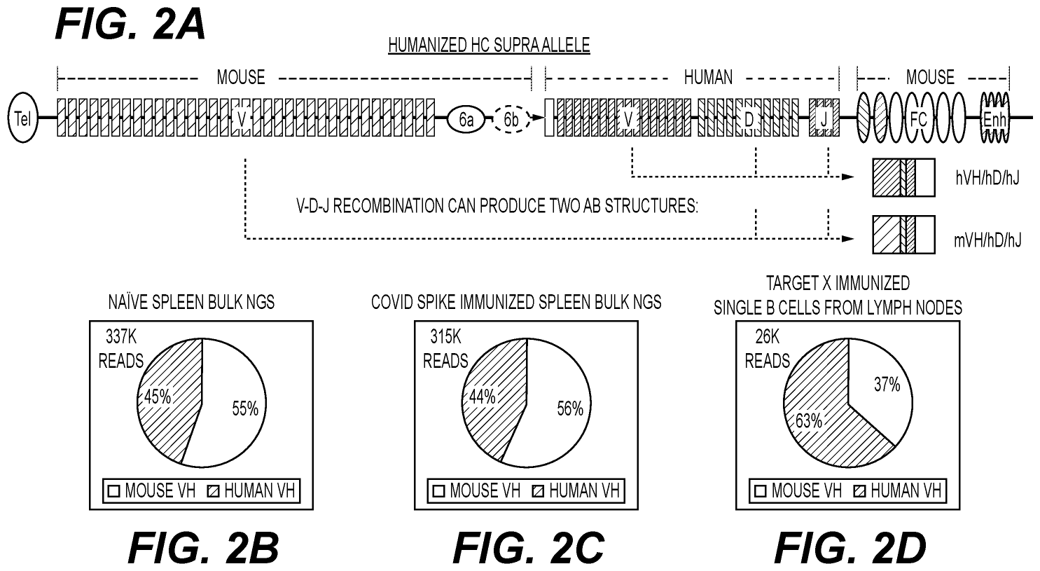

The chimeric Ig heavy chain locus described herein allows for the expression of an antibody repertoire that utilizes human VH segments and mouse VH segments in the resultant heavy chains, through either hVH-D-J recombination or mVH-D-J recombination at the altered heavy chain locus, as illustrated schematically in FIG. 2A. The resultant chimeric antibodies may be well-suited for reagent and/or diagnostic use in their chimeric format. Moreover, the chimeric antibodies can be converted to fully human antibodies using standard methods, as described herein, allowing their potential use for therapy. Still further, in another embodiment, the chimeric Ig heavy chain locus can serve as the starting point for creating an Ig heavy chain locus comprising only human VH, DH, and JH variable segments through excision of the endogenous mouse VH segments.

Various aspects of the disclosure are described in further detail below. Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook el al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

### 1. Supra-Diversity Approach

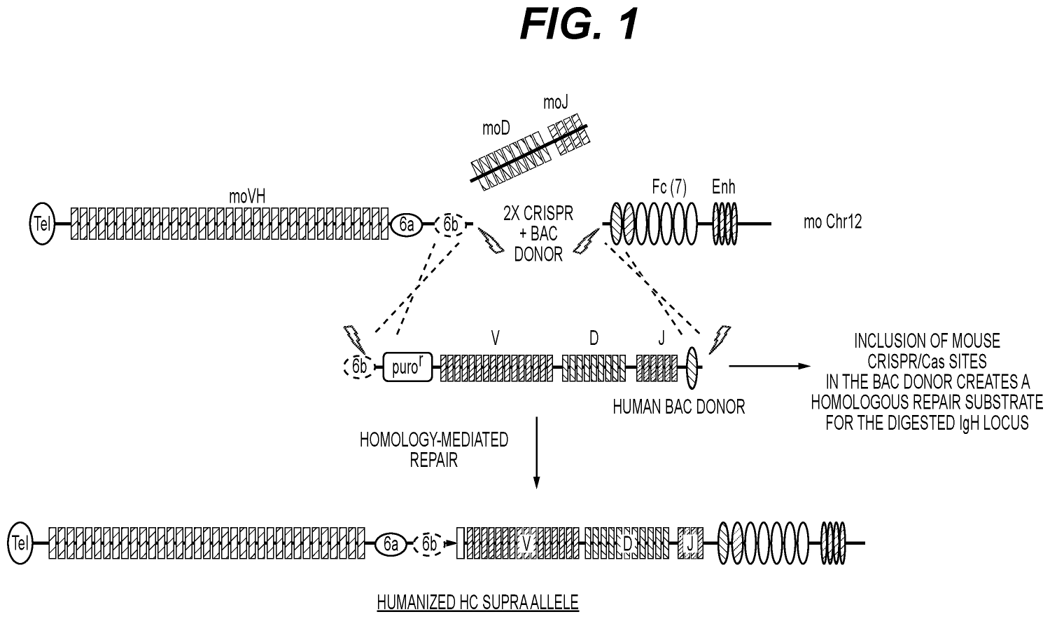

An embodiment of the approach for creating a modified endogenous Ig locus that generates heavy chains using either human or mouse V segments is illustrated schematically in FIG. 1. The approach involves a simultaneous dual modification at the endogenous heavy chain locus: (i) a modification that removes (deletes, excises, knocks-out) the endogenous mouse D and J segments (also referred to herein as mDH and mJH) downstream of the Adam6b gene and upstream of the mouse IgM constant region segments; and (ii) a modification that inserts (knocks-in, swaps in) a human Ig heavy chain variable region transgene that comprises human V. D and J segments (also referred to herein as hVH, hDH and hJH) at the site of deletion of mDH and mJH. Alternatively, the two modifications can be performed in two separate steps as described herein. As illustrated in FIG. 2A, the resultant chimeric heavy chain locus is capable of both mVH-hDH-hJH and hVH-hDH-hJH recombinations, thereby generating heavy chains that incorporate either a mouse VH segment or a human VH segment.

Accordingly, in one aspect, the disclosure pertains to a method of preparing a chimeric immunoglobulin heavy chain locus in a mouse cell, the method comprising:
(a) deleting mouse D segments and J segments downstream (i.e., 3′) of Adam6b and upstream (i.e., 5′) of IgM in the endogenous mouse immunoglobulin (Ig) heavy chain locus; and
(b) introducing a human heavy chain variable region transgene into the endogenous mouse Ig heavy chain locus downstream (i.e., 3′) of Adam6b and upstream (i.e., 5′) of IgM, wherein the transgene comprises a plurality of unrearranged human variable segments (VHs), a plurality of human D segments (DH) and a plurality of human J segments (JH) to thereby prepare a chimeric Ig heavy chain locus in the mouse cell,
   wherein a mouse comprising the chimeric Ig heavy chain locus expresses an antibody repertoire comprising mouse VH segments and antibodies comprising human VH segments, each linked to human DH and JH segments.

In one embodiment, steps (a) and (b) are conducted simultaneously, e.g., using CRISPR-Cas9-mediated knock-out/knock-in technology. In another embodiment, the mouse D segments and mouse J segments are deleted by CRISPR-Cas9-mediated gene editing (i.e., step (a)) and the human heavy chain variable region transgene is introduced into the mouse Ig heavy chain locus by Cre-Lox-mediated recombination (i.e., step (b)). These technologies for recombination are well established in the art and can be applied at the Ig locus using standard methods. Use of recombination approaches to create a Supra allele of the disclosure is described in further detail in Section III below and in Example 1.

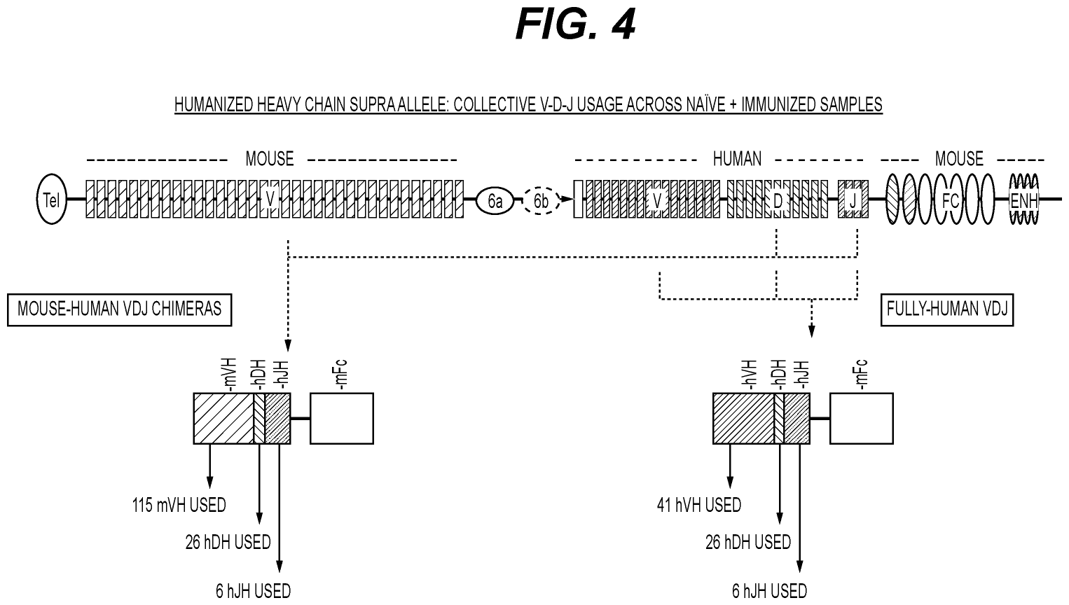

The Supra-diversity mouse of the disclosure generates an antibody repertoire comprising heavy chains that use either mouse VH or human VH, as illustrated schematically in FIG. 4 and described in detail in Example 2. Analysis of the antibody mRNA expressed in the mouse showed that essentially all human VH, DH and JH segments included in the transgene were utilized in the antibody repertoire. The structure and construction of the human Ig heavy chain variable region transgene is described in further detail in Section II below. Additionally, over 100 endogenous mouse VH segments were shown by mRNA analysis to be utilized in the antibody repertoire. Accordingly, in an embodiment, a transgenic mouse of the disclosure comprises a chimeric Ig heavy chain locus that expresses an antibody repertoire comprising at least 100 different mouse VH segments.

The modified endogenous heavy chain locus preserves the germline location, configuration and sequences of the ADAM6 gene complex that includes Adam6a and Adam6b. These genes have been demonstrated to be necessary for male fertility in mice and thus preserving the native structure of the ADAM6 genes within the Ig locus is a beneficial feature of the Supra allele that may provide better fertility than transgenic approaches known in the art that delete the ADAM6 genes from the Ig locus and then reinsert them elsewhere in the mouse genome (e.g., as described in PCT Publication WO 2013/079953).

The ADAM6 gene complex includes two mouse DH segments. 1-3 and 3-1 (as illustrated in FIG. 1) embedded within it, separate from the remainder of the endogenous mouse DH segments that are deleted. These 1-3 and 3-1 mDH segments remain functional and active. Thus, the Supra allele is also capable of generating chimeric heavy chains comprising mVH-mDH-hJH as a further means of generating diversity. It is noted, however, that ascertaining with any certainty the usage of an mDH segment from within the ADAM6 complex can be challenging due to the short length of those mDH segments, as well as their high homology with human DH homologs present in the Supra allele.

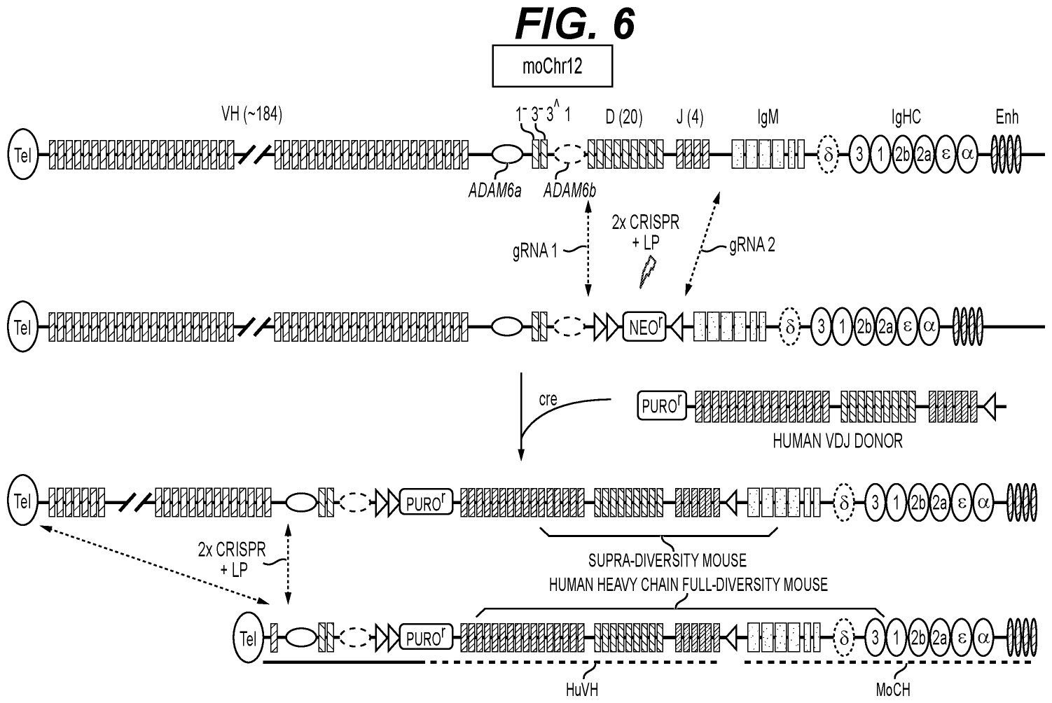

In another embodiment, the Supra allele serves as the starting point for creating an Ig heavy chain locus utilizing only human VH, DH, and JH variable segments through excision (deletion, knocking out) of the endogenous mouse VH segments, while still preserving the germline location, configuration and sequences of the ADAM6 gene complex, including Adam6a and Adam6b. As illustrated schematically in FIG. 6, the endogenous mouse VH segments of the Supra allele can be deleted by standard recombination, (e.g., using CRISPR/Cas9-mediated recombination or other suitable recombination technology available in the art), thereby resulting in an Ig heavy chain locus containing a full-diversity of human VH segments while lacking endogenous mouse VH segments such that a transgenic mouse carrying the Ig heavy chain locus expresses an antibody repertoire comprising a full-diversity of human VH segments and lacking mouse VH segments. Accordingly, in an embodiment, mouse VH segments are deleted from a Supra allele of the disclosure by standard recombinant methods to create an Ig heavy chain locus containing only human VH segments.

In another aspect, the disclosure provides a method of preparing an immunoglobulin (Ig) heavy chain locus in a mouse cell wherein the Supra allele is an intermediate, the method comprising:
(a) deleting mouse D segments and J segments downstream (i.e., 3′) of Adam6b and upstream (i.e., 5′) of IgM in the endogenous mouse immunoglobulin (Ig) heavy chain locus;
(b) introducing a human heavy chain variable region transgene into the endogenous mouse Ig heavy chain locus downstream (i.e., 3′) of Adam6b and upstream (i.e., 5′) of IgM, wherein the transgene comprises a plurality of unrearranged human variable segments (VHs), a plurality of human D segments (DH) and a plurality of human J segments (JH) to thereby prepare a chimeric Ig heavy chain locus; and
(c) deleting mouse V segments upstream (i.e., 5′) of Adamba in the mouse cell,
wherein a mouse comprising the Ig heavy chain locus expresses an antibody repertoire comprising human VH segments, each linked to human DH and JH segments, and lacking mouse VH segments. As illustrated in FIG. 6, the approach allows for introduction of a full-diversity of human VH segments while still preserving the germline location, configuration and sequences of the ADAM6 gene complex, including Adam6a and Adam6b.

### II. Heavy Chain Transgene Construct Preparation

The transgene constructs of the disclosure can be prepared using standard recombinant DNA techniques. Cloning vectors containing polylinkers are useful as starting vectors for insertion of DNA fragments of interest. Suitable cloning vectors are well established in the art. Moreover, plasmids or other vectors (e.g., YACs and BACs) carrying human unrearranged heavy chain immunoglobulin sequences have been described in the art (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; and U.S. Pat. Nos. 5,939,598; 6,075,181; 6,114,598; 6,150,584 and 6,162,963, all to Kucherlapati et al.) and can be used as a source of heavy chain V, D and J region sequences. Moreover, databases are established in the art disclosing human heavy chain V, D and J sequences. In an embodiment, desired sequences can be synthesized by standard methods or obtained by standard recombinant DNA methods. The appropriate DNA fragments are then operatively linked through ligation into a cloning vector, followed by characterization of the vector (e.g., by restriction fragment analysis or sequencing or the like) to ensure proper arrangement of the fragments.

In an embodiment, the chimeric Ig heavy chain locus of the disclosure comprises over twenty different human VH segments (e.g., a transgenic mouse carrying the chimeric Ig locus expresses an antibody repertoire comprising at least twenty different human VH segments). In another embodiment, the chimeric Ig heavy chain locus of the disclosure comprises over thirty different human VH segments (e.g., a transgenic mouse carrying the chimeric Ig locus expresses an antibody repertoire comprising at least thirty different human VH segments). In another embodiment, the chimeric Ig heavy chain locus of the disclosure comprises over forty different human VH segments (e.g., a transgenic mouse carrying the chimeric Ig locus expresses an antibody repertoire comprising at least forty different human VH segments).

In an embodiment, the chimeric Ig heavy chain locus of the disclosure comprises human VH segments 3-74, 3-73, 3-72, 2-70, 1-69, 3-66, 3-64, 4-61, 4-59, 1-58, 3-53, 5-51, 3-49, 3-48, 1-46, 1-45, 3-43, 4-39, 4-34, 3-33, 4-31, 3-30, 4-28, 2-26, 1-24, 3-23, 3-21, 3-20, 1-18, 3-15, 3-13, 3-11, 3-9, 1-8, 3-7, 2-5, 7-4-1, 4-4, 1-3, 1-2 and 6-1. For example, the transgene can comprise human VH segments 3-74*01, 3-73*01, 3-72*01, 2-70*03, 1-69*01, 3-66*01, 3-64*01, 4-61*01, 4-59*01, 1-58*01, 3-53*01, 5-51*01, 3-49*01, 3-48*01, 1-46*01, 1-45*01, 3-43*01, 4-39*01, 4-34*01, 3-33*01, 4-31*02, 3-30*01, 4-28*01, 2-26*01, 1-24*01, 3-23*01, 3-21*01, 3-20*01, 1-18*01, 3-15*01, 3-13*04, 3-11*01, 3-9*02, 1-8*01, 3-7*02, 2-5*02, 7-4-1*02, 4-4*05, 1-3*02, 1-2*01 and 6-1*01.

In an embodiment, a mouse comprising the chimeric Ig heavy chain locus expresses an antibody repertoire comprising human VH segments 3-74, 3-73, 3-72, 2-70, 1-69, 3-66, 3-64, 4-61, 4-59, 1-58, 3-53, 5-51, 3-49, 3-48, 1-46, 1-45, 3-43, 4-39, 4-34, 3-33, 4-31, 3-30, 4-28, 2-26, 1-24, 3-23, 3-21, 3-20, 1-18, 3-15, 3-13, 3-11, 3-9, 1-8, 3-7, 2-5, 7-4-1, 4-4, 1-3, 1-2 and 6-1. For example, a mouse of the disclosure can express an antibody repertoire comprising human VH segments 3-74*01, 3-73*01, 3-72*01, 2-70*03, 1-69*01, 3-66*01, 3-64*01, 4-61*01, 4-59*01, 1-58*01, 3-53*01, 5-51*01, 3-49*01, 3-48*01, 1-46*01, 1-45*01, 3-43*01, 4-39*01, 4-34*01, 3-33*01, 4-31*02, 3-30*01, 4-28*01, 2-26*01, 1-24*01, 3-23*01, 3-21*01, 3-20*01, 1-18*01, 3-15*01, 3-13*04, 3-11*01, 3-9*02, 1-8*01, 3-7*02, 2-5*02, 7-4-1*02, 4-4*05, 1-3*02, 1-2*01 and 6-1*01.

In an embodiment, the human Ig heavy chain transgene comprises at least fifteen different human D segments, more preferably at least 26 different human D segments or all 27 human D segments identified to date. In an embodiment, a mouse comprising the chimeric Ig heavy chain locus expresses an antibody repertoire comprising at least 15 and more preferably at least 26 different human D segments.

In an embodiment, the human Ig heavy chain transgene comprises six different human J segments, i.e., the human J1-J6 segments. In an embodiment, a mouse comprising the chimeric Ig heavy chain locus expresses an antibody repertoire comprising six different JH segments (i.e., human J1-J6).

In an embodiment, the transgene construct is carried on a bacterial artificial chromosome (BAC). BAC technology for carrying Ig transgenes is well-established in the art.

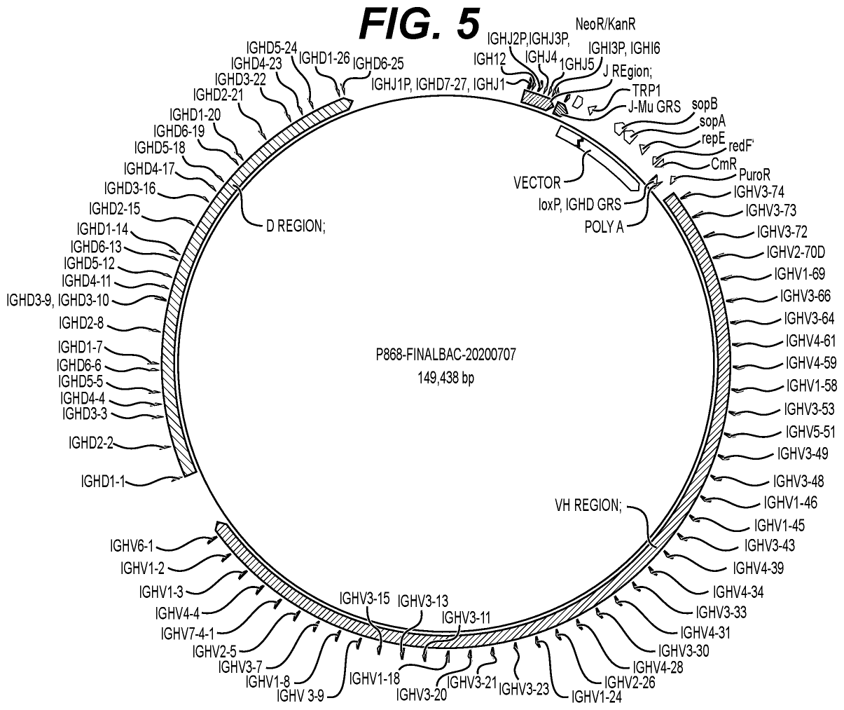

A non-limiting example of a human Ig heavy chain variable region transgene vector of the disclosure is illustrated schematically in FIG. 5. In an embodiment, the human Ig heavy chain variable region transgene construct comprises the nucleotide sequence shown in SEQ ID NO: 1.

The nucleotide sequence of the transgene construct can be further optimized for intended purposes. For example, the construct can be altered for codon optimization (e.g., to increase expression of the encoded regions).

The transgene construct can further comprise sequences that allow for targeted insertion of the transgene into a specific locus, e.g., an endogenous mouse heavy chain locus. Knock-in technology for replacing an endogenous locus with a targeted transgene is well established in the art and described further in Section III below. In a preferred embodiment, the transgene construct comprises recombination sequences (Guide Recombination Sequences, or GRS) allowing for the transgene to be knocked-in to the endogenous mouse heavy chain locus.

To prepare the transgene construct for transfection, microinjection or other technique for transgenesis, the transgene construct can be isolated from the vector in which it is carried by cleavage with appropriate restriction enzymes to release the transgene construct fragment. The fragment can be isolated using standard techniques, such as by pulse field gel electrophoresis on an agarose gel, followed by isolation of the fragment from the agarose gel, such as by [beta]-agarase digestion or by electroelution. For example, the agarose gel slice containing the transgene construct fragment can be excised from the gel and the agarose can be digested with [beta]-agarase (e.g., from Takara), using standard methodology. Alternatively, preparation of the transgene for knock-in purposes may be carried out by standard BAC or plasmid purification techniques, isolating the closed circular form for direct transfection or introduction into recipient mouse cells or embryos.

### III. Preparation of Transgenic Mice

Another aspect of the disclosure pertains to a transgenic mouse that comprises a chimeric endogenous immunoglobulin heavy chain locus prepared according to a method of disclosure such that the animal expresses an immune repertoire that comprises antibodies that utilize human VH segments (e.g., hVH-hDH-hJH-mC antibodies) as well as antibodies that use mouse VH segments (e.g., mVH-hDH-hJH-mC antibodies). The transgenic mice of the disclosure are prepared using standard methods known in the art for deleting exogenous genomic sequences and introducing exogenous nucleic acid into the genome of mouse cell, followed by preparation of a transgenic mouse from the transgenic mouse cell.

Accordingly, in one aspect, the disclosure provides a transgenic mouse cell comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHS), a plurality of human D segments (DH) and a plurality of human J segments (JH),
   wherein the transgenic mouse cell comprising the chimeric Ig heavy chain locus expresses an antibody repertoire comprising mouse VH segments and human VH segments, each linked to human DH and JH segments.

In another aspect, the disclosure provides a transgenic mouse comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHs), a plurality of human D segments (DH) and a plurality of human J segments (JH),
   wherein the transgenic mouse comprising the chimeric Ig heavy chain locus expresses an antibody repertoire comprising mouse VH segments and human VH segments, each linked to human DH and JH segments.

In a preferred approach, steps (a) and (b) of the method of the disclosure are conducted simultaneously to remove the endogenous mouse DH and DJ segments and swap in the human Ig heavy chain transgene, preferably using CRISPR/Cas9-mediated recombination for a combined knock-out/knock-in approach. For example, the transgene construct can include flanking Guide Recombination Sequences (GRS) to facilitate CRISPR/Cas9-mediated recombination. These are 500-1500 bp sequences which flank the transgene insert, and have specific homology to endogenous mouse sequences that adjoin specific CRISPR/Cas9 cleavage sites in the mouse genome. Appending the same CRISPR/CAS cleavage sites to the ends of the GRS flanking sequences allows for CRISPR/CAS mediated digestion to simultaneously cleave the endogenous mouse genome as well as the transgene donor (e.g., circular BAC vector). In this manner, the cleaved ends of the mouse CRISPR/CAS sites are available for homologous recombination mediated repair via the similarly cleaved and linearized transgene donor insert, resulting in a site-specific knock-in.

Alternatively, the two modifications can be performed as two separate steps, i.e., a knock-out step and a knock-in step. For example, the endogenous mouse heavy chain D and J segments can be removed from the endogenous mouse Ig locus by standard knock-out technology, preferably using CRISPR-Cas9-mediated recombination. The human Ig heavy chain variable region transgene can then be inserted into the endogenous mouse Ig heavy chain locus by standard knock-in technology. For example, in one knock-in approach, typically loxP flanking sites are included in the construct such that these sites facilitate recombination between host loxP flanking sites and loxP flanking sites in the transgene donor upon expression of Cre recombinase. Recombination is performed in mouse embryonic stem cells and then the embryonic stem cells with the modification of interest are implanted into a viable blastocyst, which then grows into a mature chimeric mouse with some cells having the original blastocyst cell genetic information and other cells having the modifications introduced to the embryonic stem cells. Subsequent offspring of the chimeric mouse will then have the gene knock-in. Knock-in technology is summarized in, for example. Manis (2007) New Engl. J. Med. 357:2426-2429.

Southern blot analysis. PCR or other such technique for analyzing genomic DNA is used to detect the presence of a unique nucleic acid fragment that would not be present in the non-transgenic animal but would be present in the transgenic animal. Selective breeding of transgenic offspring allows for homozygosity of the transgene to be achieved.

A transgenic mouse of the disclosure carrying a chimeric Ig heavy chain locus can be cross-bred with a mouse that carries an immunoglobulin light chain transgene (e.g., a human Ig light chain transgene) to thereby produce a mouse that expresses antibodies comprising the transgene-derived light chain (e.g., human light chain) paired with a heavy chain derived from the chimeric heavy chain locus. Immunoglobulin light chain transgenic mice are well-established in the art.

### IV. Use of Transgenic Mice

The transgenic mice of the disclosure are useful for generating antibodies to a wide variety of antigens of interest. For mice carrying only the chimeric Ig heavy chain locus and an endogenous mouse light chain locus, the mouse will produce chimeric heavy chain/mouse light chain antibodies that, if desired, can be reverse engineered to pair the chimeric heavy chain with a light chain of another species and/or to replace the mouse constant region(s) with a human constant region(s). Alternatively, for mice carrying both a chimeric Ig heavy chain locus and a human light chain Ig transgene, chimeric heavy chain/human light chain antibodies can be prepared in the host mouse. Chimeric antibodies that are not further humanized or fully human may still be useful as in vitro reagents and for use in diagnostic assays. Antibodies intended for therapeutic use in humans typically are reverse engineered to be fully human.

Additional or alternative recombinant engineering can be conducted on an antibody of interest isolated from a transgenic mouse of the disclosure having a chimeric Ig heavy chain locus. For example. for antibodies that utilize mVH-hDH-hJH variable regions or mVH-mDH-hJH variable regions, the unique CDR3 generated by the recombination events can be isolated and grafted into another antibody backbone according to methods well-established in the art.

Accordingly, in another aspect, the disclosure pertains to a method of generating antibodies to an antigen of interest, the method comprising administering the antigen of interest to a transgenic mouse of the disclosure. In an embodiment, the antigen is administered to the mouse such that antibodies that bind to the antigen of interest are generated in the mouse. In an embodiment, the transgenic mouse comprises both a chimeric Ig heavy chain locus as described herein and a human Ig light chain transgene and the antigen is administered to the mouse such that hVH-containing and mVH-containing chimeric antibodies are generated in the mouse. In an embodiment, the method can further comprise isolating an antibody of interest from the host mouse. In an embodiment, the method can further comprise isolating nucleic acid encoding an antibody of interest from the mouse and replacing mouse constant region sequences within the nucleic acid with human constant region sequences. Additional or alternative recombinant engineering of the resultant antibody (e.g., to reverse engineer mouse sequences to human sequences) is contemplated.

Transgenic animals can be immunized with an antigen(s) of interest by standard methodologies known in the art and antibodies generated in the animals can be isolated and characterized also by standard established methods. Polyclonal antibodies can be directly isolated form the host animal and monoclonal antibodies can be prepared by standard methods, such as hybridoma technology. Procedures for making monoclonal antibodies using hybridomas are well established in the art (see, e.g., U.S. Pat. No. 4,977,081, PCT Publication WO 97/16537, and European Patent No. 491057B1, the disclosures of which are incorporated herein by reference). Alternatively, in vitro production of monoclonal antibodies from cloned cDNA molecules is also established in the art (see e.g., Andris-Widhopf et al. (2000) J. Immunol. Methods 242:159; and Burton (1995) Immunotechnology 1:87, the disclosures of which are incorporated herein by reference). B cell clones from the immunized transgenic mice can be isolated and cDNAs encoding the antibodies can be isolated and cloned by standard molecular biology techniques into expression vectors. Further recombinant engineering of the cloned Ig cDNAs is also possible and well established in the art.

### V. Definitions

As used herein, the term “chimeric”, as in “chimeric Ig locus”, is intended to refer to nucleic acid sequences that are derived from two different species, such as a human and a mouse. Thus, an endogenous mouse Ig locus is chimeric when it includes nucleic acid sequences from another species, such as a human.

As used herein, the term “operatively linked” is intended to describe the configuration of a nucleic acid sequence that is placed into a functional relationship with another nucleic acid sequence. For example, a promoter or enhancer is operatively linked to a coding sequence if it affects the transcription of the sequence. With respect to the joining of two protein coding regions, operatively linked means that the nucleic acid sequences being linked are contiguous and in reading frame. For splice donor/acceptor and RSS sequences, operatively linked means that the sequences are capable of effecting their functional purposes.

As used herein, the term “Supra-diversity mouse” is intended to refer to a chimeric mouse of the disclosure that has its endogenous heavy chain locus altered by insertion of a human Ig heavy chain transgene such that the mouse expresses an antibody repertoire with greater V region diversity (e.g., diversity of VH usage) than mice with an unaltered endogenous mouse HC locus and mice with a humanized VDJ locus in which the mouse V segments have been inactivated. An altered Ig heavy chain allele prepared according to the methods of the disclosure is also referred to herein as a “Supra allele”, leading to the generation of a Supra-diversity mouse.

As used herein, the term “transgene” refers to a gene that is introduced as an exogenous source to a site within a host genome (e.g., mouse heavy chain Ig locus).

As used herein, the term “transgene construct” refers to a nucleic acid preparation suitable for introduction into the genome of a host animal.

As used herein, the term “transgenic mouse” refers to a mouse comprising cells having a transgene, as defined herein. The transgene may be present in all or some cells of the mouse.

As used herein, the term “unrearranged” with respect to an immunoglobulin V segment refers to an immunoglobulin V segment that is in its germline configuration wherein the V segment is not recombined so as to be immediately adjacent to a D or J segment.

The present invention is further illustrated by the following examples, which should not be construed as further limiting. The contents of figures and all references, patents and published patent applications cited throughout this application are expressly incorporated herein by reference.

### EXAMPLES

### Example 1: Preparation of Supra-Diversity Transgenic Mouse

In this example, the preparation of a Supra-diversity transgenic mouse having a chimeric endogenous heavy chain Ig locus is described, wherein mouse D and J segments are deleted from the endogenous locus and human V. D and J segments are introduced at the site while maintaining the presence and function of the mouse V segments such that both hVH-D-J and mVH-D-J recombination can occur. Given the use of both mVH and hVH in forming the antibody repertoire of the Supra-diversity mouse, it represents a tool for generation of a greater diversity of VH sequences than either a mouse with an intact endogenous mouse heavy chain locus alone or a mouse only with a heavy chain locus that has been fully humanized (e.g., human sequences inserted and mouse sequences, including mVH, inactivated).

### Deletion of Endogenous Mouse D and J Segments

As illustrated schematically in FIG. 1, the Supra-diversity mouse was prepared using a process that involves deletion of the endogenous mouse D and J segments from the endogenous mouse Ig heavy chain locus on mouse chromosome 12. Mouse D and J segments were deleted downstream of the Adam6b gene and upstream of the mouse IgM region, which preserved the ADAM6 gene region (including Adam6a and Adam6b) necessary for fertility in the mice. This strategy maintained the mouse 1-3 and 3-1 D segments that are embedded within the ADAM6 gene region, while deleting all mouse D segments downstream of the ADAM6 gene region. The deletion interval of the mouse IGH locus on chromosome 12 created by this process corresponds to base pairs 113,391,744-113,446,387 (per the NCBI Reference Sequence: NC_000078.7; GRCm39; sec https://www.ncbi.nlm.nih.gov/nucleotide/NC_000078.7).

Deletion of a portion of the endogenous mouse heavy chain locus at the region indicated in FIG. 1 was accomplished in mouse embryonic stem (ES) cells using standard CRISPR-Cas9-mediated recombination including guide RNAs (gRNAs), which is a well-established method in the art for deleting genomic sequences. ES cells having the proper recombination at the heavy chain locus were identified by standard methods. Recombinant cells comprised the puromycin resistance gene, allowing for puromycin selection. Genomic DNA analysis also can be used to confirm proper recombination.

### Introduction of Human Ig Heavy Chain Variable Region Transgene

Also as illustrated schematically in FIG. 1, the process for preparing a Supra-diversity mouse involves the introduction of a human Ig heavy chain variable region transgene into the endogenous mouse Ig heavy chain locus at the site from which the mouse D and J segments were deleted. The human Ig heavy chain transgene was designed using a bacterial artificial chromosome (BAC) as the vector and including a plurality of human heavy chain V, D and J segments. The following forty-one human VH segments were included in the transgene in their unrearranged germline configuration: 3-74, 3-73, 3-72, 2-70, 1-69, 3-66, 3-64, 4-61, 4-59, 1-58, 3-53, 5-51, 3-49, 3-48, 1-46, 1-45, 3-43, 4-39, 4-34, 3-33, 4-31, 3-30, 4-28, 2-26, 1-24, 3-23, 3-21, 3-20, 1-18, 3-15, 3-13, 3-11, 3-9, 1-8, 3-7, 2-5, 7-4-1, 4-4, 1-3, 1-2 and 6-1. The transgene also included 27 human DH segments in their germline configuration and all six human JH segments in their germline configuration. The 27 DH segments used represent the entire human DH repertoire that exists in the heavy chain locus from human chromosome 14, including the distal-most DH segments.

The BAC vector transgene construct is illustrated schematically in FIG. 5. The human Ig Heavy Chain BAC was created by a combination of gene synthesis and recombineering. The human VH array of 41 segments was a fully synthetic construct inserted into a BAC vector backbone. This VH donor was then used in a recombineering scheme, recombining the VH array with human germline sequence for D and J elements, using the human BAC clone CTD-257202 as a substrate. The mouse-specific GRS homology domains were appended to both ends of the recombineered BAC, along with mouse-specific CRISPR-Cas9 guide recognition sites to facilitate the GRS recombination method. The specific guides used and incorporated into the BAC construct for the GRS recombination method are as follows (protospacer adjacent motif (PAM) sequence in parentheses):

moIgH JEmu-1:
(SEQ ID NO: 2)
TTATACAGTATCCGATGCAT(AGG)
*moIgH D1-1:
(SEQ ID NO: 3)
ATCATGATATCCCACAAGTA(TGG)

The human Ig heavy chain variable region transgene construct was introduced into the endogenous mouse Ig heavy chain locus of ES cells that had undergone deletion of the endogenous D and J segments, as illustrated schematically in FIG. 1. Standard knock-in technology using the GRS method of CRISPR-Cas9 mediated recombination, was used to introduce the transgene into the appropriate site within the endogenous mouse heavy chain locus. ES cells having the proper recombination at the heavy chain locus were identified by standard methods. Introduction of the human Ig transgene allowed for puromycin selection. Following selection, the puro cassette can be removed, for example by Flp recombination or other means established in the art, so as not to interfere with donor DNA gene expression. Genomic DNA analysis also can be used to confirm proper knock-in of the transgene.

Following creation of the Supra allele at the endogenous mouse Ig heavy chain locus within the mouse ES cell as described above, the transgenic Supra-diversity mouse was generated from the ES cells by standard methods well established in the art.

### Example 2: Characterization of Supra-Diversity Transgenic Mouse

In this example, the Supra-Diversity mouse prepared as described in Example 1 was characterized as to its VH segment usage, both in naïve and immunized animals. The schematic diagram of FIG. 2A illustrates how the Supra allele can generate recombinant V-D-J units that are derived from different parts of the knock-in structure. This is based on the inherent property of the Ig loci to undergo stochastic recombination between the varied VH, DH and JH coding sequences during B cell development, forming functional V-D-J products that serve to encode the variable portion of the Ig heavy chain.

In a first set of experiments, sequence analysis following RT-PCR of RNA from the Supra mice was used to assign the proportion of fully human VDJ recombination products vs. mVH/hDH/hJH products using the Enpicom IGX platform for Ig sequence analysis. Naïve, unimmunized mice were examined, as well as mice immunized with different immunogens. The results showed that in naïve, unimmunized mice, human VH usage was 45% versus mouse VH usage of 55% (FIG. 2B). For mice immunized with COVID-19 spike protein, human VH usage was 44% versus mouse VH usage of 56% (FIG. 2C). For mice immunized with an undisclosed immunogen (“Target X”), human VH usage was 63% versus mouse VH usage of 37% (FIG. 2D). The data demonstrate that both types of recombination products were readily recovered, although the proportion varied with the source of RNA used for the RT-PCR+sequencing. Single B cells isolated from immunized lymph nodes (“Target X” experiment) appeared to have a larger proportion of fully human recombination products than was seen in whole spleen processed in bulk.

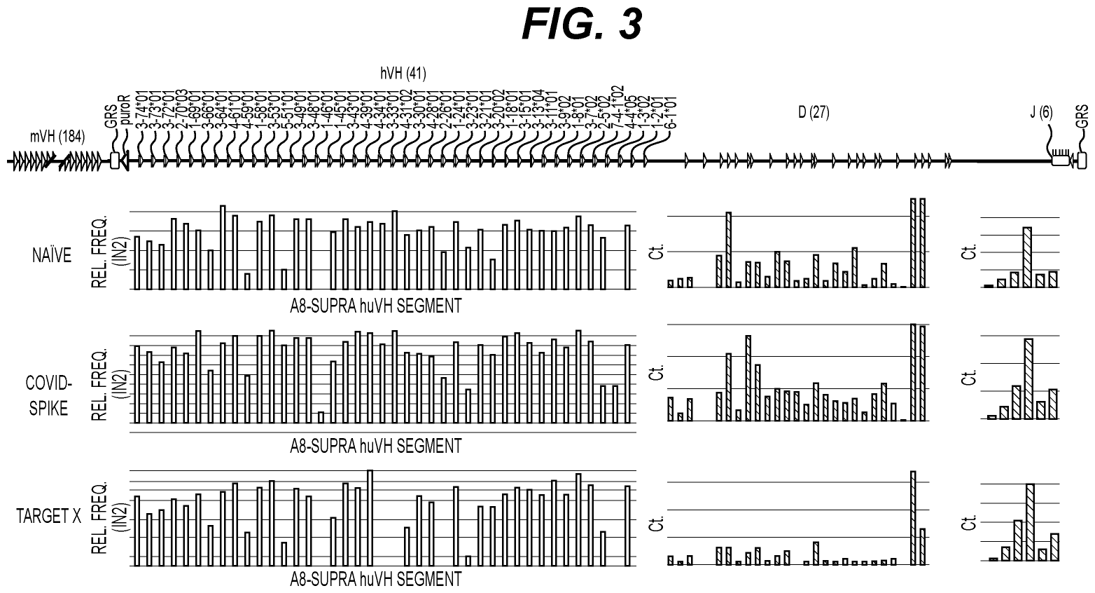

IGX bioinformatics analysis was used to further characterize the specific V regions used in the Supra-diversity mouse. The relative frequency of different human V, D and J segment usage is illustrated in FIG. 3, showing results for naïve, unimmunized mice and for mice immunized with either COVID-19 spike protein or Target X immunogen. The results demonstrate that between the collective data of all three sets of mice, all of the input human VH segments are utilized at some level, as were the most all of the human D and all J segments, confirming the functionality of the human donor transgene.

A more quantitative summary of the human VH, D, and J usage as determined by the IGX platform is shown below in Tables 1-3, respectively. Counts correspond to the number of individually identified VH, DH, or JH segments within the NGS sequence runs. The Naïve and the Spike-immunized samples were bulk NGS runs from whole spleen RNA. The Target X-immunized sample was run in single-cell mode using 10× methodology (https://www.10xgenomics.com/).

TABLE 1
Human VH usage in Supra-diversity mice as determined by IGX platform

| Supra A8 Naïve | | Supra A8 Spike Immnzd | | Supra A8 Target X Immnzd | |
|---|---|---|---|---|---|
| Count | hIGHV Call | Count | hIGHV Call | Count | hIGHV Call |
| 41374 | 4-61_hs | 14464 | 4-34_hs | 1055 | 3-33_hs |
| 20113 | 3-30_hs*1 | 11779 | 3-30_hs | 739 | 7-4-1_hs |
| 10307 | 5-51_hs*1 | 11092 | 5-51_hs | 490 | 5-51_hs |
| 9561 | 4-59_hs*1 | 10649 | 7-4-1_hs | 488 | 3-7_hs |
| 8274 | 7-4-1_hs*2 | 10030 | 3-66_hs | 404 | 4-39_hs |
| 5604 | 1-46_hs*1 | 8042 | 3-33_hs | 391 | 4-59_hs |
| 5584 | 2-70D_hs*3 | 7991 | 3-11_hs | 356 | 4-4_hs |
| 5337 | 4-39_hs*1 | 5502 | 3-13_hs | 323 | 6-1_hs |
| 5291 | 3-48_hs*1 | 5457 | 4-59_hs | 309 | 3-21_hs |
| 4430 | 3-11_hs*1 | 5142 | 3-53_hs | 290 | 4-34_hs |
| 3877 | 3-53_hs*1 | 5049 | 3-48_hs | 289 | 3-11_hs |
| 3521 | 3-21_hs*1 | 4348 | 1-46_hs | 288 | 3-53_hs |
| 3335 | 3-33_hs*1 | 3784 | 3-7_hs | 269 | 3-48_hs |
| 2565 | 1-69_hs*1 | 3003 | 3-21_hs | 248 | 3-9_hs |
| 2537 | 4-31_hs*2 | 2954 | 4-4_hs | 207 | 4-61_hs |
| 2382 | 3-13_hs*4 | 2844 | 4-39_hs | 180 | 2-5_hs |
| 2340 | 4-4_hs*5 | 2708 | _3-9_hs | 178 | 3-66_hs |
| 2136 | 6-1_hs*1 | 2541 | 4-61_hs | 173 | 3-13_hs |
| 1656 | 1-24_hs*1 | 2376 | 4-31_hs | 172 | 1-8_hs |
| 1591 | 4-34_hs*1 | 2121 | 1-18_hs | 160 | 2-26_hs |
| 1430 | 2-5_hs*2 | 2015 | 6-1_hs | 153 | 1-46_hs |
| 1037 | _3-9_hs*2 | 1915 | 3-49_hs | 150 | 3-74_hs |
| 1030 | 3-66_hs*1 | 1673 | 2-70D_hs | 144 | 4-30-4_hs |
| 1024 | 1-18_hs*1 | 1643 | 3-74_hs | 121 | 2-70_hs |
| 1010 | 2-26_hs*1 | 1631 | 2-5_hs | 103 | 1-24_hs |
| 831 | 1-8_hs*1 | 916 | 3-73_hs | 77 | 1-69_hs |
| 746 | 3-7_hs*2 | 904 | 4-28_hs | 74 | 1-18_hs |
| 624 | 3-43_hs*1 | 867 | 1-8_hs | 69 | 3-15_hs |
| 467 | 4-28_hs*1 | 787 | 1-69_hs | 59 | 3-72_hs |
| 309 | 3-74_hs*1 | 783 | 2-26_hs | 44 | 3-73_hs |
| 284 | 1-3_hs*2 | 659 | 3-15_hs | 32 | 3-43_hs |
| 172 | 3-73_hs*1 | 542 | 1-24_hs | 18 | 3-64_hs |
| 104 | 3-72_hs*1 | 310 | 3-43_hs | 16 | 4-28_hs |
| 66 | 3-20_hs*2 | 275 | 3-72_hs | 12 | 1-3_hs |
| 40 | 3-64_hs*1 | 119 | 3-64_hs | 11 | 1-58_hs |
| 32 | 3-23_hs*1 | 61 | 1-58_hs | 5 | 3-49_hs |
| 10 | 3-15_hs*1 | 47 | 3-23_hs | 2 | 3-20_hs |
| 2 | 3-49_hs*1 | 20 | 1-3_hs | | |
| 1 | 1-58_hs*1 | 13 | 3-20_hs | | |
| | | 3 | 1-2_hs | | |
| | | 1 | 1-45_hs | | |

TABLE 2
DH usage in Supra-diversity mice as determined by IGX platform

| Supra A8 Naïve | | Supra A8 Spike Immnzd | | Supra A8 Targ. X Immnzd | |
|---|---|---|---|---|---|
| Freq | D Seg | Freq | D Seg | Freq | D Seg |
| 55413 | IGHD7-27_hs*1 | 43764 | 1-26_*1 | 64 | IGHD1-26*01 |
| 26317 | IGHD1-7_hs*1 | 26378 | 3-9_*1 | 55 | IGHD7-27*01 |
| 11456 | IGHD1-20_hs*1 | 29165 | 7-27_*1 | 15 | IGHD3-10*01 |
| 10086 | IGHD6-6_hs*1 | 11482 | 4-23_*1 | 15 | IGHD3-9*01 |
| 9789 | IGHD4-23_hs*1 | 10035 | 5-12_*1 | 14 | IGHD3-16*01 |
| 9302 | IGHD6-13_hs*1 | 11560 | 3-16_*1 | 11 | IGHD1-7*01 |
| 8812 | IGHD3-9_hs*1 | 8994 | 1-14_*1 | 11 | IGHD1-1*01 |
| 8055 | IGHD5-18_hs*1 | 20912 | 1-7_*1 | 10 | IGHD6-6*01 |
| 5873 | IGHD6-19_hs*1 | 17351 | 3-10_*1 | 9 | IGHD5-12*01 |
| 5842 | IGHD1-1_hs*1 | 7071 | 1-1_*1 | 6 | IGHD6-13*01 |
| 3762 | IGHD2-2_hs*1 | 8337 | 3-22_*1 | 5 | IGHD3-3*01 |
| 3652 | IGHD4-11_hs*1 | 5644 | 6-19_*1 | 5 | IGHD4-23*01 |
| 2943 | IGHD4-17_hs*1 | 7892 | 4-17_*1 | 4 | IGHD6-19*01 |
| 2836 | IGHD3-3_hs*1 | 5422 | 5-24_*1 | 3 | IGHD2-21*01 |
| 2696 | IGHD1-14_hs*1 | 6667 | 3-3_*1 | 3 | IGHD5-24*01 |
| 2687 | IGHD2-15_hs*1 | 8620 | 6-6_*1 | 3 | IGHD2-15*01 |
| 2662 | IGHD3-22_hs*1 | 9107 | 6-13_*1 | 3 | IGHD4-11*01 |
| 1922 | IGHD2-8_hs*1 | 7394 | 4-11_*1 | 2 | IGHD1-20*01 |
| 1236 | IGHD5-24_hs*1 | 6189 | 5-18_*1 | 2 | IGHD5-18*01 |
| | | 5074 | 2-15_*1 | 2 | IGHD2-21*02 |
| | | 3033 | 2-8_*1 | 2 | IGHD3-16*02 |
| | | 2584 | 2-21_*2 | 1 | IGHD2-8*02 |
| | | 6840 | 1-20_*1 | 1 | IGHD3-22*01 |
| | | 2164 | 2-2_*1 | 2 | IGHD2-2 |
| | | 143 | 6-25_*1 | 1 | IGHD4-17*01 |
| | | 7 | 5-5_*1 | | |

TABLE 3
JH usage in Supra-diversity mice as determined by IGX platform

| Supra A8 Naive | | Supra A8 Spike Immnzd | | Supra A8 Targ. X Immnzd | |
|---|---|---|---|---|---|
| Count | hJH Call | Count | hJH Call | Count | hJH Call |
| 173518 | IGHJ4_hs*2 | 143274 | IGHJ4_hs*2 | 1033 | IGHJ4_hs*2 |
| 46891 | IGHJ6_hs*2 | 59336 | IGHJ3_hs*2 | 525 | IGHJ3_hs*2 |
| 44347 | IGHJ3_hs*2 | 53522 | IGHJ6_hs*2 | 358 | IGHJ6_hs*2 |
| 38525 | IGHJ5_hs*2 | 31326 | IGHJ5_hs*2 | 178 | IGHJ2_hs*1 |
| 24166 | IGHJ2_hs*1 | 21634 | IGHJ2_hs*1 | 155 | IGHJ5_hs*2 |
| 5447 | IGHJ1_hs*1 | 5135 | IGHJ1_hs*1 | 30 | IGHJ1_hs*1 |

The amino acid and nucleotide sequences of the top 20 identified clonotypes in the naïve, unimmunized spleen sample were further analyzed via the NCBI “Ig BLAST” program. Human transcripts made up 59.7% of the total, whereas mouse transcripts made up 40.3%. This confirmed that the IGX program can identify both fully human (hVH/hDH/hJH) transcripts and mouse-human hybrid (mVH/hDH/hJH) transcripts.

To assess whether the usage of mouse VH segments in the Supra-diversity mouse reflects that of a “normal” mouse, the most frequently seen VH segments were compared between: (i) the Supra-diversity mouse; (ii)wild-type C57/B16 mouse spleen RNA sequenced in-house; and (iii) a published reference for the normal mouse Ig repertoire from C57B1/6 wild-type spleens (Rettig et al. (2018) PLOS One 13(1):e0190982). The rank order of the 14 most-to-least frequently used VH segments in the three groups is shown below in Table 4:

TABLE 4
Rank order of VH segment usage

| | | WT Zai naïve |
|---|---|---|
| PLoS moVH | SupraA8 Naïve moVH | moVH |
| IGHV1-80¹ | IGHV3-8 | IGHV1-15 |
| IGHV1-26² | IGHV1-80¹ | IGHV1-64⁴ |
| IGHV6-3 | IGHV1-18¹ | IGHV8-8 |
| IGHV1-53¹ | IGHV1-53¹ | IGHV1-26² |
| IGHV1-55¹ | IGHV1-61³ | IGHV14-2 |
| IGHV3-6 | IGHV1-55¹ | IGHV1-50² |
| IGHV1-92 | IGHV1-67 | IGHV1-52 |
| IGHV1-18¹ | IGHV2-6 | IGHV1-78² |
| IGHV9-3¹ | IGHV1-54 | IGHV1-22 |
| IGHV1-50² | IGHV1-59 | IGHV1-61³ |
| IGHV1-64⁴ | IGHV1-64⁴ | IGHV1-9² |
| IGHV6-6 | IGHV1-74³ | IGHV1-74³ |
| IGHV1-8² | IGHV9-3¹ | IGHV12-3 |
| IGHV1-78² | IGHV1-12 | IGHV1-76 |

Coding
PLoS + Supra A8¹
PLoS + WT²
Supra A8 + WT³
PLoS + Supra A8 + WT⁴

There were a number of top ‘hits’ (6/14) from the published naïve mouse repertoire (“PLOS”) that were also seen in the Supra-diversity mouse (VH segments 1-80, 1-53, 1-55, 1-18, 9-3 and 1-64). Additionally, there were VH's common to Supra-diversity mouse and the in-house sequenced C57 WT sample, “WT Zai naïve moVH” (VH segments 1-61, 1-74 and 1-64). There also were common hits between the published repertoire (“PLOS”) and the in-house sequenced sample (“WT”) (VH segments 1-26, 1-9, 1-50, 1-78 and 1-64). Overall, the results present a usage profile that is generally concordant, but with normal variation, which is typical in analyzing naïve repertoires.

The extent of mouse VH usage was also examined in the Supra-diversity mouse through NGS and bioinformatics analysis. A summary of the mouse VH usage from NGS sequencing on the MiSeq platform, using the IGX MiXCR application, is shown below in Table 5:

TABLE 5
Mouse VH segment usage in Supra-diversity mouse

| moVH Segment | Count |
|---|---|
| IGHV3-8 | 28255 |
| IGHV1-80 | 18870 |
| IGHV1-18 | 17107 |
| IGHV1-53 | 13534 |
| IGHV1-61 | 10792 |
| IGHV1-55 | 10698 |
| IGHV1-67 | 5727 |
| IGHV2-6 | 5651 |
| IGHV1-54 | 5431 |
| IGHV1-59 | 4570 |
| IGHV1-64 | 4405 |
| IGHV1-74 | 4363 |
| IGHV9-3 | 4196 |
| IGHV1-12 | 3193 |
| IGHV1-19 | 3176 |
| IGHV1-23 | 3142 |
| IGHV1-34 | 2610 |
| IGHV1-14 | 2569 |
| IGHV10-3 | 2562 |
| IGHV8-5 | 2103 |
| IGHV1-69 | 1919 |
| IGHV1-82 | 1908 |
| IGHV5-9 | 1671 |
| IGHV1-73 | 1540 |
| IGHV1-77 | 1339 |
| IGHV1-7 | 1288 |
| IGHV1-9 | 1185 |
| IGHV1-5 | 1099 |
| IGHV1-62 | 1048 |
| IGHV7-2 | 1023 |
| IGHV2-9 | 1006 |
| IGHV3-1 | 975 |
| IGHV9-2 | 945 |
| IGHV6-6 | 919 |
| IGHV8-8 | 867 |
| IGHV1-39 | 790 |
| IGHV14-4 | 749 |
| IGHV5-12 | 735 |
| IGHV3-6 | 731 |
| IGHV1-84 | 628 |
| IGHV15-2 | 517 |
| IGHV3-2 | 504 |
| IGHV1-26 | 500 |
| IGHV2-2 | 470 |
| IGHV2-6-8 | 444 |
| IGHV1-56 | 441 |
| IGHV1-22 | 406 |
| IGHV1-36 | 402 |
| IGHV8-12 | 393 |
| IGHV5-17 | 383 |
| IGHV14-3 | 376 |
| IGHV2-3 | 373 |
| IGHV6-7 | 302 |
| IGHV1-76 | 294 |
| IGHV2-4 | 262 |
| IGHV1-2 | 244 |
| IGHV14-2 | 240 |
| IGHV5-6 | 233 |
| IGHV1-86 | 229 |
| IGHV5-2 | 219 |
| IGHV1-4 | 209 |
| IGHV1-78 | 198 |
| IGHV1-66 | 175 |
| IGHV1-50 | 156 |
| IGHV5-9-1 | 151 |
| IGHV1-83 | 148 |
| IGHV1-81 | 137 |
| IGHV7-3 | 116 |
| IGHV9-1 | 115 |
| IGHV6-3 | 113 |
| IGHV1-85 | 107 |
| IGHV2-9-1 | 103 |
| IGHV8-11 | 96 |
| IGHV1-71 | 87 |
| IGHV1-42 | 85 |
| IGHV1-52 | 85 |
| IGHV1-72 | 72 |
| IGHV12-3 | 65 |
| IGHV2-5 | 62 |
| IGHV7-1 | 60 |
| IGHV8-7 | 56 |
| IGHV6-5 | 50 |
| IGHV5-4 | 49 |
| IGHV1-75 | 48 |
| IGHV13-2 | 41 |
| IGHV1-47 | 39 |
| IGHV12-1 | 38 |
| IGHV4-2 | 37 |
| IGHV1-25 | 34 |
| IGHV1-49 | 32 |
| IGHV9-4 | 31 |
| IGHV1-58 | 20 |
| IGHV1-28 | 18 |
| IGHV1-62-3 | 15 |
| IGHV1-62-2 | 13 |
| IGHV3-5 | 11 |
| IGHV8-13 | 11 |
| IGHV14-1 | 10 |
| IGHV1-63 | 10 |
| IGHV1-17 | 6 |
| IGHV1-15 | 5 |
| IGHV1-62-1 | 5 |
| IGHV4-1 | 5 |
| IGHV12-2 | 4 |
| IGHV3-7 | 4 |
| IGHV1-21 | 2 |
| IGHV1-37 | 2 |
| IGHV1-70 | 2 |
| IGHV1-79 | 2 |
| IGHV5-15 | 2 |
| IGHV7-4 | 2 |
| IGHV8-4 | 2 |
| IGHV10-1 | 1 |
| IGHV5-12-4 | 1 |
| IGHV8-2 | 1 |

The results showed that the number of expressed mouse VH segments in the Supra-diversity mouse is approximately 115. The number of expressed mouse VH segments identified from the in-house C57B1/6 wild-type naïve spleen sequencing was 114. Moreover, the published repertoire reports approximately 132 mouse VH segments (Rettig et al. (2018) PLOS One 13(1):e0190982). Based on this, the VH usage frequency in the Supra-diversity mice appears to be normal.

The VH usage in the Supra-diversity mouse is summarized schematically in FIG. 4, which illustrates the usage of 115 mouse VH segments that combine with 26 human VD segments and 6 human VJ segments, as well as 41 human VH segments that combine with 26 human VD segments and 6 human VJ segments. These results demonstrate that whether the initial VH domain derives from human or mouse, there is widespread use of all available VH, DH and JH segments in the Supra-diversity mouse.

To examine CDR3 diversity in the Supra-diversity mouse, the top 48 clonotypes using mouse VH 1-64 were compared between the Supra-diversity mouse and a WT-C57B1/6 mouse. When the CDR3 domains were compared, there clearly were some common sequence motifs among the CDR3 domains from the Supra vs. WT mouse, but it was also clear that there was at the same time a significant diversity or divergence between CDR3s in those two strains. These results provide evidence that the Supra-diversity mouse generates unique combinations of VH and CDR3 that are not typical or are not found in either human or wild-type mouse sources.

In conclusion, the variable region usage analysis of the Supra-diversity mouse demonstrated that variable region sequences from either human or mouse are efficiently used and recombined to form functional V-D-J domains, with virtually all available component VH, DH and JH segments being used in the functional rearrangements. Moreover, the knock-in of the human donor BAC transgene still allows for recovery of the same mouse VH segments that are found in wild-type mice. Sequence analysis indicated that both chimeric mVH/hDH/hJH and fully human hVH/hDH/hJH rearrangements are found, with the frequency of hVH vs. mVH usage being near equivalent in certain instances and in other instances showing a preference for fully human rearrangements. Overall, the results support the use of the Supra-diversity mouse as a novel source of antibodies with a unique repertoire that is not represented in either normal wild-type mice or normal human samples.

SUMMARY OF SEQUENCE LISTING

| # | SEQUENCE |
|---|---|
| 1 | TCTGTGCCGTGCGGGTGAGCGGCGCGTCCAGACGGGCGGGTGCACGTTTCTTTTTCTGACC |
| | ATTTCCCTGTCAGCTCTTTTAGATCGGGGCTGCCGTTTGAAAAGTTTTATCATCGCCAGAC |
| | ATGCCATAAGGAATATCTCCACCGCCTCTAACTGTCCCTAACTCGGTGTCTTTCTTATAAC |
| | TACCATTACTCAGTGACGCGTAACTTACACGCGCCTCGTATCTTTTAATGATGGAATAATT |
| | TGGGAATTTACTCTGTGTTTATTTATTTTTATGTTTTGTATTTGGATTTTAGAAAGTAAAT |
| | AAAGAAGGTAGAAGAGTTACGGAATGAAGAAAAAAAAATAAACAAAGGTTTAAAAAATTTC |
| | AACAAAAAGCGTACTTTACATATATATTTATTAGACAAGAAAAGCAGATTAAATAGATATA |
| | CATTCGATTAACGATAAGTAAAATGTAAAATCACAGGATTTTCGTGTGTGGTCTACTACAC |
| | AGACAAGATGAAACAATTCGGCATTAATACCTGAGAGCAGGATGAGCAAGATAAAAGGTAG |
| | TATTTGTTGGCGATCCCCCTAGAGTCTTTTACATCTTCGGAAAACAAAAACTATTTTTTCT |
| | TTAATTTCTTTTTTTACTTTCTATTTTTAATTTATATATTTATATTAAAAAATTTAAATTA |
| | TAATTATTTTTATAGCACGTGATGAAAAGGACCCAGGTGGCACTTGGCGTACGCTGAAGAT |
| | CCTCGGCCTAGATTAGAATTGGCGCGGTGCAGGATCACCGGAGCAACGGTAGGCTAGCGTC |
| | GACAGCGACACACTTGCATCGGATGCAGCCCGGTTAACGTGCCGGCACGGCCTGGGTAACC |
| | AGGTATTTTGTCCACATAACCGTGCGCAAAATGTTGTGGATAAGCAGGACACAGCAGCAAT |
| | CCACAGCAGGCATACAACCGCACACCGAGGTTACTCCGTTCTACAGGTTACGACGACATGT |
| | CAATACTTGCCCTTGACAGGCATTGATGGAATCGTAGTCTCACGCTGATAGTCTGATCGAC |
| | AATACAAGTGGGACCGTGGTCCCAGACCGATAATCAGACCGACAACACGAGTGGGATCGTG |
| | GTCCCAGACTAATAATCAGACCGACGATACGAGTGGGACCGTGGTCCCAGACTAATAATCA |
| | GACCGACGATACGAGTGGGACCGTGGTTCCAGACTAATAATCAGACCGACGATACGAGTGG |
| | GACCGTGGTCCCAGACTAATAATCAGACCGACGATACGAGTGGGACCATGGTCCCAGACTA |
| | ATAATCAGACCGACGATACGAGTGGGACCGTGGTCCCAGTCTGATTATCAGACCGACGATA |
| | CGAGTGGGACCGTGGTCCCAGACTAATAATCAGACCGACGATACGAGTGGGACCGTGGTCC |
| | CAGACTAATAATCAGACCGACGATACGAGTGGGACCGTGGTCCCAGTCTGATTATCAGACC |
| | GACGATACAAGTGGAACAGTGGGCCCAGAGAGAATATTCAGGCCAGTTATGCTTTCTGGCC |
| | TGTAACAAAGGACATTAAGTAAAGACAGATAAACGTAGACTAAAACGTGGTCGCATCAGGG |
| | TGCTGGCTTTTCAAGTTCCTTAAGAATGGCCTCAATTTTCTCTATACACTCAGTTGGAACA |
| | CGAGATCTGTCCAGGTTAAGCACCATTTTATCGCCCTTATACAATACTGTCGCTCCAGGAG |
| | CAAACTGATGTCGTGAGCTTAAACTAGTTCTTGATGCAGATGACGTTTTAAGCACAGAAGT |
| | TAAAAGAGTGATAACTTCTTCAGCTTCAAATATCACCCCAGCTTTTTTCTGCTCATGAAGG |
| | TTAGATGCCTGCTGCTTAAGTAATTCCTCTTTATCTGTAAAGGCTTTTTGAAGTGCATCAC |
| | CTGACCGGGCAGATAGTTCACCGGGGTGAGAAAAAAGAGCAACAACTGATTTAGGCAATTT |
| | GGCGGTGTTGATACAGCGGGTAATAATCTTACGTGAAATATTTTCCGCATCAGCCAGCGCA |
| | GAAATATTTCCAGCAAATTCATTCTGCAATCGGCTTGCATAACGCTGACCACGTTCATAAG |
| | CACTTGTTGGGCGATAATCGTTACCCAATCTGGATAATGCAGCCATCTGCTCATCATCCAG |
| | CTCGCCAACCAGAACACGATAATCACTTTCGGTAAGTGCAGCAGCTTTACGACGGCGACTC |
| | CCATCGGCAATTTCTATGACACCAGATACTCTTCGACCGAACGCCGGTGTCTGTTGACCAG |
| | TCAGTAGAAAAGAAGGGATGAGATCATCCAGTGCGTCCTCAGTAAGCAGCTCCTGGTCACG |
| | TTCATTACCTGACCATACCCGAGAGGTCTTCTCAACACTATCACCCCGGAGCACTTCAAGA |
| | GTAAACTTCACATCCCGACCACATACAGGCAAAGTAATGGCATTACCGCGAGCCATTACTC |
| | CTACGCGCGCAATTAACGAATCCACCATCGGGGCAGCTGGTGTCGATAACGAAGTATCTTC |
| | AACCGGTTGAGTATTGAGCGTATGTTTTGGAATAACAGGCGCACGCTTCATTATCTAATCT |
| | CCCAGCGTGGTTTAATCAGACGATCGAAAATTTCATTGCAGACAGGTTCCCAAATAGAAAG |
| | AGCATTTCTCCAGGCACCAGTTGAAGAGCGTTGATCAATGGCCTGTTCAAAAACAGTTCTC |
| | ATTCGGATCTGACCTTTACCAACTTCATCCGTTTCACGTACAACATTTTTTAGAACCATGC |
| | TTCCCCAGGCATCCCGAATTTGCTCCTCCATCCACGGGGACTGAGAGCCATTACTATTGCT |
| | GTATTTGGTAAGCAAAATACGTACATCAGGCTCGAACCCTTTAAGATCAACGTTCTTGAGC |
| | AGATCACGAAGCATATCGAAAAACTGCAGTGCGGAGGTGTAGTCAAACAACTCAGCAGGCG |
| | TGGGAACAATCAGCACATCAGCAGCACATACGACATTAATCGTGCCGATACCCAGGTTAGG |
| | CGCGCTGTCAATAACTATGACATCATAGTCATGAGCAACAGTTTCAATGGCCAGTCGGAGC |
| | ATCAGGTGTGGATCGGTGGGCAGTTTACCTTCATCAAATTTGCCCATTAACTCAGTTTCAA |
| | TACGGTGCAGAGCCAGACAGGAAGGAATAATGTCAAGCCCCGGCCAGCAAGTGGGCTTTAT |
| | TGCATAAGTGACATCGTCCTTTTCCCCAAGATAGAAAGGCAGGAGAGTGTCTTCTGCATGA |
| | ATATGAAGATCTGGTACCCATCCGTGATACATTGAGGCTGTTCCCTGGGGGTCGTTACCTT |
| | CCACGAGCAAAACACGTAGCCCCTTCAGAGCCAGATCCTGAGCAAGATGAACAGAAACTGA |
| | GGTTTTGTAAACGCCACCTTTATGGGCAGCAACCCCGATCACCGGTGGAAATACGTCTTCA |
| | GCACGTCGCAATCGCGTACCAAACACATCACGCATATGATTAATTTGTTCAATTGTATAAC |
| | CAACACGTTGCTCAACCCGTCCTCGAATTTCCATATCCGGGTGCGGTAGTCGCCCTGCTTT |
| | CTCGGCATCTCTGATAGCCTGAGAAGAAACCCCAACTAAATCCGCTGCTTCACCTATTCTC |
| | CAGCGCCGGGTTATTTTCCTCGCTTCCGGGCTGTCATCATTAAACTGTGCAATGGCGATAG |
| | CCTTCGTCATTTCATGACCAGCGTTTATGCACTGGTTAAGTGTTTCCATGAGTTTCATTCT |
| | GAACATCCTTTAATCATTGCTTTGCGTTTTTTTATTAAATCTTGCAATTTACTGCAAAGCA |
| | ACAACAAAATCGCAAAGTCATCAAAAAACCGCAAAGTTGTTTAAAATAAGAGCAACACTAC |
| | AAAAGGAGATAAGAAGAGCACATACCTCAGTCACTTATTATCACTAGCGCTCGCCGCAGCC |
| | GTGTAACCGAGCATAGCGAGCGAACTGGCGAGGAAGCAAAGAAGAACTGTTCTGTCAGATA |
| | GCTCTTACGCTCAGCGCAAGAAGAAATATCCACCGTGGGAAAAACTCCAGGTAGAGGTACA |
| | CACGCGGATAGCCAATTCAGAGTAATAAACTGTGATAATCAACCCTCATCAATGATGACGA |
| | ACTAACCCCCGATATCAGGTCACATGACGAAGGGAAAGAGAAGGAAATCAACTGTGACAAA |
| | CTGCCCTCAAATTTGGCTTCCTTAAAAATTACAGTTCAAAAAGTATGAGAAAATCCATGCA |
| | GGCTGAAGGAAACAGCAAAACTGTGACAAATTACCCTCAGTAGGTCAGAACAAATGTGACG |
| | AACCACCCTCAAATCTGTGACAGATAACCCTCAGACTATCCTGTCGTCATGGAAGTGATAT |
| | CGCGGAAGGAAAATACGATATGAGTCGTCTGGCGGCCTTTCTTTTTCTCAATGTATGAGAG |
| | GCGCATTGGAGTTCTGCTGTTGATCTCATTAACACAGACCTGCAGGAAGCGGCGGCGGAAG |
| | TCAGGCATACGCTGGTAACTTTGAGGCAGCTGGTAACGCTCTATGATCCAGTCGATTTTCA |
| | GAGAGACGATGCCTGAGCCATCCGGCTTACGATACTGACACAGGGATTCGTATAAACGCAT |
| | GGCATACGGATTGGTGATTTCTTTTGTTTCACTAAGCCGAAACTGCGTAAACCGGTTCTGT |
| | AACCCGATAAAGAAGGGAATGAGATATGGGTTGATATGTACACTGTAAAGCCCTCTGGATG |
| | GACTGTGCGCACGTTTGATAAACCAAGGAAAAGATTCATAGCCTTTTTCATCGCCGGCATC |
| | CTCTTCAGGGCGATAAAAAACCACTTCCTTCCCCGCGAAACTCTTCAATGCCTGCCGTATA |
| | TCCTTACTGGCTTCCGCAGAGGTCAATCCGAATATTTCAGCATATTTAGCAACATGGATCT |
| | CGCAGATACCGTCATGTTCCTGTAGGGTGCCATCAGATTTTCTGATCTGGTCAACGAACAG |
| | ATACAGCATACGTTTTTGATCCCGGGAGAGACTATATGCCGCCTCAGTGAGGTCGTTTGAC |
| | TGGACGATTCGCGGGCTATTTTTACGTTTCTTGTGATTGATAACCGCTGTTTCCGCCATGA |
| | CAGATCCATGTGAAGTGTGACAAGTTTTTAGATTGTCACACTAAATAAAAAAGAGTCAATA |
| | AGCAGGGATAACTTTGTGAAAAAACAGCTTCTTCTGAGGGCAATTTGTCACAGGGTTAAGG |
| | GCAATTTGTCACAGACAGGACTGTCATTTGAGGGTGATTTGTCACACTGAAAGGGCAATTT |
| | GTCACAACACCTTCTCTAGAACCAGCATGGATAAAGGCCTACAAGGCGCTCTAAAAAAGAA |
| | GATCTAAAAACTATAAAAAAAATAATTATAAAAATATCCCCGTGGATAAGTGGATAACCCC |
| | AAGGGAAGTTTTTTCAGGCATCGTGTGTAAGCAGAATATATAAGTGCTGTTCCCTGGTGCT |
| | TCCTCGCTCACTCGAGGGCTTCGCCCTGTCGCTCGACTGCGGCGAGCACTACTGGCTGTAA |
| | AAGGACAGACCACATCATGGTTCTGTGTTCATTAGGTTGTTCTGTCCATTGCTGACATAAT |
| | CCGCTCCACTTCAACGTAACACCGCACGAAGATTTCTATTGTTCCTGAAGGCATATTCAAA |
| | TCGTTTTCGTTACCGCTTGCAGGCATCATGACAGAACACTACTTCCTATAAACGCTACACA |
| | GGCTCCTGAGATTAATAATGCGGATCTCTACGATAATGGGAGATTTTCCCGACTGTTTCGT |
| | TCGCTTCTCAGTGGATAACAGCCAGCTTCTCTGTTTAACAGACAAAAACAGCATATCCACT |
| | CAGTTCCACATTTCCATATAAAGGCCAAGGCATTTATTCTCAGGATAATTGTTTCAGCATC |
| | GCAACCGCATCAGACTCCGGCATCGCAAACTGCACCCGGTGCCGGGCAGCCACATCCAGCG |
| | CAAAAACCTTCGTGTAGACTTCCGTTGAACTGATGGACTTATGTCCCATCAGGCTTTGCAG |
| | AACTTTCAGCGGTATACCGGCATACAGCATGTGCATCGCATAGGAATGGCGGAACGTATGT |
| | GGTGTGACCGGAACAGAGAACGTCACACCGTCAGCAGCAGCGGCGGCAACCGCCTCCCCAA |
| | TCCAGGTCCTGACCGTTCTGTCCGTCACTTCCCAGATCCGCGCTTTCTCTGTCCTTCCTGT |
| | GCGACGGTTACGCCGCTCCATGAGCTTATCGCAGAATAAATACCTGTGACGGAAGATCACT |
| | TCGCAGAATAAATAAATCCTGGTGTCCCTGTTGATACCGGGAAGCCCTGGGCCAACTTTTG |
| | GCGAAAATGAGACGTTGATCGGCACGTAAGAGGTTCCAACTTTCACCATAATGAAATAAGA |
| | TCACTACCGGGCGTATTTTTTGAGTTATCGAGATTTTCAGGAGCTAAGGAAGCTAAAATGG |
| | AGAAAAAAATCACTGGATATACCACCGTTGATATATCCCAATGGCATCGTAAAGAACATTT |
| | TGAGGCATTTCAGTCAGTTGCTCAATGTACCTATAACCAGACCGTTCAGCTGGATATTACG |
| | GCCTTTTTAAAAACCGTAAAGAAAAATAAGCACAAGTTTTATCCGGCCTTTATTCACATTC |
| | TTGCCCGCCTGATGAATGCTCATCCAGAATTCCGTATGGCAATGAAAGACGGTGAGCTGGT |
| | GATATGGGATAGTGTTCACCCTTGTTACACCGTTTTCCATGAGCAAACTGAAACGTTTTCA |
| | TCGCTCTGGAGTGAATACCACGACGATTTCCGGCAGTTTCTACACATATATTCGCAAGATG |
| | TAGCGTGTTACGGTGAAAATCTGGCCTATTTCCCTAAAGGGTTTATTGAGAATATGTTTTT |
| | CGTCTCAGCCAATCCCTGGGTGAGTTTCACCAGTTTTGATTTAAACGTGGCCAATATGGAC |
| | AACTTCTTCGCCCCCGTTTTCACCATGGGCAAATATTATACGCAAGGCGACAAGGTGCTGA |
| | TGCCGCTGGCGATTCAGGTTCATCATGCCGTTTGTGATGGCTTCCATGTCGGCAGAATGCT |
| | TAATGAATTACAACAGTACTGCGATGAGTGGCAGGGGGGGGCGTAAGGCGCGCCATAACTT |
| | CGTATAGCATACATTATACGAAGTTATACCATCATGATATCCCACAAGTATGGAAGATACA |
| | TAAGTAGATGGCCAGACTATACAGGAAAGAAGCAAAACTTGGGCTAACAGAAGAGCAAACA |
| | CAGAGCTCAGACAGAACTACCTGGCAATGCGACTGGGCACACTGAAAGCACTGGGCATCAG |
| | CACTGAGCCCCAAATATGCACTCAGGATCCTCTGCATAATAATGTGACATAACAGGAAGGT |
| | TAGAACAGGCCAAAAGAGGAAACAGAACAAATGCCCCCAACCAAAGAAGTATAAACAAATT |
| | GGGAAGAGTAAAGAAGGATTGTAAGGATTGAGTACCACACAGAACATGCTCTTAATGGCCT |
| | CAATGCTGAAGCTAGGAAGAACTAAGTTAAAAGAAACATGTTCAACGGGATTCCCTGTCAC |
| | TGGACTTCACAACAAGCAAAATTCAATCTTTCTGTTAAGGAGATGAGAAGAGAATATCTGA |
| | ACCTTGTGTTGACAGTGCCCCACCCCGACTGTCAGGCTGTGGGAAATGCCAGAGCAATCAC |
| | TAGGAACACACAAGGATGAGGGAGACGAGGGTTAGGACACAATTCTCGAGCGCGCCGCACA |
| | CAAAAACCAACACACAGATCATGAAAATAAAGCTCTTTTATTGGTACCGAATTCGCCAGGG |
| | AGCTCTCAGACGTCGCTTGGTCGGTCTTTATTCGAACCCCAGAGTCCCGCTCAGGCACCGG |
| | GCTTGCGGGTCATGCACCAGGTGCGCGGTCCTTCGGGCACCTCGACGTCGGCGGTGACGGT |
| | GAAGCCGAGCCGCTCGTAGAAGGGGAGGTTGCGGGGCGCGGAGGTCTCCAGGAAGGCGGGC |
| | ACCCCGGCGCGCTCGGCCGCCTCCACTCCGGGGAGCACGACGGCGCTGCCCAGACCCTTGC |
| | CCTGGTGGTCGGGCGAGACGCCGACGGTGGCCAGGAACCACGCGGGCTCCTTGGGCCGGTG |
| | CGGCGCCAGGAGGCCTTCCATCTGTTGCTGCGCGGCCAGCCGGGAACCGCTCAACTCGGCC |
| | ATGCGCGGGCCGATCTCGGCGAACACCGCCCCCGCTTCGACGCTCTCCGGCGTGGTCCAGA |
| | CCGCCACCGCGGCGCCGTCGTCCGCGACCCACACCTTGCCGATGTCGAGCCCGACGCGCGT |
| | GAGGAAGAGTTCTTGCAGCTCGGTGACCCGCTCGATGTGGCGGTCCGGGTCGACGGTGTGG |
| | CGCGTGGCGGGGTAGTCGGCGAACGCGGCGGCGAGGGTGCGTACGGCCCGGGGGACGTCGT |
| | CGCGGGTGGCGAGGCGCACCGTGGGCTTGTACTCGGTCATGGTTTAGTTCCTCACCTTGTC |
| | GTATTATACTATGCCGATATACTATGCCGATGATTAATTGTCAACACGTGCTGCTGCAGGT |
| | CGAAAGGCCCGGAGATGAGGAAGAGGAGAACAGCGCGGCAGACGTGCGCTTTTGAAGCGTG |
| | CAGAATGCCGGGCCTCCGGAGGACCTTCGGGCGCCCGCCCCGCCCCTGAGCCCGCCCCTGA |
| | GCCCGCCCCCGGACCCACCCCTTCCCAGCCTCTGAGCCCAGAAAGCGAAGGAGCAAAGCTG |
| | CTATTGGCCGCTGCCCCAAAGGCCTACCCGCTTCCATTGCTCAGCGGTGCTGTCCATCTGC |
| | ACGAGACTAGTGAGACGTGCTACTTCCATTTGTCACGTCCTGCACGACGCGAGCTGCGGGG |
| | CGGGGGGGAACTTCCTGACTAGGGGAGGAGTGGAAGGTGGCGCGAAGGGGCCACCAAAGAA |
| | CGGAGCCGGTTGGCGCCTACCGGTGGATGTGGAATGTGTGCGAGCCAGAGGCCACTTGTGT |
| | AGCGCCAAGTGCCCAGCGGGGCTGCTAAAGCGCATGCTCCAGACTGCCTTGGGAAAAGCGC |
| | CTCCCCTACCCGGTAGCACGACTCGTACGTACGTACGAAGTTCCTATTCTCTAGAAAGTAT |
| | AGGAACTTCTTATAAAATGTATTATGAGGAAACAAGTTGAAAATTAGACAATGTAGGAGTG |
| | ACAGAAAGATAGATATGAGTATGTTGAATGTCAGAGATACCTGAAAGTTTATCTACCTGAA |
| | CCCTAGTTCTCTCCATAGTTTAAGGTAAACAGGAGAGTGCAGGAAAATCATCCATATTCTG |
| | ATTAGGCAGTGGCTTCTGCAAACCACACTAGGCCTGGCCGGCTGTGTCCTGGAGTTGGCTA |
| | AGGGAGGAGTCAGGGCCAGTGGTGAGAAGTGCAGGCCCAGATACCAGAACTCACTCATCCC |
| | AGACATGAGCTCTTAGATACACAGAGAGCCCATCCATGTGTGGATTTATCTTACATCTGTA |
| | AGTAGAGAACATTGACTCTTACAGAACATAATTTACACACATAGGTAAATCTGAAATAAGG |
| | TGATCAGTGTGAAGATTTTATCACAGCACAGTTTCATAATAAGCACAATTTCTCAAATCCC |
| | ATTGTTGTCACCCATCTTCCTCAGGACACTTTCATCTGCCCTGGGTCCTGCTCTTTCTTCA |
| | GGTGTCTCACCCCAGAGCTTGATATATAGTAGGAGACATGCAAATAGGGCCCTCACTCTGC |
| | TGAAGAAAACCAGCCCTGCAGCTCTGGGAGAGGAGCCCCAGCCCTGGGATTCCCAGCTGTT |
| | TCTGCTTGCTGATCAGGACTGCACACAGAGAACTCACCATGGAGTTTGGGCTGAGCTGGGT |
| | TTTCCTTGTTGCTATTTTAAAAGGTGATTCATGGAGAACTGGAGATATGGAGTGTGAATGG |
| | ACATGAGTGAGATAAGCAGTGGATGTGTGTGGCAGTTTCTGACCAGGGTGTCTCTGTGTTT |
| | GCAGGTGTCCAGTGTGAGGTGCAGCTGGTGGAGTCCGGGGGAGGCTTAGTTCAGCCTGGGG |
| | GGTCCCTGAGACTCTCCTGTGCAGCCTCTGGATTCACCTTCAGTAGCTACTGGATGCACTG |
| | GGTCCGCCAAGCTCCAGGGAAGGGGCTGGTGTGGGTCTCACGTATTAATAGTGATGGGAGT |
| | AGCACAAGCTACGCGGACTCCGTGAAGGGCCGATTCACCATCTCCAGAGACAACGCCAAGA |
| | ACACGCTGTATCTGCAAATGAACAGTCTGAGAGCCGAGGACACGGCTGTGTATTACTGTGC |
| | AAGAGACACAGTGAGGGGAAGTCAATGTGAGCCCAGACACAAACCTCGCTGCAGGGGCATC |
| | TGAGACCACGAGGGGGTGTCCTGGGCCCTGTGAACTGGGCTGCTCTCCGTGGCAGCGGCTG |
| | GTGGTGCTAAAGGCTGATTTTCTCTCAGCATCTGGGGCTGATTCATCAAGTTTCCTCAGAG |
| | AACCTTTCAGATTTACAATTCTGTACTTACGTTTAATGTCTCTGAATGTGACACTTTCCTT |
| | CCCTGGTGTGTCTTTGTTTTTGTGACAAGAGGACACATTCTCACCTCCACAGAAGCCCGAG |
| | TGTCACTTTGGGGACAGAAATGACCCTGCCCTGGTCACCAGAATCAGAGTCCCGAGGAAGC |
| | CCAGGAGGACCTGGGAAGTGTTTTTCAATCAGACTCAGGGCAGGCGTCTCCGTGGGAATCT |
| | CTGATTGGAACAGGCTTTGGGATTCAGATTGGGACCAAGAGGGAGGCTCACCCAGGGCCAG |
| | GGTCCTTAGAATCCTGACAGTTTTCACAGTAACCCCATCGTCCTTTAAAACTGAACATCTA |
| | ACTCAGAACTGACCCATTTGGTCCTTTCTCTGTAATCCATTTTCCTTTTCTCTAGGCTTCA |
| | TTCTTACACTTCCCTTTTCACCTTCATTCTGAAAATGGAGGGTGTGCTTCCTGTGGTCTAA |
| | ACCACAGGCCTCAGATGCATTACCTGGAACTCAGGAATTTCTTGAAAAAAATCAGTGCTAT |
| | TTTGGGGTCAGAACAATCCTCAAAAATGGTCCCCGGAAAGAAGCCCAAATTTAATTGCACC |
| | AGATTGTTGAACGATTTATGCTACAAAACAGTGTCACATGTCAGAAATGAGCACAGTGTAA |
| | CATCTACATGGTTTGTTAAGAGACACAAATGGTCAAGTAGAACAATCAGGTAATTAGGCTG |
| | TCGAAGGCAACACTGCCAAATGACAGCATTTCCGTGGAAACTGCGTGTACATCCAGGACTG |
| | CACCTGTGAACGATGACATCATACCCTTCACAGTGTCGAGGAAAGAGACATCACTCAAACA |
| | GACAAGCCAAGGGACTTCAGAAAATATAAGGGGAAATACAGTGTGCAAATATGTAAAAAAT |
| | GCAATAAGATGATTACTCCTAAATGAATATCAAGACACAATCACATAATATGAAATTAAAT |
| | TTTCCTGAATGATAGGATTACTACCAATCACCCCCCAGGACACCCTCATCTACTCTGTGCA |
| | CAGCCTTCTCGTCAGGCGTCCCAGCCCAGACCTTGCTATGTAGCAGAAGACATGCAAATAA |
| | GACCCCCCTTTTTGCTGATGAAAAGCAGCCCAGCCCTGACCCTGCAGCTCTGGGAGAGGAG |
| | CTCCAGCCTTGGGATTCCCAGCTGTCTCCACTCGGTGATCGGCACTGAATACAGGAGACTC |
| | ACCATGGAGTTTGGGCTGAGCTGGGTTTTCCTTGTTGCTATTTTAAAAGGTGATTCATGGG |
| | GAACTAGAGATACTGAGTGTGAGTGGACATGAGTGAGAGAAACAGTGGACGTGTGTGGCAC |
| | TTTCTGACCAGGGTGTCTCTGTGTTTGCAGGTGTCCAGTGTGAGGTGCAGCTGGTGGAGTC |
| | TGGGGGAGGCTTGGTCCAGCCTGGGGGGTCCCTGAAACTCTCCTGTGCAGCCTCTGGGTTC |
| | ACCTTCAGTGGCTCTGCTATGCACTGGGTCCGCCAGGCTTCCGGGAAAGGGCTGGAGTGGG |
| | TTGGCCGTATTAGAAGCAAAGCTAACAGTTACGCGACAGCATATGCTGCGTCGGTGAAAGG |
| | CAGGTTCACCATCTCCAGAGATGATTCAAAGAACACGGCGTATCTGCAAATGAACAGCCTG |
| | AAAACCGAGGACACGGCCGTGTATTACTGTACTAGACACACAGTGAGGGGAGGTCAGTGTG |
| | AGCCCGGACACAAACCTCCCTGCAGGGGCGCGCGGGGCTACCAGGGGGCGCTCGGGACTCA |
| | CTGAGGGCGGGACAGGTCCCAGGAACAGGTGCAGCGGTAGGTTTTCTTTCTCCTCAGCTGG |
| | AGAAGTCAGGTTTGTGTTTTCAGAACTCTGGAGTCTTACAGGTTGCTACATTTTCATACAG |
| | TTATTAGTATGTATTTATTATCATTGGTATTTAAGTTTTAATAATTTTAAACCTTTTATGT |
| | AGTGTTATTTTTTAAAACTGTTTACTTTCATTTGCAGTTATTCTTCCAGAGTTTCATTAAC |
| | ATCTATTGCTATGAGCAACTACATAGCTATGAGAGCATAAATTTACACCTGTAGACGTAGG |
| | TCTAAATGCCACAAACCTGTGCATAAATGTATAGTGACTTATATTTAACATTATAATAAGA |
| | TAATTTTTAAAATATATCCTAAACGATCAAACTTACTGATGAACTAAATATAAATTATCAG |
| | AGTAATGCATAATTGATTTCAATAATTTTATATTGTTTATATTAATTAATATCTATTTCTT |
| | TACTGAAACATAATATATTGGTCATTTCAAAATAGCTACGATACATTTCAAATGGCCTTGA |
| | TGCTAATAATGAAAAGATTTTGAGGTGATTAATATGCTAATTAGTTATATTTAATTATTCC |
| | ATATTGGGTCCCACACACTGTTAAACAACCAAATCTCATGAGAACTCATTTACTATCATAG |
| | CAAGGGGACCGTGCTAACCATTATTCATAAATCCGGACCCACGATACAACCACCGGGCCCC |
| | GCCTCAAACATTAGGGAAGCAAAATCACATACAGTTGTTATGACAGGTGGGACATCCTGAA |
| | AACCTCTCTAGGCATGTCCCACACGGCCCTGGAGCTGTCTCAGGGGAGCAGTCTCCTCCAG |
| | TGTTTAGACGCACAGGCACGGATAATAGGGCTAAGTCTGGCCAGATGTGTGATATTCAACA |
| | CATTGCACAACTGCTCTGTTCTGTATGTAATTTATCTTCTCTACAAATGTAACATTGATGG |
| | TTGCATTAAATATATTCTGCAAATATGTAAAAATAAAATAAGATGATGATTGCTAAATGAT |
| | TATCAAGGCACAATCACATAATCCGAAGTTACAATTTCCTGAGAGATAGGATTACTGCCCA |
| | TGCTTACCAGGACACTCACATCTGCTCTGGGCACTGCCTTCTCCTCAGGCGTCCCACCCCA |
| | GAGCTTGCTATATAGTAGGAGACATGCAAATAGGGTCCTCCCTCTGCTGATGAAAACCAGC |
| | CCAGCCCTGACCCTGCAGCTCTGAGAGCGGAGCCCCAGCCCCAGAATTCCCAGGTGTTTTC |
| | ATTTGGTGATCAGCACTGAACACAGAGGACTCACCATGGAGTTTGGGCTGAGCTGGGTTTT |
| | CCTTGTTGTTATTTTACAAGGTGATTTATGGAGAACTAGAGATGTTAAGTGTGAGTGGACG |
| | TGAGTGAGAGAAACAGTGGATTTGTGTGACAGTTTCTGACCAGGGTGTCTCTGTGTTTGCA |
| | GGTGTCCAGTGTGAGGTGCAGCTGGTGGAGTCTGGGGGAGGCTTGGTCCAGCCTGGAGGGT |
| | CCCTGAGACTCTCCTGTGCAGCCTCTGGATTCACCTTCAGTGACCACTACATGGACTGGGT |
| | CCGCCAGGCTCCAGGGAAGGGGCTGGAGTGGGTTGGCCGTACTAGAAACAAAGCTAACAGT |
| | TACACCACAGAATACGCCGCGTCTGTGAAAGGCAGATTCACCATCTCAAGAGATGATTCAA |
| | AGAACTCACTGTATCTGCAAATGAACAGCCTGAAAACCGAGGACACGGCCGTGTATTACTG |
| | TGCTAGAGACACAGCGAGGGGAGGTCAGTGTGAGCCCGGACACAAACCTCCCTGCAGGGGC |
| | GCGTGGGGCCACCAGGGGGCGGTCGGGAACCACTGAGGACGGGACAGGTCCCAGGAGCAGG |
| | TGCAGGGGGCGGTTTCCTGTCAGCTGCAGGAGGCGGGTTTGTTTTTGCAGGACTCTGGAGC |
| | CTTATGAGGTTGCACTATTTTATTATGGTTATTTATCATGTGTTTTTTATTGATATTTGTG |
| | TTTTAGTAATTTTTACATTTATATGTAGGGGTATTTTTTAAAAATACATAACTTCAAGAAA |
| | TAATTCTTCCTAGTAATTTACACCTATTCCTCTAAAGTTTTATTAACACCTGTTGATATCA |
| | GCAACTACATAGCTATGCAACATTAATTTACATCTATAAACATATGTGTGAATACATGAAA |
| | CTATGCATACATGTGTAGTCATTTATATTTAACATTATAATAAAATAATAAATTAAATAAA |
| | ATAATTGAATTAAATTAAAAGAATTAAACTTAATGATGAACTAAATATAAATTAGAGTAAT |
| | CCATAATTGATTTCAATCCTTCTCTATGGTTTACATAAATCGATGTCTACTTGTAAGCTTA |
| | AGTATAGTGAATTGGTCATTTTAAAATAGCCAAGAACAAATTTCAAATGTTCTTGTCACCA |
| | AAAAATGATAAGGATTTGAAGATTTGAGGTGATATATAAGGAGTCTTCCAATTCCCTTGTC |
| | CAAAGCATCCGCTCATTCATGGGCTGAGAAAAGGGAAGTCATTCATGCAGTCTACTCTCTC |
| | CACAGAACTGACGGGGCACAAGGACAACATCGATTTTTCATGGAACATGCCTCTAGGAATG |
| | CAGCTGTGTGCACACACACTGCTAAGCACACACTTCTTTACATAATTACTTGTAACTGTAT |
| | TTTCTTATTTATTCTCTCCAATTTTTTTACACAAATTCATCACTTTTCCCCATAATCAAAG |
| | AGGATTTTGATCAGAATGCTTGTGGGGAGCCCCTTGCCTGCCAGATGCCCAACATCACTAC |
| | TCTTGAAGGGAGGAGGAACGGCAGCTCTCTTGATTTCTACTCTAATCCTCTAGGACTAAAA |
| | CCAGAAGGTTGCATGTCCAGTGCGGGAGCATCGAAGAAGATCCTGTCTGTAGAAGCAGGAG |
| | CGTCAAGACTTGACTGAGAGCCATGGTGCTGAAATGAGATAGATTCCCTGATGGAGAGCAC |
| | ACGTGGACCCCCACACCTGAGGGCTCACTGCTCCTCACCACAGATGCACTCCCCTACTGAG |
| | TCCTGAGACCTGAGTGCACCCCATAGAGTAGGGCTCAGATGAGGGGATGCAAATCTCCACC |
| | AGCTCCACCCTCCCCTGGGTTCAAAAGACGAGGACAGGGCCTCGCTCAGTGAATCCTGCTC |
| | TCCACCATGGACATACTTTGTTCCACGCTCCTGCTACTGACTGTCCCGTCCTGTGAGTGCT |
| | GTGGTCAGGTAGTACTTCAGAAGCAAAAAATCTATTCTCTCCTTTGTGGGCTTCATCTTCT |
| | TATGTCTTCTCCACAGGGGTCTTATCCCAGGTCACCTTGAAGGAGTCTGGTCCTGCGCTGG |
| | TGAAACCCACACAGACCCTCACACTGACCTGCACCTTCTCTGGGTTCTCACTCAGCACTAG |
| | TGGAATGCGTGTGAGCTGGATCCGTCAGCCCCCAGGGAAGGCCCTGGAGTGGCTTGCACGC |
| | ATTGATTGGGATGATGATAAATTCTACAGCACATCTCTGAAGACCAGGCTCACCATCTCCA |
| | AGGACACCTCCAAAAACCAGGTGGTCCTTACAATGACCAACATGGACCCTGTGGACACGGC |
| | CGTGTATTACTGTGCACGGATACCACAGAGACACAGCCCAGGGCGCCTCCTGTACAAGAAC |
| | CCAGGCTGCTTCTCAGTGGTGCTCCCTCCCCACCTCTGCAGAACAGGATAGTGTGGCTGAG |
| | ATGCCATTTCCTGCCAGGGCCTGCGTTTCCCATCCCCATCTGACTCAGAGCCTTGTTTTCC |
| | TCCCTCTTCTTTACTAATAAATGGCATGTCCCCTGTTAGTGGTTCGTGCAAGCAGAAGCTG |
| | TATCCTGTTTGACAAAGATTCAGCATGAAAGGTTCCTGTTACCTAAAAAAAAATAGACAGA |
| | TGAGACTTAATTAACCTAAATAATTTTTTTCACAACAACAGAGTGAATACACAATTTGCAG |
| | AATGACAGAAAACTTTTGCACACTTTGTCTGTGACAGGGAACTAATATGAAGAATTTGCAA |
| | GGAACTCAAACAACTCTACAACAACAACAGCAACGAGAACCAAATAACCCCATTAAAATGA |
| | GCAAAGAACATGAGTAGACATTTTCAAAAGAACACATAGAAATGGATAATAAATATATAAA |
| | CAATGCTCAACATCACTAACCATCAGGGAAATGCAAATTAAAACCACAATAAGATATCATC |
| | TTCCACCAGTCACAATGACTGTTACTAAAAACTCAAATAATATCAGATGTTGCTGAGGATG |
| | GGAAATAAAGGCAACTCTTAGACATTGTTGATGAGGATGTAGACGAGTACAAACTCAAATT |
| | CCTATTTCTATTTCAACCCCTGATTCCTACTGTCAATGGGAGGGAAGTCTCAGAACCAATC |
| | ACACATCAGACGGCAAATCTGTCAACCAAGAGTCTTTCCACTGAAGGACCTGGGAGGTCAG |
| | GACCCTCAGGAAAGTGCTGGGGACCCTGTCTTGGGAGTGCCCAGCAGATCTCAGAACTCTC |
| | CATGGGTCCTGCTGGACACTCATGTAGGGTAACGAGTGGCCACCTTTTCAGTGTTACCAGT |
| | GAGCTCTGAGTGTTCCTAATGGGACCAGGATGGGTCTAGGTGCCTGCTCAATGTCAGAGAC |
| | AGCAATGGTCCCACAAAAAACCCAGGTAATCTTTAGGCCAATAAAATGTGGGTTCACAGTG |
| | AGGAGTGCATCCTGGGGTTGGGGTTTGTTCTGCAGCGGGAAGAGCGCTGTGCACAGAAAGC |
| | TTAGAAATGGGGCAAGAGATGCTTTTCCTCAGGCAGGATTTAGGGCTTGGTCTCTCAGCAT |
| | CCCACACTTGTACAGCTGATGTGGCATCTGTGTTTTCTTTCTCATCCTAGATCAGGCTTTG |
| | AGCTGTGAAATACCCTGCCTCATGCATATGCAAATAACCTGAGGTCTTCTGAGATAAATAT |
| | AGATATATTGGTGCCCTGAGAGCATCACATAACAACCACATTCCTCCTCTGAAGAAGCCCC |
| | TGGGAGCACAGCTCATCACCATGGACTGGACCTGGAGGTTCCTCTTTGTGGTGGCAGCAGC |
| | TACAGGTAAGGGGCTTCCTAGTCCTAAGGCTGAGGAAGGGATCCTGGTTTAGTTAAAGAGG |
| | ATTTTATTCACCCCTGTGTCCTCTCCACAGGTGTCCAGTCCCAGGTGCAGCTGGTGCAGTC |
| | TGGGGCTGAGGTGAAGAAGCCTGGGTCCTCGGTGAAGGTCTCCTGCAAGGCTTCTGGAGGC |
| | ACCTTCAGCAGCTATGCTATCAGCTGGGTGCGACAGGCCCCTGGACAAGGGCTTGAGTGGA |
| | TGGGAGGGATCATCCCTATCTTTGGTACAGCAAACTACGCACAGAAGTTCCAGGGCAGAGT |
| | CACGATTACCGCGGACGAATCCACGAGCACAGCCTACATGGAGCTGAGCAGCCTGAGATCT |
| | GAGGACACGGCCGTGTATTACTGTGCGAGAGACACAGTGTGAAAACCCACATCCTGAGAGT |
| | GTCAGAAACCCTGAGGGAGAAGGCAGCTGTGCCGGGCTGAGGAGATGACAGGGGTTATTAG |
| | GTTTAAGGCTGTTTACAAAATGGGTTATATATTTGAGAAAAAAAGAACAGTAGAAACAAGT |
| | ACATACTCTAATTTTAAGATAAATATTCCATTCAAGAGTCGTAATATAAGCCAAATTCACA |
| | GAGTGGAAAAGGCCACACTCTATAACGTTGATACAAACATTCCATGAAGGTGCTACTGTGA |
| | ACAAGTTTTCAAATTGGATGAATACATGATTTGGAGCAAGGTTATTTGATCATGTGGTGAG |
| | ACTAAGAATGATTCTTAAAAAGTGCCAAAAGTTTCCTTCAAATGTTTCTGTCACTCCTTAT |
| | CATAAAGTTTATTTTACAGCAGTTTTAGGATTACAAAGAAATTGCACAGGAGGCGTGAGAA |
| | TTCCCATGACTCCCTGCCCTACACAGGCACAGCCTCCTCCACTACGACCATCCTGCACCGC |
| | AGTCACAAATCAGTTACAATGGAGGAATCTCCAAGGACGCTTGGTTCTTTCTTTTTCTGGT |
| | GATCTCCTAATATAACAAGCCTAAGTATCTCAAGATTCCACGGTTTTTTCAGTGTTTTCTA |
| | GAACTGATATTAGTCAGAGGGAAAGTGGGTAAGGCTATTACTATTTGAACTCTTTCTTCCA |
| | TTATACTTGAATAAAGCAATAACGAAGAAATAAATGAATATTTTTGCTAAAATGGAGCAAT |
| | AAAAAGACTGATATTGACAGAAGAAATATGACTGACTTCTGAAAACACACATGAACCATGG |
| | TTCTCTCTGCATATTTAGGTGAATTACAGAAAGTTGTCATAACAGATGGGGAATCCTGCAG |
| | ACTTCACTAGGCATGGTCCACGCTGCCCTGGAGTTGTCTCAGGGGAGCTGCCTCCTCCGGT |
| | GATTAGAGCACAGGCCCAGATAATAGGATTACATTTTTTTAGATGTGTAAACTTAGACGCA |
| | CTGCACAGCTGCTGTATTCTCTATGTAAATTATCTTCTGTAAAATACAACATTAAAGGCTG |
| | CATTAAATATATTGTGTAAATATGTAAAAATAAAATCAGATTATGAGAGCTAAATGTTAAT |
| | CAAGGCACAATCACATAATATAAAATTATATTTTCCTGAATGATGGAATTACTACCAATCT |
| | CCCCCAGGAGACTTCATCTGCACTGGGCCCGGCCTCTCCTCAGATGTCCCATCACAGAGCT |
| | TGCTATATAATGGGGGACATGCAAATAGGGCCCTCCCTCTGCTGATGAAAACCAGCCCAGC |
| | CCTGACCCTGCAGCTCTGGGAGAGGAGCCCAGCACTGGGATTCCGAGGTGTTTCCATTCAG |
| | TGATCTGCACTGAACACAGAGGACTCGCCATGGAGTTTGGGCTGAGCTGGGTTTTCCTTGT |
| | TGCTATTTTAAAAGGTGATTCATGGAGAACTAGAGATATTGAGTGTGAGTGAACACGAGTG |
| | AGAGAAACAGTGGATATGTGTGGCAGTTTCTAACCAATGTCTCTGTGTTTGCAGGTGTCCA |
| | GTGTGAGGTGCAGCTGGTGGAGTCTGGGGGAGGCTTGGTCCAGCCTGGGGGGTCCCTGAGA |
| | CTCTCCTGTGCAGCCTCTGGATTCACCGTCAGTAGCAACTACATGAGCTGGGTCCGCCAGG |
| | CTCCAGGGAAGGGGCTGGAGTGGGTCTCAGTTATTTATAGCGGTGGTAGCACATACTACGC |
| | AGACTCCGTGAAGGGCAGATTCACCATCTCCAGAGACAATTCCAAGAACACGCTGTATCTT |
| | CAAATGAACAGCCTGAGAGCCGAGGACACGGCTGTGTATTACTGTGCGAGAGACACAGTGA |
| | GGGGAGGCCATTGTGCGCCCAGACACAAACCTCCCTGCAGGAACGCTGGGGGAATCAGCGG |
| | CAGGGGGCGCTCAGGAGCCACTGATCAGAGTCAGCCCCGGAGGCAGGTGCAGGTGGAGGCT |
| | GTTTCCTGTCAGGATGTGGGACTTCATCTTCTTCCAACAGTTTCTCTAATGAACCTCTCTA |
| | ATTTTAGAATTCTGTGGTTCCTAATGTCATCTCTACATATTTTCAAAAGATCATTTTAACA |
| | TGAGGACATAACCTCTCATGCACCAAATGCACATTGATGCTTACAAAGATGAAAAGTTCTC |
| | AACCATTGTGACCAGGATCGCAGTCCTGAGGAAGCTCACGGGTGTCTGATGAGTCTCCTCC |
| | ATTCAGGCCCAGGACAGAAACCTCAGAGGGATTCCTGGACCAGAACGGCAGGGATTCTGAT |
| | CACAGCCAATAGAGAGGCTGGGCCAGGGTCAGTGTCCTGTAGAAGCTCACAGGGTTCAAGT |
| | CTGACCCTTCTCCTGACCCTAAAGCCAATGAGCATCAGCACTGATCTGGTGCTGCTTTTGC |
| | TCCCAATACATGTTCTTTCTTTGGAGTGTTTGTTCTCCCTTTTTTATTTGCTTTTCTTTCT |
| | TCCTGAAAAAGAAACACATGGTCTCTGTGATCTACACTCCAGGGCTCAAGGCATTTTCTTA |
| | GAACTCAGGCAAGGCTCAGGCTTATTACAAAAAAACTGAAATTTTAGACAAAAAGAGGGTG |
| | ATAGGAAGGAACAAATAAATATGTTAAATGTCAAATATACCTAAAAATTTATTTGTCTGAC |
| | CCCTAGTTTTCTCCGTATTTTTAGGTAAATGCAGCAAAATCACACAGGTTGTCGTGGCAGG |
| | AAGTGGATTCTGCAAACCACACTAGGCCCGTTTATCTCTGTCCTAGAGTTGGTTAAAAGAG |
| | CAACTGAGGCCAGCTGTGAGGAGCATAGGCCCGGGTACTAGGACTCACTCATGCCAGATAT |
| | AAGCCCTTAGACACATACATAGCCCCTCCATGTGTGGGTTCACTTTTACATCTGTACATGA |
| | AGAAACCACTGATTCCTAAATAACATAATTTATACACATAGGTAAAAATAATTAAAAATGT |
| | GATAGTTATTAAGTGTTTATCACACAACAATTTCACAATAAAACAGCATTTTCCCAAATGT |
| | AATCATTGTCATCGAAATCCCCAAGGACACTCTCATCTGCCCTGGGCCCTGCCCTCTCCTC |
| | AGGCATCTCACCCCAGAGCTTGCTATATAGTAGGAGACATGCAAATAGGTCCCTCCCTCTC |
| | CTGATGAAAACCAGCCCAGCCCTGACTCCGCAGCTCTGGGAGAGGAGCCCCCGCCCTGGGA |
| | TTCCCAGGTGTTTTCATTTGGTGATCAGCACTGAACACAGAAGAGTCATGACGGAGTTTGG |
| | GCTGAGCTGGGTTTTCCTTGTTGCTATTTTTAAAGGTGATTCATGAGGAAATAGAGATATT |
| | GAGTGTGAGTGGACATGAGTGAGAGAAACAGTGGATTTGTGTGGCAGTTTCTGACCTTGGT |
| | GTCTCTGTGTTTGCAGGTGTCCAGTGTGAGGTGCAGCTGGTGGAGTCTGGGGGAGGCTTGG |
| | TCCAGCCTGGGGGGTCCCTGAGACTCTCCTGTGCAGCCTCTGGATTCACCTTCAGTAGCTA |
| | TGCTATGCACTGGGTCCGCCAGGCTCCAGGGAAGGGACTGGAATATGTTTCAGCTATTAGT |
| | AGTAATGGGGGTAGCACATATTATGCAAACTCTGTGAAGGGCAGATTCACCATCTCCAGAG |
| | ACAATTCCAAGAACACGCTGTATCTTCAAATGGGCAGCCTGAGAGCTGAGGACATGGCTGT |
| | GTATTACTGTGCGAGAGACACAGTGAGGAGAAGTTAATGTGGGACCATGCAGAAACCTCCC |
| | TGCGGGAACGCTGGGGAAAGTCATCTGCAGGGGGCGCTCAGGAGCCACTGATCAGAGTCAG |
| | CCCCAGCGGCAGGTGCAGATGAAGGCTGATTTCCTGTCACGATGTGGGACTTCATCTTCTT |
| | AAAGTTTCTCTACTGAACCTAAGTTCGGAATTCTGTGATTACTAGGGTCATTTCTACATAT |
| | TTTTAAAATGATTGTTTTAATATGAAAACCTATTCTCCTATGCAGAAAATGCAGATTGATC |
| | CTCACAGAGGAGATGAAAAGTTCTCAACCATGGTCACCACTGTCAGAGTCCTGAGGAAGCT |
| | CAGGGCTTCCTGGTGAGTCTTCTCCAGTCAGACTGAGGACAGGAACTCCAGAAAGAATCCT |
| | GACTAGAACTGAGGTATTGCTCCTCACAAGAGAACTCAAGCTGCGGGGGGTCTGTTCCTGC |
| | AGACCCCTGACCCGGTGGCGGATGAATAAAACGTACACTGACACACAGATATTCTGTTTTG |
| | CCAGTTCAGCTGAGGGTCCGAGGCAACTTACAGACTCCAAGAAGAATCCTGTAAAGAACGG |
| | CAGCAGTGGCCCTGAGCAGCTCGCACTCCAGGCGTGTATTGAGTATAGAATTAACAACAGA |
| | AGCTCTGACAACACACTTGTGGATAATTAACATGGTTAAGAGAGTAGTACATATTTTCAAT |
| | CAGAAGTGTTTAGAGGCCAGGACATATCTTCACGGTCACACATTGAGAAGGATGTAGATAT |
| | GTCCCACTACCTTCTCCTGAGATCTCAGACAGAATCCCAGATTTCAAAAGGACACAGAAGG |
| | ACAGCTCTCAGGTGCTTTTAAAAAATGACCCACTTCCAGGGACAGGGAGCTTCCCTATAAC |
| | CATGGTGGATGTTCTGAACTACAATAAACATTGGATGGATCCAGGATTGTTTGAAGTCACT |
| | GTCATTATTACATTCAGCTGCTGTTTCAATGTGTCTGAAGTAGTAAATGACAATTTAGATG |
| | ACAATTTATATGAATCTTCAAGGGTAGAACAATATTGACCATATTCCAAAATCTGTCCTTG |
| | ATCCATGATCACACTCATCTCCCAGACCAGGTCCTTCAGCACGTCTCTTTACCTGAAAGAA |
| | GAGGACTCTGGGCTTGGAGAGGGGAGACCCCAAGAAGACAACTGAGTTCTCAAAGGGCACA |
| | GCCAGCATCCTACTCCCAGGGCGAGCCCAAAAGACTGGGGCCTCCCTCCTCCTTTTTCACC |
| | TCTCCGTACAAAGGCACCACCCACATGCAAATCCTTACTTAAGCACCCACAGGAAACCACC |
| | ACACATTTCCTTAAATTCAGGTTCCAGCTCACATGGGAAATACTTTCTGAGAGTCCTGGAC |
| | CTCCTGTGCAAGAACATGAAACACCTGTGGTTCTTCCTCCTCCTGGTGGCAGCCCCGAGAT |
| | GTGAGTGTCTCAGGGATCCAGACATGGGGGTATGGGAGGTGCCTCTGATCCCAGGGCTCAC |
| | TGTGGGTCTCTCTGTTCACAGGGGTCCTGTCCCAGGTGCAGCTGCAGGAGTCGGGCCCAGG |
| | ACTGGTGAAGCCTTCGGAGACCCTGTCCCTCACCTGCACTGTCTCTGGTGGCTCCGTCAGC |
| | AGTGGTAGTTACTACTGGAGCTGGATCCGGCAGCCCCCAGGGAAGGGACTGGAGTGGATTG |
| | GGTATATCTATTACAGTGGGAGCACCAACTACAACCCCTCCCTCAAGAGTCGAGTCACCAT |
| | ATCAGTAGACACGTCCAAGAACCAGTTCTCCCTGAAGCTGAGCTCTGTGACCGCTGCGGAC |
| | ACGGCCGTGTATTACTGTGCGAGAGACACAGTGAGGGGAGGTGAGTGTGAGCCCAGACACA |
| | AACCTCCCTGCATGGACGCGGAGGGGACCGGCGCAGGTGCTGCTCAGGACCAGCAGGTGGC |
| | GCGCGGGGCCCCCAGAGCATGAGGCCGGGTCAGGAGCAGGTGCAGGGAGGGCGGGGCTTCC |
| | TCATCTGCTCAGTGGTCTCCGTCCTCGCCAGCACCTCGCTGTCACCAGGGCTCCTCTTTCT |
| | TTATTATCTGTGGTTCTGCTTCCTCACATTCTTGTGCCAGGAAAGAAACGAGGAAGACAAA |
| | TTTTCGTCTATAGTTGAAGCTTCACCAATTACTAGGAACTTGCCTACAAGTTCCTGCATGA |
| | CCCATTATAACTTATCGATTAAAAAATATATATTCTAATGCTTCTCACCATCTCTTGATTT |
| | GTATCATCAACTGAATTGTACCCTCTTTGAAATTCATATGATGAAACCTTAAATTCAATGG |
| | ATCTATATTGGAATTTTAATGAAATAATTAAGGTTAAATGTGGTCATAATTGTAAGACCCT |
| | AATGCAATAGACGTGTTGTCTTTATAAGAAGAGGAAGAGACACCAGAGACCTCTCACTTTT |
| | CACGTGCAGGCAGAGAAGAGGCCATGTGGAGACATAGTGCACTAGAAGGTGGCCCAGTGCA |
| | AGCCAGGAAGAAGCCGCGCCAAGAACCAGCCCTGCCAGCACACTAATCTTCAACATACATA |
| | TTTTCAATCAGAAGTGTTTAGAGGCCAGGACACATCTTCAAGGTCACACATTGAGAAGGAT |
| | GTAGATATGTCCCACTACCTTCTCCTGAGATCTCAGACAGAATCCCAGATTTCAAAAGGAC |
| | ACAGAAGGACAGCTCTCAGGTGCTTTTAAAAAATGACCCACTTCCAGGGACAGGGAGCTTC |
| | CCTATAACCATGGTGGATGTTCTGAACTACAATAAACATTGGATGGATCCAGGATTGTTTG |
| | AAGTCACTGTCATTATTACATTCAGCTGCTGTTTCAATGTGTCTGAAGTAGTAAATGACAA |
| | TTTAGATGACAATTTATATGAATCTTCAAGGGTAGAACAATATTGACCATATTCCAAAATC |
| | TGTCCTTGATCCATGATCACACTCATCTCCCAGACCAGGTCCTTCAGCACGTCTCTTTACC |
| | TGAAAGAAGAGGACTCTGGGCTTGGAGAGGGGAGACCCCAAGAAGACAACTGAGTTCTCAA |
| | AGGGCACAGCCAGCATCCTACTCCCAGGGCGAGCCCAAAAGACTGGGGCCTCCCTCCTCCT |
| | TTTTCACCTCTCCATACAAAGGCACCACCCACATGCAAATCCTCACTTAAGCACCCACAGG |
| | AAACCACCACACATTTCCTTAAATTCAGGTTCCAGCTCACATGGGAAATACTTTCTGAGAG |
| | TCCTGGACCTCCTGTGCAAGAACATGAAACATCTGTGGTTCTTCCTTCTCCTGGTGGCAGC |
| | TCCCAGATGTGAGTATCTCAGGGATCCAGACATGGGGATATGGGAGGTGCCTCTGATCCCA |
| | GGGCTCACTGTGGGTCTCTCTGTTCACAGGGGTCCTGTCCCAGGTGCAGCTGCAGGAGTCG |
| | GGCCCAGGACTGGTGAAGCCTTCGGAGACCCTGTCCCTCACCTGCACTGTCTCTGGTGGCT |
| | CCATCAGTAGTTACTACTGGAGCTGGATCCGGCAGCCCCCAGGGAAGGGACTGGAGTGGAT |
| | TGGGTATATCTATTACAGTGGGAGCACCAACTACAACCCCTCCCTCAAGAGTCGAGTCACC |
| | ATATCAGTAGACACGTCCAAGAACCAGTTCTCCCTGAAGCTGAGCTCTGTGACCGCTGCGG |
| | ACACGGCCGTGTATTACTGTGCGAGAGACACAGTGAGGGGAGGTGAGTGTGAGCCCAGACA |
| | AAAACCTCCGTGCAGGGAGGCGGAGGGGACCGGCGCAGGTGCTGCTCAGCGCCAGCAGGGG |
| | GCGCGCGGGGCCCACAGAGCAGGAGGCCCGGTCAGGAGCAGGTGCAGGGAGGGCGGGGCTT |
| | CCTCATCTGCTCAGTGGTCTCCCTCCTCGCCAGCACCTCAGCTGTCCCCAGGGGTCCTCTT |
| | TCTTTATTATCTGTGGTTCTGCTTCCTCACATTCTTGTGCCAAGAAAGAAATGAGGAAGAC |
| | AAATTTTCGTCTGTAGTTGAAGTTTCACCAATTACTAGGAACTTTCCTAGAAGTTCCTGCA |
| | TGGCCCATTATAGCTTACTGATTAAAAAATATATATTCTAACGCTTCTCAGCATCTCTTGA |
| | TTTGTGTCATCAACTGAATTGTGCCCTCTTTGAAATTCATATGCAGAAACCTTAAATTCAA |
| | TTGATGTATATTGGAATTTTAATGAAATAATTAAGGTTAAATGTGGTCATAAGTGTAAGAC |
| | TCTAATTCAACAGACGTGTCGTCTTTATAAGAAGAGGAAGAGACACCAGAGACCTCTCACT |
| | TTTCACGTGCAGGCAGAGAAGAGGCCATGTGGAGACGTAATGCACTAGAAGGTGGCCCAGT |
| | GCAAGCCAGGAAGAAGCCTCACCAAGAACCAACCCTGCCAGAACATTGATCTTCAACGCAG |
| | TGAGTTCATCAGGAACCCCAAACTTCTCTTACGTCTTCTGATTCCTGTTGTCCATGAGATG |
| | AGAAATTCAGCTCTAATTGTACATCACAGGGCAAATCTGTAAACCAGGAGTGTTTCTATTG |
| | AGGATCATGGTGGATCAGGATTCCAGGCAGGTGCTGGAGACACTGTCTCAGGAGCGCCCAG |
| | ATGATCTCAGGGGGACCTGCTGGACACTCACGTGGGACATCAGCAGTCACTTTCTCAGAGT |
| | AACCAGTGAGCTGTGCTGGTGCCTGATGGGACTAGGATGGGGTCAAGGCACCTTCTCAGTG |
| | TCATGGAGAGTGATTGTTCCAGAAATCATCCAGGTGGTCTCTACGCTAATCAAATATGGGT |
| | TCACAGGGAGGAACATGTGCTCTGGGTGCTTGGTCTTCAGTGAAAGGACGTCTGGCCACCA |
| | AAAGTTTGTAAATGGAGCAGGGCATGCATTTCCTCAAGGAGGATTAGGGCTTGGAGCATCA |
| | GCATCCCACTCTTGTAAGGCTGATGTGTCATTTACCTTCCCTTTCTTATCCCAAATCAGGG |
| | TCTTCAGCTATGAAATGCTCTGACTCATGAATATGCAAATAACCTGAGATGCACTGAGGTA |
| | AATATGGATATTTGTCAGCCCTGAGAGCATCATCCAGAAACCACATCCCTCCGCTAGAGAA |
| | GCCCCTGACGGCACAGTTCCTCACTATGGACTGGATTTGGAGGATCCTCTTCTTGGTGGGA |
| | GCAGCGACAGGCAAGGAGATGCCAAGTCCCAGTGATGAGGAGGGGATTGAGTCCAGTCAAG |
| | GTGGCTTTCATCCACTCCTGTGTTCTCTCCACAGGTGCCCACTCCCAAATGCAGCTGGTGC |
| | AGTCTGGGCCTGAGGTGAAGAAGCCTGGGACCTCAGTGAAGGTCTCCTGCAAGGCTTCTGG |
| | ATTCACCTTTACTAGCTCTGCTGTGCAGTGGGTGCGACAGGCTCGTGGACAACGCCTTGAG |
| | TGGATAGGATGGATCGTCGTTGGCAGTGGTAACACAAACTACGCACAGAAGTTCCAGGAAA |
| | GAGTCACCATTACCAGGGACATGTCCACAAGCACAGCCTACATGGAGCTGAGCAGCCTGAG |
| | ATCCGAGGACACGGCCGTGTATTACTGTGCGGCAGACACAGTGTGAAAACCCACATCCTGA |
| | GAGTGTCAGAAACGCCAGGAAGGAGGCAGCTGTACTGGCATGGAGGGGATGACAAAGGTTA |
| | TTAGATTGAAGATTTTCTTAGAAAACGACTTCAAGTCATTAAAGAAGAGGAACAACATAAA |
| | TGTGTATTTGTGAAATTTTAATTGAGAGATTTTTCATACAACATTTATTCTGTAAGCTATT |
| | TCAGGGATTGGAATATGAATCAAATTAATAAAGCTGATATAGACATCCTCTGAAGGCATCT |
| | TCGTAAACATCAATTTCTGAATCAGTGTTGTAAATATTTTGGAACACAGACACAAGATCAC |
| | ATTTTTACTCTACTTTTATCTCTATTTTTAAAAAATGCCAAAAAGAATCTTATTTTGTGCA |
| | TGCCCCATTTTGAATTCCCACCGTCAATGCATGATAGTTCTTGGTTTTCCTCATTCAAGTT |
| | GTCATTTGTCATTAAGAGTGTTGTGTGTTTTAACCATTCTAATAGGTGAGTAACGGTATCT |
| | AATTTTTATTTAAATGCACATGTCCCTAAAAAATTCATATTTAACAATTTTTATATAATTT |
| | TTGGTGAGATGCCTCTCGTGATATTTGGTTCATTTTTTAAATGCATTTTTTATCAGTTGTA |
| | AGTATGCTTGCATATTGATTATAAAAGTCATTTAACAAATTAAAATAATTCATTTAACAAA |
| | TATGTATACTTGAATAAAGCAATAACAAAGAAATAAATGAATATTTTTGCTAAAATGGAGC |
| | AATAAAAAGACTGATATTGACAGAAGAAATATGACTGACTTCTGAAAATACACACACATGA |
| | GCCGTGGTTCTCTCTACATATTTAGATAAATTACAGAAAGTTGTCATAACTGATGGGGAAT |
| | CCTGCAGACTTCACTAGGCATAGTCCACACTGCCCTGGAGTTGTCTCAGGGGAGCTGCCTC |
| | CTCCAGTGGTTAGAGCACAGGCCCAGGTAATAGGACTCATTTTTTTAGATGTGTAATTTTA |
| | GACACACTGCACAACTGCTGTGTTCTCTGTGCAAATTATCTCCTGTAAAATGTAACATTGA |
| | AACCTGCCTTAAATATATTGTGTAAATATGTAAAAATAAAATCAGATTGTGAGAGCTAAAT |
| | GCTAATCAAGGCGCAATCACGTAATATACAATTATATTTTCCTGAATGATGGAATTAATAC |
| | CAATCTCCCCCAGGACACTTCATCTGCACGGAGCCCGGCCTCTCCTCAGATGTCCCACCCC |
| | AGAGCTTGCTATATAGTCGGGGACATGCAAATAGGGCCCTCCCTCTGCTGATGAAAACCAG |
| | CCCAGCTGACCCTGCAGCTCTGGGAGAGGAGCCCAGCACTGGGATTCCGAGGTGTTTCCAT |
| | TCGGTGATCAGCACTGAACACAGAGGACTCACCATGGAGTTTTGGCTGAGCTGGGTTTTCC |
| | TTGTTGCTATTTTAAAAGGTGATTCATGGAGAACTAGAGATATCGAGTGTGAGTGAACACG |
| | AGTGAGAGAAACAGTGGATATGTGTGGCAGTTTCTAACCAATGTCTCTGTGTTTGCAGGTG |
| | TCCAGTGTGAGGTGCAGCTGGTGGAGTCTGGAGGAGGCTTGATCCAGCCTGGGGGGTCCCT |
| | GAGACTCTCCTGTGCAGCCTCTGGGTTCACCGTCAGTAGCAACTACATGAGCTGGGTCCGC |
| | CAGGCTCCAGGGAAGGGGCTGGAGTGGGTCTCAGTTATTTATAGCGGTGGTAGCACATACT |
| | ACGCAGACTCCGTGAAGGGCCGATTCACCATCTCCAGAGACAATTCCAAGAACACGCTGTA |
| | TCTTCAAATGAACAGCCTGAGAGCCGAGGACACGGCCGTGTATTACTGTGCGAGAGACACA |
| | GTGAGGGGAGGCCATTGTGCGCCCAGACACAAACCTCCCTGCAGGAACGCTGGGGAAATCA |
| | GCGGCAGGGGGCGCTCAGGAGCCACTGATCAGAGTCAGCCCCGGAGGCAGGTGCAGATGGA |
| | GGCTGATTTCCTGTCAGGATGTGGGACTCTGTCTTCTTCTGACGGTTCCCCAGGGAACCTC |
| | TCTAAGTTTAGCATTCTGTGCCTATGAACGTCTTCTCTAAGTATTTGAAAGAGATTATTTT |
| | AATATGAAGAGCAGTTCTCACTCGCACAAAATGTGGATTGATGCTTACTGGGATGAAAAGT |
| | CCTCAAACATGGTCACCACGATCAGAGTCTGAGTGAGCTCAGGGCTTCCTGCTGAGTCTCC |
| | TCCTATCAGACCAAGGACAGGGACCTCAGTGAGGTTCCCCGTCTAGAACAGTCTTTATGGA |
| | TACTGATTGTGGGCGGCAAGCCACCCAGGTGCCGACGCAAGAGACCGAGGACACGAGCTGT |
| | TCCAGTACAATAAAATATAAAACAAGAATAGTTATACCAGATATAGATCTTAGATATGATT |
| | ATATATGAATATCATTAATCATTAGTTGGTAGCAATTACTCTTTATTCCAATATTATAATA |
| | ATCCTCACTCTACAATCATAACCTAGGAAAAGCCAGGCCATACAGAGATAGGAGCTGAGGG |
| | GACATAGTGAGAAGTGACCAGAAGATTGGGTGCAGCAATGGGCATCCCTTCAGGTCCATCA |
| | GCAAGTCAGAGACCTAAACAAATATTCCCTATTCATGTTGAATTGGCAGAGTGTAAAAATC |
| | TAGAAGACAAAATGGCCAGGAAAAATGTGCGATTTTGCATGGGTGAAGGTCTGGCAAGTTA |
| | ATCATCTTGAGGACTGTTGAGGTCCTGAGGTGGTCCTGCCCTTGCTTGATGCCCAGGCCGT |
| | AGTCCAACTCACACGAAATTGCCAGGGAATAGACAGTAGTTTTTTTAGTAGTTCTTGTTAT |
| | CAGGCATAAGTGCATTTGAATTTTCTCTTCATGGCCTTTCCTGGCACTATTTCTCATTTTT |
| | TTTAACACACATAGTTTCAACTAGATTTATCACCTTCACAGGGTCACAGAGAAGGGTGGAA |
| | GAAGGGAGGCCCTGTATGGGTCTCGAAGAAACATGGAAAAGAGTGGAGAGGGACAATAGCA |
| | GGGTGTAAGGAATTATTGAGACCTTACTCTGCCCCTCCCAGGAGGCTCAGGCCAGCCTTTT |
| | TCTGCATTTGAGGTTCTGGGTTATAAACGCTGTAGACTCCTCCCTTCAGGGCAGGGTGACA |
| | ACTATGCAAATGCAAGTGGGGGCCTCCCCACTTAAACCCAGGGCTCCCCTCCACAGTGAGT |
| | CTCCCTCACTGCCCAGCTGGGATCTCAGGGCTTCATTTTCTGTCCTCCACCATCATGGGGT |
| | CAACCGCCATCCTCGCCCTCCTCCTGGCTGTTCTCCAAGGTCAGTCCTGCCGAGGGCTTGA |
| | GGTCACAGAGGAGAACGGGTGGAAAGGAGCCCCTGATTCAAATTTTGTGTCTCCCCCACAG |
| | GAGTCTGTTCCGAGGTGCAGCTGGTGCAGTCTGGAGCAGAGGTGAAAAAGCCCGGGGAGTC |
| | TCTGAAGATCTCCTGTAAGGGTTCTGGATACAGCTTTACCAGCTACTGGATCGGCTGGGTG |
| | CGCCAGATGCCCGGGAAAGGCCTGGAGTGGATGGGGATCATCTATCCTGGTGACTCTGATA |
| | CCAGATACAGCCCGTCCTTCCAAGGCCAGGTCACCATCTCAGCCGACAAGTCCATCAGCAC |
| | CGCCTACCTGCAGTGGAGCAGCCTGAAGGCCTCGGACACCGCCATGTATTACTGTGCGAGA |
| | CACACAGTGAGAGAAACCAGCCCCGAGCCCGTCTAAAACCCTCCACACCGCAGGTGCAGAA |
| | TGAGCTGCTAGAGACTCACTCCCCAGGGGCCTCTCTATTCATCCGGGGAGGAAACACTGGC |
| | TGTTTGTGTCCTCAGGAGCAAGAACCAGAGAACAATGTGGGAGGGTTCCCAGCCCCTAAGG |
| | CAACTGTATAGGGGACCTGACCATGGGAGGTGGATTCTCTGACGGGGCTCTTGTGTGTTCT |
| | ACAAGGTTGTTCATGGTGTATATTAGATGGTTAACATCAAAAGGCTGCCTAACAGGCACCT |
| | CTCCAATATGACAGTATTTTAATTAGTGAAAATTTTACACAGTTCATCATTGCTTGCTTGC |
| | CTTCCTCCCTCCTGTCCACTCTCACTCACTCCTTCTTTTATTTTCTACTTAATTTTACAAA |
| | ATCATTTAACCCCTTTTTGAACTATTAATAGGTTATCTTTGTTTGGTGATTGTTTTCCTTT |
| | CAATAATATGTACTGAATAATTCATCTTTGTGCCAATTCATAAGTATTCTGGTGTAATAAA |
| | GACTTCTTTCATAAAAATTGGATAAATTAAAATAAAGATAAATTTTTAAAAACATACGATC |
| | TATCAAAACTGAACCATAAAGAAATAAAAACTCTGGGTTGGGTGTGTTTGCTCATTCCTGT |
| | AATCCCAGCACTTTGGGAGGCCATGGCCGGTTTACAAGCAAAAATTGAAAGTTAGACAAGA |
| | AGGAGTGATAGAAAGAGAAAATGTATATTAAATTTCAGAAATATTTAAGAATGTATCTGCC |
| | TGAACCCTAGTTCTCACCATATCTTTAGGTGAATGCTAAAATGCAGCAAAATCACGCATGT |
| | TCTCACTACAGAAAGTGGGTTCTACAAACCACACTCGGCACATTTAGCTTTGTCCTGGAGT |
| | TGGTTCAGGGAGTTATTGGGGCCAGTGATGAGGAGCACAGGCCAAGATACCAGCGATCACT |
| | TATCCCAAACATGAGCTCTAACATACACACTTAGTCCCTTTTCCGTGTGTGGTTTACTTCC |
| | ACATCTGTACATGGAGAGACCACTGACTGACAAAATATAATTTATACAAATATGTAAAATT |
| | AAATAGGGTGATCAGTTCAAGGTGTTTATCACAGCATAATTTTACAATAAGACAGCATATT |
| | TCCCAAATACCATCATTGTCACCAAACTCCTTCAAGGCACAGTCATCTTATCTGGGCCCCG |
| | TCCTCTCCTCAGGTGTCCCACCCCAGAGCTTGGTATATAGTAGGAGACATGCAAATAAGGC |
| | CCTCCCTCTGCTGATGAAAATGAGCCCAGCCCTGACCCTGCAGCTCTGGGAGAGGAGCCCC |
| | AGCCGTGAGATTCCCAGGAGTTTCCACTTGGTGATCAGCACTGAACACAGACCACCAACCA |
| | TGGAGTTTGGGCTTAGCTGGGTTTTCCTTGTTGCTATTTTAAAAGGTAATTCATGGTGTAC |
| | TAGAGATACTGAGTGTGAGGGGACATGAGTGGTAGAAACAGTGGATATGTGTGGCAGTTTC |
| | TGACCTTGGTGTTTCTGTGTTTGCAGGTGTCCAATGTGAGGTGCAGCTGGTGGAGTCTGGG |
| | GGAGGCTTGGTACAGCCAGGGCGGTCCCTGAGACTCTCCTGTACAGCTTCTGGATTCACCT |
| | TTGGTGATTATGCTATGAGCTGGTTCCGCCAGGCTCCAGGGAAGGGGCTGGAGTGGGTAGG |
| | TTTCATTAGAAGCAAAGCTTATGGTGGGACAACAGAATACACCGCGTCTGTGAAAGGCAGA |
| | TTCACCATCTCAAGAGATGGTTCCAAAAGCATCGCCTATCTGCAAATGAACAGCCTGAAAA |
| | CCGAGGACACAGCCGTGTATTACTGTACTAGAGACACAGTGAGGGGAGGTCAGTGTGAGCC |
| | CAGACACAAACCTCCCTGCAGGGGCGCACAGAGCCACCAGGGGGCGCTAGGGACCGACTGA |
| | GTACGGGACAGGTCCCAGGAGCAGGTGCAGGGGGAGGTTTCCTTTTTCCTTGGCTGGAAAA |
| | GTCACCTTTATCTTCCCAGGACTCGAGCCTTCTAGGCTGTGATATTTTATTACTTGTATTT |
| | ACTGTTCATTTATTATCATTAGTTTTTAAATTTTGGTAATTTTTACAACTCTATGGATATA |
| | TTTTTAAGTGTATACTTTCAAGAAATAAACATTCCTAATTATTTGCACTGATTCTCCCAGA |
| | GTTTTATTAACATTTGTTGACATCAGCAACTACATAGCTATAGGGACAAACACTTTTAACG |
| | ATAGACAGTTGTTTAGGCCTGAAACCCCGTTTATACTAAAAATTTACAAAAATTAGCCTGG |
| | CGTGGTGAGGGGCGCCTGTAATCCCAGCTACTCGGGAGGCTGAGCAAGGAGAATTGCTTGA |
| | ATCCGGGAAGCGGAAGTCACAGTGAGTGGAGTGGACTGCCACTACACTCCAGCCTGGCGAC |
| | AGAGCGAGACTTTGTATGAAAGAAAGAAAAGAAAAGGAAGAAAGGAAGAAAGGAAGGAAGG |
| | AAGGAAGGAAGGAAGGAAGGAAGGAAGGAAGGAAGGAAGGAAAAGAGAGAAAGAAAGAGAG |
| | AGAAGCAAACAAATGGAAACTTAGACAAGGAAAGAGTAATAGAAAGACAGAAAAAATAAGT |
| | TCAATGTCAGAAGTACCTGAAAATTAATGTGCCTGGATCCTAGTTCTCTCCATATTTTCAG |
| | AAGAGTGCTGGAGGGCAGCAAAACCACACATGCTCTTATTACGGAATGTGGGTTCTGATAA |
| | AAACACTAGACACATCCAGCTTTGTCCTGGAGTTGGTTTAGGGGGATGTCAGAGACAGTGA |
| | TGAAGAGCACAGGGCCAGATACCGGGGTTCACTCATCCCAGACATGAGCTCCGAGACGCAT |
| | ACAGAGCCCCCCCATGTGTGGGTTTACTTCCACTTCTGTAAATGGAGAAAATATTGTCTCC |
| | TACAGAACATAGTTTACATGAATATTTAAAATGAAATAGGGTGATTAGTGCAAAGTGTTTA |
| | TCACAGCACAATTTCATAATAAGACAGCATATTTTCCAAATGCAATCATTGCCAGCAAACT |
| | TCTACAGGGCACCGTCGTCTTATCTGGGTACAGCCTACTCCTCAAGGGTCCCACCCTAGAG |
| | CTTGCTATATAGTAGGAGATATGCAAATAGGGCCCTCCCTCTACTGATGAAAACCAACCCA |
| | ACCCTGACCCTGCAGCTCTCAGAGAGGTGCCTTAGCCCTGGATTCCAAGGCATTTCCACTT |
| | GGTGATCAGCACTGAACACAGAGGACTCACCATGGAGTTGGGGCTGTGCTGGGTTTTCCTT |
| | GTTGCTATTTTAGAAGGTGATTCATGGAAAACTAGAGAGATTTAGTGTGTGTGGATATGAG |
| | TGAGAGAAACAGTGGATATGTGTGGCAGTTTCTGACCTTGGTGTCTCTTTGTTTGCAGGTG |
| | TCCAGTGTGAGGTGCAGCTGGTGGAGTCTGGGGGAGGCTTGGTACAGCCTGGGGGGTCCCT |
| | GAGACTCTCCTGTGCAGCCTCTGGATTCACCTTCAGTAGCTATAGCATGAACTGGGTCCGC |
| | CAGGCTCCAGGGAAGGGGCTGGAGTGGGTTTCATACATTAGTAGTAGTAGTAGTACCATAT |
| | ACTACGCAGACTCTGTGAAGGGCCGATTCACCATCTCCAGAGACAATGCCAAGAACTCACT |
| | GTATCTGCAAATGAACAGCCTGAGAGCCGAGGACACGGCTGTGTATTACTGTGCGAGAGAC |
| | ACAGTGAGGGGAAGTCAGTGTGAGCCCAGACACAAACCTCCCTGCAGGGGTCCCTTGGGAC |
| | CACCAGGGGGCGACAGCACACTGAGCACGGGGCTGTCTCCAGGGCAGGTGCAGGTGCTGCT |
| | GAGGGCTGGCTTCCTGTGATGGCCTGGGGCGGCCTCATTGTCAAATTTCCCCGGGGAACTT |
| | CTCCAGATTTACAATCCTGTACTAATATTTGATGTCTCTAAATGCAACCTTTTTTTTCCTT |
| | TTTGTGTCTGTTTTTTTTTTTTAAAACAGGAGGACACATCCTCACCTCCACAGAAGCCACA |
| | GTGTCACTTTGGGGGCAGAAATAATCCTTTCGTGGTCAACAGGGTGAGAGTTTTGAGGAAT |
| | CCCAGGGAAACCTGGGGAATGTTTTCCAATTAGACTCAGGGCAGAGACCTCCATGGGAATC |
| | TCTGATTAGAACAGGCTTTGAGTTCTGATGGGAGCCAAGAGAGAGGCTCACCCAGGGTCAG |
| | GGTTCTTAAAACCTGATGGTTTTCACAGCAATCCCCCTTCATCTTGTGAAACTGGGCACAT |
| | CTGACTCAGACTGATTCAGTTGACCCTCTTTCTGCTAATCCATTTTCCTTCCCAGTAGACT |
| | TGATTCTCACAGATCCCTTTCTTCTTCTCTTTCCTGAAAACAGAGGATGTGTTTTCTGTAG |
| | TCTAAATTCCAAGGCTCAGGTCCGCAGGTTATTTTGGGAACATGATTTCATAAAGAACTCC |
| | AAACTCTTGTTTCAACTTCTGACTCCTCGTTTCTGTGATATAAGAAAACCATTTCCAATTA |
| | TGCATCTCAGGGCAATTCTGTAAACCCAGAGCATTTCTGCTGAAGATCCTGGGGAATCAAG |
| | ACACCGGGCAGGTGATGGAGACACTGTCTCAGGTGCGCCCAACGAATCTCAGAGGAACCTG |
| | CTGGAGAGTCACGTGGAACATCTACAGTCAGTTTCTCAGAGTCAACAGTGAGCTGTGTTGG |
| | TGCCTGAGGGGACCATGATGGGGCCAAGGCACGTGCTCAGTGTCGTGGACAGTGATGGTCC |
| | AGAAATGATCTAGATGGTCTTGACGCTAATGAAATATGGGTTCAGAGTGAGGAGCATAATC |
| | TGTGGGGACTTGTTCTTCAGTGAAAGGATCCTGTCCGCAAACAGAAATGGAGCAGGACATG |
| | CATTTCTTCAAGCAGGATTAGGGCTTGGACCATCAGCATCCCACTCCTGTGTGGCAGATGG |
| | GACATCTATCTTCTTTCTCAACCTCGATCAGGCTTTGAGGTATGAAATAATCTGTCTCATG |
| | AATATGCAAATAACCTTAGATCTACTGAGGTAAATATGGATACATCTGGGCCCTGAAAGCA |
| | TCATCCAACAACCACATCCCTTCTCTACAGAAGCCTCTGAGAGGAAAGTTCTTCACCATGG |
| | ACTGGACCTGGAGGGTCTTCTGCTTGCTGGCTGTAGCTCCAGGTAAAGGGCCAACTGGTTC |
| | CAGGGCTGAGGAAGGGATTTTTTCCAGTTTAGAGGACTGTCATTCTCTACTGTGTCCTCTC |
| | CGCAGGTGCTCACTCCCAGGTGCAGCTGGTGCAGTCTGGGGCTGAGGTGAAGAAGCCTGGG |
| | GCCTCAGTGAAGGTTTCCTGCAAGGCATCTGGATACACCTTCACCAGCTACTATATGCACT |
| | GGGTGCGACAGGCCCCTGGACAAGGGCTTGAGTGGATGGGAATAATCAACCCTAGTGGTGG |
| | TAGCACAAGCTACGCACAGAAGTTCCAGGGCAGAGTCACCATGACCAGGGACACGTCCACG |
| | AGCACAGTCTACATGGAGCTGAGCAGCCTGAGATCTGAGGACACGGCCGTGTATTACTGTG |
| | CGAGAGACACAGTGTGAGAAACCACATCCTCAGAGTGTCAGAAACCCTGAGGGAGGAGTCA |
| | GCTGTGCTGAGCTGAGAAAATGACAGGGGTTATTCAGTTTAAGACTGTTTAGAAAACGGGT |
| | TATATATTTGAGAACAAAGAACAATAGAAACACAATCGAATTGTAAGAGAAATATTCCATT |
| | CAAGAGCCACCACATAAGCCAAACTGACAGAGTGGGAAAGGCCACACTCAGTAAAGTTGAT |
| | ACAAACATACCATAAAGGTGCTACTATGAACAAGTTTTTGAATTAGATGAATAAATCATTT |
| | GGAGCAAGGTTATTTGGTCATATGTTAAGAGTAAGCATGATTCTTACAAAGTGGGAAAATT |
| | GTCTTTCAAATGTTTCTGTCACTTCTTACCATAAAGTTCATTTTAGAGGTTTTAGGATTAC |
| | AGTGAAATTGCACAGAAGGTGTGAGAATTCCCATGAATCCCTGCCCCGCACGGACACCGCC |
| | TCCTCCACTACAGCCATCCTGCCCCACAGTCACAAATAAGTCACAATGGATGAATCTACAA |
| | GAACTCTTGGTTCTTTCTTTTTCTGGTGATCCCCTAATATAACAAGCCTAAATTATCTTGG |
| | AACACCCAGGTATTTTCAATGGCTTTCTAGAAGTGATATTAGTCAGAGGGAAAGTGAGTGA |
| | GGCTATTACTATTTGAGCACTTTCTTCCAAAATCCAGTTTCAGTGCTCACTGAATGGAGTT |
| | GGAAATAAAACCCACAATTATCCATAACACAAATTCCTTGGAAGTTATTTTGGGATAATGA |
| | GTTCATAACCTGTAGACCAAGAGTCCAAGAGTGTTTCTATTGAAGAGCCTGGGGGATCAAG |
| | ACACCAGGCAGGTGATGCAGACACTGTCTAAAGAGTGCCCAGCGGCTCTCAGAGGGACCTA |
| | CTGGATACTCACGTGGGACATCAGCAATCACTTTCTCAGAGTCACCAGTGAGCTGTGCTGG |
| | TTCCTGAAGGGTCCAGGATAGGGCCAAGGCACCTGCTCTGTGTCGTGGAGAGTGATTGTTC |
| | CAGAAATCATAGAGGTGGTCTCTATGCTTATAAAATCTATGTTCACAGTGAGAAGTCTGTT |
| | CTGAGAGGGCTTATTCTTCAGTGAAAGGACCTCTGCCCACAAATGTTCATAAATGGAGCAG |
| | GGCATTCATTTCCTCAAGCAGGATCAGGGCTTGAGTCATCAGCATCTCACTCTTGCAAGGC |
| | TGATGTGTCGTTTGTCTTCCCTTTCTTATCATCGACCAGGCTTTGAGCTATGAAATGCCCT |
| | GTCTCATCAATATGCAAATAACCTGAGATCGACTGAGGTAAATATGGATATGTCTGTGCCC |
| | TGAGAGCATCACCCAACAACCACATCCCTCCTCTAGAGAATCCCCTGAAAGCACAGCTCCT |
| | CACCATGGACTGGACCTGGAGAATCCTCTTCTTGGTGGCAGCAGCCACAGGTAAGGGGCTC |
| | CCAAGTCCCAGTGATGAGGAGGGGATTGAGTCCAGTCAAGGTGGCTTTTATCCACTCCTGT |
| | GTCCCCTCCACAGATGCCTACTCCCAGATGCAGCTGGTGCAGTCTGGGGCTGAGGTGAAGA |
| | AGACTGGGTCCTCAGTGAAGGTTTCCTGCAAGGCTTCCGGATACACCTTCACCTACCGCTA |
| | CCTGCACTGGGTGCGACAGGCCCCCGGACAAGCGCTTGAGTGGATGGGATGGATCACACCT |
| | TTCAATGGTAACACCAACTACGCACAGAAATTCCAGGACAGAGTCACCATTACTAGGGACA |
| | GGTCTATGAGCACAGCCTACATGGAGCTGAGCAGCCTGAGATCTGAGGACACAGCCATGTA |
| | TTACTGTGCAAGATACACAGTGTGAAAACCCACATCCTGAGACCGTCAGAAACCCCAAGGA |
| | GGAGGCAGCTTCACTGAATGAGGAGGTTACAGGGCTTACGATGTTTAAAGTTGTTCAGAAA |
| | ATAGGCTAAGCAATTGAGGAATATGAGTAATAGAAATATGTATGCACTCTATACAGGAAAT |
| | ATTTCTAATAATTGTCACCCTATATGCAAAATTCGCAGAGAGGTAAAAGCAGAAATCAGTC |
| | AAGCTGATGCAAAGTTCCCCACGTAGGCTTTGTGCAGATGTAAGTTCTAAAATCAGATAGA |
| | TAAATAATTTGGAGCAAGATTGCTTGATAACATGGCTAATGCTGAATATGATTCCTAAAAA |
| | CTGGCCAAAATATATTCCAATTTGTCTCTGCCACCTCTCTTACATAAAATGTATTAAAAAG |
| | TAGTTTTAAGACCACAGCAAAATTGAACAGAAGGTGCAGAGATTTCCTATGTGCCCCTGCT |
| | TCACACATGCACAGCCTTCCCCACTGTCACCATCCTGCCCCAGAGTCATCAATAAGTTACA |
| | ATGGATGAACTTACATGGATGGATTGGTTCTTTCCTCTTCCGGCGGTCCCTTGGCATACCA |
| | AGTCTAAACTATCTTGAAGCACAACAGGTTCTTCGAGTGGGTTCCTGGGAATGAAGCCAGT |
| | TAGAGGAAAAGTGGGTGGGGCTATTCCTATTAGGAGTCTTTTTTAATGTTTGTCAAATGGT |
| | GAACATATTGTGTGAAGATTATATGTCTGTATTACTTCATTAAAGCTATTATAAATAAAAG |
| | TCTAATGTGGTAGAAAAAGATGAAGAGAGAAATAAAAATAATACAAGAAAAGTCATGAACT |
| | CCTGAATGAATTAACCCTTAGTTTTTCTCTATTACTTATAAAAACACCAAGATACAGCCAA |
| | ATAATATCACGATATCATTATAAGAAGAGTGTTTTGTAAACCTCACTGGGAATTTATAGCT |
| | CTTTCCTAGAGTTAATTTTGGGAACAGTTGGATCCAATTGTGAGAAATGCAGGCTGGACAC |
| | TGAGACTGGCTCTTATGAGATGTGAGCTCTTGTCTATGTCACATGGTCCTTCCATACTTGG |
| | GGGTTTACATTCACATCTGTAAATGAAGGAAACATTGACTCTCAAAGAACATATTTCATGT |
| | GCATGTAAAAGTATGAATGCTAGTGAGAATTAATTACTTATGAAGTATAATCACCCACATC |
| | CACTCTTGGACACAGCCCACTCTGAGGCATCTGTTACAGAACTCATTATATAGTAGGAGAC |
| | ATGCAAATAGGGTCCTCCCTCTGCTGATGAAAACCAGCCCAGCCCTGACCCTGCAGCTCTG |
| | GGAGAGGAGCCCCAGCCCTGAGATTCCCAGGTGTTTCCATTCGGTGATCAGCACTGAACAC |
| | AGAGAACGCACCATGGAGTTTGGACTGAGCTGGGTTTTCCTTGTTGCTATTTTAAAAGGTG |
| | ATTCATGGATAAATAGAGATGTTGAGTGTGAGTGAACATGAGTGAGAGAAACAGTGGATAT |
| | GTGTGGCAGTGTCTGACCAGGGTGTCTCTGTGTTTGCAGGTGTCCAGTGTGAAGTGCAGCT |
| | GGTGGAGTCTGGGGGAGTCGTGGTACAGCCTGGGGGGTCCCTGAGACTCTCCTGTGCAGCC |
| | TCTGGATTCACCTTTGATGATTATACCATGCACTGGGTCCGTCAAGCTCCGGGGAAGGGTC |
| | TGGAGTGGGTCTCTCTTATTAGTTGGGATGGTGGTAGCACATACTATGCAGACTCTGTGAA |
| | GGGCCGATTCACCATCTCCAGAGACAACAGCAAAAACTCCCTGTATCTGCAAATGAACAGT |
| | CTGAGAACTGAGGACACCGCCTTGTATTACTGTGCAAAAGATACACAGTGAGGGGAAGTCA |
| | GCGAGAGCCCAGACAAAAACCTTGCTGCAGGAAGACAGGAGGGGCCTGGGCTGCAGAGGCC |
| | ACTCAAGACACACTGAGCATAGGGTTAACTCTGGGACAAGTTGCTCAGGAAGGTTAAGAGC |
| | TGGTTTCCTTTCAGAGTCTTCACAATTTCTCCATCTAACAGTTTCCCCAGGAACCCTGTCT |
| | AGATCTGTGATCTGGATCTGCTGAAACTGCCTGTGTCACCTTCCTCACCTGTGACTTTGGG |
| | GGAGCTGATTGTGGACACTCCAGTGTGTGGGATTTCTTGGTGACAGCAATTGTGTCTTCTG |
| | TCTAGGCATGTCTAGGGCTGGCCATCAGGAAGGGCAGGCTGGAATTTTTGGAAAGAGGCGC |
| | ACCTGCCATCCACCAGGAAATTTTGTTGTCTTTTGTTCTGCTAGAATTAAATCAGACACAC |
| | CAAGGTTAACTAGCACTATCTTCCTAGCTTGAGAAACTTGATGGCAGGCTTGAATAACACC |
| | TGTATGAAGCCATCAGAGCAACAACTAGATTAATGTCTGCTAGAATTAAATCAGGCACACC |
| | AAGGTTAACTAGCACTATCTTCCTAGCTCGAGAAACTTGATAGCAGGCTTGAATAACACTT |
| | GTATGAAACCATCAGAGCAACACCTAGAATAGTGTCTGATTTAATAATTGAGACTATGGTC |
| | TAGCCAAGGAGCAGAAGTGTTTAGAGGCCAGGACACATCTTCAAGGTCACACATTGAGAAG |
| | GATGGAGATATGTCCCACTACCTTCTCCTACGATCTCAGACAGAATCCCAAATTTCAAAAG |
| | GACACAGAAGGACAGCTCTCAGGTGCTTTTAAAAAATGACCCACTTCCAGGGACAGTGAGC |
| | TTCCCTGTAACCATGGTGGATGTTCTGAACTACAATAAACATTGGATGGATCCAGTATTGT |
| | TTGAAGTCACTGTCATTATTACATTCAGCTGTTGTTTCAATGTGTCTGAAAGGGTAAATGA |
| | CTATTTAGATGGCCTGGGTGTGTGGTTGGTTTTATATGAATCTTTAAGGGTTGAACAGTAC |
| | TGACCCTATTCCAAAATCTGTCCTTGATCCAGGATCACACTCATCTCTCAGACCAGCTCCT |
| | TCAGCACATCTCTTTACCTGGAAGAAGAGGACTCTGGGCTTGGAGAGGGGAGGCCCCAAGA |
| | AGAGAACTGAGTTCTCAAAGGGCACAGCCAGCATTCTCCTCCCAGGGTGAGCTCAAAAGAC |
| | TGGCGCCTCTCTCATCCCTTTTCACTGCTCCGTACAAACGCACCACCCCCATGCAAATCCT |
| | CACTTAGGCGCCCACAGGAAGCCACCACACATTTCCTTAAATTCAGGTCCAACTCATAAGG |
| | GAAATGCTTTCTGAGAGTCATGGATCTCATGTGCAAGAAAATGAAGCACCTGTGGTTCTTC |
| | CTCCTGCTGGTGGCGGCTCCCAGATGTGAGTGTTTCTAGGATGCAGACATGGAGATATGGG |
| | AGGCTGCCTCTGATCCCAGGGCTCACTGTGGGTTTTTCTGTTCACAGGGGTCCTGTCCCAG |
| | CTGCAGCTGCAGGAGTCGGGCCCAGGACTGGTGAAGCCTTCGGAGACCCTGTCCCTCACCT |
| | GCACTGTCTCTGGTGGCTCCATCAGCAGTAGTAGTTACTACTGGGGCTGGATCCGCCAGCC |
| | CCCAGGGAAGGGGCTGGAGTGGATTGGGAGTATCTATTATAGTGGGAGCACCTACTACAAC |
| | CCGTCCCTCAAGAGTCGAGTCACCATATCCGTAGACACGTCCAAGAACCAGTTCTCCCTGA |
| | AGCTGAGCTCTGTGACCGCCGCAGACACGGCTGTGTATTACTGTGCGAGACACACAGTGAG |
| | GGGAGGTGAGTGTGAGCCCAGACAAAAACCTCCCTGCAGGGAGGCTGAGGGGGCGGGCGCA |
| | GGTGCAGCTCAGGGCCAGCAGGGGGCGTGCGGAGCTCACGGAATACAAGGCCGGGTCAGGA |
| | GCAGGTGCAGGGTGAGCGGGGCTTGCTCATCTTCTCAGAGATCATCATCTCCCTCCTCGCC |
| | AGCACCTCAGCTTTCCGTAGAGGTCCTCTTTCTTTATTGTGTGTGGTTCTACTTCCTCACA |
| | TCCTTGTGCCAGGAAAGAAAGGAGTAAGGCAAATTTTCCTGTTACAATTGAAGTTTCACCA |
| | ATTACTAAGAACTTTCCTGCAAGTACCTGCACAGCCCATTATACCTTATTTATATATGTAT |
| | ATATTCTAATGCTTCTCACCATCTCTTGATTTGTGTCATCAATTTAATTGTGCCCTTTTTG |
| | AAATTCATATGCTGAAACTTTAAATCCAATGGATCTATATCGGAATTTTAATGGTATAATT |
| | AATGTTAAATGTGGTCATAAATGAGACCCTAATGCAATAGAGCTGTTGTCTTTATAAGAAG |
| | AGGAAGAGACACCAGATACCTCTCACTTCTCACATGCACTCAGAGAAGAGGCCACGTGGAG |
| | ACATAGTGCACTAGAAGGTGGGCCTCTGCAAGCCAGGAAGAAGCCGCACCAAGAACCAACC |
| | CTGCCAGCACCTTGATCTTCCAGAAATGTTTAGAGGTCAGGCCATATTCTCAGGGTTACAC |
| | ATTGAGAAGGATGGAGATATATTCTACTACCTTCTCCTGAGATCTCACACACAATCTCAAA |
| | TTTCAAAAGGTCTCAGAAGGGCAGCTCTCAGGTACTATTTAAAAATAACCCACTTCCTGGG |
| | ACAGGTAGCATCCTTCTAACCATGATGGATGTTCTGAACTACAGTACACATTGCATGGATC |
| | CAGGTTTGTCTCAATTCACTGTGATTATTACACTCAGCAGCTGTTTCAATATGTCTGAAGG |
| | GGTAAATGACAATTTAGGTGACCTGGGTGTATGGTTGGTGTTATATGAATCTTTAAATGTA |
| | GAACAGTATTAACTGTATTCCAAAATCTGTCTTTGATCCATGATCACACTTGTCTCCCAGA |
| | CCAGCTCCTTCAGCACATTTCCTACCTGGAAGAAGAGGACTCTGGGTTTGGTGAGGGGAGG |
| | CCACAGGAAGAGAACTGAGTTCTCAGAGGGCACAGCCAGCATACACCTCCCAGGGTGAGCC |
| | CAAAAGACTGGGGCCTCCCTCATCCCTTTTTACCTATCCATACAAAGGCACCACCCACATG |
| | CAAATCCTCACTTAGGCACCCACAGGAAATGACTACACATTTCCTTAAATTCAGGGTCCAG |
| | CTCACATGGGAAGTGCTTTCTGAGAGTCATGGACCTCCTGCACAAGAACATGAAACACCTG |
| | TGGTTCTTCCTCCTCCTGGTGGCAGCTCCCAGATGTGAGTGTCTCAGGAATGCGGATATGA |
| | AGATATGAGATGCTGCCTCTGATCCCAGGGCTCACTGTGGGTTTCTCTGTTCACAGGGGTC |
| | CTGTCCCAGGTGCAGCTACAGCAGTGGGGCGCAGGACTGTTGAAGCCTTCGGAGACCCTGT |
| | CCCTCACCTGCGCTGTCTATGGTGGGTCCTTCAGTGGTTACTACTGGAGCTGGATCCGCCA |
| | GCCCCCAGGGAAGGGGCTGGAGTGGATTGGGGAAATCAATCATAGTGGAAGCACCAACTAC |
| | AACCCGTCCCTCAAGAGTCGAGTCACCATATCAGTAGACACGTCCAAGAACCAGTTCTCCC |
| | TGAAGCTGAGCTCTGTGACCGCCGCGGACACGGCTGTGTATTACTGTGCGAGAGGCACAGT |
| | GAGGGGAGGTGAGTGTGAGCCCAGACAAAAACCTCCCTGCAGGTAGGCAGAGGGGGCGGGC |
| | GCAGGTACTGCTCAAGACCAGCAGGTGGCGCGCGGCGCCCACAGATCCCGAGGCCGGGTCC |
| | GGAGCAGGTGCAAGGAGGGGGGGGCTTCCTCAACAGCTCAGTGGTCTGTCTCCTCGCCAGC |
| | ACCTCAGATGTCCCCAGGACTCTCTTTCTTTATTATCTGTGGTTCTGCTTCCTCACATCCT |
| | TGTGGCAGGGAAGAAAGGAGGAAGACAATTTTTCTGTTTACTGTTGAGGTTTCACCAATTA |
| | CTAGAAACTTTCCTACAAGTTCCTGCATGACTCATTTTGCCATATATGGATTCTCACCATC |
| | TCTTGATTTGTTTCATCAATCGAATTGTGCCCTATTTGAAATTAACTTACTGAAACCTTAA |
| | ATCCAATGGATCTATACTGGAATTTAATGATGTAATTGAGGTTAAATGTGGTCAAAGTTGT |
| | GAGACCCTAATGCAATAAACCGTTGTCTTTATAAGAAGAGGAAGAGACACCAGAGACCTCT |
| | CACTTTTCACGTGCACACAGAGAAGAGGCCATGTGGAGACGTAGTGCACTAGAAGGTGGCC |
| | CTGTGCAAGCCAGGAAGAAGCCATACTAAGAACCAATTTTGCCAGCTCCTTGATCTTCGAC |
| | ATTCAGACTGTAGAATTGTAAGAATTACACTCGAATAAAGCAATAAAGAAACTGATGAGAT |
| | AATATTTGACTGAATTGCATCAATAAATAGATCGATATTAACACAAGGAATATAACTGATT |
| | TCCAAAAACATACACATGAACCATGGTTCACTCTGCGTATTTAGATAAATTACAGAAAGTT |
| | GTCATAACAGATGGGGAATCCTGCAGACTTCACTAGGCATGGGCCATGCTGCCCTGGAGTT |
| | GTCTCAGGGGAGCTGCCTCCTCCAGAGGTTAGAGCACAGGCCCAGGTAATAGGACTAAATT |
| | TTTAGATGTGTTATCTTAGACACACTGCACAACTGCTGTGTTCTCTATGTAAATTATCTCC |
| | TGTAAAATATAACATTGAAGCCTGCATTAAATATATTGTGTAAATATGTAAGAATAAAAGA |
| | AAGTTATGAGAGCTAAGTGTTAATCAAGGCACAAGCATATAAGATATAACTATATTTTCCT |
| | GAATGATGGAATTACTACCAGTCTCCCCCAGGACACTTCATCTGCCCTGAGCCCAGCCTCT |
| | CCTCAGATGTCCCACCCAGAGCTTGCTATATAGTGGGGGACATGCAAATAGGGCCCTCCCT |
| | CTACTGATGAAAACCAGCCCAGCCCTGACCCTGCAGCTCTGGGAGAGGAGCCCAGCACTAG |
| | AAGTCGGCGGTGTTTCCATTCGGTGATCAGCACTGAACACAGAGGACTCACCATGGAGTTT |
| | GGGCTGAGCTGGGTTTTCCTCGTTGCTCTTTTAAGAGGTGATTCATGGAGAAATAGAGAGA |
| | CTGAGTGTGAGTGAACATGAGTGAGAAAAACTGGATTTGTGTGGCATTTTCTGATAACGGT |
| | GTCCTTCTGTTTGCAGGTGTCCAGTGTCAGGTGCAGCTGGTGGAGTCTGGGGGAGGCGTGG |
| | TCCAGCCTGGGAGGTCCCTGAGACTCTCCTGTGCAGCGTCTGGATTCACCTTCAGTAGCTA |
| | TGGCATGCACTGGGTCCGCCAGGCTCCAGGCAAGGGGCTGGAGTGGGTGGCAGTTATATGG |
| | TATGATGGAAGTAATAAATACTATGCAGACTCCGTGAAGGGCCGATTCACCATCTCCAGAG |
| | ACAATTCCAAGAACACGCTGTATCTGCAAATGAACAGCCTGAGAGCCGAGGACACGGCTGT |
| | GTATTACTGTGCGAGAGACACAGTGAGGGGAGGTCATTGTGCGCCCAGACACAAACCTCCC |
| | TGCAGGAACGCTGGGGGGAAATCAGCTGCAGGGGGCGCTCAGGAGCCACTGATCAGAGTCA |
| | GCCCTGGAGGCAGGTGCAGATGGAGGCTGTTTCCTGTCAGGATGTGGGACTTTGTCTTCTT |
| | CTGACAGTTCCCCAGGGAACCTCTTAAATTTAGAAAACTGTGCCTAACAATGTCTTCTCTA |
| | TGCATATGAGGACCTTTTCTCCCTGGCACAAAATGCAGATTGACGCTGACACGGATGAAAA |
| | TTCCTCAACCATGGTCACAAGGATCAGAGTCCTGAGTAACCTCAGGGCTTCCTGGTGATTC |
| | TTCTCCAATCAGACCCAGGACAGGGACCTCCGTGAGATTCCCTGACTGGAACAGTCTTTAT |
| | GGATCCTGGTCACAGACAATAGAGAGGCTGAACCAGGGTCAGCGTCATGTAGAACGTCACA |
| | GATTTCACGTCTGATCCTTCTCCTGACACGAAAGTATGCAAATCAGTATCAGCACCGATCT |
| | GGTGCTTCTTTTGTTCCTAATCCATTTACTTTCTTTTTTCGTCGTTTTTCTCCTTTTTCCA |
| | TTTGTTTTTCCTGCTTTTTGCAAAAGGAAGATGTTTTCCCTGTGAGATGCAGGGGATGACA |
| | ATTTTGGGAGATGGCTGGAACATCCAATATCCTCAGGGCCGACCATCTTTCATTACAGGAA |
| | GAAAGTTCTGAAAATGTCACTGGGGTGAACCACATTGTGCTGGGCTTGGTTCAGGGAGCAG |
| | TCAGGCCCAGTGTTGTGACCTTCAACCACAGAATCCTGAGGCTGCCTCAAAGTCCCCATCA |
| | GTTCCCGGACTCGCTATGTTTCTCGATCGTATCAGTGCATCCGGAGCTCCCTGGTGGCTTT |
| | AGTGATTCCTTGCTTGCCATGCTGAGGTCTCGCTGTAGATTATGTTGGGTTTTCTAAGACC |
| | GTTTTGCTATTGTAAGATCCACTCCCTGGGACAGAGCGATTCCCTCTAAACCTGATGGAGG |
| | TTCTGAACTACAAATAACGTTAAGTGAATCCTGGTGTGTCTGAACTCAAGTGATTGTTACA |
| | TTAAGCTGCTGTTGCAATCTGTTTCCTCACCTGGGAAAAGAGGAGCCAGGACATAGTGAGT |
| | TGAGGCCCCAGGAAGATAACTGAATTCTCAGAGGGCACAGCCAGCATCCTCTTCCCAGGGA |
| | GAGTCTAAAAGACTGGGGCCTCCCTCATCCCTTTTCACCTGTCCATACAGAGGCACCACCC |
| | ACATGCAAATCTCACTTAGGCACCCACAGAAAACCACCACACATTTCCTTAAATTCAGGGT |
| | CCTGCTCACATGGGAAATACTTTCTGAGAGTCCTGGACCTCCTGTGCAAGAACATGAAACA |
| | CCTGTGGTTCTTCCTCCTGCTGGTGGCAGCTCCCAGATGTGAGTGTCTCAAGGCTGCAGAC |
| | ATGGAGATATGGGAGGTGCCTCTGATCCCAGGGCTCACTGTGTGTCTCTCTGTTCACAGGG |
| | GTCCTGCCCCAGGTGCAGCTGCAGGAGTCGGGCCCAGGACTGGTGAAGCCTTCACAGACCC |
| | TGTCCCTCACCTGTACTGTCTCTGGTGGCTCCATCAGCAGTGGTGGTTACTACTGGAGCTG |
| | GATCCGCCAGCACCCAGGGAAGGGCCTGGAGTGGATTGGGTACATCTATTACAGTGGGAGC |
| | ACCTACTACAACCCGTCCCTCAAGAGTCGAGTTACCATATCAGTAGACACGTCTAAGAACC |
| | AGTTCTCCCTGAAGCTGAGCTCTGTGACTGCCGCGGACACGGCCGTGTATTACTGTGCGAG |
| | AGACACAATGAGGGGAGGTGAGTGTGAGCCCAGACACAAACCTCCCTGCAGGGAGGCGGAG |
| | GGGACCGGCGCAGTTGCTGCTCAGGACCAGCAGGGGGTGCGCGGGGCCCACAGAGCATGAG |
| | GCCGGGTCAGGAGCAGGTGCAGGGAGGGGGGGGCTTCCTCATCTGCTCAGTGCTCTCCCTC |
| | CTCGCCAGCACCTCAGCTGTCCCCAGGGCTCCTCTTTCTTTATTATCTGTGGTTCTGCTTC |
| | CTCACATCCTTGTGGTAGGAAAGGAAAGAGGAAGGCAAATTTTCCTCTTAGAGTCAAAGTG |
| | TCACTAATTACTAGGAACTTTCCTACAAGTTCCTGAATGTCCCATTTTTCCTTCTTAATTA |
| | AAAAAAATATATATTCTAATACTTCTCACCATCTCTTGATTTGTGTCATCAGTTGAATTGT |
| | GCTGTCTTTGAAATTCAAATGCTGAAACCTTAAATCCAATTGATCTATATTGGAATTTTAA |
| | GGATGGAATTAAGGTTAAATGTGATCATAAGTCTGAGATTCTAATGCAATAGATCTGTTGT |
| | CTTTATAAGAAGTGGAAGAGTCACCAGAGACCTCTCACTTTTCCCGTGCACGCAGAGAAGA |
| | GGCCATGTGGAGACATAGTGGACTAGAAGGTGCAAGCCAAGAAGAAGCCGCACCAAGAACC |
| | AACCCTGCCAGCACCTGGACCTTGGACATTCAATTACACTCGAATAAAGCAATAAAGAAAC |
| | TGATGAGATAATATTTGACTGAATTGCAGCAATAAATAGATCGATATTAACACAAGGAATA |
| | TAACTGACTTCCAAAAACATACACATGAACCGTGGTTCACTCTGCGTATTTAGGTAAATAA |
| | CAGAAAGTTGTCATAACAGATGGGGAATCCTGCAGACTTCACTAGGCATGGGCCATGCTGC |
| | CCTGGAGTTGTCTCAGGGGAGCTGCCTCCTCCAGAGGTTAGAGCACAGGCCCAGGTAATAG |
| | GACTAAATTTTTAGATGTGTTATCTTAGACACACTGCACAACTGCTGTGTTCTCTATGTAA |
| | ATTATCTCCTGTAAAATATAACATTGAAGCCTGCATTAAATATATTGTGTAAATATGTAAG |
| | AATAAAAGAAAGTTATGAGAGCTAAGTGTTAATCAAGGCACAAGCATATAAGATATAACTA |
| | TATTTTCCTGAATGATGGAATTACTACCAGTCTCCCCCAGGACACTTCATCTGCCCTGAGC |
| | CCAGCCTCTCCTCAGATGTCCCACCCAGAGCTTGCTATATAGTGGGGGACATGCAAATAGG |
| | GCCCTCCCTCTACTGATGAAAACCAGCCCAGCCCTGACCCTGCAGCTCTGGGAGAGGAGCC |
| | CAGCACTAGAAGTCGGCGGTGTTTCCATTCGGTGATCAGCACTGAACACAGAGGACTCACC |
| | ATGGAGTTTGGGCTGAGCTGGGTTTTCCTCGTTGCTCTTTTAAGAGGTGATTCATGGAGAA |
| | ATAGAGAGACTGAGTGTGAGTGAACATGAGTGAGAAAAACTGGATTTGTGTGGCATTTTCT |
| | GATAACGGTGTCCTTCTGTTTGCAGGTGTCCAGTGTCAGGTGCAGCTGGTGGAGTCTGGGG |
| | GAGGCGTGGTCCAGCCTGGGAGGTCCCTGAGACTCTCCTGTGCAGCCTCTGGATTCACCTT |
| | CAGTAGCTATGCTATGCACTGGGTCCGCCAGGCTCCAGGCAAGGGGCTAGAGTGGGTGGCA |
| | GTTATATCATATGATGGAAGTAATAAATACTACGCAGACTCCGTGAAGGGCCGATTCACCA |
| | TCTCCAGAGACAATTCCAAGAACACGCTGTATCTGCAAATGAACAGCCTGAGAGCTGAGGA |
| | CACGGCTGTGTATTACTGTGCGAGAGACACAGTGAGGGGAAGTCATTGTGCGCCCAGACAC |
| | AAACCTCCCTGCAGGAACGCTGGCGGGAAATCAGCGGCAGGGGGCGCTCAGGAGCCACTGA |
| | TCAGAGTCAGCCCTGGAGGCAGGTGCAGATGGAGGCTGTTTCCTGTCAGGATGTGGGACTT |
| | TGTCTTCTTCTGACAGTTCCCCAGGGAACCTCTTAAATTTAGAAAACTGTGCCTAACAATG |
| | TCTTCTCTATGCATATGAGGACCTTTTCTCCCTGGCACAAAATGCAGATTGACGCTGACAC |
| | GGATGAAAATTCCTCAACCATGGTCACAAGGATCAGAGTCCTGAGTAACCTCAGGGCTTCC |
| | TGGTGAGTCTTCTCCAATCAGACCCAGGACAGGGACCTCCGTGAGATTCCCTGACTGGAAC |
| | AGTCTTTATGGATCCTGGTCACAGACAATAGAGAGGCTGAACCAGGGTCAGCGTCATGTAG |
| | AACCTCACAGATTTCACGTCTGATCCTTCTCCTGACACGAAAGTATGCAAATCAGTATCAG |
| | CACCGATCTGGTGCTTCTTTTGTTCCTAATCCATTTACTTTATTTTTTCGTCGTTTTTCTC |
| | CTTTTTCCATTTGTTTTTCCTGCTTTTTGCAAAAGGAAGATGTTTTCCCTGTGAGATGCAG |
| | GGGATGACAATTTTGGGAGATGGCTGGAACATCCAATATCCTCAGGGCCGGCCATCCACAC |
| | CGTGCTTTCATTACAGGAAGAAAGATCTGAAAATGTCACTGGGGTGAACCATATTGTGCTG |
| | GGCTTGGTTCAGGAAGCAGTCAGGCCCAGTGTTGTGACCTTCAACCACAGAATCCTAAAAA |
| | ATAATAAAAGAGGCTCCCCCAAAGTCCCCATCAGTTCCTGGACTCGCCATGTGTCTGGGCC |
| | GTATCAGTGCATCCGGAGCTCCCTGGTGGCTTTAGGGATTCCTTGCTTGCCATGCTGAGGT |
| | CTCGCTATAGATTATGTTGGGTTTTCTGAGGCCGTTTTGCTATTTAAGACCCACTCCCTGG |
| | CACAGAGCGATTCCCTCTAAACCTGATAGAGGTTCTGAACTACAAATAACGTTAAGTGAAT |
| | CCTGGTGTGTCTGAACTCACATGATTGTTACATTAAGCTGCTGTTGCAATCTGTTTCCTCA |
| | CCTGGGAAAAGAGGAGCCAGGACATAGTGAGTTGAGGCCCCAGGAAGATAACTGAATTCTC |
| | AGAGGGCACAACCAGCATCCTCCTTCCAGGGAGAGCCTAAAAGACTGGGGCCTCCCTCATC |
| | CCTTTTCACCTCTCCATACAGAGGCACCACCCACATGCAAATCTCACTTAGGCACCCAAGG |
| | GAAACCATCACACATTTCCTTAAATTCAGGGTCCTGCTCACATGGGAAATACTTTCTGAGA |
| | GTCCTGGACCTCCTGTGCAAGAACATGAAACACCTGTGGTTCTTCCTCCTGCTGGGGCAG |
| | CTCCCAGATGTGAGTGTCTCAAGGCTGCAGACATGGAGATATGGGAGGTGCCTCTGAGCCC |
| | AGGGCTCACTGTGGGTCTCTCTGTTCACAGGGGTCCTGTCCCAGGTGCAGCTGCAGGAGTC |
| | GGGCCCAGGACTGGTGAAGCCTTCGGACACCCTGTCCCTCACCTGCGCTGTCTCTGGTTAC |
| | TCCATCAGCAGTAGTAACTGGTGGGGCTGGATCCGGCAGCCCCCAGGGAAGGGACTGGAGT |
| | GGATTGGGTACATCTATTATAGTGGGAGCACCTACTACAACCCGTCCCTCAAGAGTCGAGT |
| | CACCATGTCAGTAGACACGTCCAAGAACCAGTTCTCCCTGAAGCTGAGCTCTGTGACCGCC |
| | GTGGACACGGCCGTGTATTACTGTGCGAGAAACACAGTGAGGGGAGGTGAGTGTGAGCCCA |
| | GACACAAACCTCCCTGCAGGGAGGCGGAGGAGCCGGCGCAGGTGCTGCTCAGGACGAGCAG |
| | GGGGCGCGCGGGGCCCACAGAGCAGGAGGCCGGGTCAGGAGCAGGTGCAGGGAGGGCGGGG |
| | CTTCCTCATCAGCTCAGTGCTCTCCCTCCTCCCCAGCACCTCAGCTGTCCCCAGGACTCCT |
| | CTTTCTTTATTATCTGTGGTTCTACTTCCTCACATCCTTGCTGCAGGTTTTAACAGTCACT |
| | GGCACCCTTTTTTCAACAAATTCCCACAAGGCCCATTTTACCTTCTTGACAAGCAAAATAT |
| | TCTAAGGCTTCTCACTATCCCTTGATTGGCGTCATCTACTGCATCGTGTCTTTCTCTTAAA |
| | TTCATGTGATGAAACCCCAATCCCAGTGGCTCTGTATTGGACAGAGGGATTTTAAGAGGGC |
| | AAATAAGTTAACTGTGGCCATAAATGTGGGACCCTAATCCAGTAGGACTCTTGTCCTTATG |
| | AGAAGTGGAAGACATCAGAGGCCTCTCTCTCCACATGCACACAGAGGAGAGTCCATGTGGG |
| | GATGCGCTGCATGAGAAGGTGGCCCTTTGCAAGCCTGGAAGAGGCCTCTCAAAAAACTTAC |
| | CCTTTCTGCACCCAGATCTTGGACGTCAAGACTCCAGAACAATAAGAAAATTAATATTTGT |
| | TGCTCACTCATGCGCTGGAAGAGGGATGTCAGAGATGCAGTCTACTCTTCCTAAAGGGCTG |
| | ACAGGGCATAAAGACAAGACTGATTATCCATGGAACATTCCTATAGGAATGCACCTATGTG |
| | CAGACACACAAATAAGCTGTGTGTCTTGCAGTGTGTAAATGGCTGAGATGCCATTTACACT |
| | TCTTTACACAATACATTTTTAAATGTTTTGTTGATGTATTCTCTATCATTCAAAAAAATCA |
| | TCACTTTCCCCCTAAACAAAGAGGATTTTTATCAGAACACTTATGGGAAGCCCCTTGCTCT |
| | CCCAGATTCCCACCCTCATTTCTCCTGAAGGAAGAAAGAAAACCATCTCTATTGATTTCCA |
| | CTCTCATCCTCTAGGACTAAAACCAGAAAGCTGCATGCTCCCTGGGTGAGCCTTAGAGAAG |
| | ACCCTGTCTGTAGGAGCAGGAATCTCAGAGCCTTGGCTGAGAGGCATGGTCCTGAAATGAG |
| | ATGGAGTCCCCCATGGAGATCCCACGTGGACGCCCACACCTGAGGGCTCACTGCTCCTCAC |
| | CACAGATGCACTCCCCTACTGAGTCCTGAGACCTGAGTGCACCCCATAGAGTAGGACTCAG |
| | ATGAGGGAATGCAAATCTCCACCAGCTCCACCCTCCTCTGGGTTCAAAAGCTGAGCACGGG |
| | GCCTCGCTCAGTGACTCCTGTGCCCCACCATGGACACACTTTGCTACACACTCCTGCTGCT |
| | GACCACCCCTTCCTGTGAGTGCTGTGGTCAGGGACTTCCTCAGAAGTGAAACATCAGTTGT |
| | CTCCTTTGTGGGCTTCATCTTCTTATGTCTTCTCCACAGGGGTCTTGTCCCAGGTCACCTT |
| | GAAGGAGTCTGGTCCTGTGCTGGTGAAACCCACAGAGACCCTCACGCTGACCTGCACCGTC |
| | TCTGGGTTCTCACTCAGCAATGCTAGAATGGGTGTGAGCTGGATCCGTCAGCCCCCAGGGA |
| | AGGCCCTGGAGTGGCTTGCACACATTTTTTCGAATGACGAAAAATCCTACAGCACATCTCT |
| | GAAGAGCAGGCTCACCATCTCCAAGGACACCTCCAAAAGCCAGGTGGTCCTTACCATGACC |
| | AACATGGACCCTGTGGACACAGCCACATATTACTGTGCACGGATACCACAGAGACACAGCC |
| | CAGGATGCCTCCTGTACAAGAACCTAGCTGCATCTCAGTGGTGCTCCCTCCCTACCTCTGC |
| | AGAACAGGAAAGTGTGACTGAGATGCCATTTCCTGCCAGGGCTTGTGTTTCCTATACCAAC |
| | CAGACTCAGAGCCCTGTCTGTTTTCCTATTCTGTACTATTAAATGGCATGTTCCCTGTTAG |
| | TGATTCACGCAAGCAGAGGCTGTATCCTGTTTGACAAAGATTGAGCATGAAAGATTCCCAT |
| | TACCTGGGCCACATGCATCACTGATATGTGGCCACTTATTTCTTGAGCTCATATCCTTCCA |
| | AGGGGCTGAATACAAATATCTATAACATAGGCCATCTTAAACTGCAGGAAGTAATGTTGGA |
| | GAAGAATGTGGAGATAAAAGAGCAGGATGATTAATTAAAATCCAGATACCATCTTTTTTTG |
| | CAGAGAAAAATTTACACTAATAAAGTAATTTTATGAAACAACAAGAAAGATGGAATGTGTC |
| | CTCATCATGGAGTGTCTCACTTGCAGTGCACAAATAAATTTCTAAAAATTTTATAGGGAAG |
| | GTGTGATAGATGAGGCTGATTTCCACACAGGGAGTGATTAAATACCCAGGTAAGTATAAAA |
| | TCCAACACTTGGAAAGGAAAATGCCTCAGCTTGACATCAGAAACCCAGCTCAGTAAGTCTG |
| | AGGAGCAATGCGGACAGGGAGCACCCAGCACAGGACCCAGCCCTGGGGCAGGTGCACAGGA |
| | GGCTGGGGCAGGGTTTTCTCTCAGGAATTGAATGTTCCTTATTTCAAAGCAATAATGACCT |
| | AACATTTAAATAAGAATTTAGCAAGTACTGATGTGCCTGTAAGTATTTTATTGTATACGCA |
| | GCCTATACCTAACCCAATAATTTAATGCAAACCGGATTTAAAAGGAGAAATGTCTAGGTCT |
| | TTCAAATGTATTTCTAGTTAGGAATTGAGGGCGGGTTTTATTAATTCAATGGGTGTTACTG |
| | TCCGGAGACACACTCATCCCAGAAGTTAGATGTGCAGAGGGCAAGGCTCAGGAAAAGTTCA |
| | GGTAGACAGTGAGCCATATGAACAGGGAATCACATTGAGGACCATGTCCTGTGAGATTCTG |
| | GGTTTCTGTGAGAGGAGTTCTGTCTTCATGGACTTCTGGGCATGTCAGAGGACAAATATTA |
| | AACAAAGTTCAGGGCAGGGAGCTCAGCATCCCACTGTGGCGTGGTCCACATGTCACCTATC |
| | TTCTTCCTCAGGGTGGCGTGGCCTGAGCTATGAAAAACCTGCCTCATGAATATGCAAATGC |
| | ACTGCTGTCTACCGAGGTAAATACAGATCTTTCCTTGCCCAGAGACCATCACACAACAGCC |
| | ACATCCCTCCCCTACAGAAGCCCCCAGAGCGCAGCACCTCACCATGGACTGCACCTGGAGG |
| | ATCCTCTTCTTGGTGGCAGCAGCTACAGGCAAGAGAATCCTTAGTTCCAGGGCTGATGAGG |
| | GGACTGGGTCCAGTTAAGTGGTGTCTCATCCACTCCTCTGTCCTCTCCACAGGCACCCACG |
| | CCCAGGTCCAGCTGGTACAGTCTGGGGCTGAGGTGAAGAAGCCTGGGGCCTCAGTGAAGGT |
| | CTCCTGCAAGGTTTCCGGATACACCCTCACTGAATTATCCATGCACTGGGTGCGACAGGCT |
| | CCTGGAAAAGGGCTTGAGTGGATGGGAGGTTTTGATCCTGAAGATGGTGAAACAATCTACG |
| | CACAGAAGTTCCAGGGCAGAGTCACCATGACCGAGGACACATCTACAGACACAGCCTACAT |
| | GGAGCTGAGCAGCCTGAGATCTGAGGACACGGCCGTGTATTACTGTGCAACAGACACAGTG |
| | TGAAAACCCACATCCTGAGAGCGTCAGAAACCCTGAGGAATGAGGCAGCTGTGCTGAGGCT |
| | GAGGAGATGACAGGATTTATGAAGTTTAAATCTGTTTAGAAAATGGGTTGAGTAATTGAGT |
| | AAAACAGCAATGGAAAGATGTGTACACTGTCATTATGTAGGAAATAGTATTTTCAACTGTC |
| | ACCCTATAAGTAACACTCACAGAGTGGGAAAGGCAGCCATCAATCAAGCTGATGCAAACAT |
| | TCCCATGGGGGCTTTGTGGGGACATACATTTTATAATTGGATCTATAAATCATTAGAAACA |
| | GGATTGCTTGATCTCATGGTAAGACTAAATATAATTTCCAAGAAGTGGCCGAAATTTCTTC |
| | CAAAATGTCTTTGCCCTTCCTTTTACACTGAATTTATTTTAAAGGAGTTTTAGGATCACAA |
| | CAAGTTTGAGTAGAAGAAACAGAGTTCCCATGCACTCCTGCCCCCCGACGCTCAGCCTTCT |
| | CTGCTATCAACACCCTGCACCAGTGTCATAAATCGGTTACAGAGGATGAATCTCCACAGAC |
| | ACATTGGTTGTTTCCTTTTCTGGTGGTCCCTGGTATAACAAGCCTCAACTATCTTGAAGCA |
| | CCCCAGGTTCAAGTGGATTCTTCAAGTGGGTTACTGGGAATGATGCCAGGTAGAAGGAAAG |
| | TGGGTGGGACCATTCCTCTTTGTGCTTTTCTTTATATTTGTTAGGTAATCACCACAGTGTG |
| | TACACATCACACCATGTTCCCATTACAGAGAAAAGGTTCTGCGAACCTCACGAGCTGTGAC |
| | CCCTGTGTGCTGGGCTTGGTTCAGGGAGAAGTCAGGTCCAGTGGTGAGAAGCACAGGCCCA |
| | GATGCCCAGGCTCACTCTGACCAAATGTGAGCACTGGGGACATTGTAAAACCCACCTGTGC |
| | TTTTGCTGATAATTTTTCATCTTTAACATGGAAATAATATTGATACTATATACCATGGTTT |
| | CTCTGCGTATGTAAAAATAAAAGATGATTGGTGCTAACTTTAAAAATATGCAGTTTATGTA |
| | GATCTATGGTACCTCAATAAAACTGTTTTAAAATAAAAATTACAAAATTATAAGATTTTTA |
| | GGTTTTAAGGTTTAAGTTTATCACAAAACAAACTGACAATAGGAAAGCACAATTTCCCAAT |
| | GCTTTCAATATCACAGATCTCCCCGAGGACATTCTGACATGCTCTGAGCCCCACTATCTCC |
| | AAAGGCCTCTCACCCCAGAGCTTACTATATAGTAGGAGATATGCAAATAGAGCCCTCCGTC |
| | TGCTGATGAAAACCAGCCCAGCCCTGACCCTGCAGCTCTGAGAGAGGAGCCCAGCCCTGGG |
| | ATTTTCAGGTGTTTTCATTTGGTGATCAGGACTGAACAGAGAGAACTCACCATGGAGTTTG |
| | GGCTGAGCTGGCTTTTTCTTGTGGCTAAAATAAAAGGTAATTCATGGAGAAATAGAAAAAT |
| | TGAGTGTGAATGGATAAGAGTGAGAGAAACAGTGGATACGTGTGGCAGTTTCTGACCAGGG |
| | TTTCTTTTTGTTTGCAGGTGTCCAGTGTGAGGTGCAGCTGTTGGAGTCTGGGGGAGGCTTG |
| | GTACAGCCTGGGGGGTCCCTGAGACTCTCCTGTGCAGCCTCTGGATTCACCTTTAGCAGCT |
| | ATGCCATGAGCTGGGTCCGCCAGGCTCCAGGGAAGGGGCTGGAGTGGGTCTCAGCTATTAG |
| | TGGTAGTGGTGGTAGCACATACTACGCAGACTCCGTGAAGGGCCGGTTCACCATCTCCAGA |
| | GACAATTCCAAGAACACGCTGTATCTGCAAATGAACAGCCTGAGAGCCGAGGACACGGCCG |
| | TATATTACTGTGCGAAAGACACAGTGAGGGGAAGTCATTGTGAGCCCAGACACAAACCTCC |
| | CTGCAGGAACGATGGGGGGGAAATCAGCGGCAGGGGGCGCTCAGGACCCGCTGATCAGAGT |
| | CATCCCCAGAGGCAGGTGCAGATGGAGGCTGTTTCCTGTCAGGGTGTGGGACTTCATCTTC |
| | TTCTGACAGTTTCTCTAGTGAACCTCTCTAACCTCAGAATTCTGTGCTTACTAATGTCATC |
| | TCTACGTATTTTTTAAAAGATCATTTTAATATGAGCACCTATTCTCACACGCACCAAATGC |
| | AGATTGACGCTTACAGAGATGAAAAGTTCTCAACCATGGTCACCAGGATGAGAGTCCTGAG |
| | GAAACTCAGGGGTGCCTGGTGACTCTTCCGCAATCAGTCCTGGGACAGAAACCTCAGGGAC |
| | AGCCTTTATGAGTTTTGATCACATCTAACAGAGAGGATGGGCCAGGGCCAGAGTCATGTAG |
| | AACCTCACAGGTTCCACGTCTGACCCTTCTCCTGACACTAAAGCCAATCAGCATCAGCACT |
| | GACCTGGTGCTCCTTTTGCTCCCAATCCATGTTCTTTCTTTGGAGAGTTTCTTCTCCCTTT |
| | TTTATTTGCTTTTCCTGTTTCCTGAAAAAGAAACAGATGGTCCCCTTGGTCCACATTCCAG |
| | GGCTCAAGGCATTTTCTTGGAGCTCAGGTGGGGCTCAGGCTGTGGCTCTAAAATGTATTAC |
| | AAGCAAATGGAAATTTAGACAAGCACGGAGTAATAGAAAGATACAAACATATGTTAAATGT |
| | CAGAAGTATCTGAAAATTAGTGTGTCTGGACCCTAGTTCTCTCCATATTTTCAGGTGAGTG |
| | CTGGTGGCAACATCACACATGCTCTTACTACAGAAAGTGGGTTCTGAAAACCACACCAGGC |
| | ACGTCCAGCTTTGTCCTGGAGTTGGTTTAGGGGGATGTCACAGCCAGTGACGAGGAGCACA |
| | GGGCCAGATACCAGCGTTCACTTATCCCAGACATGAACTCCTGGATGCATACAGAGCCCCC |
| | TCCACTTGTGGGTTTACTTCCACTTCTGTAAAAGGAGAAAATATTGACTCCTACAGAACAT |
| | AATTTACACAAATATTTACAGATGAAATAGGGTGATCAGGGCAAAGTGTTTATCACAGCAC |
| | AATTTCATAAGACAGCATATTTTCCAAATACCATCGTCAGCAAACATCTGCAGGGCACCGT |
| | CTTATTATCTGGGTACAGCCTATTCCTCCAGCGTCCCACCCTAGAGCTTGTTATATAGTAG |
| | GAGATATGCAAATAGGGACCTCCCTCTACTGATGAAAACCAACCGAACCCTGACCCTGCAG |
| | CTCTGAGAGAGGAGCCTTAGCCCTGGATTCCAAGGCCTATCCACTTGGTGATCAGCACTGA |
| | GCACCGAGGATTCACCATGGAACTGGGGCTCCGCTGGGTTTTCCTTGTTGCTATTTTAGAA |
| | GGTGAATCATGGAAAAGTAGAGAGATTTAGTGTGTGTGGATATGAGTGAGAGAAACGGTGG |
| | ATGTGTGTGACAGTTTCTGACCAATGTCTCTCTGTTTGCAGGTGTCCAGTGTGAGGTGCAG |
| | CTGGTGGAGTCTGGGGGAGGCCTGGTCAAGCCTGGGGGGTCCCTGAGACTCTCCTGTGCAG |
| | CCTCTGGATTCACCTTCAGTAGCTATAGCATGAACTGGGTCCGCCAGGCTCCAGGGAAGGG |
| | GCTGGAGTGGGTCTCATCCATTAGTAGTAGTAGTAGTTACATATACTACGCAGACTCAGTG |
| | AAGGGCCGATTCACCATCTCCAGAGACAACGCCAAGAACTCACTGTATCTGCAAATGAACA |
| | GCCTGAGAGCCGAGGACACGGCTGTGTATTACTGTGCGAGAGACACAGTGAGGGGAAGTCA |
| | GTGTGAGCCCAGACACAAACCTCCCTGCAGGGGTCCCCAGGACCACCAGGGGGCGCCCGGG |
| | ACACTGTGCACGGGGCTGTCTCCAGGGCAGGTGCAGGTGCTGCTGAGGGCTGGCTTCCTGT |
| | CATGGCCTGCGGCGGCCTCATTGTCAAATTTCCCCAGGGAACTTCTCCAGATTTACAATTC |
| | TGTACTGACATTTCATGTCTCTAAATGTAAAACTTTTTTGTCCTTTTTGTTTCTTTGTTTT |
| | TGTAACAGGAGGACACACCCTCACCTCCACAGAAACCACAGTGTCACTTTGGGGGCAGATG |
| | ATCCTTCTGTGGTCAGCAGGATGAAAGTTCCGAGGAATCTCAGGGGAACCCGAAGAGTGTT |
| | TGCCAGTTAGACTCAGGGCAGCGACCTCCACGGGAATCTCTGATTAGAACAGGCTTTGAGT |
| | TCTGATAGGAGCCAAGAGAGACGCTCACACAGGGTCAGGGTCCTTAAAGCCTGATGGTTTT |
| | CACAGCTATCCCCCCTGGTCTTGTAAAACTGTGTACATATGACTCAGACTGATTCACTTGA |
| | CCCCCTTTCTGCTAATCCATTTTCCTTCTCTGCATACTTGATTCTCACAATTCCCTTTCTT |
| | CTCTTCCCCGAAAACTGAGGATGTGTTTTCTGTAGTCTAAATTCCAGGGCTCAGGTCTGCA |
| | GGACCTGGGTACAAACGGTGAACATATTGTGTGAAGATTATTTGTCAATATTATCTCATTA |
| | AAACTATTACAAATAAAAGTGTATAATTTGGTAAAAAAAATGAAGAGAGAAATAAAAATAA |
| | TACAAGAAAAGTCATGAACTCCTGACTCCACACGTGAGTTAACCCTCAGTTTTTCTATGTT |
| | ACTTAGAAAAACACTAAGATACAGCCAAAGAATATCATGATATCTTTATAAGAAGAGGGTT |
| | TTGTAACCCTCACTCGAAATTTCTAGCTCTGTCCTAGAGTTAATTTAGGGAGCAGTCAGAT |
| | CCAGCTGTGAGAAACACGGGCCAGACAGTGAGACTGGCTTTTACCAGATGTGAGCTCTTAG |
| | ACATGTCACATGGCCCTTCCGTGCTTGGGGGTTTACCTTCACATCTGTAAATGAAGGAAAC |
| | TTTGACTCCCACAGAACATAATTCATGTGCATCTATAAGTAGAAATGCTAATGAGAATTAA |
| | TTATTTATGAAGTATAATCACTCGCATCCACTCTGGGACACAGCCTACTCTGAGGCATCCC |
| | TTCCAGAAGTCACTATATAGTAGGAGACATGCAAATGGGGTCCTCCCTCTGCCGATGAAAA |
| | CCAGCCCAGCCCTGACCCTGCAGCTCTGGGAGAGGAGCCCCAGCCCTGAGATTCCCACGTG |
| | TTTCCATTCAGTGATCAGCACTGAACACAGAGGACTCGCCATGGAGTTTGGGCTGAGCTGG |
| | GTTTTCCTTGTTGCTATTTTAAAAGGTGATTCATGGATCAATAGAGATGTTGAGTGTGAGT |
| | GAACACGAGTGAGAGAAACAGTGGATTTGTGTGGCAGTTTCTGACCAGGGTGTCTCTGTGT |
| | TTGCAGGTGTCCAGTGTGAGGTGCAGCTGGTGGAGTCTGGGGGAGGTGTGGTACGGCCTGG |
| | GGGGTCCCTGAGACTCTCCTTTGCAGCCTCTGGATTCACCTTTGATGATTATGGCATGAGC |
| | TGGGTCCGCCAAGCTCCAGGGAAGGGGCTGGAGTGGGTCTCTGGTATTAATTGGAATGGTG |
| | GTAGCACAGGTTATGCAGACTCTGTGAAGGGCCGATTCACCATCTCCAGAGACAACGCCAA |
| | GAACTCCCTGTATCTGCAAATGAACAGTCTGAGAGCCGAGGACACGGCCTTGTATCACTGT |
| | GCGAGAGACACAGTGAGGGGAAGCCAGTGAGAGCCCAGACACAAACGTCCCTGCAGGAAGA |
| | CAGGAGGGGCCTGGGCTGCAGAGGGCGCTCAGGACACATTAAGAACACAGTAAACTCAGGG |
| | ACCAGGTGCCCAGGGAGGTTAAGGGCTGGTTTCCTTTCAGAGACTTAGCTGTTTCTCCATC |
| | TAACAGTTTCCCCAGAACCCTGCCTAGATTTGTGATCTGTATCTGCTCAAACTATCTGTGT |
| | CACCTTCCTCACCTGTGACTTTGAGGAAGCTTAGTGTGGACATTCCAGTGGGTGGAGAGAT |
| | CTTGGTGACAGCAATTGTGTGTTCTGTCTAGGCATGTCTATGGCTGGCCATCAGGAACGAC |
| | AGGCTGGAATTCTTGGAAAGAGGCACACCTGCCATCCATCAGGAAATTTTGTTGTCTTCTG |
| | TTCTGCTAGAATTAAATCAGGCACGCCCAGGTTAACTACTACTATCTTCCTAGCTTGAGAA |
| | AATTAATGGTGGGCTTGAATAACACCTATATGAAATCATCAGAGCAACACCTAGATTCGAG |
| | TCTCATTTAATAATTGAGACTATGTTCTAGCCAAGGAGACACATAAAACTGGTGATTGCCG |
| | TGGTCAGTTCATATCATATATTTGTCAGTAGAATCTGGCTGGTATTATGAACAGCTTTGCC |
| | AACACATGTGTTAGTGTTGGTCTTCGGAGAAGCACATCTGCAGGGGGCGCTCAGGACACAC |
| | AGAGCACAGGCTTCCGCCCCAGAGCAGGTGAAGGAGGCTGGGGAGGGGTTCCTCTCAGGGC |
| | CTGGGACTTCCTTTAAAAAATCTAAAATAAGTATTTCACAAGGACTGCCGATGTTTATATA |
| | AATATCCTATTCAATTGTGAGCATTTATGAAACTCGATGTTGTAATGAGAACCACTTTTAC |
| | AATGGGAATTTCAAACTTCCCTAGACATCTTAATAGTAAGCAGCTGGAGGTCAGGAGGAGA |
| | TCCTTTCTTATAAATAAGTGCAATTTTTGGAGAAACACACTCATTCCCAAAATAGCACATT |
| | CACATATTAAGGTCTAGAAATGATTCGAGTTGCCCCTGAGACAGTCAAATGTGGGTTCTAA |
| | GTGAGGTGCGTGTCCTGGGGGAGCTTGTTCTCCAGTGGGGGAAGCTCTGTCAACACAGAGT |
| | TCAGGGATGGGTAGGGGATGCGTGGCCTCTAACAGGATTACGGCTTGAACCCTCAGCTTCT |
| | ACAATTGTGTCGTCCATGTGTCATGTATTTGCTCTTTCTCATCCTGGGTCAGGAATTGGGC |
| | TATTAAATAGCATCCTTCATGAATATGCAAATAACTGAGGTGAATATAGATATCTGTGTGC |
| | CCTGAGAGCATCACCCAAAAACCACACCCCTCCTTGGGAGAATCCCCTAGATCACAGCTCC |
| | TCACCATGGACTGGACCTGGAGCATCCTTTTCTTGGTGGCAGCAGCAACAGGTAACGGACT |
| | CCCCAGTCCCAGGGCTGAGAGAGAAACCAGGCCAGTCATGTGAGACTTCACCCACTCCTGT |
| | GTCCTCTCCACAGGTGCCCACTCCCAGGTTCAGCTGGTGCAGTCTGGAGCTGAGGTGAAGA |
| | AGCCTGGGGCCTCAGTGAAGGTCTCCTGCAAGGCTTCTGGTTACACCTTTACCAGCTATGG |
| | TATCAGCTGGGTGCGACAGGCCCCTGGACAAGGGCTTGAGTGGATGGGATGGATCAGCGCT |
| | TACAATGGTAACACAAACTATGCACAGAAGCTCCAGGGCAGAGTCACCATGACCACAGACA |
| | CATCCACGAGCACAGCCTACATGGAGCTGAGGAGCCTGAGATCTGACGACACGGCCGTGTA |
| | TTACTGTGCGAGAGACACAGTGTGAAAACCCACATCCTGAGGGTTTCAGAAACCCCAGGGA |
| | GGAGGCAGCTGCACTGAATTTGAGGAGATTACAGGGCTTACAATGTTTAAAGTTGTTTAGA |
| | AAATGAGCTGAGCAATTGAGGAATGTGAGTAATGGAAACATGGATGCACTCTATATAGGAA |
| | ATGTTTCTTTCAACAGTCACCCTATATGCAAAATTCAGAATGGTAAAGGCAGCAATCAGTG |
| | AGGCTGACGCAAATATTCCCATGGAGGCCTTGTGCAGACATACGTTTTAAAATCAGATAGA |
| | TAAATAATTTGGAACAAGATTGCTGGTAACGTGGCTAAGACTAAATATGATTCCTAAAAAC |
| | TGGCCAAAATCTATTCCAAATTGTCTCTGCCACTCCTTTTACATAAATGTATTAAAAAGTA |
| | GTTTTAAGACCACAGCAAAATTGGACAGAAGGTGCAGAGAGTTCTCATGTGCCCCTGCTTC |
| | ACCATGCACAGCCTTTCCCACTGTCACCATCCTGCCCCAGAGTCATCAATAAGTTACAATG |
| | GATGAACTTACATGGGCGGATTGGTTCTTTCCTCTTCTGGTGGTCTCTTGGCATACCAAGC |
| | CCAAATTATCTTGAAGCACCATAGGTTCTACTGTAATGCCTAACCTTGTTTTTTACTCTAG |
| | TTTGCTACTTTAAATTTTCCCTTTTTTTGTCTCCTTAATTGCCAAGCAAACAAATGAAAAC |
| | TTGCACAAGAAATAAGTGACATAAAGATAGAAAAAATATTAAATTTCAGAAACACCTAATA |
| | ATTTATCTTCGTGAACCCTAGTTCTCACCATATTTTTAGGTGAATGCTAGAATGCAGCAAA |
| | ATTACACATGCTCTCAATACAGAAAGTGGGTTTCACAAACCACACTAGGCATGCTCAGCTC |
| | TGTCCTGGAGTTGGGTTAGGGAGTAATATAGGGCCAGTGGATGAGGAGCACAGGCCTAGAT |
| | ACTGGGGCTCACTAACCTCAGGTATGAGCTCTTAGATACATACAAAGCCCCTCCACGCATG |
| | GGTTTACTTCCCCATCTGTAAATTGAGAAACCATTGACCCCTAAAAATATGATTTACACAA |
| | ATATGTAAAAATGTAAGAGAGTGATTAGTGCAAAGTGTTTATCACAGCACAATTTCATAAC |
| | AAGACAGCAAGTTTTCCAAACAGCATCATTGTCATTAGATTCCTGCAGGGCATCATTACCT |
| | TATCTGGGCCCTGCCCTCTGCTCAGGCATCCCACCCCAGAGCTTGCTATATAGTAGGTGAC |
| | ATGCAAATAGGGCCCTCCCTCTCCTGATGAAAACCAGCCCAGTCCTGACCCTGCAGCTCTG |
| | GGAGAGGAGCCCCAGCCTTGGGATTCCCAAGTGTTTTCATTCAGTGATCAGGACTGAACAC |
| | AGAGGACTCACCATGGAGTTTGGGCTGAGCTGGATTTTCCTTGCTGCTATTTTAAAAGGTG |
| | ATTTATGGAGAACTAGAGAGATTAAGTGTGAGTGGACGTGAGTGAGAGAAACAGTGGATAT |
| | GTGTGGCAGTTTCTGATCTTAGTGTCTCTGTGTTTGCAGGTGTCCAGTGTGAGGTGCAGCT |
| | GGTGGAGTCTGGGGGAGGCTTGGTAAAGCCTGGGGGGTCCCTTAGACTCTCCTGTGCAGCC |
| | TCTGGATTCACTTTCAGTAACGCCTGGATGAGCTGGGTCCGCCAGGCTCCAGGGAAGGGGC |
| | TGGAGTGGGTTGGCCGTATTAAAAGCAAAACTGATGGTGGGACAACAGACTACGCTGCACC |
| | CGTGAAAGGCAGATTCACCATCTCAAGAGATGATTCAAAAAACACGCTGTATCTGCAAATG |
| | AACAGCCTGAAAACCGAGGACACAGCCGTGTATTACTGTACCACAGACACAGTGAGGGGAG |
| | GTCAGTGTGAGCCCGGACACAAACCTCCCTGCAGGGGCGCGCGGGGCCACCAGGGGGCGCT |
| | CGTGACCCACTGAGGGCGGGACAGGTCCCAGGAGCAGGTGCCGGGAGAGGTTTCCTTTCTC |
| | CTCAGCTGGAAAAGTCAGGTTTATCTTCGCAGGACTCTGGAGTCTTCTAGGCTGTGATATT |
| | TTGTTACTTATATTTATTATGAATTTTATCATTAATACTTAAATTTTAGTAATTATTAACA |
| | TTCTACATATTATTATATTTTTAAGTATATACTTTCAAGAAATAAACATTCCTAATTGCAT |
| | AATAATTATTGTAACATAATTATTGTAACATATAATTATATCCTAAATGCATAATAATTAT |
| | TGTAACAATCACATTCTCATTTTGTATTTGAATTTTCATATTTATTTTACTAAGATTTTAA |
| | TTTTAATATAAAATATTTTTCTACAATTGTCTGTTTTCCATTTTTGTGAGATAAAATTAGC |
| | ATATACAAAAATGCACAGATTCTCCAGGTACAGTTAGAGGGTTTCAGGCAAATGTGCACAT |
| | ACTTGTCTACAGCAGCTAAGTGAGGGTGAGGAACAGGTGAGGTCCATCTCCCCACAAAGTG |
| | TCCTCTTAGTGCTTCCAGTTAGTTCTCACATAAGGATTTTTTTTTATTTCAACTTTATAAT |
| | TTAGATACAGAGGGTATTTATTACAAGTAAACAAGTGAAATTTTAGACAAAAAAGGGATGA |
| | TAAGAAGGAACAAATAAATACATTAAATGTCAGATACACCAAAAATGTATCTGCCTGACGC |
| | CTAGTTGTCTCTGTATTTTTAGGTAAATGCAGCAAAATCACACAGGTTGTCGTGGCAGGAA |
| | GTGGATTCTGCAAACCACACTAGGCCGTTTATCTCTGTCCTGTAGTTGGTTCAGAGCAACT |
| | GAGGCCAGCTGTGAGGCGCATAGGCCCAGGTACTAGGACTCACTCATGCCAGATATAAGCC |
| | CTGAGACACGTACATAGCCCCTCCATGTGTGGGTTCACTTTTACATCTGTACATGAAGAAA |
| | CCACTGACTCCTAAATAACATCATCTATACACATAGGTAAAAAAATTAAAAATATGATAGT |
| | TGTTAAATGTTTATCGCAGAACAATTTCAAAATAAGGCAGCATTTTCCCAAATACAATCAT |
| | TGTCATCAAAATCCCCCAGGACGCTCTCATCTACTCTGCGCCCTGCCTTCACCTCAGATGT |
| | CCCACCCCAGAGCTTGCTATATAGTAACAGACATGCAAATAGTTGACTCCCTCTCCTGATG |
| | AAAACCAGCCCAGCCCTGACCCTGCAGCTCTGGGAGTGGAGCCCCAGCCTTGGGATTCCCA |
| | AGTGTTTGTATTCAGTGATCAGGACTGAACACACAGGACTCACCATGGAGTTGGGGCTGAG |
| | CTGGGTTTTCCTTGTTGCTATATTAGAAGGTGATTCATGGAGAACTAGAGATATTGAGTGT |
| | GAATGGGCATGAATGAGAGAAACAGTGGGTATGTGTGGCAATTTCTGACTTTTGTGTCTCT |
| | GTGTTTGCAGGTGTCCAGTGTGAGGTGCAGCTGGTGGAGTCTGGGGGAGGCTTGGTACAGC |
| | CTGGGGGGTCCCTGAGACTCTCCTGTGCAGCCTCTGGATTCACCTTCAGTAGCTACGACAT |
| | GCACTGGGTCCGCCAAGCTACAGGAAAAGGTCTGGAATGGGTCTCAGCTATTGGTACTGCT |
| | GGTGACACATACTATCCAGGCTCCGTGAAGGGCCGATTCACCATCTCCAGAGAAAATGCCA |
| | AGAACTCCTTGTATCTTCAAATGAACAGCCTGAGAGCCGGGGACACGGCTGTGTATTACTG |
| | TGCAAGAGACACAGTGAGGGGAAGTCAGTATGAGCCCAGACACAAACCTCCCTGCAGAATG |
| | CCTGGGGGAAATCAGCTGCAGGGGGCGCACAGGACCCACTGATCAGAGTCATCCCCAGAGG |
| | CAGGTGCAGATGGAGGCTGGTTTCCTGTCAGGATGTGGGACTTCATCTTTTTAGAGTTTCT |
| | CTAGGGAACCTCTCTAAGTTCAGAATTCTGTGCTTACCAATGCCATCCCTACATATTTTTA |
| | AAATGATTATTTTAATATGAAAACCTATTCTCTTATGCACGAAACACAGACTGATGCTTAC |
| | AGAGATGAAAAGCCCTCAACCATTGTCACCAGGATCAGAGTATTGAGGAAACTCAGGGGTA |
| | CCTGGTGGGTCTTCTCCACTCAGACTCAGGACAGAAACCTCAGTGAGTTTCCCTGACTAGG |
| | ACGGTCTTTAGGAATTGTGATCGCAGCCAATAGAGAGTCTGGGCAAGGATCAGTGTCATGT |
| | AGAACTTCACAGGTTTCACTTCTGACCCTTCTCCTGACACTAAAGTATACAACTTAGTGTC |
| | AGCACTGATCTGGGGCCCCTTTTGCTCTTAGCCCATTCTATTTCTTTTTATTTGTTGTTGT |
| | TGTTCTTGCTTTTCCTTGTAGTGTTCCTGCTCCCTGTAAAGTGGGGATGTGGCTCTTGCTG |
| | CCAAAGCTCCAGGTCTCAAGCCCATTCCCTGCAGCTCAAAGCAGTAACAAAAATGAGTATA |
| | TTGGCTGAGGAAGAGCAAGAAAGAGATGAATATTGACACTTGAATAATCACGGACTCCTGA |
| | AAATACACACATGTGAACACTGATTGCATATTTCAGGTAAATACTAGAAAAAGCAAAGTCA |
| | CATAAAGTCGTTATGACAGGTGGGATACCCTGCAAATCACACTAGGCATGTCCCACACTGC |
| | CCTGGAGCTGTCTCAGGGGAGCAGTCTCCTCCAGTGTTTAGAGGCACAGGCACGGATAATA |
| | GGGCTGACGCTGTCCAGATGTGTGATATTGGACACATTGCACAACTGCTCTGTTATGTATG |
| | TAATTCATCTTCTCTAAAAATGTAACATTGACACTTGCACTGAATATATTCTGCAAATATG |
| | TAAACATTAAATAAGATGATGACTGCTAATTGATCATCAAGTCACAATCACATAATCTGAA |
| | GTTATATTTTCCTGAGAGATAGGATTACCTCCAGTGTTTTCCGGGACCCTCTCATCTGCTC |
| | TGGGCACTGCCCTCTCCTCCAGCGTCCCACTAGAGCTTGCTATATAGTAGGAGACATGCAA |
| | ATAGGGCCCTCCCTCTGCTGATAAAAACCAGCCGAGCCCAGACCCTGCAGCTCTGGGAGAA |
| | GAGCCCCAGCCCCAGAATTCCCAGGAGTTTCCATTCGGTGATCAGCACTGAACACAGAGGA |
| | CTCACCATGGAGTTTGGGCTGAGCTGGGTTTTCCTTGTTGCTATAATAAAAGGTGATTTAT |
| | GGAGAACTAGAGACATTGAGTGGACGTGAGTGAGATAAGCAGTGAATATATGTGGCAGTTT |
| | CTGACCAGGTTGTCTCTGTGTTTGCAGGTGTCCAGTGTCAGGTGCAGCTGGTGGAGTCTGG |
| | GGGAGGCTTGGTCAAGCCTGGAGGGTCCCTGAGACTCTCCTGTGCAGCCTCTGGATTCACC |
| | TTCAGTGACTACTACATGAGCTGGATCCGCCAGGCTCCAGGGAAGGGGCTGGAGTGGGTTT |
| | CATACATTAGTAGTAGTGGTAGTACCATATACTACGCAGACTCTGTGAAGGGCCGATTCAC |
| | CATCTCCAGGGACAACGCCAAGAACTCACTGTATCTGCAAATGAACAGCCTGAGAGCCGAG |
| | GACACGGCCGTGTATTACTGTGCGAGAGACACAGTGAGGGGAGGTCAGTGTGAGCCCAGAC |
| | ACAAACCTCCCTGCAGGGGTCCCCAGGACCACCAGGGGGCGCCCGGGACACTGTGCACGGG |
| | GCTGTCTCCAGGGCAGGTGCAGGTGCTGCTGAGGGCTGGCTTCCTGTCATGGCCTGGGGCT |
| | GCCTCATTGTCAAATTTCCCCAGGAACTTCTCCAGATTTACAATTCTGTACTAACATTTGA |
| | TGTCTCTAAATGCAATACTTTTTTTGTCCTTTTTGTTTCTTTGTTTTTTTGCAACAGGAGT |
| | ACATATCCTCAGCTCCACAGAAGCCAGGGTGTCACTTTGGGGGCAGAAATAATCCTTTCAT |
| | GGTTACCAGGATAAGAGTCCTGAGGAATCCCAGGGAAACCTGGAGAGTGTTTTCCAGTTAG |
| | ACTCAGGGCAGAGACCTCCATGGGAATCTCTGATTAGAACAGGCCTTGAGCTCTGACGGGA |
| | GCCAAGAGAGAGGCTCGCCCAGGGTCAGAGTCCTTAAAACCTGATGGTTTTCACAGCTATC |
| | CCCCCTCGTCTTGTAAACTCAGACTGATTCAGTTGACCCTCTTTCTGCTAATCCATTTCCT |
| | TCTCTGTAGGTTTGATTCTCACAGTTCGCTTTCTTCTTCTCTTCCCTGAAAACAGACGATG |
| | TGTTTTCTGTAGTCAAAATCCCAGGGCTCAGGTCTGCAGGACCTGGGTAGGCTACGGGTCA |
| | AATTTTGAATGTATTGTTTGAAGATTGTTATTTGTCAATATTACCTCATTAAAACTATTAA |
| | AAATGGAAGTGTATAATTTGGTAGAAAAGGATTAAAAGAAAGATAAAAATAATACAAGAAA |
| | AGTCACAGACTCCTGAATCCACATGTGAATTAGCACTCAGTTTTTCTATATTATTTATAAA |
| | AACACTAAGATACAGCCAAATAATATCATGATATCCTTATAAGAAGAGTGTTTCGTAAACC |
| | TCACTGGGAATTTCTAGCTCTGTCCTAGAGTTAATTTAGGGAGCAGTCGGATCCAGTCCTG |
| | AGCAACACAGGCCAGACACTGAGACTTACCACATCTTACCAGATGTGAGCTCTTAGACACG |
| | TCACATGGCCTCTCCATGCTTGGGGGTTTACCTTCACATCTGTAAATGAAGGAAACATTGA |
| | CTGCCACAGAACATACTTCATGTGCATCTATACAGTAGAAATGCTAATAAGAATTAATTGT |
| | TTATGAAGTGTAATCACTCTGGGACACAGCCCACTCAGAGGCATCCCTTCCAGAACCCGCT |
| | ATATAGTAGGAGACATGCAAATAGGGCCCTCCCTCTGCTGATGAAAACCAGCCCAGCCCTG |
| | ACCCTGCAGCTCTGGGAGAGGAGCCCCAGCCCTGAGATTCCCAGGTGTTTCCATTCAGTGA |
| | TCAGCACTGAACACAGAGGACTCACCATGGAGTTGGGACTGAGCTGGATTTTCCTTTTGGC |
| | TATTTTAAAAGGTGATTCATGGAGAAATAGAGAGATTGAGTGTGAGTGGACATGAGTGGAT |
| | TTGTGTGGCAGTTTCTGACCTTGGTGTCTCTGTGTTTGCAGGTGTCCAGTGTGAAGTGCAG |
| | CTGGTGGAGTCTGGGGGAGGCTTGGTACAGCCTGGCAGGTCCCTGAGACTCTCCTGTGCAG |
| | CCTCTGGATTCACCTCTGATGATTATGCCATGCACTGGGTCCGGCAAGCTCCAGGGAAGGG |
| | CCTGGAGTGGGTCTCAGGTATTAGTTGGAATAGTGGTAGCATAGGCTATGCGGACTCTGTG |
| | AAGGGCCGATTCACCATCTCCAGAGACAACGCCAAGAACTCCCTGTATCTGCAAATGAACA |
| | GTCTGAGAGCTGAGGACACGGCCTTGTATTACTGTGCAAAAGATACACAGTGAGGGGAAGT |
| | CAGCGAGAGCCCAGACAAAAACCTCCTGCAGGAAGACAGGAGGGGCCTGGGCTGCAGAGGG |
| | CACTCAAGACACACTGAAAACACGGTTAACACTGGGACAAGTTGCTCAGGAAGGTTAAGGG |
| | CTGGTTTCCTTTCAGAGTCTTTGCTGTTTCTGTATCTAACAGTTTCCCCAGGAACCCTGTC |
| | TAGATTTGCGATCTTGATCTGCTTAAACTGTCTCTGTGTCATTTTTCTTACCTGTGACTTT |
| | AGGGGAGCTGATCGTGGACACTCCAGTGTGTGGAGATTTCCTGGTGACAGCAATTGTGTGT |
| | TCTGTCTAAGCATGTCTAGGGCCGGTCATCAGGAAGGACAGGAAGGACAGGCTGGAATTCT |
| | TGGAAACAGCTGCACCTGCCATCCACCAGGGAACTTTATTGTCTTCTGTTCTGCTAAAATT |
| | AAATCAGGCACACCAAGTTTAACTAGCACTATCTTCCTAGCTTAAGAAAATTGATGGCAGG |
| | TTTCAATAACACCTATATTAAACCTTCAGAGCAACACCTACATTAGTGTCTGATTTGATAA |
| | TTGAGACTATGTTCTAGCCAAGCAGACACATAAAACTGGTGATTGCCATGCTTAGTTCATA |
| | TTTCATATTTATCAATAGAATCTGGCTGGTGTTATAAACAGATTTGCCAACACATGTGTGT |
| | TAGTGTTTGTCTTCAAATTCCTATTTCTATTTAAACTCCCGATTCCTACTCTCAGTGGGAG |
| | GGAAAACTCACAGCCAATCACACATCACAGGACAAATCTGTAAACGAAGAGTCATTCCTCT |
| | GAAGGTCCTGGGTGTTCAGGACTCTCAGGCAGGTGCTGAGGACCCTGTCTTGGGAGTGCCC |
| | AGCAGATCTCAGAACCCTACATGGGGCCTGCTGGACACTCATGTGGGATAACTAGTCGCCA |
| | CTTATTCAGAGTTACCAGTGAGCTTTGACTGTTCCGAATGGGACCAGCATGGAGTCAAGGT |
| | GCCTGCTCAATGTCAGAGACAGCGATGGTCTCAGAAACAATCCAGGTAATCTCTAGGCCAA |
| | TAAAATGTGGATTCACAGTGAGAAGTACATCCTGGAGGTGGAGCTTGTTCTTCAGTGGGAA |
| | GAGTGCTGTGCACAGAAAGCTTAGAAATGGGGAAGGGGGTGCGTTTCCTCAGGCAGGATTA |
| | GGGCTTCGTCCCTCAGCGTCCCACTCTTGTATGGCTGATGTGGCATCTGTGTTTTCTTTCT |
| | CATACTAGATAAGGCTTTGAGCTGTGAAATACCCTGCCTCATGAATATGCAAATAACCTGA |
| | GCTCTTCTGAGGTAAATATAGGTATATTGGTGCCCTGAGAGCATCACTCAACAACCACATC |
| | TGTCCTCTAGAGAAAACCCTGTGAGCACAGCTCCTCACCATGGACTGGACCTGGAGGATCC |
| | TCTTCTTGGTGGCAGCAGCTACAAGTAAGGGGCTTCCTAGTCTCAAAGCTGAGGAACGGAT |
| | CCTGGTTCAGTCAAAGAGGATTTTATTCTCTCCTGTGTTCTCTCCACAGGTGCCCACTCCC |
| | AGGTGCAGCTGGTGCAGTCTGGGGCTGAGGTGAAGAAGCCTGGGGCCTCAGTGAAGGTCTC |
| | CTGCAAGGCTTCTGGATACACCTTCACCAGTTATGATATCAACTGGGTGCGACAGGCCACT |
| | GGACAAGGGCTTGAGTGGATGGGATGGATGAACCCTAACAGTGGTAACACAGGCTATGCAC |
| | AGAAGTTCCAGGGCAGAGTCACCATGACCAGGAACACCTCCATAAGCACAGCCTACATGGA |
| | GCTGAGCAGCCTGAGATCTGAGGACACGGCCGTGTATTACTGTGCGAGAGGCACAGTGTGA |
| | AAAACCACATCCTCAGAGAGTCAGAAACCCTAGGGGAGAAGGCAGCTGTGCTGGGCTGAGG |
| | AGATGACAGGGGTTATCAGGTTTAAGGCTTTTTTGAAAATGGGTTATATATTTGAGAAAAA |
| | AATAACAATAGAAACAAGTACACACTCTAATTTTAAGAGATATATTCAATTCAAGAATTGT |
| | AGAAGCCGAATTCACAGTGGGAAAGGCCACACTCAATAAAGTTGATAAAAACATTCCAGGA |
| | AGGTGCTACTGTGAACAAGTTTTCAAATTAGATGAATAAATAATTTGGAGCAATGTTATTT |
| | GATCATGAGGTGAGACAGTATGATTCTTAAAAAGTGGAAAAAATTCTTTCAAATGTTTCTG |
| | CCACTCTTTATCATAAAATTAATTTTAGAGCAGTTTTAGGATTACAATGAAATTGCACAGA |
| | AGGCATGAGAATTCCCATGACTCCCTGCCCTACACAGGCAGAGCCTCCTCCACTATGACCA |
| | TCCAGCACCACAGTCACAAATCAGTTACAATGGAGGAATCTCCAAGGACATTTGATTCTTT |
| | CTTTTTTGGTGATGCCCTAATATAACAAGCCTAATTTATCTCGGAATACCACAAGTTTTGT |
| | TCAGTGGATTTCTAGGAATCATATTAGTTAGAGGGAAAGTTGGTGAGGCTCTTACTATTTG |
| | AACTCATTCTTCCAAAATCCACAAAGGTTTTTTGGTAAGACCCAGAGTCACAGAGAATTCA |
| | AATATTGTTAAGCTGCTTAATAGAAAAACAAATTATGGTAAATGTGTTCACTGGAATACTA |
| | CCCATGATTTATAATAAATAAATGCCTGACACACAGAACAGCAGCAAAACCACACATGCTC |
| | TTATTACAGAAAGTGGCTTCTGAAAACCACACCGGGCATGTACAGCTTTGTCCTGGAGTTG |
| | GTTTAGGGGGATGTCAGAGCCAGTGACGAGAAGCACAGGGCCAGATGGCAGCGTTCACTCA |
| | TCCCAGACATGAGCTCCTGGGTGCATACAGAGCCCCCCCATGTGTGGGTTTACTTCCACTT |
| | CTGTAAAAGGAGAAAATACTGACTCCTACAGAGCATAATTTACACATTTTTTAAAAAATGT |
| | AATAGGGTGATCAGGGCAAAGTGTTTATCACAGCACAATTTCATAAGACAGCATATTTTCC |
| | AAATACCATCATTGTCAGCAAACTTCTGCAGAGCACCGTCTTCTTATATGGGTACAGCCTA |
| | TTCCTCCAGCATCCCACTAGAGCTTCTTATATAGTAGGAGACATGCAAATAGGGCCCTCCC |
| | TCTACTGATGAAAACCAACCCAACCCTGACCCTGCAGGTCTCAGAGAGGAGCCTTAGCCCT |
| | GGACTCCAAGGCCTTTCCACTTGGTGATCAGCACTGAGCACAGAGGACTCACCATGGAGTT |
| | GGGGCTGAGCTGGGTTTTCCTTGTTGCTATTTTAGAAGGTGATTCATGGAAAACTAGGAAG |
| | ATTGAGTGTGTGTGGATATGAGTGTGAGAAACAGTGGATTTGTGTGGCAGTTTCTGACCTT |
| | GGTGTCTCTTTGTTTGCAGGTGTCCAGTGTGAGGTGCAGCTGGTGGAGTCTGGGGGAGGCT |
| | TGGTCCAGCCTGGGGGGTCCCTGAGACTCTCCTGTGCAGCCTCTGGATTCACCTTTAGTAG |
| | CTATTGGATGAGCTGGGTCCGCCAGGCTCCAGGGAAAGGGCTGGAGTGGGCGGCCAACATA |
| | AAGCAAGATGGAAGTGAGAAATACTATGTGGACTCTGTGAAGGGCCGATTCACCATCTCCA |
| | GAGACAACGCCAAGAACTCACTGTATCTGCAAATGAACAGCCTGAGAGCCGAGGACACGGC |
| | CGTGTATTACTGTGCGAGAGACACAGTGAGGGGAAGTCAGTGTGAGCCCAGACACAAACCT |
| | CCCTGCAGGGGTCCCTTGGGACCACCAGGGGGCGACAGGGCATTGAGCACGGGGCTGTCTC |
| | CAGGGCAGGTGCAGGTGCTGCTGAGGGCTGGCTTCCTGTCGCGGTCTGGGGCTGCCTCGTT |
| | GTCAAATTTCCCCAGGAACTTCTCCCAGATTACAATTCTGTACTGACATTTCATGTCTCTA |
| | AATGCAATACTTTTTTTGTCCTTTTTGTTTCTTTGTTTTTTTGCAACAGGAGTACATATCC |
| | TCAGCTCCACAGAAGCCAGGGTGTCACTTTGGGGGCAGAAATAATCCTTTCGTGGTCAACA |
| | GGATGAGAGTCCTGAGGAATCCCAGGGAAACCTGGAGAGTGTTTTCCAATTAGACTCAGGG |
| | CAGAGACCTCCATGGGAATTTCTGATTAGAACGGGCTTTGAGCTCTGATGGGAGCCAAGAG |
| | ACAGGCTCACCCAGGGTCGGGGTCCTTAAAGCCTGATGGTTTTCACAGCTATCCCCCCTCG |
| | TCTTGTAAACTCAGACTGATTCAGTTGACCCTTTTTCTGCTAATCCATTTTGCTTCTCTGT |
| | AGGTTTGATTCTCACAGTTCGCTTTCTTCTTCTCTTCCCTGAGAACAAACGATGTGTTTTC |
| | TGTTTTCAGGGAAAATCCCAGGGCTCAGGTCTGCAGGACCTGGGTAGGCTTGGAAAAGGTA |
| | AATGTGGTCTCCGTGACCTCAATTCAAGGGCTGAAGCCCTTTCCCTGTAGCTCAGCTGGGG |
| | CTCAGGCTGTGGCTACTGCAGCCATGTGGAAGAGGCTGAAGGGACTTTCTTCACTCTCCTT |
| | GCTCAGGACCATCCACTGTATTGTGTATAGGCTTCTCTGGAAATGCAAGTGGCCATTTGTA |
| | GTGAAAGAAATATGTTTGTCTGGTTAAAATGGGAGGTGGATGTAGAGTTAATTGGCTGCTA |
| | CATAAACTGTCCTTCTCCACCAGTGCTTTTAGGATGAGATTGTGAAATTTGTAAGAATCAA |
| | AATGGAGTCACATATGTTAAAACCCTGACAAATGGATTCAGGAAGTGTAGGGAGAATTCTT |
| | ACACACATATCCCTGACAACAAGAACTATCATAAAATAGTTCTTGCAAAAAGACCAACATG |
| | ACCTCATAATCATGACTTCTGCAAAGACTTCTACTCAGAATCTACTTGCCCAGCCTTAGAT |
| | TAATGCCATCTGAATTACACTGATCATGTTACTATCACTGCTCCTCACCACAGATGCAACA |
| | CCCTCCTGAGTCCTGAAACCTGACTCCATCCCATAGAGTAGGGCACAGATGAGGGGAATGC |
| | AAATCTCCACCAGCTCCACCCTCCTCTGGGTTGAAAAAGCCGAGCACAGGTCCCAGCTCAG |
| | TGACTCCTGTGCCCCACCATGGACACACTTTGCTCCACGCTCCTGCTGCTGACCATCCCTT |
| | CATGTGAGTGCTGTGGTCAGGGACTCCTTCACGGGTGAAACATCAGTTTTCTTGTTTGTGG |
| | GCTTCATCTTCTTATGCTTTCTCCACAGGGGTCTTGTCCCAGATCACCTTGAAGGAGTCTG |
| | GTCCTACGCTGGTGAAACCCACACAGACCCTCACGCTGACCTGCACCTTCTCTGGGTTCTC |
| | ACTCAGCACTAGTGGAGTGGGTGTGGGCTGGATCCGTCAGCCCCCAGGAAAGGCCCTGGAG |
| | TGGCTTGCACTCATTTATTGGGATGATGATAAGCGCTACAGCCCATCTCTGAAGAGCAGGC |
| | TCACCATCACCAAGGACACCTCCAAAAACCAGGTGGTCCTTACAATGACCAACATGGACCC |
| | TGTGGACACAGCCACATATTACTGTGCACACAGACCACAAAGACACAGCCCAGGGCACCTC |
| | CTGTACAAAAACCCAGGCTGCTTCTCATTGGTGCTCCCTCCCCACCTCTGCAGAACAGGAA |
| | AGTGCAGCTGAGATACGTTTTCCTGCCAGGGCCTGCATTTCCCATCCCCATTAGACTCAGA |
| | GCCCTGTCTTCCTCCTTCTTCTTTAATAATAAATGGCATGACTCCTGTTAATAGTTCATAG |
| | AAGCAGAAGCTGAGTCCTGTTTGTCAAACATTCAGCATGAAATGTTCATGTTACCTGGGCC |
| | AGATGCATCACTGGTATGTGGCCGCCAGTTTAATGGGCTCATACTACTCCAAGCGGCCGAA |
| | TACAAAACAGCAGAACATATGCCTTTATTAAACTGCAGGAAATGACACTGGAGAAAAATGT |
| | GGGGATAAGGGAGCAGGAGGAATTGTGAAAATTCAGATAATAGCTTTTCTCGTAGAGAGAA |
| | AGGTGCGTTAATAAAATAATTTTATAAAACAATAAAACAGATGGAATGTGTCCCCATCACG |
| | GAGTGTCTCACAGTAACAGTGCAGAAATAAATTTCTAAAAATGTCGTAGGGTAGGTGTGAT |
| | AGACAAGGCTTATTTCCACACAGGGAGCCCATAAATACCCATGTAATTAAAAATCCAGCTC |
| | TTAGAAAGGGAGTGCCTCAACTTGACATCAGAAACCCACGTCAGTGAGTCTGAGGAGCAAT |
| | GTGCTCTGCAGTGCAAGGGCTGGGCTGCAGGGGACGCTCAAAGCCCACCCAGTACAAGTTT |
| | CAGCCCCAGAGCAGGTGCACAGGAGGCTGGGGAGGGATTCCTCTCGAGATCTGTGTCATTC |
| | TTTAAAAAATCTAAAATACATATTTGACAACTGAACATTCTATAAATATCCTATTCAATTG |
| | CGAGCATTTATCAAACTTGATGTTGTAATGAGAACCACTTTTACACTGGAGATTTCTAACC |
| | CTTCTAGATATCTTAATAGTATGCAGCTGGAGGTTAAGGAAACTACTTTTCTTTTCTATAA |
| | ATAAGTGAAACTTTTGGAGAAACACACTCATCCCCCAAATAATACATTCATGTATTAAAGC |
| | CTAGAAATGCTTATATTACCTCTGAGCCATTTGAGTGTGGGTTCCCAGTGAAGTCCTGTTC |
| | TAGGGAAAATTGTTAGCCAATGGGAGAAGCTCTGTCAACATAGAGCTTAGGGATATGGCAG |
| | GGCTCACATGGCCTCTAAGGGGATTACAGCTTGAACCTTGAGCATCCTCCGTGTGTCATCT |
| | CTCTGCTCTTTCTCATGCAATATCAGGTATGAAATAGGATCACTCATGAATATGCAAATAA |
| | CTGAGGTAAATATAGCTATCTTTGGGCCCTGAGAGCATCACCCAACAACCACACCCCTCCT |
| | AAGAAGAAGCCCCTAGACCACAGCTCCACACCATGGACTGGACCTGGAGGATCCTCTTCTT |
| | GGTGGCAGCAGCAACAGGTAAGGGGCTCCCCAGTCACTGGGCTGAGGGAGAAACCAGCACA |
| | GTCAAGTGAGACTTCATGCACTCCCATCTCCTCTCCACAGGTGCCCACTCCCAGGTGCAGC |
| | TGGTGCAATCTGGGTCTGAGTTGAAGAAGCCTGGGGCCTCAGTGAAGGTTTCCTGCAAGGC |
| | TTCTGGATACACCTTCACTAGCTATGCTATGAATTGGGTGCGACAGGCCCCTGGACAAGGG |
| | CTTGAGTGGATGGGATGGATCAACACCAACACTGGGAACCCAACGTATGCCCAGGGCTTCA |
| | CAGGACGGTTTGTCTTCTCCTTGGACACCTCTGTCAGCACGGCATATCTGCAGATCAGCAG |
| | CCTAAAGGCTGAGGACACTGCCGTGTATTACTGTGCGAGAGACACAGTGTGGAAACCCACA |
| | TCCTGAGAGTGTCAAAAACCCTGTGGGAGGAGGCAGCTGTGCTGAGCTGAGGCAGTGACAG |
| | GGACAACGTGGCTGCACCCTTTGTAGGGTTATAAACATACATATATATGCACCCAACAGTG |
| | GAGTACCCAGATATATCAAGCAAACATTATTAGAGCTAAACAGAGAGATAGACCCAATACA |
| | GTAAAAGCTGGAGACTTTAACACCCCACCCTCAGCATTGGACAGATCTTCCAGACAGAAAA |
| | TTAACAATGAAATCTTAGACTAAATGTACATGACAGACCAAATGGACATAATGGATATTGA |
| | CAGAACACTTCATCCAATGGCTGTAGAGTGCACAGTCTTTTCCTAAACATATGGATCAGTC |
| | TCAAGGATAGACCATATAGTAGACCAGAAAATTAGTCATTTTTTAATTGAGAAGATATCAA |
| | GTACCTTCTCTGATCATAATGGAAGAAAACTAAATTAATAAGAGGAATCTTGGAAAACTAT |
| | GCAAACACATGGAAATTAAACAATATGCTCCCTAATTAAACAATGTGCTCCCAAATTGAAC |
| | CACATGCTCCAAAATGGGTTAATAAAGACATAAAAAATTTTAAAATTTATTGAAAGAAATG |
| | ATAAAGAAGACAAAGCATATGAAAACCTATGAAATACAGTGAAAGCAGTATTAAGAGGAAA |
| | GTTTATAGCTCAGGCCTGTAATCCCAGCATTTTGGGAGGCTAAGGCAAGTGGATCACCTGA |
| | GGTCGGGACATTGAGACTAGCTTGGCCAACATGGTGAAACCTTATCTCTACTAAAAATATA |
| | AAAATTAGACGGGGGTGGTTGTGCGTGCCTGTACTCCCAGCTACTCGGGAGGCTGAGGCAG |
| | GAGAATCACTTGAACCCAGGAGGCGGAGGTTGCGGTGAGCCGAGATCGGGACACTGCACTC |
| | TAGCCTGGGCAAAGGAGCAAAAGTTCATCTAAAAAATTTATTTTAATTTAAAAATTTTAAA |
| | AAAATGGCCCACTCCCTAGAACAGAGAGATTCCCTCTAAACATGATGGAGGTCCCGAACTA |
| | TACATTAAGTGAATCCTGGTGTGTCTGAACTCACATGATTATTACGTTAAGCTGCTGTTCC |
| | AATCTACTTCCTCACCTGGGAAAAGAGGAGCCAGGGCATGGCTAGTTGAGGCCCCAGGAAG |
| | AGAACTGAGTTCTCAAAGGGCAAAGCAAGCATCCTCATCCCAGGGCGAGCCTAAAAGACTG |
| | GGGCCTCCCTCATCCCTTTTCACCTCTTTATACAAAGGCACCACCTACATGCAAATCCTCA |
| | CTTAGGCACCCACAGGAAACCACCACACATTTCCTTAAATTCAGGGTCCAGCTCACATGGG |
| | AAATACTTTCTGAGAGTCATGGACCTCCTGCACAAGAACATGAAACACCTGTGGTTCTTCC |
| | TCCTCCTGGTGGCAGCCCCGAGATGTGAGTGTCTCAAGGCTGCAGACATGGGGGTATGGGA |
| | GGTGCCTCTGATCCCAGGGCTCACTGTGGGTCTCTCTGTTCACAGGGGTCCTGTCTCAGGT |
| | GCAGCTGCAGGAGTTGGGCCCAGGACTGGTGAAGCCTCCGGGGACCCTGTCCCTCACCTGC |
| | GCTGTCTCTGGTGGCTCCATCAGCAGTAGTAACTGGTGGAGTTGGGTCCGCCAGCCCCCAG |
| | GGAAGGGGCTGGAGTGGATTGGGGAAATCTATCATAGTGGGAGCACCAACTACAACCCGTC |
| | CCTCAAGAGTCGAGTCACCATATCAGTAGACAAGTCCAAGAACCAGTTCTCCCTGAAGCTG |
| | AGCTCTGTGACCGCCGCGGACACGGCCGTGTATTACTGTGCGAGAGACACAGTGAGGGGAG |
| | GTGAGTGTGAGCCCAGACACAAACCTCCCTGCAGGGAGGCGGAGGGGACCGGCGCAGGTGC |
| | TGCTCAGAGCCAGCAGGGGGCGCGCGGGGCCCACAGAGCAAGAGGCCGGGTCTGGAGCAGG |
| | TGCAGGGAGGGCGGGGCTTCCTCATCAGCTCAGTGCTCTCCCTCCTCGCCAGGACCTCAGC |
| | TGTCCCCAGGCCTCCTCTTTCTTTATTATCTGTGGTTCTGCTTCCTCACATCCTCGCTGCA |
| | GGCAAGAAAGAAGGGAGAAATTTCTCTGTTTACCATTAAGGTTTTAACAGTCACTGGCACC |
| | TTTTTTTCAACAAATTCCCACAAGGCCCATTTTACCTTCTTGAAAAAATATTTCAAGGCTT |
| | CCCACCATCTCTTGATTGGCGTCATCGGCTGCATCGTGTCCTGCTCTTAAATTCATGTGAT |
| | GAAACCCCAATCCCAGTGGCTCTGTATTTGGAGAGAGGGATTTTAAGAAGGCAAATAAGTT |
| | AACTGTGGTCATAAATGTGGGACCCTAATCCAGTAGGACTCTTGTCCTTATGGGAAGTGGA |
| | AGACATCAGAGGCCTCTCTCTCCACATGCACACAGAGGAGAGGCCATGTGGGGACGCGCTG |
| | CATGAGAAGGTGGCCCTTTGCAAGCCTGGAAGAGGGCTCTCAAAAAACTAACCCTTTCTGC |
| | ACCCAGATCTTGGACCAGGAGCCTCTCTGCAGTGCAAGGGCTGGGCTGCAGGGGGCGCTCA |
| | AGGCCCACCCAGCACAGGCTCCAGCCCCAGAGCAGGTGCACAGGAGGCTGCGGATGGAGTC |
| | CCCTTAAGATCTGTGTCATTCTTCTAAAAAAATCTAAAATAAGTATTTGACAAAAACTGCT |
| | GAAGAGTCCATAAATATCCTATTCAATTGCAAGAATTTATCAATTTACACTGGAGATTTCT |
| | AACCCTGCTACATATCTTAATAGTAAGCATCTGGAGGTCAATTAAGCTTTTATTTTATATA |
| | AATAAGTGCAACTTTTGGAGAAACACACTCATCCCCCAAATAACACATTCATGTATTAAAG |
| | CCTAGAAATACTTTAAATTACCTCTGAGCTATTCAAGTGTGGGTTCCCAGTGAAGTCCTGT |
| | TCTAGGGAAAATTGTTCTCCAGTGGGAGAAGCTCTGTCAACATACAGCTTAGGGATATGGC |
| | AGGGCACACATGGCCTCTAAGGGGATTATAGCTTGAACCCTTAGCATCCTCCTGTTGGGTA |
| | ATCCATGTGTCATCTCTCCACTCTTTCTCATGCTGTGTTAGGTATGAAATAGCATCGCTCA |
| | TGAATATGCAAATAACTGAGATGACTATAGATATCTTTGTGCCCTGAGAGCATCACCCAAC |
| | AACCACATCCCTCCTCAGAAGCCCCCAGAGCACAACGCCTCACCATGGACTGGACCTGGAG |
| | GATCCTCTTTTTGGTGGCAGCAGCCACAGGTAAGGGGCTGCCAAATCCCAGTGAGGAGGAA |
| | GGGATCGAGGCCAGTCAAGGGGAATTCCATCCACTCCTGTGTCTTCTCTACAGGTGTCCAC |
| | TCCCAGGTTCAGCTGGTGCAGTCTGGGGCTGAGGTGAAGAAGCCTGGGGCCTCAGTGAAGG |
| | TTTCCTGCAAGGCTTCTGGATACACCTTCACTAGCTATGCTATGCATTGGGTGCGCCAGGC |
| | CCCCGGACAAAGGCTTGAGTGGATGGGATGGAGCAACGCTGGCAATGGTAACACAAAATAT |
| | TCACAGGAGTTCCAGGGCAGAGTCACCATTACCAGGGACACATCCGCGAGCACAGCCTACA |
| | TGGAGCTGAGCAGCCTGAGATCTGAGGACATGGCTGTGTATTACTGTGCGAGAGACACAGT |
| | GTGAAAACCCACATCCTGAGAGTGTCAGAAACCCCAGGGGGGAAGCAGCTGTGCTGGCATG |
| | GAGGAAATGACAAAGATTATTAGATTGAAGACTTTCTCAGAAAATGATATTAAGTCATTAA |
| | GGAAAAGGAACAATATAAACATGTATTTGAGAAATTTTAATTATTTGAGAGATTTTTCATA |
| | CAATATTTATTCTGCAAGCAAATTTCAGGGATTGAATTAATAAAACTGATACAGAACTTCC |
| | TCTGTAGGTATCTGTGTAAACATCAATTTCTGAATCAGTGTTGTAAATATTTTGGAACACA |
| | CACACAAATCACATTTTATCTCTACTTTTATCTCTATTATTAAAAATGCCAAAAAAACTCA |
| | TTTTGTGCATGTAGCATTTTGAATTCCCACCATCAATGCATGATAGTTCTTGGTTTTCCAC |
| | ATTCATATTGCCATTTATCATTATGAGAATTGTGTGTTTTAACCATTCTAATAGGTGAGTA |
| | ATGGTATCTAATTTTTAGTTAAATGCACATTTCCGTAATAAAAATTCACATTTAACAATTT |
| | TCATATAATTTTTGCCGAGATGCCTCTTCTCATATTTGGTTCATTTTTAACTGCATTGTTT |
| | TCTTTTGATTAGTTGTAAGTTTACTTGCATATTGATTATAAAATCATTTAACAAATTAAAA |
| | GAATTCATTTAACAAATATGTGACTTGGAAGTATTTTCTCCAAGTCTGCGGCTGTCTTTTA |
| | CTCCCTTATCAGCACAGGCTGCAGCCCTGGAGCAGGTGCAAGGGAGGCTGGGGAGGGGTTC |
| | CTCCCAGGGTCTGATGTCTTCCTTTTCTCGGACAAACATGCTTTAATAAGTTAAACAAGAC |
| | TTTAGTAAAGACTATTGATGTGTCTTTGTGTCTTTCAGTATACAGTTCTATTTGTAGGATT |
| | TATCTAACCTAACAAGTCAATGAGAATCACATGTAAAAGGAGAAATTTCTAGGATTTTCAG |
| | ATATCTTAATAGGTAGGAGATGGAGAAAAGGGATGGTTTTATTAATTCAGTGCTTGCCAAT |
| | CTTAACAGAGACAGTAGTAAGACATGCAGAAAGCAAAGCCCAGAAAAGTATGAAGGTGTCA |
| | AAGTGCCATTTAAGTATGGGTTCACTTGGAGGACCATGTTCTGCGGGAACTTGTTTTCAGC |
| | AGACAATCTATTTTAGCAGAGTTCTGGGCATACAAGGGGACACACATCATTAAACAAGGAT |
| | TGGGACAGGGACTTCAGCGTCCCACTGTTGCATGGCCCATAAATTATGTGTGTTCTCTTTC |
| | TCATCTTGGATCAAGTCTAGAGCTATGAAATAGTATCCCTCATGAATATGCAAATAACCTG |
| | AGATTTACTGAAGTAAATACAGATCTGTCCTGTGCCCTGAGAGCATCACCCAGCAACCACA |
| | TCTGTCCTCTAGAGAATCCCCTGAGAGCTCCGTTCCTCACCATGGACTGGACCTGGAGGAT |
| | CCTCTTCTTGGTGGCAGCAGCCACAGGTAAGAGGCTCCCTAGTCCCAGTGATGAGAAAGAG |
| | ATTGAGTCCAGTCCAGGGAGATCTCATCCACTTCTGTGTTCTCTCCACAGGAGCCCACTCC |
| | CAGGTGCAGCTGGTGCAGTCTGGGGCTGAGGTGAAGAAGCCTGGGGCCTCAGTGAAGGTCT |
| | CCTGCAAGGCTTCTGGATACACCTTCACCGGCTACTATATGCACTGGGTGCGACAGGCCCC |
| | TGGACAAGGGCTTGAGTGGATGGGACGGATCAACCCTAACAGTGGTGGCACAAACTATGCA |
| | CAGAAGTTTCAGGGCAGGGTCACCAGTACCAGGGACACGTCCATCAGCACAGCCTACATGG |
| | AGCTGAGCAGGCTGAGATCTGACGACACGGTCGTGTATTACTGTGCGAGAGACACAGTGTG |
| | AAAACCCACATCCTGAGGGTGTCAGAAACCCCAGGGAGGAGGCAGCTGTGCTGGGGCTGAG |
| | AAATGAAAGGGATTACTATTTTTAATGTTGTTTACAGTATGTCATTAATAAATTGAAAAAA |
| | AGTAACAATAGAAGTATATACTCTAATTATATGGGAACTTTGTTTTTTCAGTTTTTTCATT |
| | TTTTTTTTTTTTTTTGGTTTGTTTGTGACAGAGTCTCACTCTGCCACCCAGGCTGGAGTGT |
| | AACGGCACAATCTCAGCTCACTACAACCTCCACCTCCCAGGTTCAAGCAATTCTCCTGCCT |
| | CGGCCTCCAGAGTAGTTGGGATTACAGGCACCCGCCACCATGCCCGGTGAATTTTTGTATT |
| | TTTAGTAGAGACGGGGTTTCACCATGTTAGCTAGGCTGGTCTCAAACTGCTGATCTCAGGT |
| | GATCTACCCTCCTCAGCCTCCCAAAGTCCTGGGATTACAGGCGTGAGCCACTGCGCCTGGC |
| | CCAATTATATGGGAATTGTTTATATAATTATCACCCTATAAGCAAAATTCATGGAGGAGGA |
| | AAAGCTCTACTGAAGAAAGCTGATACCGGCATTCCCATGAAAGTATCTGTGTAGAAGTAAG |
| | TATTAAAATCAGTTGAATAGGCAAGGCATGGTGGCTCACGCCTATAATCCCAGCACTTTGG |
| | GAGACCGAGGCAGGTGGATCCCCTTGGGGTTTGGGATGAGCTGCCCTTGCCCACTGTGCTC |
| | TGTGGACCTCCCTTTAGAAGCTCACAGCTCCCTGCACTCGGCTCCATCCTGCCCCACCACA |
| | CAGAAGCAAAACCCCTCTCCTTTCCACTGCAGGCTTTTCCTGGACCAGAATGCTGACCTGC |
| | TGCCCTTCACTCCCGAAGTGGTGGGACTGCCTGGGGTGGTGTGGGTGTTGAGCCTTCTTAC |
| | TCTAGGGACCTGGCACCTGGCCCCAGGGGCACAGGGATGGTGCATCTGCCTAGGGATGCCT |
| | CCTCATGCCAGGGGGTGGGGGTTAGTACCATCGGCCCTCAGGATTTGTTGCATGAATGAGT |
| | GAATGGGTGAATAAATGAAGGGGATCTGATCTATGAATAAGGGTATATAGACTTTGGTTGA |
| | TGTAGGACGCCAAATGCTGGAATTTCAGAGTCATCACACCCAGGGGCCCTGCCTCTGAGCT |
| | CCTCTTTGCATCCAATCTGCTGAAGAACATGGCTCTAGGGAAACCCAGTTGTAGACCTGAG |
| | GGCCCCGGCTCTTCAATGAGCCATCTCCGTCCCGGGGCCTTATATCAGCAAGTGACGCACA |
| | CATGCAAATGCCAGGGTGTGGTTTCCTGTTTAAATGTAGCCTCCCCCGCTGCAGAACTGCA |
| | GAGCCTGCTGAATTCTGGCTGACCAGGGCAGTCACCAGAGCTCCAGACAATGTCTGTCTCC |
| | TTCCTCATCTTCCTGCCCGTGCTGGGCCTCCCATGGGGTCAGTGTCAGGGAGATGCCGTAT |
| | TCACAGCAGCATTCACAGACTGAGGGGTGTTTCACTTTGCTGTTTCCTTTTGTCTCCAGGT |
| | GTCCTGTCACAGGTACAGCTGCAGCAGTCAGGTCCAGGACTGGTGAAGCCCTCGCAGACCC |
| | TCTCACTCACCTGTGCCATCTCCGGGGACAGTGTCTCTAGCAACAGTGCTGCTTGGAACTG |
| | GATCAGGCAGTCCCCATCGAGAGGCCTTGAGTGGCTGGGAAGGACATACTACAGGTCCAAG |
| | TGGTATAATGATTATGCAGTATCTGTGAAAAGTCGAATAACCATCAACCCAGACACATCCA |
| | AGAACCAGTTCTCCCTGCAGCTGAACTCTGTGACTCCCGAGGACACGGCTGTGTATTACTG |
| | TGCAAGAGACACAGTGAGGGGAAGTCAGTGTGAGCCCAGACACAAACCTCCCTGCAGGGAT |
| | GCTCAGGACCCCAGAAGGCACCCAGCACTACCAGCGCAGGGCCCAGACCAGGAGCAGGTGT |
| | GGAGTTAAGCAAAAATGGAACTTCTTGCTGTGTCTTAAACTGTTGTTGTTTTTTTTTTTTT |
| | TTTGGCTCAGCAACAGAGATCATAGAAAACCCTTTTTCATATTTTTGAAATCTGTTCTTAG |
| | TCTAATGGAGATTCTCTAATATGTGACAATGTTTTTCTCTTGCTGTTTTTGGAATTCTTTG |
| | TCTTTGACTTTTGACAACTTGACTTTTGACAGTGTGCCTCAAAGAAGTTCTATTTTGGGTT |
| | CTGTGAACCTCCTGGATCTGGGAAGTTTTCAGCTATGATTTCATTAAACGTGTTTTCTACA |
| | CCATTTCCCTACTCTTTTGGAATACCCATAATGCAAATATTTGTTCACTTAATTGTGTCCC |
| | ATAAATGCTGGGGATTTTCTTCATTCCTTTTTACTCTTTTTTTCTTTTTATTCATCTGCCT |
| | GAATTATTTCAAAAGATCTGTCTTCAACTTCAGAAACTCTTTTGCTTGGCCTAGTCTAATC |
| | TTGAAGGTCTCAATTGTACTTTTAATTTCATTCATTGAATTCTTCAACTCTGGAATTTCTG |
| | TTGGTTCTTTTTTATGATACTTATCTCTTTGTTGAATTCGAACACAAAATAGTGACAGCAG |
| | AGATGACGGCAAGTTGGCATTTTTCTTTTCTAGCAATAAAACTTAAAGCTGACTCAAGGAG |
| | AAATGGAAATCATAATTGGAACAGTAATCCTCAAGAAAGCATTAAGATTATTAAATAATTG |
| | CCCTCACAGATGACTTCAGGCCAAGATGGCTTTATGGGTGAAGTTTAGACTTTCACAAAAC |
| | TAATCAGTTCCCATAAGAACTGCTCCAGGATTTGGAGGAACATGGGAAAGTCTATTAAAGG |
| | GATCACAATTCACAGTCCCCAGAGTAAAACATGGGCTAACTTGCATTTTGGCAAAGAGCCA |
| | AATGTTATAAATGACATCCTAGAAGGCCAAATTCTGTCCATCTCGTTGAACAAGGACTTAC |
| | ACCAGGAATTTAGAACTATTTATAGCTCATCCCACCACTCAGGCCAATGATGACCCATGAT |
| | CATCTCACCAGAAATGGAAAGACTCAGATGATTAATAGAGTCTCAATTTCTCTGAGACATC |
| | TAAGAGCCCAGCCCAAGCCCAGACCCAGGAGGGCACCCAGGCCTGGACAGAGAACACTGAT |
| | ATCACACCAGCCCTCCAGAGGGAAGCAGAGACTCCTTCAAGCTCTGGAAACACAGGCCCAG |
| | ACAGCTGCCTAAAGTTGGGCAGGCTTCACTGCAAACCCAAATCATGAAGCTAGGTAACACC |
| | TTTACAGATTCTTTACATTTAAAAATCATCAAAACAAGAGTAAATAATAAACTCAAATAAT |
| | ATTAATCTAATATGTAAAGGTCTTGTACCATTATTATGCAAACAACATACATAAGCTAATA |
| | AGAAAAAGAACAAATCCCTTAAGAAATCGGCAAAAAGGATATAACACAATTTCTAAAAGAA |
| | AACAAATGGCTAGCACACATAAGGAAAACACTTTGTGAACAGACATTCTTCAGAACATTAT |
| | TTATAATTATAAAATAGTTGAAAGCAAGATAGTGCCTGAAGAAATTATGGTGCATACATTA |
| | GTGGGACTATTCTGCAAACATTCCCAATTATACTTGTCACATATCTGTGATAACGTGACAG |
| | CCAGCATTCATGGGGTGACCTCATTTGGTAAAAGGGTGCAAAGCTCAACACGCATTGTGAG |
| | ATGACTGTGGTGTAAAATTAGTGGGATTATTCCGCAAACATTCCCAATTATACTTACCGCA |
| | TATCTGTGATAACATGACAGCATTCATGGGGTGACCTCATTTGGTAAAAGGGTGCAAAGCT |
| | CAACACGCATTGTGAGATGACTGGTGTAAATACAAAGACCAAACTGTGAAAAGGAGTCCAT |
| | CAATTAATCGATGCTTACCTTCAGTTTTGGGCTAATTTTTAAAGTATGCTATAAGCATATG |
| | CTCCTGTTATAACAGAATGGAGGGATTATGAGAGATGATGCAGGTGTGTCCTGGGCCTCCC |
| | CTGGCCCACTGGGCCCTAGAGATGCCTTCCCAGGCATCGCTGTCAGGGCTTCCCTCAGAGG |
| | GAGTCCTGTATTGACCTCACCACCAAGGTCTGGAGCAGGGGATCCTTAGATATTGGTTGGG |
| | GTTATCTCACCTTAGGTCTGAATTTGGGGTTGTCTTAGACTGTTTTGTGCTGTTAGAATAG |
| | AATACCCAAGACTGGGAAATTTATACTGAACGGAAATTTATTTCTCACAGTTCTAGAGGCT |
| | GTGAAGTCCAAGAGCACAGGTGCCAGAGCAAGTCCAAGAGCAAGGGAAAGTCCAAAGCAAG |
| | TCCAGGAGCATCTGGCGAGGACCTTCTTGCTGTGTCATCACATGGCGGAAGGCAAGAAAGA |
| | GAGCAAGAGGGGGCCGAACTCACCCTTTTATAACAGCACCAATCCCACCCATGAGGTGGGG |
| | ACCTTATGACCTAATCACTCTTCATACTGTTACAATGGCAATGAAATTTCAACATGAGTTT |
| | TGGAGGAGAGAAGCATTCAAACCACAGCAAGGGTGCTCCTACCTCCTCTCTCAGGGCATCT |
| | GCAGAAAGAGCTGCAACTGCACGTCCTTCCTCCGTCCATCCTCCATCCCTTCCCAATGTCC |
| | GTGCATATCCTGTGACCCAGGAGGTCTGGCATAGGGGGTGCTCCTGCCTTAGGTCTGAGGC |
| | CCTGTCTGAAGAGGGGTAGGTGAGGAGGCCATCTGATGGTCTGGGCCAAGACAGTCACAGG |
| | ACGCATCATTTATCATCAAGGAGGCTGAGGGTTGAGTCTCCAGGTCCAGGGAACTCCCCAC |
| | AAAGTGGGAACCCTGCCCAGCTCCACACAGCCTCTGCTGGGGGACCCTGCTCTGGTGCAGA |
| | GCCTGGGGACAGGTCTTGAGCTCAGCCAGAGTCTGCCTCCCTGTCATTTAGGAACTAAACC |
| | AAGCGGCAGGATGCTGGAGCCCAGCCCCCATCTGACCTTACAGGGCCAAGGCTGGGGCCCT |
| | GGGTTCCCCTCAAGGCGCAGCAGGACTGGAGCCCCAGGCAGTGCAGGAGTGGCCAAAGCTG |
| | GGGCTTCCTCCAGAGCCCCCAAGCATCACGGCACCAAGAAGGGTAGGACCCTGGCCTGAGG |
| | AATTGGCACCAAAGCCCCAGAAACTACCCTGGACACCATGGAGAGAGGCCTGGAGGGGAAG |
| | CACCAGGCACTGCCTCCCCTTCTGATCCCACCTGAGGTGGCTGCCAAGCCCAGAGAGCCGC |
| | TCTGATGTCCCCCAGCCCTGCAGCCCAGGGATACCTGTACTGTGCCCCTGGGGGACCCCTG |
| | GCCAGTCTGTGCAAAGAAGTCACCACCCTACACTCAGAGACAGTGGGGGTCCTCGTCCCAC |
| | ATCCTCAGAGCATGGCCCGGCTGCTGCAGGGATGGTCTCCTGGTCCTCAGAGCATGGCCCG |
| | GCTGCTGCAGGGATGGTCTCCTGGTCCTCAGAGCATGGCCCAGCTGCTGCAGGGATGGTCT |
| | CCTGGAGGCCCCCCAGTGCTCTATTGTCAGGGCTCCCTCCACCCCCCGCACCAAGAGAGAG |
| | CCAGACCCCAGCAAGGCTTCCAGTGGCTTCAGGTCACACCCCTAGGCTGACCCCAGCCCCA |
| | TTAACACCTGCCTGAGAAAGCTCCACGCACCAGAACTGACCGTCTGCTCCAACTCTTGACC |
| | TCCCGTTCTCAGGGCGTCTGCTGAAAAGGCTGCAACTGCACATCCTTCCTCCGTCCGTTCC |
| | CGATGTCCGTGTGTCTCCTGTGGCCAGGAAGGTCTTTCTCGGGACCTGAGAGCCGCTCCCT |
| | GAAGTGTCCCCATTGGGAAGGATGGGGCCTGTGTCTCCAGGCTCTGGGAGGACAGAATCCT |
| | GACCTCAACAGTGGCCGGCACGGACACAACTGGCCCCATCCCGGGGACGCTGACCAGCGCT |
| | GGGCAACTTTTCCCTTCCCCGACGACTGAGCCCCGAGCACCCTCCCTGCTCCCCTACCACC |
| | TCCCTTTACAAGGCTGTGGCCTCTGCACAGATGATAATGGAGCTTGGCTCATTCCCCTAGA |
| | GTCGGTAGGGAGTTAAGGACAAAACTCAGTTTCCTCCACCTGAACTCAAGTCTGCCTATGT |
| | TTACCTAATCACACCTGGTGGACAGTTTGGACAAACTTGCACACTCAGAGACACAGACACT |
| | TCTAGAAATCATTATCTCCCTGCCCCGGGGACCCCACTCCAGCAGAAGTCTGCTAGGCACT |
| | GGCCTGGGCCCTCCTGCTGTCCTAGGAGGCTGCTGACCTCCTGCCTGGCTCCTGTCCCCAG |
| | GTCCAGAGTCAGAGCAGACTCCAGGGACGCTGCAGGCTAGGAAGCCGCCCCCTCCAGGCGA |
| | GGGTCTAGTGCAGGTGCCCAGGACAAGAAAGATTGTGAATGCAGGAATGACTGGGCCACAC |
| | CCCTCCCGTGCACGCCCCCTCCTGCCCTGCACCCCACAGCCCAGCCCCCCGTGCTGGATGC |
| | CCCCCCACAGCAGAGGTGCTGTTCTGTGATCCCCTGGGAAAGACGCCCTCAACCTCCACCC |
| | TGTCCCACGGCCCAAGGAAGACAAGACACAGGCCCTCTCCTCACAGTCTCCCCACCTGGCT |
| | CCTGCTGGGACCCTCAAGGTGTGAACAGGGAGGATGGTTGTCTGGGTGGCCCCTAGGAGCC |
| | CAGATCTTCACTCCACAGACCCCAACCCAAGCACCCCCTTCTGCAGGGCCCAGCTCATCCC |
| | CCTCCTCCTCCCTCTGCTCTCCTCTCGTCGCCTCTACGGGAAATCCGGGACTCAGCAGTAA |
| | CCCTCAGGAAGCAGGGCCCAGGCGCCGTTTAATAGGAGGCTTCCTCACAATGAAACTTTTA |
| | GAAAGCCTTGACTACAATGATGACCTTGGTGTGGCTGTGAACACTGTCAGCTCCCACAGCT |
| | GCTGCAGCAAAAAATGTCCATAGACAGGGTGGGGGCCCGGGGTCGTCTGCTGTCCTGCTCA |
| | GCCCACAGCACGCATGGAGGATCTGAGGTGCCACACCTGACGCCCAGGCCAGAACATGCCT |
| | CCCTCCAGGGTGACCTGCCATGTCCTGCATTGCTGGAGGGACAGGGGCAGCCTATGAGGAT |
| | CTGGGGCCAGGAGATGAATCCTATTAACCCAGAGGAAAACTAACAGGACCCAAGCACCCTC |
| | CCCGTTGAAGCTGACCTGCCCAGAGGGGCCTGGGCCCACCCCACACACCGGGGCGGAATGT |
| | GTACAGGCCCCGGTCTCTGTGGGTGTTCCGCTAACTGGGGCTCCCAGTGCTCACCCCACAA |
| | CTAAAGCGAGCCCCAGCCTCCAGAGCCCCCGAAGGAGATGCCGCCCACAAGCCCAGCCCCC |
| | ATCCAGGAGGCCCCAGAGCTCAGGGCGCCGGGGCAGATTCTGAACAGCCCCGAGTCACGGT |
| | GGGTACAACTGGAACGACCACCGTGAGAAAAACTGTGTCCAAAACTCTCTCCTGGCCCCTG |
| | CTGGAGGCCGCGCCAGAGAGGGGAGCAGCCGCCCCGAACCTAGGTCCTGCTCAGCTCACAC |
| | GACCCCCAGCAGCCAGAGCACAGTGGAGTCCCCACTGAACCCCACTGAATGGTGAGGACGG |
| | GGACCAGGGCTCCAGGGGGTCATGGAAGGGGCTGGACCCCATCCTACTGCTATGGTCCCAG |
| | TGCTCCTGGCCAGAACTGACCCTACCACCGACAAGAGTCCCTCAGGGAAACGGGGGTCACT |
| | GGCACCTCCCAGCATCAACCCCAGGCAGCACAGGCATAAACCCCACATCCAGAGCCGACTC |
| | CAGGAGCAGAGACACCCCAGTACCCTGGGGGACACCGACCCTGATGACTCCCCACTGGAAT |
| | CCACCCCAGAGTCCACCAGGACCAAAGACCCCGCCCCGGTCTCTGTCCCTCACTCAGGACC |
| | TGCTGCGGGGGGGGCCATGAGACCAGACTCGGGCTTAGGGAACACCACTGTGGCCCCAACC |
| | TCGACCAGGCCACAGGCCCTTCCTTCCTGCCCTGCGGCAGCACAGACTTTGGGGTCTGTGC |
| | AGAGAGGAATCACAGAGGCCCCAGGCTGAGGTGGTGGGGGTGGAAGACCCCCAGGAGGTGG |
| | CCCACTTCCCTTCCTCCCAGCTGGAACCCACCATGACCTTCTTAAGATAGGGGTGTCATCC |
| | GAGGCAGGTCCTCCATGGAGCTCCCTTCAGGCTCCTCCCTGGTCCTCACTAGGCCTCAGTC |
| | CCGGCTGTGGGAATGCAGCCACCACAGGCACACCAGGCAGCCCAGACCCAGCCAGCCTGCA |
| | GTGCCCAAGCCCACATTCTGGAGCAGAGCAGGCTGTGTCTGGGAGAGTCTGGGCTCCCCAC |
| | CGCCCCCCGCACACCCCACCCACCCCTGTCCAGGCCCTATGCAGGAGGGTCAGAGCCCCCC |
| | ATGGGGTATGGACTTAGGGTCTCACTCACGCGGCTCCCCTCCTGGGTGAAGGGGTCTCATG |
| | CCCAGATCCCCACAGCAGAGCTGGTCAAAGGTGGAGGCAGTGGCCCCAGGGCCACCCTGAC |
| | CTGGACCCTCAGGCTCCTCTAGCCCTGGCTGCCCTGCTGTCCCTGGGAGGCCTGGACTCCA |
| | CCAGACCACAGGTCCAGGGCACCGCCCATAGGTGCTGCCCACACTCAGTTCACAGGAAGAA |
| | GATAAGCTCCAGACCCCCAAGACTGGGACCTGCCTTCCTGCCACCGCTTGTAGCTCCAGAC |
| | CTCCGTGCCTCCCCCGACCACTTACACACGGGCCAGGGAGCTGTTCCACAAAGATCAACCC |
| | CAAACCGGGACCGCCTGGCACTCGGGCCGCTGCCACTTCCCTCTCCATTTGCTCCCAGCAC |
| | CTCTGTGCTCCCTCCCTCCTCCCTCCTTCAGGGGAACAGCCTGTGCAGCCCCTCCCTGCAC |
| | CCCACACCCTGGGGAGGCCCAACCCTGCCTCCAGCCCTTTCTCCCCCGCTGCTCTTCCTGC |
| | CCATCCAGACAACCCTGGGGTCCCATCCCTGCAGCCTACACCCTGGTCTCCACCCAGACCC |
| | CTGTCTCTCCCTCCAGACACCCCTCCCAGGCCAACCCTGCACATGCAGGCCCTCCCCTTTT |
| | CTGCTGCCAGAGCCTCAGTTTCTACCCTCTGTGCCTACCCCCTGCCTCCTCCTGCCCACAA |
| | CTCGAGCTCTTCCTCTCCTGGGGCCCCTGAGCCATGGCACTGACCGTGCACTCCCACCCCC |
| | ACACTGCCCATGCCCTCACCTTCCTCCTGGACACTCTGACCCTGCTCCCCTCTTGGACCCA |
| | GCCCTGGTATTTCCAGGACAAAGGCTCACCCAAGTCTTCCCCATGCAGGCCCTTGCCCTCA |
| | CTGCCCGGTTACACGGCAGCCTCCTGTGCACAGAAGCAGGGAGCTCAGCCCTTCCACAGGC |
| | AGAAGGCACTGAAAGAAATCGGCCTCCAGCACCCTGATGCACGTCCGCCTGTGTCTCTCAC |
| | TGCCCGCACCTGCAGGGAGGCTCGGCACTCCCTGTAAAGACGAGGGATCCAGGCAGCAACA |
| | TCATGGGAGAATGCAGGGCTCCCAGACAGCCCAGCCCTCTCGCAGGCCTCTCCTGGGAAGA |
| | GACCTGCAGCCACCACTGAACAGCCACGGAGCCCGCTGGATAGTAACTGAGTCAGTGACCG |
| | ACCTGGAGGGCAGGGGAGCAGTGAACCGGAGCCCAGACCATAGGGACAGAGACCAGCCGCT |
| | GACATCCCGAGCCCCTCACTGGCGGCCCCAGAACACCGCGTGGAAACAGAACAGACCCACA |
| | TTCCCACCTGGAACAGGGCAGACACTGCTGAGCCCCCAGCACCAGCCCTGAGAAACACCAG |
| | GCAACGGCATCAGAGGGGGCTCCTGAGAAAGAAAGGAGGGGAGGTCTCCTTCACCAGCAAG |
| | TACTTCCCTTGACCAAAAACAGGGTCCACGCAACTCCCCCAGGACAAAGGAGGAGCCCCCT |
| | GTACAGCACTGGGCTCAGAGTCCTCTCCAACACACCCTGAGTTTCAGACAAAAACCCCCTG |
| | GAAATCATAGTATCAGCAGGAGAACTAGCCAGAGACAGCAAGAGGGGACTCAGTGACTCCC |
| | GCGGGGACAGGAGGATTTTGTGGGGGCTCGTGTCACTGTGAGGATATTGTAGTAGTACCAG |
| | CTGCTATGCCCACAGTGACACAGCCCCATTCCCAAAGCCCTGCTGTAAACGCTTCCACTTC |
| | TGGAGCTGAGGGGCTGGGGGGAGCGTCTGGGAAGTAGGGCCTAGGGGTGGCCATCAATGCC |
| | CAAAACGCACCAGACTCCCCCCCAGACATCACCCCACTGGCCAGTGAGCAGAGTAAACAGA |
| | AAATGAGAAGCAGCTGGGAAGCTTGCACAGGCCCCAAGGAAAGAGCTTTGGCGGGTGTGCA |
| | AGAGGGGATGCGGGCAGAGCCTGAGCAGGGCCTTTTGCTGTTTCTGCTTTCCTGTGCAGAT |
| | AGTTCCATAAACTGGTGTTCAAGATCGATGGCTGGGAGTGAGCCCAGGAGGACAGTGTGGG |
| | AAGGGCACAGGGAAGGAGAAGCAGCCGCTATCCTACACTGTCATCTTTCAAGAGTTTGCCC |
| | TGTGCCCACAATGCTGCATCATGGGATGCTTAACAGCTGATGTAGACACAGCTAAAGAGAG |
| | AATCAGTGAAATGGATTTGCAGCACAGATCTGAATAAATTCTCCAGAATGTGGAGCCACAC |
| | AGAAGCAAGCACAAGGAAAGTGCCTGATGCAAGGGCAAAGTACAGTGTGTACCTTCAGGCT |
| | GGGCACAGACACTCTGAAAAGCCTTGGCAGGAACTCCCTGCAACAAAGCAGAGCCCTGCAG |
| | GCAATGCCAGCTCCAGAGCCCTCCCTGAGAGCCTCATGGGCAAAGATGTGCAGAACATATG |
| | TTTGTCATAGCCCCAAACTGAGAATGAAGCAAACAGCCATCTGAAGGAAAACAGGCAAATA |
| | AACGATGGCAGGTTCATGAAATGCAAACCCAGACAGCCAGAAGGACAACAGTGAGGGTTAC |
| | AGGTGACTCTGTGGTTGAGTTCATGACAATGCTGAGTAATTGGAGTAACAAAGGAAAGTCC |
| | AAAAAATACTTTCAATGTGATTTCTTCTAAATAAAATTTACAGCCGGCAAAATGAACTATC |
| | TTCTTAAGGGATAAACTTTCCACTAGGAAAACTATAAGGAAAATCAAGAAAAGGATGATCA |
| | CATAAACACAGTGGTCGTTACTTCTACTGGGGAAGGAAGAGGGTATGAACTGAGACACACA |
| | GGGTTGGCAAGTCTCCTAACAAGAAGAGAACAAATACATTACAGTACCTTGAAAACAGCAG |
| | TTAAAATTCTAAATTGCAAGAAGAGGAAAATGCACACAGCTGTGTTTAGAAAATTCTCAGT |
| | CCAGCACTGTTCATAATAGCAAAGACATTAACCCAGGTTGGATAAATAAACGATGACACAG |
| | GCAATTGCACAATGATACAGACATACATTCAGTATATGAGACATTGATGATGTATCCCCAA |
| | AGAAATGACTTTAAAGAGAAAAGGCCTGATATGTGGTGGCACTCACCTCCCTGGGCATCCC |
| | CGGACAGGCTGCAGGCACACTGTGTGGCAGGGCAGGCTGGTACCTGCTGGCAGCTCCTGGG |
| | GCCTGATGTGGAGCAGGCACAGAGCCGTATCCCCCCGAGGACATATACCCCCAAGGACGGC |
| | ACAGTTGGTACATTCCGGAGACAAGCAACTCAGCCACACTCCCAGGCCAGAGCCCGAGAGG |
| | GACGCCCATGCACAGGGAGGCAGAGCCCAGCTCCTCCACAGCCAGCAGCACCTGTGCAGGG |
| | GCCGCCATCTGGCAGGCACAGAGCATGGGCTGGGAGGAGGGGCAGGGACACCAGGCAGGGT |
| | TGGCACCAACTGAAAATTACAGAAGTCTCATACATCTACCTCAGCCTTGCCTGACCTGGGC |
| | CTCACCTGACCTGGACCTCACCTGGCCTGGACCTCACCTGGCCTAGACCTCACCTCTGGGC |
| | TTCACCTGAGCTCGGCCTCACCTGACTTGGACCTTGCCTGTCCTGAGCTCACATGATCTGG |
| | GCCTCACCTGACCTGGGTTTCACCTGACCTGGGCTTCACCTGACCTGGGCCTCATCTGACC |
| | TGGGCCTCACTGGCCTGGACCTCACCTGGCCTGGGCTTCACCTGGCCTCAGGCCTCATCTG |
| | CACCTGCTCCAGGTCTTGCTGGAACCTCAGTAGCACTGAGGCTGCAGGGGCTCATCCAGGG |
| | TTGCAGAATGACTCTAGAACCTCCCACATCTCAGCTTTCTGGGTGGAGGCACCTGGTGGCC |
| | CAGGGAATATAAAAAGCCTGAATGATGCCTGCGTGATTTGGGGGCAATTTATAAACCCAAA |
| | AGGACATGGCCATGCAGCGGGTAGGGACAATACAGACAGATATCAGCCTGAAATGGAGCCT |
| | CAGGGCACAGGTGGGCACGGACACTGTCCACCTAAGCCAGGGGCAGACCCGAGTGTCCCCG |
| | CAGTAGACCTGAGAGCGCTGGGCCCACAGCCTCCCCTCGGTGCCCTGCTACCTCCTCAGGT |
| | CAGCCCTGGACATCCCGGGTTTCCCCAGGCCTGGCGGTAGGTTTGGGGTGAGGTCTGTGTC |
| | ACTGTGGTATTACGATTTTTGGAGTGGTTATTATACCCACAGTGTCACAGAGTCCATCAAA |
| | AACCCATCCCTGGGAACCTTCTGCCACAGCCCTCCCTGTGGGGCACCGCTGCGTGCCATGT |
| | TAGGATTTTGACTGAGGACACAGCACCATGGGTATGGTGGCTACCGCAGCAGTGCAGCCTG |
| | TGACCCAAACACACAGGGCAGCAGGCACAACAGACAAGCCCACAAGTGACCACCCTGAGCT |
| | CCTGCCTGCCAGCCCTGGAGACCATGAAACAGATGGCCAGGATTATCCCATAGGTCAGCCA |
| | GACCTCAGTCCAACAGGTCTGCATCGCTGCTGCCCTCCAATACCAGTCCGGATGGGGACAG |
| | GGCCGGCCCACATTACCATTTGCTGCCATCCGGCCAACAGTCCCAGAAGCCCCTCCCTCAA |
| | GGCTGGGCCACATGTGTGGACCCTGAGAGCCCCCCATGTCTGAGTAGGGGCACCAGGAAGG |
| | TGGGGCTGGCCCTGTGCACTGTCACTGCCCCTGTGGTCCCTGGCCTGCCTGGCCCTGACAC |
| | CTGGGCCTCTCCTGGGTCATTTCCAAGACAGAAGACATTCCCAGGACAGCTGGAGCTGGGA |
| | GTCCATCATCCTGCCTGGCCATCCTGAGTCCTGCGCCTTTCCAAACCTCACCCGGGAAGCC |
| | AACAGAGGAATCACCTCCCACAGGCAGAGACAAAGACCTTCCAGAAATCTCTGTCTCTCTC |
| | CCCAGTGGGCACCCTCTTCCAGGGCAGTCCTCAGTGATATCACAGTGGGAACCCACATCTG |
| | GATCGGGACTGCCCCCAGAACACAAGATGGCCCACAGGGACAGCCCCACAGCCCAGCCCTT |
| | CCCAGACCCCTAAAAGGCGTCCCACCCCCTGCATCTGCCCCAGGGCTCAAACTCCAGGAGG |
| | ACTGACTCCTGCACACCCTCCTGCCAGACATCACCTCAGCCCCTCCTGGAAGGGACAGGAG |
| | CGCGCAAGGGTGAGTCAGACCCTCCTGCCCTCGATGGCAGGCGGAGAAGATTCAGAAAGGT |
| | CTGAGATCCCCAGGACGCAGCACCACTGTCAATGGGGGCCCCAGACGCCTGGACCAGGGCC |
| | TGCGTGGGAAAGGCCTCTGGGCACACTCAGGGGCTTTTTGTGAAGGGTCCTCCTACTGTGT |
| | GACTACAGTAACTACCACAGTGATGAACCCAGCAGCAAAAACTGACCGGACTCCCAAGGTT |
| | TATGCACACTTCTCCGCTCAGAGCTCTCCAGGATCAGAAGAGCCGGGCCCAAGGGTTTCTG |
| | CCCAGACCCTCGGCCTCTAGGGACATCTTGGCCATGACAGCCCATGGGCTGGTGCCCCACA |
| | CATCGTCTGCCTTCAAACAAGGGCTTCAGAGGGCTCTGAGGTGACCTCACTGATGACCACA |
| | GGTGCCCTGGCCCCTTCCCCGCCAGCTGCACCAGACCCCGTCCTGACAGATGCCCCGATTC |
| | CAACAGCCAATTCCTGGGGCCAGGAATCGCTGTAGACACCAGCCTCCTTCCAACACCTCTT |
| | GCCAATTGCCTGGATTCCCATCCCGGTTGGAATCAAGAGGACAGCATCCCCCAGGCTCCCA |
| | ACAGGCAGGACTCCCACACCCTCCTCTGAGAGGCCGCTGTGTTCCGTAGGGCCAGGCTGCA |
| | GACAGTCCCCCTCACCTGCCACTAGACAAATGCCTGCTGTAGATGTCCCCACCTGGAAAAG |
| | ACCACTCATGGAGCCCCCAGCCCCAGGTACAGCCATAGAGAGAGTCTCTGAGGCCCCTAAG |
| | AAGTAGCCATGCCCAGTTCTGCCGGGACCCTCGGCCAGGCTGACAGGAGTGGACGCTGGAG |
| | CTGGGCCCACACTGGGCCACATAGGAGCTCACCAGTGAGGGCAGGAGAGCACATGCCGGGG |
| | AGCACCCAGCCTCCTGCTGACCAGAGGCCCGTCCCAGAGCCCAGGAGGCTGCAGAGGCCTC |
| | TCCAGGGGGACACTGTGCATGTCTGGTCCCTGAGCAGCCCCCCATGTCCCCAGTCCTGGGG |
| | GCCCCTGGCACAGCTGTCTGGACCCTCTCTATTCCCTGGGAAGCTCCTCCTGACAGCCCCG |
| | CCTCCAGTTCCAGGTGTGGTTATTGTCAGGGGGTGTCAGACTGTGGTGGATACAGCTATGG |
| | TTACCACAGTGGTGCTGCCCATAGCAGCAACCAGGCCAAGTAGACAGGCCCCTGCTGTGCA |
| | GCCCCAGGCCTCCAGCTCACCTGCTTCTCCTGGGGCTCTCAAGGCTGCTGTTTTCTGCACT |
| | CTCCCCTCTGTGGGGAGGGTTCCCTCAGTGGGAGATCTGTTCTCAACATCCCAGGGCCTCA |
| | TTCCTGCAAGGAAGGCCAATGGATGGGCAACCTCACATGCCGCGGCTAAGATAGGGTGGGC |
| | AGCCTGGCGGGGACAGGACATCCTGCTGGGGTATCTGTCACTGTGCCTAGTGGGGCACTGG |
| | CTCCCAAACAACGCAGTCCTCGCCAAAATCCCCACGGCCTCCCCCGCTAGGGGCTGGCCTG |
| | ATCTCCTGCAGTCCTAGGAGGCTGCTGACCTCCAGAATGGCTCCGTCCCCAGTTCCAGGGC |
| | GAGAGCAGATCCCAGGCCGGCTGCAGACTGGGAGGCCACCCCCTCCTTCCCAGGGTTCACT |
| | GCAGGTGACCAGGGCAGGAAATGGCCTGAACACAGGGATAACCGGGCCATCCCCCAACAGA |
| | GTCCACCCCCTCCTGCTCTGTACCCCGCACCCCCAAGGCCAGCCCATGACATCCGACAACC |
| | CCACACCAGAGTCACTGCCCGGTGCTGCCCTAGGGAGGACCCCTCAGCCCCCACCCTGTCT |
| | AGAGGACTGGGGAGGACAGGACACGCCCTCTCCTTATGGTTCCCCCACCTGGCTCTGGCTG |
| | GGACCCTTGGGGTGTGGACAGAAAGGACGCTTGCCTGATTGGCCCCCAGGAGCCCAGAACT |
| | TCTCTCCAGGGACCCCAGCCCGAGCACCCCCTTACCCAGGACCCAGCCCTGCCCCTCCTCC |
| | CATCTGCTCTCCTCTCATCACCCCATGGGAATCCAGAATCCCCAGGAAGCCATCAGGAAGG |
| | GCTGAGGGAGGAAGTGGGGCCACTGCACCACCAGGCAGGAGGCTCCGTCTTTGTGAACCCA |
| | GGGAGGTGCCAGCCTCCTAGAGGGTATGGTCCACCCTGCCTATGGCTCCCACAGTGGCAGG |
| | CTGCAGGGAAGGACCAGGGACGGTGTGGGGGAGGGCTCAGGGCCCCGCGGGTGCTCCATCT |
| | TGGATGAGCCCATCTCTCTCACCCACGGACTCACCCACCTCCTCTCCACCCTGGCCACACG |
| | TCGTCCACACCATCCTAAGTCCCACCTACACCAGAGCCGGCACAGCCAGTGCAGACAGAGG |
| | CTGGGGTGCAGGGGGGCCGCCAGGGCAGCTTTGGGGAGGGAAGGATGGAGGAAGGGGAGTT |
| | CAGTGAAGAGGCCCCCCTCCCCTGGGTCCAGGATCCTCCTCTGGGACCCCCGGATCCCATC |
| | CCCTCCAGGCTCTGGGAGGAGAAGCAGGATGGGAGAATCTGTGCGGGACCCTCTCACAGTG |
| | GAATACCTCCACAGCGGCTCAGGCAAGACCCAAAAGCCCCTCAGTGAGCCCTCCACTGCAG |
| | TCCTGGGCCTGGGTAGCAGCCCCTCCCACAGAGGATGAACCCAGCACCCCGAGGATGTCCT |
| | GCCAGGGGGAGCTCAGAGCCATGAAGGAGCAGGATATGGGACCCCCGATACAGGCACAGAC |
| | CTCAGCTCCATTCAGGACTGCCACGTCCTGCCCTGGGAGGAACCCCTTTCTCTAGTCCCTG |
| | CAGGCCAGGAGGCAGCTGACTCCTGACTTGGACGCCTATTCCAGACACCAGACAGAGGGGC |
| | AGGCCCCCCAGAACCAGGGATGAGGACGCCCCGTCAAGGCCAGAAAAGACCAAGTTGTGCT |
| | GAGCCCAGCAAGGGAAGGTCCCCAAACAAACCAGGAAGTTTCTGAAGGTGTCTGTGTCACA |
| | GTGGAGTATAGCAGCTCGTCCCACAGTGACACTCGCCAGGCCAGAAACCCCATCCCAAGTC |
| | AGCGGAATGCAGAGAGAGCAGGGAGGACATGTTTAGGATCTGAGGCCGCACCTGACACCCA |
| | GGCCAGCAGACGTCTCCTGTCCATGGCACCCTGCCATGTCCTGCATTTCTGGAAGAACAAG |
| | GGCAGGCTGAAGGGGGTCCAGGACCAGGAGATGGGTCCCCTCTACCCAGAGAAGGAGCCAG |
| | GCAGGACACAAGCCCCCTCCCCATTGAGGCTGACCTGCCCAGAGGGTCCTGGGCCCACCCC |
| | ACACACCGGGGCGGAATGTGTGCAGGCCTCGGTCTCTGTGGGTGTTCCGCTAGCTGGGGCT |
| | CACAGTGCTCACCCCACACCTAAAACGAGCCACAGCCTCAGAGCCCCTGAAGGAGACCCCG |
| | CCCACAAGCCCAGCCCCCACCCAGGAGGCCCCAGAGCACAGGGCGCCCCGTCGGATTCTGA |
| | ACAGCCCCGAGTCACAGTGGGTATAACTGGAACTACCACTGTGAGAAAAGCTTCGTCCAAA |
| | ACGGTCTCCTGGCCACAGTCGGAGGCCCCGCCAGAGAGGGGAGCAGCCACCCCAAACCCAT |
| | GTTCTGCCGGCTCCCATGACCCCGTGCACCTGGAGCCCCACAGTGTCCCCACTGGATGGGA |
| | GGACAAGGGCCGGGGGCTCCGGCGGGTCGGGGCAGGGGCTTGATGGCTTCCTTCTGCCGTG |
| | GCTCCAGTGCCCCTGGCTGGAGTTGACCCTTCTGACAAGTGTCCTCAGAGAGTCAGGGATC |
| | AGTGGCACCTCCCAACATCAACCCCACGCAGCCCAGGCACAAACCCCACATCCAGGGCCAA |
| | CTCCAGGAACAGAGACACCCCAATACCCTGGGGGACCCCAACCCTGATGACTCCCGTCCCA |
| | TCTCTGTCCCTCACTTGGGGCCTGCTGCGGGGCGAGCACTTGGGAGCAAACTCAGGCTTAG |
| | GGGACACCACTGTGGGCCTGACCTCGAGCAGGCCACAGACCCTTCCCTCCTGCCCTGGTGC |
| | AGCACAGACTTTGGGGTCTGGGCAGGGAGGAACTTCTGGCAGGTCACCAAGCACAGAGCCC |
| | CCAGGCTGAGGTGGCCCCAGGGGGAACCCCAGCAGGTGGCCCACTACCCTTCCTCCCAGCT |
| | GGACCCCATGTCTTCCCCAAGATAGGGGTGCCATCCAAGGCAGGTCCTCCATGGAGCCCCC |
| | TTCAGGCTCCTCTCCAGACCCCACTGGGCCTCAGTCCCCACTCTAGGAATGCAGCCACCAC |
| | GGGCACACCAGGCAGCCCAGGCCCAGCCACCCTGCAGTGCCCAAGCCCACACCCTGGAGGA |
| | GAGCAGGGTGCGTCTGGGAGGGGCTGGGCTCCCCACCCCCACCCCCACCTGCACACCCCAC |
| | CCACCCTTGCCCGGGCCCCCTGCAGGAGGGTCAGAGCCCCCATGGGATATGGACTTAGGGT |
| | CTCACTCACGCACCTCCCCTCCTGGGAGAAGGGGTCTCATGCCCAGATCCCCCCAGCAGCG |
| | CTGGTCACAGGTAGAGGCAGTGGCCCCAGGGCCACCCTGACCTGGCCCCTCAGGCTCCTCT |
| | AGCCCTGGCTGCCCTGCTGTCCCTGGGAGGCCTGGGCTCCACCAGACCACAGGTCTAGGGC |
| | ACCGCCCACACTGGGGCCGCCCACACACAGCTCACAGGAAGAAGATAAGCTCCAGACCCCC |
| | AGGCCCGGGACCTGCCTTGCTGCTACGACTTCCTGCCCCAGACCTCGTTGCCCTCCCCCGT |
| | CCACTTACACACAGGCCAGGAAGCTGTTCCCACACAGACCAACCCCAGACGGGGACCACCT |
| | GGCACTCAGGTCACTGCCATTTCCTTCTCCATTCACTTCCAATGCCTCTGTGCTTCCTCCC |
| | TCCTCCTTCCTTCGGGGGAGCACCCTGTGCAGCTCCTCCCTGCAGTCCACACCCTGGGGAG |
| | ACCCGACCCTGCAGCCCACACCCTGGGGAGACCTGACCCTCCTCCAGCCCTTTCTCCCCCG |
| | CTGCTCTTGCCACCCACCAAGACAGCCCTGGGGTCCTGTCCCTACAGCCCCCACCCAGTTC |
| | TCTACCTAGACCCGTCTTCCTCCCTCTAAACACCTCTCCCAGGCCAACCCTACACCTGCAG |
| | GCCCTCCCCTCCACTGCCAAAGACCCTCAGTTTCTCCTGCCTGTGCCCACCCCCGTGCTCC |
| | TCCTGCCCACAGCTCGAGCTCTTCCTCTCCTAGGGCCCCTGAGGGATGGCATTGACCGTGC |
| | CCTCGCACCCACACACTGCCCATGCCCTCACATTCCTCCTGGCCACTCCAGCCCCACTCCC |
| | CTCTCAGGCCTGGCTCTGGTATTTCTGGGACAAAGCCTTACCCAAGTCTTTCCCATGCAGG |
| | CCTGGGCCCTTACCCTCACTGCCCGGTTACAGGGCAGCCTCCTGTGCACAGAAGCAGGGAG |
| | CTCAGCCCTTCCACAGGCAGAAGGCACTGAAAGAAATCGGCCTCCAGCGCCTTGACACACG |
| | TCTGCCTGTGTCTCTCACTGCCCGCACCTGCAGGGAGGCTCGGCACTCCCTCTAAAGACGA |
| | GGGATCCAGGCAGCAGCATCACAGGAGAATGCAGGGCTACCAGACATCCCAGTCCTCTCAC |
| | AGGCCTCTCCTGGGAAGAGACCTGAAGACGCCCAGTCAACGGAGTCTAACACCAAACCTCC |
| | CTGGAGGCCGATGGGTAGTAACGGAGTCATTGCCAGACCTGGAGGCAGGGGAGCAGTGAGC |
| | CCGAGCCCACACCATAGGGCCAGAGGACAGCCACTGACATCCCAAGCCACTCACTGGTGGT |
| | CCCACAACACCCCATGGAAAGAGGACAGACCCACAGTCCCACCTGGACCAGGGCAGAGACT |
| | GCTGAGACCCAGCACCAGAACCAACCAAGAAACACCAGGCAACAGCATCAGAGGGGGCTCT |
| | GGCAGAACAGAGGAGGGGAGGTCTCCTTCACCAGCAGGCGCTTCCCTTGACCGAAGACAGG |
| | ATCCATGCAACTCCCCCAGGACAAAGGAGGAGCCCCTTGTTCAGCACTGGGCTCAGAGTCC |
| | TCTCCAAGACACCCAGAGTTTCAGACAAAAACCCCCTGGAATGCACAGTCTCAGCAGGAGA |
| | GCCAGCCAGAGCCAGCAAGATGGGGCTCAGTGACACCCGCAGGGACAGGAGGATTTTGTGG |
| | GGGCTCGTGTCACTGTGAGGATATTGTACTAATGGTGTATGCTATACCCACAGTGACACAG |
| | CCCCATTCCCAAAGCCCTACTGCAAACGCATTCCACTTCTGGGGCTGAGGGGCTGGGGGAG |
| | CGTCTGGGAAATAGGGCTCAGGGGTGTCCATCAATGCCCAAAACGCACCAGACTCCCCTCC |
| | ATACATCACACCCACCAGCCAGCGAGCAGAGTAAACAGAAAATGAGAAGCAAGCTGGGGAA |
| | GCTTGCACAGGCCCCAAGGAAAGAGCTTTGGCGGGTGTGTAAGAGGGGATGCGGGCAGAGC |
| | CTGAGCAGGGCCTTTTGCTGTTTCTGCTTTCCTGTGCAGAGAGTTCCATAAACTGGTGTTC |
| | GAGATCAATGGCTGGGAGTGAGCCCAGGAGGACAGCGTGGGAAGAGCACAGGGAAGGAGGA |
| | GCAGCCGCTATCCTACACTGTCATCTTTCGAAAGTTTGCCTTGTGCCCACACTGCTGCATC |
| | ATGGGATGCTTAACAGCTGATGTAGACACAGCTAAAGAGAGAATCAGTGAGATGGATTTGC |
| | AGCACAGATCTGAATAAATTCTCCAGAATGTGGAGCAGCACAGAAGCAAGCACACAGAAAG |
| | TGCCTGATGCAAGGACAAAGTTCAGTGGGCACCTTCAGGCATTGCTGCTGGGCACAGACAC |
| | TCTGAAAAGCCCTGGCAGGAACTCCCTGTGACAAAGCAGAACCCTCAGGCAATGCCAGCCC |
| | CAGAGCCCTCCCTGAGAGCCTCATGGGCAAAGATGTGCACAACAGGTGTTTCTCATAGCCC |
| | CAAACTGAGAGCAAAGCAAACGTCCATCTGAAGGAGAACAGGCAAATAAACGATGGCAGGT |
| | TCATGAAATGCAAACCCAGACAGCCACAAGCACAAAAGTACAGGGTTATAAGCGACTCTGG |
| | TTGAGTTCATGACAATGCTGAGTAATTGGAGTAACAAAGTAAACTCCAAAAAATACTTTCA |
| | ATGTGATTTCTTCTAAATAAAATTTACACCCTGCAAAATGAACTGTCTTCTTAAGGGATAC |
| | ATTTCCCAGTTAGAAAACCATAAAGAAAACCAAGAAAAGGATGATCACATAAACACAGTGG |
| | TGGTTACTTCTGCTGGGGAAGGAAGAGGGTATGAACTGAGATACACAGGGTGGGCAAGTCT |
| | CCTAACAAGAACAGAACGAATACATTACAGTACCTTGAAAACAGCAGTTAAACTTCTAAAT |
| | TGCAAGAAGAGGAAAATGGACACAGTTGTGTTTAGAAAATTCTCAGTCCAGCACTGTTCAT |
| | AATAGCAAAGACATTAACCCAGGTCGGATAAATAAGCGATGACACAGGCAATTGCACAATG |
| | ATACAGACATATATTTAGTATATGAGACATCGATGATGTATCCCCAAATAAACGACTTTAA |
| | AGAGATAAAGGGCTGATGTGTGGTGGCATTCACCTCCCTGGGATCCCCGGACAGGTTGCAG |
| | GCTCACTGTGCAGCAGGGCAGGCGGGTACCTGCTGGCAGTTCCTGGGGCCTGATGTGGAGC |
| | AAGCGCAGGGCCATATATCCCGGAGGACGGCACAGTCAGTGAATTCCAGAGAGAAGCAACT |
| | CAGCCACACTCCCCAGGCAGAGCCCGAGAGGGACGCCCACGCACAGGGAGGCAGAGCCCAG |
| | CACCTCCGCAGCCAGCACCACCTGTGCACGGGCCACCACCTTGCAGGCACAGAGTGGGTGC |
| | TGAGAGGAGGGGCAGGGACACCAGGCAGGGTGAGCACCCAGAGAAAACTGCAGACGCCTCA |
| | CACATCCACCTCAGCCTCCCCTGACCTGGACCTCACTGGCCTGGGCCTCACTTAACCTGGG |
| | CTTCACCTGACCTTGGCCTCACCTGACTTGGACCTCGCCTGTCCCAAGCTTTACCTGACCT |
| | GGGCCTCAACTCACCTGAACGTCTCCTGACCTGGGTTTAACCTGTCCTGGAACTCACCTGG |
| | CCTTGGCTTCCCCTGACCTGGACCTCATCTGGCCTGGGCTTCACCTGGCCTGGGCCTCACC |
| | TGACCTGGACCTCATCTGGCCTGGACCTCACCTGGCCTGGACTTCACCTGGCCTGGGCTTC |
| | ACCTGACCTGGACCTCACCTGGCCTCGGGCCTCACCTGCACCTGCTCCAGGTCTTGCTGGA |
| | GCCTGAGTAGCACTGAGGGTGCAGAAGCTCATCCAGGGTTGGGGAATGACTCTAGAAGTCT |
| | CCCACATCTGACCTTTCTGGGTGGAGGCAGCTGGTGGCCCTGGGAATATAAAAATCTCCAG |
| | AATGATGACTCTGTGATTTGTGGGCAACTTATGAACCCGAAAGGACATGGCCATGGGGTGG |
| | GTAGGGACATAGGGACAGATGCCAGCCTGAGGTGGAGCCTCAGGACACAGGTGGGCACGGA |
| | CACTATCCACATAAGCGAGGGATAGACCCGAGTGTCCCCACAGCAGACCTGAGAGCGCTGG |
| | GCCCACAGCCTCCCCTCAGAGCCCTGCTGCCTCCTCCGGTCAGCCCTGGACATCCCAGGTT |
| | TCCCCAGGCCTGCCGGTAGGTTTAGAATGAGGTCTGTGTCACTGTGGTATTACGATATTTT |
| | GACTGGTTATTATAACCACAGTGTCACAGAGTCCATCAAAAACCCATGCCTGGAAGCTTCC |
| | CGCCACAGCCCTCCCCATGGGGCCCTGCTGCCTCCTCAGGTCAGCCCCGGACATCCCGGGT |
| | TTCCCCAGGCTGGGCGGTAGGTTTGGGGTGAGGTCTGTGTCACTGTGGTATTACTATGGTT |
| | CGGGGAGTTATTATAACCACAGTGTCACAGAGTCCATCAAAAACCCATCCCTGGGAGCCTC |
| | CCGCCACAGCCCTCCCTGCAGGGGACCGGTACGTGCCATGTTAGGATTTTGATCGAGGAGA |
| | CAGCACCATGGGTATGGTGGCTACCACAGCAGTGCAGCCTGTGACCCAAACCCGCAGGGCA |
| | GCAGGCACGATGGACAGGCCCGTGACTGACCACGCTGGGCTCCAGCCTGCCAGCCCTGGAG |
| | ATCATGAAACAGATGGCCAAGGTCACCCTACAGGTCATCCAGATCTGGCTCCGAGGGGTCT |
| | GCATCGCTGCTGCCCTCCCAACGCCAGTCCAAATGGGACAGGGACGGCCTCACAGCACCAT |
| | CTGCTGCCATCAGGCCAGCGATCCCAGAAGCCCCTCCCTCAAGGCTGGGCCACATGTGTGG |
| | ACACTGAGAGCCCTCATGTCTGAGTAGGGGCACCAGGAGGGAGGGGCTGGCCCTGTGCACT |
| | GTCCCTGCCCCTGTGGTCCCTGGCCTGCCTGGCCCTGACACCTGAGCCTCTCCTGGGTCAT |
| | TTCCAAGACAGAAGACATTCCTGGGGACAGCCGGAGCTGGGCGTCGCTCATCCTGCCCGGC |
| | CGTCCTGAGTCCTGCTCATTTCCAGACCTCACCGGGGAAGCCAACAGAGGACTCGCCTCCC |
| | ACATTCAGAGACAAAGAACCTTCCAGAAATCCCTGCCTCTCTCCCCAGTGGACACCCTCTT |
| | CCAGGACAGTCCTCAGTGGCATCACAGCGGCCTGAGATCCCCAGGACGCAGCACCGCTGTC |
| | AATAGGGGCCCCAAATGCCTGGACCAGGGCCTGCGTGGGAAAGGTCTCTGGCCACACTCGG |
| | GCTTTTTGTGAAGGGCCCTCCTGCTGTGTGACTACAGTAACTACCATAGTGATGAACCCAG |
| | TGGCAAAAACTGGCTGGAAACCCAGGGGCTGTGTGCACGCCTCAGCTTGGAGCTCTCCAGG |
| | AGCACAAGAGCCGGGCCCAAGGATTTGTGCCCAGACCCTCAGCCTCTAGGGACACCTGGGC |
| | CATCTCAGCCTGGGCTGGTGCCCTGCACACCATCTTCCTCCAAATAGGGGCTTCAGAGGGC |
| | TCTGAGGTGACCTCACTCATGACCACAGGTGACCTGGCCCTTCCCTGCCAGCTATACCAGA |
| | CCCTGTCTTGACAGATGCCCCGATTCCAACAGCCAATTCCTGGGACCCTGAATAGCTGTAG |
| | ACACCAGCCTCATTCCAGTACCTCCTGCCAATTGCCTGGATTCCCATCCTGGCTGGAATCA |
| | AGAAGGCAGCATCCGCCAGGCTCCCAACAGGCAGGACTCCCGCACACCCTCCTCTGAGAGG |
| | CCGCTGTGTTCCGCAGGGCCAGGCCCTGGACAGTTCCCCTCACCTGCCACTAGAGAAACAC |
| | CTGCCATTGTCGTCCCCACCTGGAAAAGACCACTCGTGGAGCCCCCAGCCCCAGGTACAGC |
| | TGTAGAGAGAGTCCTCGAGGCCCCTAAGAAGGAGCCATGCCCAGTTCTGCCGGGACCCTCG |
| | GCCAGGCCGACAGGAGTGGACGCTGGAGCTGGGCCCACACTGGGCCACATAGGAGCTCACC |
| | AGTGAGGGCAGGAGAGCACATGCCGGGGAGCACCCAGCCTCCTGCTGACCAGAGGCCTGCC |
| | CCAGAGCCCAGGAGGCTGCAGAGGCCTCTCCAGGGAGACACTGTGCATGTCTGGTACCTAA |
| | GCAGCCCCCCACGTCCCCAGTCCTGGGGGCCCCTGGCTCAGCTGTCTGGACCCTCCCTGTT |
| | CCCTGGGAAGCTCCTCCTGACAGCCCCGCCTCCAGTTCCAGGTGTGGTTATTGTCAGGCGA |
| | TGTCAGACTGTGGTGGATATAGTGGCTACGATTACCACAGTGGTGCCGCCCATAGCAGCAA |
| | CCAGGCCAAGTAGACAGGCCCCTGCTGCGCAGCCCCAGGCATCCACTTCACCTGCTTCTCC |
| | TGGGGCTCTCAAGGCTGCTGTCTGTCCTCTGGCCCTCTGTGGGGAGGGTTCCCTCAGTGGG |
| | AGGTCTGTGCTCCAGGGCAGGGATGATTGAGATAGAAATCAAAGGCTGGCAGGGAAAGGCA |
| | GCTTCCCGCCCTGAGAGGTGCAGGCAGCACCACGGAGCCACGGAGTCACAGAGCCACGGAG |
| | CCCCCATTGTGGGCATTTGAGAGTGCTGTGCCCCCGGCAGGCCCAGCCCTGATGGGGAAGC |
| | CTGTCCCATCCCACAGCCCGGGTCCCACGGGCAGCGGGCACAGAAGCTGCCAGGTTGTCCT |
| | CTATGATCCTCATCCCTCCAGCAGCATCCCCTCCACAGTGGGGAAACTGAGGCTTGGAGCA |
| | CCACCCGGCCCCCTGGAAATGAGGCTGTGAGCCCAGACAGTGGGCCCAGAGCACTGTGAGT |
| | ACCCCGGCAGTACCTGGCTGCAGGGATCAGCCAGAGATGCCAAACCCTGAGTGACCAGCCT |
| | ACAGGAGGATCCGGCCCCACCCAGGCCACTCGATTAATGCTCAACCCCCTGCCCTGGAGAC |
| | CTCTTCCAGTACCACCAGCAGCTCAGCTTCTCAGGGCCTCATCCCTGCAAGGAAGGTCAAG |
| | GGCTGGGCCTGCCAGAAACACAGCACCCTCCCTAGCCCTGGCTAAGACAGGGTGGGCAGAC |
| | GGCTGTGGACGGGACATATTGCTGGGGCATTTCTCACTGTCACTTCTGGGTGGTAGCTCTG |
| | ACAAAAACGCAGACCCTGCCAAAATCCCCACTGCCTCCCGCTAGGGGCTGGCCTGGAATCC |
| | TGCTGTCCTAGGAGGCTGCTGACCTCCAGGATGGCTCCGTCCCCAGTTCCAGGGCGAGAGC |
| | AGATCCCAGGCAGGCTGTAGGCTGGGAGGCCACCCCTGCCCTTGCCGGGGTTGAATGCAGG |
| | TGCCCAAGGCAGGAAATGGCATGAGCACAGGGATGACCGGGACATGCCCCACCAGAGTGCG |
| | CCCCTTCCTGCTCTGCACCCTGCACCCCCCAGGCCAGCCCACGACGTCCAACAACTGGGCC |
| | TGGGTGGCAGCCCCACCCAGACAGGACAGACCCAGCACCCTGAGGAGGTCCTGCCAGGGGG |
| | AGCTAAGAGCCATGAAGGAGCAAGATATGGGGCCCCCGATACAGGCACAGATGTCAGCTCC |
| | ATCCAGGACCACCCAGCCCACACCCTGAGAGGAACGTCTGTCTCCAGCCTCTGCAGGTCGG |
| | GAGGCAGCTGACCCCTGACTTGGACCCCTATTCCAGACACCAGACAGAGGCGCAGGCCCCC |
| | CAGAACCAGGGTTGAGGGACGCCCCGTCAAAGCCAGACAAAACCAAGGGGTGTTGAGCCCA |
| | GCAAGGGAAGGCCCCCAAACAGACCAGGAGGTTTCTGAAGGTGTCTGTGTCACAGTGGGGT |
| | ATAGCAGCAGCTGGTACCACAGTGACACTCACCCAGCCAGAAACCCCATTCCAAGTCAGCG |
| | GAAGCAGAGAGAGCAGGGAGGACACGTTTAGGATCTGAGACTGCACCTGACACCCAGGCCA |
| | GCAGACGTCTCCCCTCCAGGGCACCCCACCCTGTCCTGCATTTCTGCAAGATCAGGGGCGG |
| | CCTGAGGGGGGGTCTAGGGTGAGGAGATGGGTCCCCTGTACACCAAGGAGGAGTTAGGCAG |
| | GTCCCGAGCACTCTCCCCATTGAGGCTGACCTGCCCAGAGAGTCCTGGGCCCACCCCACAC |
| | ACCGGGGCGGAATGTGTGCAGGCCTCGGTCTCTGTGGGTGTTCCGCTAGCTGGGGCTCACA |
| | GTGCTCACCCCACACCTAAAATGAGCCACAGCCTCCGGAGCCCCCGCAGGAGACCCCGCCC |
| | ACAAGCCCAGCCCCCACCCAGGAGGCCCCAGAGCTCAGGGCGCCCCGTCGGATTCCGAACA |
| | GCCCCGAGTCACAGCGGGTATAACCGGAACCACCACTGTCAGAATAGCTACGTCAAAAACT |
| | GTCCAGTGGCCACTGCCGGAGGCCCCGCCAGAGAGGGCAGCAGCCACTCTGATCCCATGTC |
| | CTGCCGGCTCCCATGACCCCCAGCACGCGGAGCCCCACAGTGTCCCCACTGGATGGGAGGA |
| | CAAGAGCTGGGGATTCCGGCGGGTCGGGGCAGGGGCTTGATCGCATCCTTCTGCCGTGGCT |
| | CCAGTGCCCCTGGCTGGAGTTGACCCTTCTGACAAGTGTCCTCAGAGAGACAGGCATCACC |
| | GGCGCCTCCCAACATCAACCCCAGGCAGCACAGGCACAAACCCCACATCCAGAGCCAACTC |
| | CAGGAGCAGAGACACCCCAATACCCTGGGGGACCCCGACCCTGATGACTTCCCACTGGAAT |
| | TCGCCGTAGAGTCCACCAGGACCAAAGACCCTGCCTCTGCCTCTGTCCCTCACTCAGGACC |
| | TGCTGCCGGGCGAGGCCTTGGGAGCAGACTTGGGCTTAGGGGACACCAGTGTGACCCCGAC |
| | CTTGACCAGGACGCAGACCTTTCCTTCCTTTCCTGGGGCAGCACAGACTTTGGGGTCTGGG |
| | CCAGGAGGAACTTCTGGCAGGTCGCCAAGCACAGAGGCCACAGGCTGAGGTGGCCCTGGAA |
| | AGACCTCCAGGAGGTGGCCACTCCCCTTCCTCCCAGCTGGACCCCATGTCCTCCCCAAGAT |
| | AAGGGTGCCATCCAAGGCAGGTGCTCCTTGGAGCCCCATTCAGACTCCTCCCTGGACCCCA |
| | CTGGGCCTCAGTCCCAGCTCTGGGGATGAAGCCACCACAAGCACACCAGGCAGCCCAGGCC |
| | CAGCCACCCTGCAGTGCCCAAGCACACACTCTGGAGCAGAGCAGGGTGCCTCTGGGAGGGG |
| | CTGAGCTCCCCACCCCACCCCCACCTGCACACCCCACCCACCCCTGCCCAGCGGCTCTGCA |
| | GGAGGGTCAGAGCCCCACATGGGGTATGGACTTAGGGTCTCACTCACGTGGCTCCCATCAT |
| | GAGTGAAGGGGCCTCAAGCCCAGGTTCCCACAGCAGCGCCTGTCGCAAGTGGAGGCAGAGG |
| | CCCGAGGGCCACCCTGACCTGGTCCCTGAGGTTCCTGCAGCCCAGGCTGCCCTGCTGTCCC |
| | TGGGAGGCCTGGGCTCCACCAGACCACAGGTCCAGGGCACCGGGTGCAGGAGCCACCCACA |
| | CACAGCTCACAGGAAGAAGATAAGCTCCAGACCCCCAGGGCCAGAACCTGCCTTCCTGCTA |
| | CTGCTTCCTGCCCCAGACCTGGGCGCCCTCCCCCGTCCACTTACACACAGGCCAGGAAGCT |
| | GTTCCCACACAGAACAACCCCAAACCAGGACCGCCTGGCACTCAGGTGGCTGCCATTTCCT |
| | TCTCCATTTGCTCCCAGCGCCTCTGTCCTCCCTGGTTCCTCCTTCGGGGGAACAGCCTGTG |
| | CAGCCAGTCCCTGCAGCCCACACCCTGGGGAGACCCAACCCTGCCTGGGGCCCTTCCAACC |
| | CTGCTGCTCTTACTGCCCACCCAGAAAACTCTGGGGTCCTGTCCCTGCAGTCCCTACCCTG |
| | GTCTCCACCCAGACCCCTGTGTATCACTCCAGACACCCCTCCCAGGCCAACCCTGCACCTG |
| | CAGGCCCTGTCCTCTTCTGTCGCTAGAGCCTCAGTTTCTCCCCCCTGTGCCCACACCCTAC |
| | CTCCTCCTGCCCACAACTCTAACTCTTCTTCTCCTGGAGCCCCTGAGCCATGGCATTGACC |
| | CTGCCCTCCCACCACCCACAGCCCATGCCCTCACCTTCCTCCTGGCCACTCCGACCCCGCC |
| | CCCTCTCAGGCCAAGCCCTGGTATTTCCAGGACAAAGGCTCACCCAAGTCTTTCCCAGGCA |
| | GGCCTGGGCTCTTGCCCTCACTTCCCGGTTACACGGGAGCCTCCTGTGCACAGAAGCAGGG |
| | AGCTCAGCCCTTCCACAGGCAGAAGGCACTGAAAGAAATCGGCCTCCAGCACCTTGACACA |
| | CGTCCCCCCGTGTCTCTCACTGCCCGCACCTGCAGGGAGGCTCCGCACTCCCTCTAAAGAC |
| | AAGGGATCCAGGCAGCAGCATCACGGGAGAATGCAGGGCTCCCAGACATCCCAGTCCTCTC |
| | ACAGGCCTCTCCTGGGAAGAGACCTGCAGCCACCACCAAACAGCCACAGAGGCTGCTGGAT |
| | AGTAACTGAGTCAATGACCGACCTGGAGGGCAGGGGAGCAGTGAGCCGGAGCCCATACCAT |
| | AGGGACAGAGACCAGCCGCTGACATCCCGAGCTCCTCAATGGTGGCCCCATAACACACCTA |
| | GGAAACATAACACACCCACAGCCCCACCTGGAACAGGGCAGAGACTGCTGAGCCCCCAGCA |
| | CCAGCCCCAAGAAACACCAGGCAACAGTATCAGAGGGGGCTCCCGAGAAAGAGAGGAGGGG |
| | AGATCTCCTTCACCATCAAATGCTTCCCTTGACCAAAAACAGGGTCCACGCAACTCCCCCA |
| | GGACAAAGGAGGAGCCCCCTATACAGCACTGGGCTCAGAGTCCTCTCTGAGACACCCTGAG |
| | TTTCAGACAACAACCCGCTGGAATGCACAGTCTCAGCAGGAGAACAGACCAAAGCCAGCAA |
| | AAGGGACCTCGGTGACACCAGTAGGGACAGGAGGATTTTGTGGGGGCTCGTGTCACTGTGA |
| | GGATATTGTAGTGGTGGTAGCTGCTACTCCCACAGTGACACAGACCCATTCCCAAAGCCCT |
| | ACTGCAAACACACCCACTCCTGGGGCTGAGGGGCTGGGGGAGCGTCTGGGAAGTAGGGTCC |
| | AGGGGTGTCTATCAATGTCCAAAATGCACCAGACTGCCCGCCAAACACCACCCCACCAGCC |
| | AGCGAGCAGGGTAAACAGAAAATGAGAGGCTCTGGGAAGCTTGCACAGGCCCCAAGGAAAG |
| | AGCTTTGGCGGGTGTGCAAGAGGGGATGCAGGCAGAGCCTGAGCAGGGCCTTTTGCTGTTT |
| | CTGCTTTCCTGTGCAGAGAGTTCCATAAACTGGTGTTCAAGATCAGTGGCTGGGAATGAGC |
| | CCAGGAGGGCAGTCTGTGGGAAGAGCACAGGGAAGGAGGAGCAGCCGCTATCCTACACTGT |
| | CATCTTTCAAAAGTTTGCCTTGTGACCACACTATTGCATCATGGGATGCTTAAGAGCTGAT |
| | GTAGACACAGCTAAAGAGAGAATCAGTGAGATGAATTTGCAGCATAGATCTGAATAAACTC |
| | TCCAGAATGTGGAGCAGTACAGAAGCAAACACACAGAAAGTGCCTGATGCAAGGACAAAGT |
| | TCAGTGGGCACCTTCAGGCATTGCTGCTGGGCACAGACACTCTGAAAAGCCCTGGCAGGAT |
| | CTCCCTGCGACAAAGCAGAACCCTCAGGCAATGCCAGCCCCAGAGCCCTCCCTGAGAGCGT |
| | CATGGGGAAAGATGTGCAGAACAGCTGATTATCATAGACTCAAACTGAGAACAGAGCAAAC |
| | GTCCATCTGAAGAACAGTCAAATAAGCAATGGTAGGTTCATGCAATGCAAACCCAGACAGC |
| | CAGGGGACAACAGTAGAGGGCTACAGGCGGCTTTGCGGTTGAGTTCATGACAATGCTGAGT |
| | AATTGGAGTAACAGAGGAAAGCCCAAAAAATACTTTTAATGTGATTTCTTCTAAATAAAAT |
| | TTACACCAGGCAAAATGAACTGTCTTCTTAAGGGATAAACTTTCCCCTGGAAAAACTACAA |
| | GGAAAATTAAGAAAACGATGATCACATAAACACAGTTGTGGTTACTTCTACTGGGGAAGGA |
| | AGAGGGTATGAGCTGAGACACACAGAGTCGGCAAGTCTCCAAGCAAGCACAGAACGAATAC |
| | ATTACAGTACCTTGAATACAGCAGTTAAACTTCTAAATCGCAAGAAGAGGAAAATGCACAC |
| | AGCTGTGTTTAGAAAATTCTCAGTCCAGCACTATTCATAATAGCAAAGACATTAACCCAGG |
| | TTGGATAAATAAATGATGACACAGGCAATTGCACAATGATACAGACATACATTTAGTACAT |
| | GAGACATCGATGATGTATCCCCAAAGAAATGACTTTAAAGAGAAAAGGCCTGATGTGTGGT |
| | GGCACTCACCTCCCTGGGATCCCCGGACAGGTTGCAGGCACACTGTGTGGCAGGGCAGGCT |
| | GGTACATGCTGGCAGCTCCTGGGGCCTGATGTGGAGCAAGCGCAGGGCTGTATACCCCCAA |
| | GGATGGCACAGTCAGTGAATTCCAGAGAGAAGCAGCTCAGCCACACTGCCCAGGCAGAGCC |
| | CGAGAGGGACGCCCACGCACAGGGAGGCAGAGCCCAGCTCCTCCACAGCCACCACCACCTG |
| | TGCACGGGCCACCACCTTGCAGGCACAGAGTGGGTGCTGAGAGGAGGGGCAGGGACACCAG |
| | GCAGGGTGAGCACCCAGAGAAAACTGCAGAAGCCTCACACATCCACCTCAGCCTCCCCTGA |
| | CCTGGACCTCACCTGGTCTGGACCTCACCTGGCCTGGGCCTCACCTGACCTGGACCTCACC |
| | TGGCCTGGGCTTCACCTGACCTGGACCTCACCTGGCCTCCGGCCTCACCTGCACCTGCTCC |
| | AGGTCTTGCTGGAACCTGAGTAGCACTGAGGCTGCAGAAGCTCATCCAGGGTTGGGGAATG |
| | ACTCTGGAACTCTCCCACATCTGACCTTTCTGGGTGGAGGCATCTGGTGGCCCTGGGAATA |
| | TAAAAAGCCCCAGAATGGTGCCTGCGTGATTTGGGGGCAATTTATGAACCCGAAAGGACAT |
| | GGCCATGGGGTGGGTAGGGACATAGGGACAGATGCCAGCCTGAGGTGGAGCCTCAGGACAC |
| | AGTTGGACGCGGACACTATCCACATAAGCGAGGGACAGACCCGAGTGTTCCTGCAGTAGAC |
| | CTGAGAGCGCTGGGCCCACAGCCTCCCCTCGGTGCCCTGCTGCCTCCTCAGGTCAGCCCTG |
| | GACATCCCGGGTTTCCCCAGGCCAGATGGTAGGTTTGAAGTGAGGTCTGTGTCACTGTGGT |
| | ATTATGATTACGTTTGGGGGAGTTATCGTTATACCCACAGCATCACACGGTCCATCAGAAA |
| | CCCATGCCACAGCCCTCCCCGCAGGGGACCGCCGCGTGCCATGTTACGATTTTGATCGAGG |
| | ACACAGCGCCATGGGTATGGTGGCTACCACAGCAGTGCAGCCCATGACCCAAACACACAGG |
| | GCAGCAGGCACAATGGACAGGCCTGTGAGTGACCATGCTGGGCTCCAGCCCGCCAGCCCCG |
| | GAGACCATGAAACAGATGGCCAAGGTCACCCCACAGTTCAGCCAGACATGGCTCCGTGGGG |
| | TCTGCATCGCTGCTGCCCTCTAACACCAGCCCAGATGGGGACAAGGCCAACCCCACATTAC |
| | CATCTCCTGCTGTCCACCCAGTGGTCCCAGAAGCCCCTCCCTCATGGCTGAGCCACATGTG |
| | TGAACCCTGAGAGCACCCCATGTCAGAGTAGGGGCAGCAGAAGGGCGGGGCTGGCCCTGTG |
| | CACTGTCCCTGCACCCATGGTCCCTCGCCTGCCTGGCCCTGACACCTGAGCCTCTTCTGAG |
| | TCATTTCTAAGATAGAAGACATTCCCGGGGACAGCCGGAGCTGGGCGTCGCTCATCCCGCC |
| | CGGCCGTCCTGAGTCCTGCTTGTTTCCAGACCTCACCAGGGAAGCCAACAGAGGACTCACC |
| | TCACACAGTCAGAGACAAAGAACCTTCCAGAAATCCCTGTCTCACTCCCCAGTGGGCACCT |
| | TCTTCCAGGACATTCCTCGGTCGCATCACAGCAGGCACCCACATCTGGATCAGGACGGCCC |
| | CCAGAACACAAGATGGCCCATGGGGACAGCCCCACAACCCAGGCCTTCCCAGACCCCTAAA |
| | AGGCGTCCCACCCCCTGCACCTGCCCCAGGGCTAAAAATCCAGGAGGCTTGACTCCCGCAT |
| | ACCCTCCAGCCAGACATCACCTCAGCCCCCTCCTGGAGGGGACAGGAGCCCGGGAGGGTGA |
| | GTCAGACCCACCTGCCCTCGATGGCAGGCGGGGAAGATTCAGAAAGGCCTGAGATCCCCAG |
| | GACGCAGCACCACTGTTCAATGGGGGCCCAGACGCCTGGACCAGGGCCTGCGTGGGAAAGG |
| | CCGCTGGGCACACTCAGGGGCTTTTTGTGAAGGCCCCTCCTACTGTGTGACTACGGTGACT |
| | ACCACAGTGATGAAACTAGCAGCAAAAACTGGCCGGACACCCAGGGACCATGCACACTTCT |
| | CAGCTTGGAGCTCTCCAGGACCAGAAGAGTCAGGTCTGAGGGTTTGTAGCCAGACCCTCGG |
| | CCTCTAGGGACACCCTGGCCATCACAGCGGATGGGCTGGTGCCCCACATGCCATCTGCTCC |
| | AAACAGGGGCTTCAGAGGGCTCTGAGGTGACTTCACTCATGACCACAGGTGCCCTGGCCCC |
| | TTCCCCGCCAGCTACACCGAACCCTGTCCCAACAGCTGCCCCAGTTCCAACAGCCAATTCC |
| | TGGGGCCCAGAATTGCTGTAGACACCAGCCTCGTTCCAGCACCTCCTGCCAATTGCCTGGA |
| | TTCACATCCTGGCTGGAATCAAGAGGGCAGCATCCGCCAGGCTCCCAACAGGCAGGACTCC |
| | CGCACACCCTCCTCTGAGAGGCCGCTGTGTTCCGCAGGGCCAGGCCCTGGACAGTTCCCCT |
| | CACCTGCCACTAGAGAAACACCTGCCATTGTCGTCCCCACCTGGAAAAGACCACTCGTGGA |
| | GCCCCCAGCCCCAGGTACAGCTGTAGAGAGACTCCCCGAGGGATCTAAGAAGGAGCCATGC |
| | GCAGTTCTGCCGGGACCCTCGGCCAGGCCGACAGGAGTGGACACTGGAGCTGGGCCCACAC |
| | TGGGCCACATAGGAGCTCACCAGTGAGGGCAGGAGAGCACATGCCGGGGAGCACCCAGCCT |
| | CCTGCTGACCAGAGGCCCGTCCCAGAGCCCAGGAGGCTGCAGAGGCCTCTCCAGGGGGACA |
| | CTGTGCATGTCTGGTCCCTGAGCAGCCCCCCACGTCCCCAGTCCTGGGGGCCCCTGGCACA |
| | GCTGTCTGGACCCTCCCTCTTCCCTGGGAAGCTCCTCCTGACAGCCCCGCCTCCAGTTCCA |
| | GGTGTGGTTATTGTCAGGGGGTGTCAGACTGTGGTGGATACAGCTATGGTTACCACAGTGG |
| | TGCTGCCCATAGCAGCAACCAGGCCAAGTAGACAGGCCCCTGCTGTGCAGCCCCAGGCCTC |
| | CAGCTCACCTGCTTCTCCTGGGGCTCTCAAGGTCACTGTTGTCTGTACTCTGCCCTCTGTG |
| | GGGAGGGTTCCCTCAGTGGGAGGTCTGTTCTCAACATCCCAGGGCCTCATGTCTGCACGGA |
| | AGGCCAATGGATGGGCAACCTCACATGCCGCGGCTAAGATAGGGTGGGCAGCCTGGCGGGG |
| | GACAGTACATACTGCTGGGGTGTCTGTCACTGTGCCTAGTGGGGCACTGGCTCCCAAACAA |
| | CGCAGTCCTCGCCAAAATCCCCACAGCCTCCCCTGCTAGGGGCTGGCCTGATCTCCTGCAG |
| | TCCTAGGAGGCTGCTGACCTCCAGAATGTCTCCGTCCCCAGTTCCAGGGCGAGAGCAGATC |
| | CCAGGCCGGCTGCAGACTGGGAGGCCACCCCCTCCTTCCCAGGGTTCACTGGAGGTGACCA |
| | AGGTAGGAAATGGCCTTAACACAGGGATGACTGCGCCATCCCCCAACAGAGTCAGCCCCCT |
| | CCTGCTCTGTACCCCGCACCCCCCAGGCCAGTCCACGAAAACCAGGGCCCCACATCAGAGT |
| | CACTGCCTGGCCCGGCCCTGGGGCGGACCCCTCAGCCCCCACCCTGTCTAGAGGACTTGGG |
| | GGGACAGGACACAGGCCCTCTCCTTATGGTTCCCCCACCTGCCTCCGGCCGGGACCCTTGG |
| | GGTGTGGACAGAAAGGACACCTGCCTAATTGGCCCCCAGGAACCCAGAACTTCTCTCCAGG |
| | GACCCCAGCCCGAGCACCCCCTTACCCAGGACCCAGCCCTGCCCCTCCTCCCCTCTGCTCT |
| | CCTCTCATCACCCCATGGGAATCCGGTATCCCCAGGAAGCCATCAGGAAGGGCTGAAGGAG |
| | GAAGCGGGGCCGTGCACCACCGGGCAGGAGGCTCCGTCTTCGTGAACCCAGGGAAGTGCCA |
| | GCCTCCTAGAGGGTATGGTCCACCCTGCCTGGGGCTCCCACCGTGGCAGGCTGCGGGGAAG |
| | GACCAGGGACGGTGTGGGGGAGGGCTCAGGGCCCTGCGGGTGCTCCTCCATCTTCGGTGAG |
| | CCTCCCCCTTCACCCACCGTCCCGCCCACCTCCTCTCCACCCTGGCTGCACGTCTTCCACA |
| | CCATCCTGAGTCCTACCTACACCAGAGCCAGCAAAGCCAGTGCAGACAAAGGCTGGGGTGC |
| | AGGGGGGCTGCCAGGGCAGCTTCGGGGAGGGAAGGATGGAGGGAGGGGAGGTCAGTGAAGA |
| | GGCCCCCTTCCCCTGGGTCCAGGATCCTCCTCTGGGACCCCCGGATCCCATCCCCTCCTGG |
| | CTCTGGGAGGAGAAGCAGGATGGGAGAATCTGTGCGGGACCCTCTCACAGTGGAATATCCC |
| | CACAGCGGCTCAGGCCAGACCCAAAAGCCCCTCAGTGAGCCCTCCACTGCAGTCCTGGGCC |
| | TGGGTAGCAGCCCCTCCCACAGAGGACAGACCCAGCACCCCGAAGAAGTCCTGCCAGGGGG |
| | AGCTCAGAGCCATGAAAGAGCAGGATATGGGGTCCCCGATACAGGCACAGACCTCAGCTCC |
| | ATCCAGGCCCACCGGGACCCACCATGGGAGGAACACCTGTCTCCGGGTTGTGAGGTAGCTG |
| | GCCTCTGTCTCGGACCCCACTCCAGACACCAGACAGAGGGGCAGGCCCCCCAAAACCAGGG |
| | TTGAGGGATGATCCGTCAAGGCAGACAAGACCAAGGGGCACTGACCCCAGCAAGGGAAGGC |
| | TCCCAAACAGACGAGGAGGTTTCTGAAGCTGTCTGTATCACAGTGGGGTATAGCAGTGGCT |
| | GGTACCACAGTGACACTCGCCAGGCCAGAAACCCCGTCCCAAGTCAGCGGAAGCAGAGAGA |
| | GCAGGGAGGACACGTTTAGGATCTGAGGCCGCACCTGACACCCAGGGCAGCAGACGTCTCC |
| | CCTCCAGGGCACCCTCCACCGTCCTGCGTTTCTTCAAGAATAGGGGCGGCCTGAGGGGGTC |
| | CAGGGCCAGGCGATAGGTCCCCTCTACCCCAAGGAGGAGCCAGGCAGGACCCGAGCACCGT |
| | CCCCATTGAGGCTGACCTGCCCAGACGGGCCTGGGCCCACCCCACACACCGGGGCGGAATG |
| | TGTGCAGGCCCCAGTCTCTGTGGGTGTTCCGCTAGCTGGGGCCCCCAGTGCTCACCCCACA |
| | CCTAAAGCGAGCCCCAGCCTCCAGAGCCCCCTAAGCATTCCCCGCCCAGCAGCCCAGCCCC |
| | TGCCCCCACCCAGGAGGCCCCAGAGCTCAGGGCGCCTGGTCGGATTCTGAACAGCCCCGAG |
| | TCACAGTGGGTATAACTGGAACGACCACCGTGAGAAAAACTGTGTCCAAAACTGACTCCTG |
| | GCAGCAGTCGGAGGCCCCGCCAGAGAGGGGAGCAGCCGGCCTGAACCCATGTCCTGCCGGT |
| | TCCCATGACCCCCAGCACCCAGAGCCCCACGGTGTCCCCGTTGGATAATGAGGACAAGGGC |
| | TGGGGGCTCCGGTGGTTTGCGGCAGGGACTTGATCACATCCTTCTGCTGTGGCCCCATTGC |
| | CTCTGGCTGGAGTTGACCCTTCTGACAAGTGTCCTCAGAAAGACAGGGATCACCGGCACCT |
| | CCCAATATCAACCCCAGGCAGCACAGACACAAACCCCACATCCAGAGCCAACTCCAGGAGC |
| | AGAGACACCCCAACACTCTGGGGGACCCCAACCGTGATAACTCCCCACTGGAATCCGCCCC |
| | AGAGTCTACCAGGACCAAAGGCCCTGCCCTGTCTCTGTCCCTCACTCAGGGCCTCCTGCAG |
| | GGCGAGCGCTTGGGAGCAGACTCGGTCTTAGGGGACACCACTGTGGGCCCCAACTTTGATG |
| | AGGCCACTGACCCTTCCTTCCTTTCCTGGGGCAGCACAGACTTTGGGGTCTGGGCAGGGAA |
| | GAACTACTGGCTGGTGGCCAATCACAGAGCCCCCAGGCCGAGGTGGCCCCAAGAAGGCCCT |
| | CAGGAGGTGGCCACTCCACTTCCTCCCAGCTGGACCCCAGGTCCTCCCCAAGATAGGGGTG |
| | CCATCCAAGGCAGGTCCTCCATGGAGCCCCCTTCAGACTCCTCCCGGGACCCCACTGGACC |
| | TCAGTCCCTGCTCTGGGAATGCAGCCACCACAAGCACACCAGGAAGCCCAGGCCCAGCCAC |
| | CCTGCAGTGGGCAAGCCCACACTCTGGAGCAGAGCAGGGTGCGTCTGGGAGGGGCTAACCT |
| | CCCCACCCCCCACCCCCCATCTGCACACAGCCACCTACCACTGCCCAGACCCTCTGCAGGA |
| | GGGCCAAGCCACCATGGGGTATGGACTTAGGGTCTCACTCACGTGCCTCCCCTCCTGGGAG |
| | AAGGGGCCTCATGCCCAGATCCCTGCAGCACTAGACACAGCTGGAGGCAGTGGCCCCAGGG |
| | CCACCCTGACCTGGCATCTAAGGCTGCTCCAGCCCAGACAGCACTGCCGTTCCTGGGAAGC |
| | CTGGGCTCCACCAGACCACAGGTCCAGGGCACAGCCCACAGGAGCCACCCACACACAGCTC |
| | ACAGGAAGAAGATAAGCTCCAGACCCCAGGGCGGGACCTGCCTTCCTGCCACCACTTACAC |
| | ACAGGCCAGGGAGCTGTTCCCACACAGATCAACCCCAAACCGGGACTGCCTGGCACTAGGG |
| | TCACTGCCATTTCCCTCTCCATTCCCTCCCAGTGCCTCTGTGCTCCCTCCTTCTGGGGAAC |
| | ACCCTGTGCAGCCCCTCCCTGCAGCCCACACGCTGGGGAGACCCCACCCTGCCTCGGGCCT |
| | TTTCTACCTGCTGCACTTGCCGCCCACCCAAACAACCCTGGGTACGTGACCCTGCAGTCCT |
| | CACCCTGATCTGCAACCAGACCCCTGTCCCTCCCTCTAAACACCCCTCCCAGGCCAACTCT |
| | GCACCTGCAGGCCCTCCGCTCTTCTGCCACAAGAGCCTCAGGTTTTCCTACCTGTGCCCAC |
| | CCCCTAACCCCTCCTGCCCACAACTTGAGTTCTTCCTCTCCTGGAGCCCTTGAGCCATGGC |
| | ACTGACCCTACACTCCCACCCACACACTGCCCATGCCATCACCTTCCTCCTGGACACTCTG |
| | ACCCCGCTCCCCTCCCTCTCAGACCCGGCCCTGGTATTTCCAGGACAAAGGCTCACCCAAG |
| | TCTTCCCCATGCAGGCCCTTGCCCTCACTGCCTGGTTACACGGGAGCCTCCTGTGCGCAGA |
| | AGCAGGGAGCTCAGCTCTTCCACAGGCAGAAGGCACTGAAAGAAATCGGCCTCCAGTGCCT |
| | TGACACACGTCCGCCTGTGTCTCTCACTGCCTGCACCTGCAGGGAGGCTCCGCACTCCCTC |
| | TAAAGATGAGGGATCCAGGCAGCAACATCACGGGAGAATGCAGGGCTCCCAGACAGCCCAG |
| | CCCTCTCGCAGGCCTCTCCTGGGAAGAGACCTGCAGCCACCACTGAACAGCCACGGAGGTC |
| | GCTGGATAGTAACCGAGTCAGTGACCGACCTGGAGGGCAGGGGAGCAGTGAACCGGAGCCC |
| | ATACCATAGGGACAGAGACCAGCCGCTAACATCCCGAGCCCCTCACTGGCGGCCCCAGAAC |
| | ACCCCGTGGAAAGAGAACAGACCCACAGTCCCACCTGGAACAGGGCAGACACTGCTGAGCC |
| | CCCAGCACCAGCCCCAAGAAACACTAGGCAACAGCATCAGAGGGGGCTCCTGAGAAAGAGA |
| | GGAGGGGAGGTCTCCTTCACCATCAAATGCTTCCCTTGACCAAAAACAGGGTCCACGCAAC |
| | TCCCCCAGGACAAAGGAGGAGCCCCCTGTACAGCACTGGGCTCAGAGTCCTCTCTGAGACA |
| | GGCTCAGTTTCAGACAACAACCCGCTGGAATGCACAGTCTCAGCAGGAGAGCCAGGCCAGA |
| | GCCAGCAAGAGGAGACTCGGTGACACCAGTCTCCTGTAGGGACAGGAGGATTTTGTGGGGG |
| | TTCGTGTCACTGTGAGCATATTGTGGTGGTGACTGCTATTCCCACAGTGACACAACCCCAT |
| | TCCTAAAGCCCTACTGCAAACGCACCCACTCCTGGGACTGAGGGGCTGGGGGAGCATCTGG |
| | GAAGTATGGCCTAGGGGTGTCCATCAATGCCCAAAATGCACCAGACTCTCCCCAAGACATC |
| | ACCCCACCAGCCAGTGAGCAGAGTAAACAGAAAATGAGAAGCAGCTGGGAAGCTTGCACAG |
| | GCCCCAAGGAAAGAGCTTTGGCAGGTGTGCAAGAGGGGATGTGGGCAGAGCCTCAGCAGGG |
| | CCTTTTGCTGTTTCTGCTTTCCTGTGCAGAGAGTTCCATAAACTGGTATTCAAGATCAATG |
| | GCTGGGAGTGAGCCCAGGAGGACAGTGTGGGAAGAGCACAGGGAAGGAGGAGCAGCCGCTA |
| | TCCTACACTGTCATCTTTTGAAAGTTTGCCCTGTGCCCACAATGCTGCATCATGGGATGCT |
| | TAACAGCTGATGTAGACACAGCTAAAGAGAGAATCAGTGAAATGCATTTGCAGCACAGATC |
| | TGAATAAATCCTCCAGAATGTGGAGCAGCACAGAAGCAAGCACACAGAAAGTGCCTGATGC |
| | CAAGGCAAAGTTCAGTGGGCACCTTCAGGCATTGCTGCTGGGCACAGACACTCTGAAAAGC |
| | ACTGGCAGGAACTGCCTGTGACAAAGCAGAACCCTCAGGCAATGCCAGCCCTAGAGCCCTT |
| | CCTGAGAACCTCATGGGCAAAGATGTGCAGAACAGCTGTTTGTCATAGCCCCAAACTATGG |
| | GGCTGGACAAAGCAAACGTCCATCTGAAGGAGAACAGACAAATAAACGATGGCAGGTTCAT |
| | GAAATACAAACTAGGACAGCCAGAGGACAACAGTAGAGAGCTACAGGCGGCTTTGCGGTTG |
| | AGTTCATGACAATGCTGAGTAATTGGAGTAACAGAGGAAAGCCCAAAAAATACTTTTAATG |
| | TGATTTCTTCTAAATAAAATTTACACCCGGCAAAATGAACTATCTTCTTAAGGGATAAACT |
| | TTCCCCTGGAAAAACTATAAGGAAAATCAAGAAAACGATGATCACATAAACACAGTGGTGG |
| | TTACTTCTACTGGGGAAGGAAGAGGGTATGAGCTGAGACACACAGAGTCGGCAAGTCTCCT |
| | AACAAGAACAGAACAAATACATTACAGTACCTTGAAAACAGCAGTTAAACTTCTAAATCGC |
| | AAGAAGAGGAAAATGCACACACCTGTGTTTAGAAAATTCTCAGTCCAGCACTGTTCATAAT |
| | AGCAAAGACATTAACCCAGGTTGGATAAATAAGCGATGACACAGGCAATTGCACAATGATA |
| | CAGACATACATTCAGTATATGAGACATCGATGATGTATCCCCAAAGAAATGACTTTAAAGA |
| | GAAAAGGCCTGATGTGTGGTGGCAATCACCTCCCTGGGCATCCCCGGACAGGCTGCAGGCT |
| | CACTGTGTGGCAGGGCAGGCAGGCACCTGCTGGCAGCTCCTGGGGCCTGATGTGGAGCAGG |
| | CACAGAGCTGTATATCCCCAAGGAAGGTACAGTCAGTGCATTCCAGAGAGAAGCAACTCAG |
| | CCACACTCCCTGGCCAGAACCCAAGATGCACACCCATGCACAGGGAGGCAGAGCCCAGCAC |
| | CTCCGCAGCCACCACCACCTGCGCACGGGCCACCACCTTGCAGGCACAGAGTGGGTGCTGA |
| | GAGGAGGGGCAGGGACACCAGGCAGGGTGAGCACCCAGAGAAAACTGCAGAAGCCTCACAC |
| | ATCCACCTCAGCCTCCCCTGACCTGGACCTCACCTGGCCTGGGCCTCACCTGACCTGGACC |
| | TCACCTGGCCTGGGCTTCACCTGGCCTGGGCTTCACCTGACCTGGACCTCACCTGGCCTCG |
| | GGCCTCACCTGGCCTGGGCTTCACCTGGCCTGGGCTTCACCTGACCTGGACCTCACCTGGC |
| | CTGGGCCTCACCTGACCTGGACCTCACCTGGCCTGGGCTTCACCTGGCCTGGGCTTCACCT |
| | GGCCTGGGCTTCACCTGACCTGGACCTCACCTGGCCTGGGCTTCACCTGACCTGGACCTCA |
| | CCTGGCCTCGGGCCTCACCTGCACCTGCTCCAGGTCTTGCTGGAGCCTGAGTAGCACTGAG |
| | GCTGTAGGGACTCATCCAGGGTTGGGGAATGACTCTGCAACTCTCCCACATCTGACCTTTC |
| | TGGGTGGAGGCACCTGGTGGCCCAGGGAATATAAAAAGCCCCAGAATGATGCCTGTGTGAT |
| | TTGGGGGCAATTTATGAACCCGAAAGGACATGGCCATGGGGTGGGTAGGGACAGTAGGGAC |
| | AGATGTCAGCCTGAGGTGAAGCCTCAGGACACAGGTGGGCATGGACAGTGTCCACCTAAGC |
| | GAGGGACAGACCCGAGTGTCCCTGCAGTAGACCTGAGAGCGCTGGGCCCACAGCCTCCCCT |
| | CGGGGCCCTGCTGCCTCCTCAGGTCAGCCCTGGACATCCCGGGTTTCCCCAGGCCTGGCGG |
| | TAGGTTTGAAGTGAGGTCTGTGTCACTGTGGTATACTATGATAGTAGTTGGTTATTACTAC |
| | CACAGTGTCACAGAGTCCATCAAAAACTCATGCCTGGGAGCCTCCCACCACAGCCCTCCCT |
| | GCGGGGGACCGCTGCATGCCGTGTTAGGATTTTGATCGAGGACACGGCGCCATGGGTATGG |
| | TGGCTACCACAGCAGTGCAGCCCATGACCCAAACACACGGGGCAGCAGAAACAATGGACAG |
| | GCCCACAAGTGACCATGATGGGCTCCAGCCCACCAGCCCCAGAGACCATGAAACAGATGGC |
| | CAAGGTCACCCTACAGGTCATCCAGATCTGGCTCCAAGGGGTCTGCATCGCTGCTGCCCTC |
| | CCAACGCCAAACCAGATGGAGACAGGGCCGGCCCCATAGCACCATCTGCTGCCGTCCACCC |
| | AGCAGTCCCGGAAGCCCCTCCCTGAACGCTGGGCCACGTGTGTGAACCCTGCGAGCCCCCC |
| | ATGTCAGAGTAGGGGCAGCAGGAGGGGGGGGCTGGCCCTGTGCACTGTCACTGCCCCTGTG |
| | GTCCCTGGCCTGCCTGGCCCTGACACCTGAGCCTCTCCTGGGTCATTTCCAAGACATTCCC |
| | AGGGACAGCCGGAGCTGGGAGTCGCTCATCCTGCCTGGCTGTCCTGAGTCCTGCTCATTTC |
| | CAGACCTCACCAGGGAAGCCAACAGAGGACTCACCTCACACAGTCAGAGACAATGAACCTT |
| | CCAGAAATCCCTGTTTCTCTCCCCAGTGAGAGAAACCCTCTTCCAGGGTTTCTCTTCTCTC |
| | CCACCCTCTTCCAGGACAGTCCTCAGCAGCATCACAGCGGGAACGCACATCTGGATCAGGA |
| | CGGCCCCCAGAACACGCGATGGCCCATGGGGACAGCCCAGCCCTTCCCAGACCCCTAAAAG |
| | GTATCCCCACCTTGCACCTGCCCCAGGGCTCAAACTCCAGGAGGCCTGACTCCTGCACACC |
| | CTCCTGCCAGATATCACCTCAGCCCCCTCCTGGAGGGGACAGGAGCCCGGGAGGGTGAGTC |
| | AGACCCACCTGCCCTCAATGGCAGGCGGGGAAGATTCAGAAAGGCCTGAGATCCCCAGGAC |
| | GCAGCACCACTGTCAATGGGGGCCCCAGACGCCTGGACCAGGGCCTGTGTGGGAAAGGCCT |
| | CTGGCCACACTCAGGGGCTTTTTGTGAAGGGCCCTCCTGCTGTGTGACTACGGTGGTAACT |
| | CCCACAGTGATGAAACCAGCAGCAAAAACTGACCGGACTCGCAGGGTTTATGCACACTTCT |
| | CGGCTCGGAGCTCTCCAGGAGCACAAGAGCCAGGCCCGAGGGTTTGTGCCCAGACCCTCGG |
| | CCTCTAGGGACACCCGGGCCATCTTAGCCGATGGGCTGATGCCCTGCACACCGTGTGCTGC |
| | CAAACAGGGGCTTCAGAGGGCTCTGAGGTGACTTCACTCATGACCACAGGTGCCCTGGTCC |
| | CTTCACTGCCAGCTGCACCAGACCCTGTTCCGAGAGATGCCCCAGTTCCAAAAGCCAATTC |
| | CTGGGGCCGGGAATTACTGTAGACACCAGCCTCATTCCAGTACCTCCTGCCAATTGCCTGG |
| | ATTCCCATCCTGGCTGGAATCAAGAGGGCAGCATCCGCCAGGCTCCCAACAGGCAGGACTC |
| | CCACACACCCTCTTCTGAGAGGCCGCTGTGTTCCGCAGGGCCAGGCCGCAGACAGTTCCCC |
| | TCACCTGCCCATGTAGAAACACCTGCCATTGTCGTCCCCACCTGGCAAAGACCACTTGTGG |
| | AGCCCCCAGCCCCAGGTACAGCTGTAGAGAGAGTCCTCGAGGCCCCTAAGAAGGAGCCATG |
| | CCCAGTTCTGCTGGGACCCTCGGCCAGGCCGACAGGAGTGGACGCTGGAGCTGGGCCCACA |
| | CTGGGCCACATAGGAGCTCACCAGTGAGGGCAGGAGAGCACATGCCGGGGAGCACCCAGCC |
| | TCCTGCTGACCAGAGACCCGTCCCAGAGCCCAGGAGGCTGCAGAGGCCTCTCCAGGGGGAC |
| | ACAGGGCATGTCTGGTCCCTGAGCAGCCCCCAGGCTCTCTAGCACTGGGGGCCCCTGGCAC |
| | AGCTGTCTGGACCCTCCCTGTTCCCTGGGAAGCTCCTCCTGACAGCCCCGCCTCCAGTTCC |
| | AGGTGTGGTTATTGTCAGGGGGTGCCAGGCCGTGGTAGAGATGGCTACAATTACCACAGTG |
| | GTGCCGCCCATAGCAGCAACCAGGCCAAGTAGACAGACCCCTGCCACGCAGCCCCAGGCCT |
| | CCAGCTCACCTGCTTCTCCTGGGGCTCTCAAGGCTGCTGTCTGCCCTCTGGCCCTCTGTGG |
| | GGAGGGTTCCCTCAGTGGGAGGTCTGTGCTCCAGGGCAGGGATGACTGAGATAGAAATCAA |
| | AGGCTGGCAGGGAAAGGCAGCTTCCCGCCCTGAGAGGTGCAGGCAGCACCACAGAGCCATG |
| | GAGTCACAGAGCCACGGAGCCCCCAGTGTGGGCGTGTGAGGGTGCTGGGCTCCCGGCAGGC |
| | CCAGCCCTGATGGGGAAGCCTGCCCCGTCCCACAGCCCAGGTCCCCAGGGGCAGCAGGCAC |
| | AGAAGCTGCCAAGCTGTGCTCTACGATCCTCATCCCTCCAGCAGCATCCACTCCACAGTGG |
| | GGAAACTGAGCCTTGGAGAACCACCCAGCCCCCTGGAAACAAGGCGGGGAGCCCAGACAGT |
| | GGGCCCAGAGCACTGTGTGTATCCTGGCACTAGGTGCAGGGACCACCCGGAGATCCCCATC |
| | ACTGAGTGGCCAGCCTGCAGAAGGACCCAACCCCAACCAGGCCGCTTGATTAAGCTCCATC |
| | CCCCTGTCCTGGGAACCTCTTCCCAGCGCCACCAACAGCTCGGCTTCCCAGGCCCTCATCC |
| | CTCCAAGGAAGGCCAAAGGCTGGGCCTGCCAGGGGCACAGTACCCTCCCTTGCCCTGGCTA |
| | AGACAGGGTGGGCAGACGGCTGCAGATAGGACATATTGCTGGGGCATCTTGCTCTGTGACT |
| | ACTGGGTACTGGCTCTCAACGCAGACCCTACCAAAATCCCCACTGCCTCCCCTGCTAGGGG |
| | CTGGCCTGGTCTCCTCCTGCTGTCCTAGGAGGCTGCTGACCTCCAGGATGGCTTCTGTCCC |
| | CAGTTCTAGGGCCAGAGCAGATCCCAGGCAGGCTGTAGGCTGGGAGGCCACCCCTGTCCTT |
| | GCCGAGGTTCAGTGCAGGCACCCAGGACAGGAAATGGCCTGAACACAGGGATGACTGTGCC |
| | ATGCCCTACCTAAGTCCGCCCCTTTCTACTCTGCAACCCCCACTCCCCAGGTCAGCCCATG |
| | ACGACCAACAACCCAACACCAGAGTCACTGCCTGGCCCTGCCCTGGGGAGGACCCCTCAGC |
| | CCCCACCCTGTCTAGAGGACTTGGGGGGACAGGACACAGGCCCTCTCCTTATGGTTCCCCC |
| | ACCTGGCTCCTGCCGGGACCCTTGGGGTGTGGACAGAAAGGACGCCTGCCTAATTGGCCCC |
| | CAGGAACCCAGAACTTCTCTCCAGGGACCCCAGCCCGAGCACCCCCTTACCCAGGACCCAG |
| | CCCTGCCCCTCCTCCCCTCTGCTCTCCTCTCATCACTCCATGGGAATCCAGAATCCCCAGG |
| | AAGCCATCAGGAAGGGCTGAAGGAGGAAGCGGGGCCGCTGCACCACCGGGCAGGAGGCTCC |
| | GTCTTCGTGAACCCAGGGAAGTGCCAGCCTCCTAGAGGGTATGGTCCACCCTGCCTGGGGC |
| | TCCCACCGTGGCAGGCTGCGGGGAAGGACCAGGGACGGTGTGGGGGAGGGCTCAGGTCCCT |
| | GCAGGTGCTCCATCTTGGATGAGCCCATCCCTCTCACCCACCGACCCGCCCACCTCCTCTC |
| | CACCCTGGCCACACGTCGTCCACACCATCCTGAGTCCCACCTACACCAGAGCCGGCAGAGC |
| | CAGTGCAGACAGAGGCTGGGGTGCAGGGGGGCCGCCAGGGCAGCTTTGGGGAGGGAGGAAT |
| | GGAGGAAGGGGAGGTCAGTGAAGAGGCCCCCCTCCCCTGGGTCTAGGATCCACCTTTGGGA |
| | CCCCCGGATCCCATCCCCTCCAGGCTCTGGGAGGAGAAGCAGGATGGGAGAATCTGTGCGG |
| | GACCCTCTCACAGTGGAATACCTCCACAGCGGCTCAGGCCAGATACAAAAGCCCCTCAGTG |
| | AGCCCTCCACTGCAGTGCTGGGCCTGGGGGCAGCCCCTCCCACAGAGGACAGACCCAGCAC |
| | CCCGAAGAAGTCCTGCCAGGGGGAGCTCAGAGCCATGAAGGAGCAAGATATGGGGACCCCA |
| | ATACTGGCACAGACCTCAGCTCCATCCAGGCCCACCAGGACCCACCATGGGTGGAACACCT |
| | GTCTCCGGCCCCTGCTGGCTGTGAGGCAGCTGGCCTCTGTCTCGGACCCCCATTCCAGACA |
| | CCAGACAGAGGGACAGGCCCCCCAGAACCAGTGTTGAGGGACACCCCTGTCCAGGGCAGCC |
| | AAGTCCAAGAGGCGCGCTGAGCCCAGCAAGGGAAGGCCCCCAAACAAACCAGGAGGTTTCT |
| | GAAGCTGTCTGTGTCACAGTCGGGTATAGCAGCGGCTACCACAATGACACTGGGCAGGACA |
| | GAAACCCCATCCCAAGTCAGCCGAAGGCAGAGAGAGCAGGCAGGACACATTTAGGATCTGA |
| | GGCCACACCTGACACTCAAGCCAACAGATGTCTCCCCTCCAGGGCGCCCTGCCCTGTTCAG |
| | TGTTCCTGAGAAAACAGGGGCAGCCTGAGGGGATCCAGGGCCAGGAGATGGGTCCCCTCTA |
| | CCCCGAGGAGGAGCCAGGCGGGAATCCCAGCCCCCTCCCCATTGAGGCCATCCTGCCCAGA |
| | GGGGCCCGGACCCACCCCACACACCCAGGCAGAATGTGTGCAGGCCTCAGGCTCTGTGGGT |
| | GCCGCTAGCTGGGGCTGCCAGTCCTCACCCCACACCTAAGGTGAGCCACAGCCGCCAGAGC |
| | CTCCACAGGAGACCCCACGCAGCAGCCCAGCCCCTACCCAGGAGGCCCCAGAGCTCAGGGC |
| | GCCTGGGTGGATTCTGAACAGCCCCGAGTCACGGTGGGTATAGTGGGAGCTACTACCACTG |
| | TGAGAAAAGCTATGTCCAAAACTGTCTCCCGGCCACTGCTGGAGGCCCAGCCAGAGAAGGG |
| | ACCAGCCGCCCGAACATACGACCTTCCCAGCCCTCATGACCCCCAGCACTTGGAGCTCCAC |
| | AGTGTCCCCATTGGATGGTGAGGACGGGGGCCGGGGCCATCTGCACCTCCCAACATCACCC |
| | CCAGGCAGCACAGGCACAAACCCCAAATCCAGAGCCGACACCAGGAACACAGACACCCCAA |
| | TACCCTGGGGGACCCTGGCCCTGGTGACTTCCCACTGGGATCCACCCCCGTGTCCACCTGG |
| | ATCAAAGACCCCACCGCTGTCTCTGTCCCTCACTCAGGGCCTGCTGAGGGGCGGGTGCTTT |
| | GGAGCAGACTCAGGTTTAGGGGCCACCATTGTGGGGCCCAACCTCGACCAGGACACAGATT |
| | TTTCTTTCCTGCCCTGGGGCAACACAGACTTTGGGGTCTGTGCAGGGAGGACCTTCTGGAA |
| | AGTCACCAAGCACAGAGCCCTGACTGAGGTGGTCTCAGGAAGACCCCCAGGAGGGGGTTTG |
| | TGCCCCTTCCTCTCATGTGGACCCCATGCCCCCCAAGATAGGGGCATCATGCAGGGCAGGT |
| | CCTCCATGCAGCCACCACTAGGCAACTCCCTGGCGCCGGTCCCCACTGCGCCTCCATCCCG |
| | GCTCTGGGGATGCAGCCACCATGGCCACACCAGGCAGCCCGGGTCCAGCAACCCTGCAGTG |
| | CCCAAGCCCTTGGCAGGATTCCCAGAGGCTGGAGCCCACCCCTCCTCATCCCCCCACACCT |
| | GCACACACACACCTACCCCCTGCCCAGTCCCCCTCCAGGAGGGTTGGAGCCGCCCATAGGG |
| | TGGGCGCTCCAGGTCTCACTCACTCGCTTCCCTTCCTGGGCAAAGGAGCCTCGTGCCCCGG |
| | TCCCCCCTGACGGCGCTGGGCACAGGTGTGGGTACTGGGCCCCAGGGCTCCTCCAGCCCCA |
| | GCTGCCCTGCTCTCCCTGGGAGGCCTGGGCACCACCAGACCACCAGTCCAGGGCACAGCCC |
| | CAGGGAGCCGCCCACTGCCAGCTCACAGGAAGAAGATAAGCTTCAGACCCTCAGGGCCGGG |
| | AGCTGCCTTCCTGCCACCCCTTCCTGCCCCAGACCTCCATGCCCTCCCCCAACCACTTACA |
| | CACAAGCCAGGGAGCTGTTTCCACACAGTTCAACCCCAAACCAGGACGGCCTGGCACTCGG |
| | GTCACTGCCATTTCTGTCTGCATTCGCTCCCAGCGCCCCTGTGTTCCCTCCCTCCTCCCTC |
| | CTTCCTTTCTTCCTGCATTGGGTTCATGCCGCAGAGTGCCAGGTGCAGGTCAGCCCTGAGC |
| | TTGGGGTCACCTCCTCACTGAAGGCAGCCTCAGGGTGCCCAGGGGCAGGCAGGGTGGGGGT |
| | GAGGCTTCCAGCTCCAACCGCTCCACTAGCCGAGACTAAGGAAGTGAGAGGCAGCCAGAAA |
| | TCCAGACCATTCCATAGCAAATGGATTTCATAAAGTTACCAGACTTCAGTTGTAAGTAACA |
| | TGAGCCCCATGCACAACAATCCCTTATGAAGGGGAAGTCAGTGTCGCCTCGGATTTCTTGA |
| | AAAACACAAAAACTTATCAATGCCTGTAAAAGTCTGTTGGAAAGAAAATATGATTCAAGAA |
| | TGTTATGCCCAACAAAGCTGGCATATTTTCTACCCGGACACACTCAGGGAATGTGGTCCCT |
| | TGAGTGCTTCTCTCACTGCGTAAATCCTACGTGGTGTTTAAGCATATTCATAAATGTGTAT |
| | GTCTATTTTTATGTGTAAGATGGTTCATTTTTATTTTATTTATTCAATATGTACAATAAAG |
| | AATATTGACAAATAGGCTGGACATGGTGGCTCCCACCTGTAATCCCAGCCCTTTGGGAGGC |
| | CGAGGCGGGCAGATCACCTGAGGTCTGGAGTTCGAGACCAGCCTGGCCAACATGATGAAAA |
| | CCCATCTCTACTAAAAATACAAAGATTAGCCAGGCATGGTGGTGCATGCCTGTAATCCCAG |
| | CCACTCAGGAGGCTGAGACAGGAGAAATGCGTGAACCCGGAAGGCGGAGGTTGCAGTGAGC |
| | CGAGATCACACCACTGCACTCCAGCCTGGCGACAGAGCAAGATTCCATCTCAAAAAAAAAA |
| | AGACAAAGAAATTTGTTTTTTTGAATAAAGACAAATTTCATCACACGAAGATAAAGATGCA |
| | AAGCTCCAGACAGGAAGGCACGGACAGCACAGTGAAGCCCGGAGCGGGCGCTGGGGGGCCA |
| | GGGGCATGGCGGGGGTGCCAGCGTCTCTCGGTGCCTACCATGGCCACTCCAGCCTGTGTTC |
| | TCACGAGGATGGCTGTGCAATGCTAGGAGCGTGTTCGAAGCTCTAGGGCAACCACTGGAAG |
| | TGAGGCTGAGGAGCAGAGCCCAGAGGCCCGTGGAGCTGATGAAAAGAAAGCTGGAGAAAGT |
| | GTTTGCTGCCTCCCAACATGGTAAGAAAAGATAGAAAGAGAGAGCACACGGCAAAGGGAGC |
| | TTGCTGAGGGACTCTTTACAATGGCTTGCACAGAGCTCAGGGGGTCTGGGAGGCTAGGGCC |
| | CTGCGCAGGGCAGTCACCCCAGCCTGCTGACCAAGGTTTGCTGCAGGCAGCTCTGGGGGTG |
| | GTTGAGGCGCGGTCCCTGGAGCCACCCCTCAAGGGAACGAGGCAGCAGAGTGGGCCAAGGC |
| | CCAGGTCGGCTGCAAGGCTGCCCAGGACTTGGGGTCCTTACATCAGCAGCCACTGATGCAG |
| | CTGGCCCAGAGAGAGGCGCCGAGCAGGTTGCCTCCAGGGGACAAACCAGGTCGGAGAGGGT |
| | GAGGCAGTGGATGGAGCCACAACAACCCCGGGCACGGGTGACACGCACGTTCATGCACATC |
| | TGACCCTTCCTCCCTCACCAAACAGGTCCCCCTGCCTTCCCCATGGTTGCGAAAAAGCAAA |
| | ATGTAGACGTTTTTTCTTTTTTAATTCATGTTTTAATTGACAAATGAAGCCGTATATATTT |
| | ATTGTGTACAACATGATGCTTTAAAATATGTATACATCGTGGAACAGCAACGTTGAGCTAA |
| | TTTAACACGCATTACTTCACATACTTGTCATCTTTTGTGGCGAGAATGCTTAAAATCCACT |
| | CTCTTAGTATTTTTTAAGAATGCAATACATTGTTGTCAACTGTGGTCACCGTCATGCATAG |
| | CCAAGCTCCCGACCTCACCCTCCTGCCAGCTCAGGCTGTGCATCCTTTCACCAGCATCCCC |
| | CACCCCGGCCCCTGGCCCTGGTAACTACCACTCTATACTCTACGTATGAGTTCAGCTTTTT |
| | AAGATTCCACAGATGAATGAGATCATACAGTATTTGCTTTCTATGCCTGGCTTATTTTAGT |
| | TAACACACTGTCCTCCAGATCCATCCGTTGTTGCAAATGACAGGGTTTCATTCTTTTTAAA |
| | GTCTAAAGAGTATTCCATTGTGTCAATGGACCTCATTTGCTTTATCCATGCATCAACTATG |
| | GACATTTAGGTTGATTCCATTTCTTAGCTGTTGTGGATGGTGCTGCAGTAAACATGGGGCT |
| | GCAGATGTCTCTTCAACATACTGACATCATGTCCTTTGGATAAATACCCAGTAGTGGGATC |
| | GCTGGATCACAATGTACAGTTTTTTTTTTAATGGAAACTTTCATTTTTTGGTGAAATTAGG |
| | AAAACAGATAAAACCCACAGAATCCAAAATATATGTGAAGATGCCAAAAACAGTTGACATT |
| | GGGCAGAGGTCACATGGAAGGAAGTGAATACATGACGGGGTGTGAGGGCCCAGAGGCAGCT |
| | GAAATACGCTTTCTAAACACAAGGACCTCTTCTGAGAGGGCAGAAGTTTTATCCTGCACAT |
| | GCAATGACCAGCACAGCTAAAATACACTTTCTAAACATGAGGACCTCTTCTGAGAGGGCAG |
| | CTTTATCCTGCAAATGCAATGACCAGCACAGGACCCAGAATAAAGAGAGTTGCCAGCGGAC |
| | GCCTGGTGTCCATGTGTCCAGGTGAGTTCGAGATGCGGACGGCGCTGGCCAGCCAGTCACA |
| | CCCTAAGTCAATCTGCTGCATGCATTTGTCCTTGCCACAGCAGAAAACGAGAAAGCCTTTG |
| | GGCTGCAAAGCTTCACAGGCTCCTCTTCTCCCGACTCCATGGAAACAGCTACAAAGAGCAG |
| | GCCCAGTAGAGCTTAATTCATGAAAATGAGTAATAAACTTGAACTGGAACAGTATCGACTT |
| | TTTAGAAACGGCAGCAAAGTGTATAAAAAATATTCACCAGAACAATATTTCCAAACGATGA |
| | GATGAGAATTTCAGCCAAGTAATCCTCCATGGATAGAAAATAATGAAGGGATTGGATTTAT |
| | GAAGGAAAATCATGGAGCTCAAATACAAGAGAAGAGAATCAAAAATGAACAGGAGGAGATA |
| | AAATATGGTTTGGCCAAAGTTACAAAATAAATTTTTTAAAAACCCTTCATCATGGCAAGTA |
| | GAAAGAGCGAGAGGAAAAACAGATCCCGTGGAAGACACAAATAGGACATGGGGAGAAAAAT |
| | GAATGAGATGAAACAGAGCAGAAATAAAATTTTACGGAACTAAAGACAAGTGATCTGAACC |
| | TGCCTGGGGCCTGGGGGACCTCGCCACCCTGAAGGGAAAGAACATGCCTGGCTGGCTTTGC |
| | CACCTGCTCATTGCAGAGCCCCACAGCTTGCAACAAACATAGGCGGTAGCCAGGGAGTGGT |
| | TACAGCAGGCCTTGAGCAAGACCCAGTGTTGTGCTGACTTCAGGTCTGACCCAGCACTGTC |
| | ATAGTGGTGGTGTCCATAGTGGTAGTGGGGGTGCTTGTGTCACTCCACCCCCATCTCCAGG |
| | AGGCTCAGAACAGACAGAGAGAGACTCCATTTGTTTGGGAGAAAGTAAGGGATGAGAACAA |
| | GAGTCTCTGCCTGGTAATCCAGAGAATTATTCTAGATCTTGGCCAAGATTATCAAAGCAGT |
| | ACCTCTATGAGTCTTTTGGGCTTGGAGTCCCCCTAAAGCAGATATAGCTAAGATCACAACA |
| | CCCAAGTCCTTTTGAATATGTGGGAAGACTTCCCAAGGACAGGAGCAAACAAACAAGCCCA |
| | GACTGCAAAAAAACAAGCCCAGACTGCAATAAACACCTCACTCTTCAATGCCCAGGCACTG |
| | AAGAACATCTCCTAGCAGCAACACCATCCAGGAAAACATGGCCTCAACCAGTGAACTAAAT |
| | AAGGCACCAGGGACCAGTCTCGGAGAAATAGAGGTATGTTATCTTTCAGAGAATTCAAAGT |
| | AGCTTTGTTGAGGAAACTCAAAGAAATTCAAGATAACACAGTGAAGGAATTCAGAATCCTA |
| | TCCGATAAATTTAACAGAGATTGAAGCAATTAAAAAGAATTAAGCAGAAATTATGGAGCTG |
| | AAAAATGCAATTGGCATACTGAAAAATGCATCAGAGTATTTTCATAGCCTCTTATATCAAG |
| | TAGAAGAAAGAATTAGTGAGCTTGAAAACAGGCTATTTGGAAAAGCACGATAAAAGGAGAC |
| | AAAAGAGAAAAGAATAAATAACAATGAAGCATATCTACAGGATCTAGAAAATAGCCTCAAA |
| | AGGCCAAATCTAAGAATTATTAGCCTTAAAGAGGAGGTAGAGAAAGAGGGATGGAGAGTTT |
| | ATTCAAAGGGATAATAACAGAAAACTTCCCAAACCTAGAGAAAGATATCAATATCCAAATG |
| | CAAGAAGGATGTAGTACACCAAGGAGATTTAATGCAAAGAAGACTACCTCAAGGCATTCAA |
| | TACTCAAACTCCCATATGACAAGGACTTTAAAAAGATCCTAAAAGCAGCAAAAGAAAAGAA |
| | ATGAATAAAATACTATGGAGCTCCAATATGTCTGGCAGCAGACTTTTCAGTGAAGACTTTA |
| | TATGCCAGGAGAGAGTGTCATAATGGATTTAAAGTGCTGAAGGAAAAAACTTTTACCCTCG |
| | AACAGTATAGCTGGTGAAATTATCCTTCAAACATGAAGGAGAAATAATTTGTTTCCAGACA |
| | AATGTTGAGGGATTTCATGAACACCAGACCTGTCTTTTAAGAAATGCTAAAGGGAGTACTT |
| | CAATCAGAAAGAAACACGTTAGTGAACAATAAGAAATCATCTGAAGGCACAAAACTCACCG |
| | GTAATAGTAAGTACACAGAAAAACACAGAATATTATAACACTGTAACTGTGGTGTGTAAAC |
| | TCCTTTTGTTTGTTTGTTTGTTTGTTTGTTTGTTTGTTTGTTTTTGTTTTTAGACGGAGTT |
| | TTGCTCCAGCCCAGGCTGGAGTGCAATGGCACAATCTCAGCTCACTGCAACTTCCACCTCC |
| | CGGGTTCAAGCAATTCTCCTGCCTCAGCCTCCCAAGTAGCTGGGATTACAGGCATGTGCTA |
| | CCATGTCCAGCTAATTTTGTATTTTAGTAGAGACGGTGTTTCACCATGTTGGTCAGGCTAG |
| | CCTTATCTTGAGTAGAAAAACTAAATGATGAAGCAATGAAAAATAATAACTACAACTTTTC |
| | AAGACATAGTACAATAAGATATAAATCATAACAAAAAGTTAAAAGGTGGAGGGATGAAGTT |
| | AAGGCAAAGAGTCTTTATTAGTTTTCTTTTTACTTGTCTGTTTATGCAAACAGTGTTAAGT |
| | TGTCATCAGTTTAAAATAATGGGTCATAAGATACTATTTGCAAGCCTCATGGTAACGTCAA |
| | ACCAAAAGCAATACAACAGATACACAAAAAACAAAAAGCAAGAAGCTAAATTACGTCATCA |
| | GAGAAAATCACCTTCACTAAAAGGAAGACGGAGAAAAGAATGAAGAGAGAGAAGACCAAAA |
| | GCAAATAGCAATATGGCAGGAGTAAGTCCTTACTTATCAATAATACCATTGAATGTAAATG |
| | GACTAAACTCTCCAATCAAAAGACATAGAGTGGCTGAATCAATTAAAGAAAAAACAAGACC |
| | CATTGATCTGTTGTCCACAAGAAACACACTTTATCTATAAAGACACACATAGACTGAAAAC |
| | AAAGGGATGGAAAAAGATACTCCACGCCAATGGAAACCAAAGAAAGAGCAGGAGTAGCTAC |
| | ACTTATATCAGGCAAAATAGATTTCAAGACAAAAACTATAAGAAGAGACAAGGTCACTAAT |
| | GATAAACAGGTCAATTCAGCAAGAGGATATAACAATTGTAAATATATATGCACCCAATGCT |
| | GGAGCACCCAGATATATAAAGCAAGTATTTACTAGAGCTAAAGAGAGAAATAGACTCCAAT |
| | GCAATAATAGCTGGAGATTTCAACATCCCACTTTCAACATTGAACAGATCCTCCAGATAGA |
| | AAATCAACAAAGAAATATTGGACTTAATCTGCACTATCGACCAAATGGATCTAACAGATAT |
| | TTACAGAACATTTCATCCAACAGCTGCAGAACACACATTCTTTTCCTCAGCACATAGATCA |
| | TTCTCAAGGATAGACCATATGTTGGGTCACAAAACAAGTTTTAAAATATTCAAATACATTG |
| | AAATAATATCAAGCATCTTCTGTGACCACAATGGACTAAAACTAGAAATCAATAACAAGAG |
| | GAATTTTGGAAACTATATAAATATATGGAAATTAATGAATGCTGAGTGGGTCAATGAAGCA |
| | ATTAAGAAGGAAACTGAAATTTTTCTTGGAACGAATGATCATGGAAACAGAAAATACCAAA |
| | ACCTATGGGATACAGCAAAAGCAGTACTAAGAGGGAAGTTTACAGCTACAAATGCTTACAT |
| | TAAAAAAGAAGAAAAACTTCAATAAAAAAACCTAACAATGCATCTTAAAGAACTAGAAAAG |
| | CAAGAGGAAATCAAATCCAAAATTAGTAGAAGAAAACAGTAAAGGTCAGAGCAGAAATAAG |
| | TAAAATTGAAATGAAGAAAACAATACAAAAGATCAATAAAACAACAGGTTGTTTTCTTGAA |
| | AAGTTAAACAAAATTGACAAACCTTTAGCCAGACTAAGAAAAAAAGACAGAAGATCCAAAT |
| | AAATAAAATCAGAGATGAAAAAGGTGACATTACAACTTACACCACAGAAATTCAAAGGATC |
| | ATTAGTGGCTACTATAAGCAACTATATGCCAATAAATTGGAAAATCTAGAAGAAATGCAGA |
| | AATTCCTAGACACATACAACCTCCCAAGATTAAACCAAGAAGAAATTCAAAACCTGAACAG |
| | ACTGATAACAAGTAATGAGATCAAAGCCGTAATAAAAAGCCTCCCAGTAAAGAGAAGCCCA |
| | GGACCCGACGGCTTCACTGCTGAATTCTACCAAACATTTAAAGTAGAACTAATACCAATCC |
| | TACTCAAACTATTCCAAAAAATAGAGGTGGAAGGAATACTTCAAAACTCATTATACGAGGC |
| | CAGTATTAACCTGACACCAAAACTAGACAAAGACACATGAAAAAAAGAAAACTACAGGCCA |
| | ATATGTCTGATGAATATTGACACAAAAATCCTCAACAAAATACTAGCAAACCAAATTCAAC |
| | TACACATTAGAAAGTTCACTCATCATGACCAAGTGGAATTTATCTAACTTGGGATGCAAAG |
| | ATGGTTCAACATATGCAAATCAATCAATGTGATACATCATATCAACAGAATGAACAACAAA |
| | AACCATTTGATCATTTAATTGATACTGAAAAAGCATTTGATAAAATTCAACATTCCTTCAT |
| | AATAAAAATTCTCTTCTATACTAGGTACAAAAGAAACTTACCTCAACATAATAAAGCCATA |
| | TATGACAGTCCCACAGTATGATACTAAATGAGGAAAAACTGAGAGCCTTTCCTCTACGATC |
| | TGGAACATGACAAAGATGCCCACTTTCATCACTGTTATTCAACATAGTACTGGAAGTCCTA |
| | GCTGGAGCGATCAGACAAGAGAAAGATATAAAAGACATCCAAATTGGAAAGGAATAAGTCA |
| | AATTATCCTCATTTGCATATGGTATGATCTTCTATTTAGAGCTAACTAAAGACTCCACCAA |
| | AAAAAGTTATTAGAACTGACGAACAAATTCAGTAAAGCTGCAGGATACAAAATCAACATAC |
| | AAAAATCAGTAGCATTTCTATATGCCAACAATGACCAATGTGAAAAAGAAATTAAAAAGTA |
| | ACCCTATTTACAATAACCACAAATAAACACCTAGGAATTAACCAAAGAGGTAAAAGATTTC |
| | TGTAATGAAAACTATAAAAACTGATGAAAGAAATTGAAGAGTACACCAAAAAATGGAAAGC |
| | AATTGCATGTTCATGGATTAGAAGAATCAGTGTTGTTATAATGTCCATACTATCCAAAGCA |
| | ATCTACAGATTCAATGCAATCCTTATCAAAATACCAATGACATCATTCACAGAAATAGAAA |
| | AAAAAAATCCTAAAATTTACGTGGAACCACAAAGACCCAGAATAGCCAAAGCTCTCCTAAG |
| | CAAAAAGAACGAAACTGTAGGAATGACATTGCCTGTCTTCAAATTCTACTACAGAGCTATA |
| | GATAGTAACCAAAACAGCGTGGTACTAGCATAAAAACAGACACAGAGACAAACAGAACAAA |
| | ATTTAAAAACCCAGAAATAAATCCACACACCTACAGCAAATTCATTTTTGACAAAGTTGCC |
| | AAGAACATACTCTGGGGAATAGATAATGATATCTCTTCAATAAATAATGTGGGGAAAACTG |
| | GATATCCATATACATAACAGTGAAACTAGACCCCTCTCTCTCTCACTATATACAAAAATCA |
| | AATCAAAATTGTTTAAGGACTTAAATCTAAGACCTCATACTATGAAACCACTGCAAGACAA |
| | CCTTGGCGGAAACTCTCCAAGACATCAGTCCAGGCAAAGATTTCTTGAGTAATATCCCACA |
| | AGCACAGACAACCAAAGCAAAAATGGACAAATGGGATCACATCAAGTTAAAAAGCTTCTGC |
| | ACAGTAAGGGAAACAACCAACAAAATGAAGAGACAACCCACAGAATGGGAGAAAATATTTG |
| | AAAAATACCCATCTGGCAAGGGATTAAAAACCAGAATATATGCAGAATATATAAGGAGCTC |
| | AAACAGTGCTATAGAAAAAAAAATCTAATAATCTGATTTAAAAATGGGAAAAATGTTAGAA |
| | TAGACATTTCTTAAAATAAGACATACAGATGGCAAACCGACATGGAACGGTGCTCAACATC |
| | ATGGATTATCACAGAAACACAATCAATCAAAACTAAAACTAAAATGTGCTATCATCTCACC |
| | CCAGTTAAAATGGCTGATATCCAGAAGACAGGCAATAACAAATGCTGGCAAGGATGTGGGG |
| | AAAAGGGAGCCCCCATACACTGTTGCTGGGATTGTAAATTAGTACAACCACTGTGGAGAGC |
| | AGCATGAAAGTTCCTCAAAAAACTGAAAGAAAGCTACCATAGGATCCAGCAATCCCACTGC |
| | TGTGTATATACTACAAAAGAAAGGAAGTCAGTATATGAAGAGGTATCTGCACTCCCATGTT |
| | TGTTGCAGCCCTGTTCACAACAGCCAAGATTTGGAAGCAACCTAAGTGTCCATCAGCAGTT |
| | GAATGTATAAAGAAAATGTGGTGCATATACACAATGGAGTATTATTCAATAATAAAAAGGA |
| | ATGAGATTGAGTCATTTGCAACAACATGGATGGAACTGGAGATCATTATGTGAAGTGAAAT |
| | AAGCCAGGCACAGAAAGACAAACATTACAATGTTCTTACTTATTAATGAGATCTAAAAATC |
| | AAAACAATTGCACCCATGTTCATAAAGAGTAAAAGGATGGTTACCAGATGCTGAGAACGGT |
| | GGTGGGGGGATAGGGAAAGGTGGCAGTGGTTAACGGGTACAAAAAAATAGAAAGAATGAAT |
| | AAGACTTACTACTTGATAGCACAGCAAGGTGGCTATAGTCAGTAATTTAGTTGTATATTTT |
| | TAATAATGAAAGGTGTATAATTGGATTGTTTCTAACACAAAGGATAATGCTTAAGAGGATG |
| | GATACCCCATTTTCCATGATGTGATTATTTCACATTGCACGCCTAGATCAAAACATCCAAT |
| | GTACCCCATAAATATATACATCTTCTATGTACCCATAAAAATTCTGTAAAATAAAATATAT |
| | AAAAAGAGGTGACAGATATGGAAGACAGGCAAAGAAGAGACGACATCCACATAATCCGAGT |
| | ACCTAAGAAAGAATGGAGTCCAGTGCATCTCAGGAGCCACCATTCTAAGCCAATTTTCTCT |
| | GGTTCTCTCAGTCACCCTACTAATACGTGGGCAATCTTGTTTTATTTCAGGATAGAGTTTT |
| | TGAAATTATAGATTTAAGTATGCTTTCTGTTCTATTACTTTTGGTAATTAATTTTAGAAAG |
| | AACTAATTTGGGCACAAATTTGAAAAAATTCTAAATCCAAAAAAAAAAAGAAAAAAACACA |
| | CACACAATCATCTATAAGGGGGATGATGACCAGTCCTAGATTTCTCACCAGCCACATTCAA |
| | GATCAGTAAATGGTAGGACAAAACCTGTAGGGTCCTTAAGGGGGAAAGAAGTAGTGGATAG |
| | TCCAGAGTCTATATACAGCCAACTGTTCTTGAAGAAAAAAGGCTGCTGAAAAGGAGTTCCA |
| | AACATTCTATAATCCATAATCTCATGATGAAACTACTAGAGGAAGACCACCAGCCATCAAA |
| | AGGTGCTTGGAGAACCCAGGGCCAAGAACCAAAAGTAAATATTAAGTGTCCTTAACTGCGA |
| | GACTAAGATAGAAATGACTGTGGGGGACCATGTGGCCTCAACAGAGGTGAAATGGTGTCTG |
| | CCTGACAAAGTGGACATTTTACAATGATCAAAACACAGAATATGAGATAGAGAGCACTTCT |
| | GAATTACTGCCTCACTCCAAATAACTCTCAGCCAAAGGACTTCAGTAAAACCAAATTGGGC |
| | ATATTAGACAGTACAAACAAATTCTAAGAAAATAATATTACTGATTACAATCACATGATGC |
| | TAGAGATGGAGGGGAAAAGGAAGAGGAAACCAGGTAATTTCATACTCGTATATAGTAAAGA |
| | ACTAAAGTACATTGTCCAAAGAAGAACAAAGAATATTTTGGAAAGTTATAAAGGTAGCCAC |
| | TACACATAGAAGATAGCAAAGAACAAGAAAACTTAAGATGGAAAACTTTTTGGAAGCATAA |
| | AAATAGAAAATATAAACTACTAAGATAAGATTGAAGCCAAACAGATCTATGAAAACAACAA |
| | ACATCAATGGCCTTAACTTGCCTATTAAAAGGAAGAGACTTTCAAATTGGACCACAAGATA |
| | AAACCCAACTCTATATAGCATATGAGTATTACACACAAAATGGGAAAAGCTGAAAAAACTT |
| | GGGCAAAATTCACCCCAAGCAAATTCCACTGTTTCCTTTGGGACAAAATGCCAAGCTCCAT |
| | GCCAGGGAAGATGATTCTCCTCAGACCTTCTCCTCACTCTCCCAGTCCTCTTAGGGAAGGA |
| | ATTGGGTGTTAGAGGAGGGAGACTCTGTCGATTATCAGCTGAAGCAGTGGTGTGCTCCTGC |
| | GTTGCTTCTGACCTGGGAAATGAAGCAGCAAGACTCTTTCTGCTGTGTCTTTGCCCAGAAG |
| | GGCCATCCCCCCAGAGCAGAGTACCCAGGCCGGCAGGAGCAGTGGTGGAAGCGTGGAAACC |
| | ACGTCTCCTACAGCAGAGACCATCAGAAGCGGAGCCTCGGGTATAAGGGAAACAACGCGTT |
| | CTCCCTAACCTGGGAGTGACAGACAGCGTCATTCCTCACAGTGATACCCTGTGTTCTAGCC |
| | ATCTGGCCCATGACAGAGCCAGCCCAGAGCCAGCCCAGAGCCAGCCCCTCACCAACCTGGA |
| | GCCTGGCCAGCTCGCCAAGCTGCACCATAGGCCTGGAAGGCGTGGAGACCTGCGGCAGTGC |
| | CCTGTCCTCCCGTGAGGCCTGCCATCCCTGCCAGGGGTCGCCTCTGGCTTCTCCTCCAGGA |
| | CCGCACGGTCCAGAGGCTCAGTGCCTGGAGTAGGTGTTGCCCCCCTGCTTCTAGGCCCAGA |
| | CCCTCCCTTGTTCCTGACCCCGGGCCTTTCCCTCTGGCTTGGACATCCAGGGCCCTGTCTC |
| | AGCTGGGGAGCTGCTCCTGCTCAAGGACTGTCTTCCGCGGGATCGAAAGGCCGCGTCCTGA |
| | ACAATGCGTGGGCCACGTGAGCGGAGCAGGCTCTAAAGGCCGCGTCCTAAACAGTGCGTGG |
| | GCCACGTGAGCGGAGCAGGCTCTAAAGGCCGCGTCCTAAACAGTGCGTGGGCCACGTGAGC |
| | GGAGCAGGCTCTAAAGGCCGCGTCCTAAACAGTGCGTGGGCCACGTGAGCGGAGCAGGCTC |
| | TAAAGGCCGCGTCCTAAACAGTGCGTGGGCCACGTGAGCGGAGCAGGCTCTAAAGGCCGCG |
| | TCCTAAACAGTGCGTGGGCCACGTGAGCGGAGCAGGCTCTAAAGGCCGCGTCCTAAACAGT |
| | GCGTGGGCCACGGGAGCGGAGCAGACTCTAAAGGCCGCGTCCTAAACAGTGCGTGGGCCAC |
| | GTGAGCGGAGCAGGCTCTAAAGGCCGCGTCCTAAACAGTGCGTGGGCCACGTGAGCGGAGC |
| | AGGCTCTAAAGGCCGCGTCCTAAACAGTGCGTGGGCCACGTGAGCGGAGCAGGCTCTAAAG |
| | GCCGCGTCCTAAACAGTGCGTGGGCCACGTGAGCGGAGCAGGCTCTAAAGGCCGCGTCCTA |
| | AACAGTGCGTGGGCCACGGGAGCGGAGCAGACTCTAAAGGCCGCGTCCTAAACAGTGTGTG |
| | GGCCACGTGAGCGGAGCAGGCTCTAAAGGCCGCGTCCTAAACAGTGCGTGGGCCACGGGAG |
| | CGGAGCAGACTCTAAAGGCCGCGTCCTAAACAGTGTGTGGGCCACGTGAGCGGAGCGCCCT |
| | CTCCACTGCCCTCGGGGCCGCAGCTCCCAGCTCAGCTCCCAGCCCTGCTCAGGGCAGCCAG |
| | GCCAGGAGGTACCATCCAGGCTAAGTGACCCTCAGGGGGGACAGGTGCCCCAGGAGATGCC |
| | AGCTGTTGGGAGAGGCTGGGGGACCAACTCGACCTGGCCTGTGGGCCCTGCCCTGGCCACC |
| | CATTGTAGGATCCAGCCGCCACGCCTGTGACACTCGTGTGCTTTCCCTGGTGTGTGCTTGT |
| | GGCAGGTGGGGGCAGAGGGTCCTCAGGCCAGAGAGCCACTCCCCCAGCGCCAGACCACCCT |
| | CTTCCTCACTCCCCCACCTCACCCCCTCACAGGTGCCTCCCAGGCCATCAGGGCCCAACCA |
| | CCCCTAAACAAATGGGTTCTCGGCCCCTCGTGGCTGGAGGTGGGTTCTCTCACCATTCCCA |
| | GCCTAAGGCTCCATCCCCATGCTGGCAGCTGTTCAACCATGTCTAGAGAGATCCACTGTCC |
| | CAGACAGCACCTCAGGGTCCCCCGTCCTGCCTGGAACCCTGTAGGAAACTCCACAAACCGC |
| | CGCCATTCTGTCCACACCCCTACAGGAGCCCCAACCCTCTCCCCACATCCAGGCTTCCCTC |
| | CCAGACCCCTCATCCCTGCCCGCACGGTGCCTGAGGGGGCCTTCTTGGGCAGCGCCTAAGC |
| | AAGCCCCCAGCACCCTTCGGCCCCTTCAAGGCACACAGGCCCCCTTTCCACCCAGCCTCAG |
| | GAAACCACCTGTGTCCTCCAACGACAGGTCCCAGCCTCCCAGCCTTTGCCTTGCCTGTTCC |
| | TCTCCCTGGAACTCTGCCCCGACACAGACCCTCCCCAGCAAGCCCGCAGGGGCACCTCCCC |
| | TGCCCCCAGACACCCTGTGCCCGTCAGTTCATCCCCAGCAGAGGCCCTCACCAGGCACACC |
| | CCCATGCTCACACCTGGCCGCAGGCCTCAGCCTCCCTGAGGGCCCCACCCAGCCCGCGTCT |
| | GGCCAGTGGTGCGTGCAAAGCCCCTCACCCAGACTCGGCGGAAGGCAGCCAGTGCAGGCCT |
| | GGGGAGGGGCTCTCCTTAGACCACCTTGCACCTTCCCTGGCACCCACCATGGGAAGAGCTG |
| | AGACTCACTGAGGACCAGCTGAGGCTCAGAGAAGGGACCCAGCACTGGTGGACACGCAGGG |
| | AGCCCACGCCAGGGCGCCGTGGTGAGTGAGGCCCAGTGCCACCCACTGAGGCCTCCCGTTC |
| | AGTGGGACGACGGTGAACAGGTGGAACCAACCAGGCAACCCCCGCCGGGCCCCACAGACGG |
| | GATCAGAGCAGGAAAGGCTTCCTGCCCCTGCAGGCCAGCGAGGAGCCCTGGCGGGGGCCAT |
| | GGCCCTCCAGGCGAGGAGGCTCCCCTGGCCACCGCCACCCGGGCCTCTCTGCTGCTGGGAA |
| | AACAAGTCAGAAAGCAAGTGGATGAGAGGTGGCGTGACAGACCCAGCTTCAGATCTGCTCT |
| | AATTTACAAAAGAAAAGGAAAAACACACTTGGCAGCCTTCAGCACTCTAATGATTCTTAAC |
| | AGCAGCAAATTATTGGCACAAGACTCCAGAGTGACTGGCAGGGTTGAGGGCTGGGGTCTCC |
| | CGCGTGTTTTGGGGCTAACAGCGGAAGGGAGAGCACTGGCAAAGGTGCTGGGGGCCCCTGG |
| | ACCCGACCCGCCCTGGAGACCGCAGCCACATCAGCCCCCAGCCCCACAGGCCCCCTACCAG |
| | CCGCAGGGTTTTGGCTGAGCTGAGAACCACTGTGCTAACTGGGGACACAGTGATTGGCAGC |
| | TCTACAAAAACCATGCTCCCCCGGGACCCCGGGCTGTGGGTTTCTGTAGCCCCTGGCTCAG |
| | GGCTGACTCACCGTGGCTGAATACTTCCAGCACTGGGGCCAGGGCACCCTGGTCACCGTCT |
| | CCTCAGGTGAGTCTGCTGTCTGGGGATAGCGGGGAGCCAGGTGTACTGGGCCAGGCAAGGG |
| | CTTTGGCTTCAGACTTGGGGACAGGTGCTCAGCAAAGGAGGTCGGCAGGAGGGCGGAGGGT |
| | GTGTTTTTGTATGGGAGAAGCAGGAGGGCAGAGGCTGTGCTACTGGTACTTCGATCTCTGG |
| | GGCCGTGGCACCCTGGTCACTGTCTCCTCAGGTGAGTCCCACTGCAGCCCCCTCCCAGTCT |
| | TCTCTGTCCAGGCACCAGGCCAGGTATCTGGGGTCTGCAGCCGGCCTGGGTCTGGCCTGAG |
| | GCCACACCAGCTGCCATCCCTGGGGTCTCCGCCATGGGCTGCATGCCAGAGCCCTGCTGTC |
| | ACTTAGCCCTGGGGCCAGCTGGAGCCCCCAAGGACAGGCAGGGACCCCGCTGGGCTTCAGC |
| | CCCGTCAGGGACCCTCCACAGGTAGCAAGCAGGCCGAGGGCAGGGACGGGAAGGAGAAGTT |
| | GTGGGCAGAGCCTGGGCTGGGGCTGGGCGCTGGCTGTTCATGTGCCGGGGACCAGGCCTGC |
| | GCTTTAGTGTGGCTACAAGTGCTTGGAGCACTGGGGCCAGGGCAGCCCGGCCACCGTCTCC |
| | CTGGGAACGTCACCCCTCCCTGCCTGGGTCTCAGCCCGGGGGTCTGTGTGGCTGGGGACAG |
| | GGACGCCGGCTGCCTCTGCTCTGTGCTTGGGCCATGTGACCCATTCGAGTGTCCTGCACGG |
| | GCACAGGTTTATGTCTGGGCAGGAACAGGGACTGTGTCCCTGTGTGATGCTTTTGATATCT |
| | GGGGCCAAGGGACAATGGTCACCGTCTCTTCAGGTAAGATGGCTTTCCTTCTGCCTCCTTT |
| | CTCTGGGCCCAGCGTCCTCTGTCCTGGAGCTGGGAGATAATGTCCGGGGGCTCCTTGGTCT |
| | GCGCTGGGCCATGTGGGGCCCTCCGGGGCTCCTTCTCCGGCTGTTTGGGACCACGTTCAGC |
| | AGAAGGCCTTTCTTTGGGAACTGGGACTCTGCTGCTGGGGCAAAGGGTGGGCAGAGTCATG |
| | CTTGTGCTGGGGACAAAATGACCTTGGGACACGGGGCTGGCTGCCACGGCCGGCCCGGGAC |
| | AGTCGGAGAGTCAGGTTTTTGTGCACCCCTTAATGGGGCCTCCCACAATGTGACTACTTTG |
| | ACTACTGGGGCCAGGGAACCCTGGTCACCGTCTCCTCAGGTGAGTCCTCACAACCTCTCTC |
| | CTGCTTTAACTCTGAAGGGTTTTGCTGCATTTTTGGGGGGAAATAAGCGTGCTGGGTCTCC |
| | TGCCAAGAGAGCCCCGGAGCAGCCTGGGGGGCTCAGGAGGATGCCCTGAGGCAACAGCGGC |
| | CACACAGACGAGGGGCAAGGGCTCCAGATGCTCCTTCCTCCTGAGCCCAGCAGCACGGGTC |
| | TCTCTGTGGCCAGGGCCACCCTGGGCCTCTGGGGTCCAATGTCCAACAACCCCCGGGCCCT |
| | CCCCGGGCTCAGTCTGAGAGGGTCCCAGGGACTTAGCGGGGTGCCAGTTCTTGCCTGGGGT |
| | CCTGGCATTGTTGTCACAATGTGACAACTGGTTCGACCCCTGGGGCCAGGGAACCCTGGTC |
| | ACCGTCTCCTCAGGTGAGTCCTCACCACCCCCTCTCTGAGTCCACTTAGGGAGACTCAGCT |
| | TGCCAGGGTCTCAGGGTCAGAGTCTTGGAGGCATTTTGGAGGTCAGGAAAGAAAGCTGGGG |
| | AGAGGGACCCTTCGAATGGGAACCCAGCCTGTCCTCCCCAAGTCCGGCCACAGATGTCGGC |
| | AGCTGGGGGGCTCCTTCGGCTGGTCTGGGGTGACCTCTCTCCGCTTCACCTGGAGCATTCT |
| | CAGGGGCTGTCGTGATGATTGCGTGGTGGGACTCTGTCCCGCTCCAAGGCACCCGCTCTCT |
| | GGGACGGGTGCCCCCCGGGGTTTTTGGACTCCTGGGGGTGACTTAGCAGCCGTCTGCTTGC |
| | AGTTGGACTTCCCAGGCCGACAGTGGTCTGGCTTCTGAGGGGTCAGGCCAGAATGTGGGGT |
| | ACGTGGGAGGCCAGCAGAGGGTTCCATGAGAAGGGCAGGACAGGGCCACGGACAGTCAGCT |
| | TCCATGTGACGCCCGGAGACAGAAGGTCTCTGGGTGGCTGGGTTTTTGTGGGGTGAGGATG |
| | GACATTCTGCCATTGTGATTACTACTACTACTACTACATGGACGTCTGGGGCAAAGGGACC |
| | ACGGTCACCGTCTCCTCAGGTAAGAATGGCCACTCTAGGGCCTTTGTTTTCTGCTACTGCC |
| | TGTGGGGTTTCCTGAGCATTGCAGGTTGGTCCTCGGGGCATGTTCCGAGGGGACCTGGGCG |
| | GACTGGCCAGGAGGGGATGGGCACTGGGGTGCCTTGAGGATCTGGGAGCCTCTGTGGATTT |
| | TCCGATGCCTTTGGAAAATGGGACTCAGGTTGGGTGCGTCTGATGGAGTAACTGAGCCTGG |
| | GGGCTTGGGGAGCCACATTTGGACGAGATGCCTGAACAAACCAGGGGTCTTAGTGATGGCT |
| | GAGGAATGTGTCTCAGGAGCGGTGTCTGTAGGACTGCAAGATCGCTGCACAGCAGCGAATC |
| | GTGAAATATTTTCTTTAGAATTATGAGGTGCGCTGTGTGATAGGGACAAAGAGTGGAGTGG |
| | GGCACTTTCTTTAGATTTGTGAGGAATGTTCCGCACTAGATTGTTTAAAACTTCATTTGTT |
| | GGAAGGAGAGCTGTCTTAGTGATTGAGTCAAGGAAGAAAGGCATCTAGCCTCGGTCTCAAA |
| | AGGGTAGTTGCTGTCTAGAGAGGTCTGGTGGAGCCTGCAAAAGTCCAGCTTTCAAAGGAAC |
| | ACAGAAGTATGTGTATGGAATATTAGAAGATGTTGCTTTTACTCTTAAGTTGGTTCCTAGG |
| | AAAAATAGTTAAATACTATGACTTTAAAATGTGAGAGGGTTTTCAAGTACTCATTTTTTTA |
| | AATGTCCAAAATTTTTGTCAATCAATTTGAGTCATGTTTGTGTAGAACTGATATTACTGAA |
| | AGTTTAACCAAGGAATAGGAATGATGCTCTTTCATACCCTATTCAGAACTGACTTTTAACA |
| | ATAATAAATTAAGTTTAAATATTTTTAAATGAATTGAGCAATGTTGAGTTGGAGTCAAGAT |
| | GGCCGATCAGAACCAGAACACCTGCAGCAGCTGTCAGGAAGCAGGTCATGTGGCAAGGCTA |
| | TTTGGGGAAGGGAAAATAAAACCACTAGGTAAACTTGTAGCTGTGGTTTGAAGAAGTGGTT |
| | TTGAAACACTCTGTCCAGCCCCACCAAACCGAAAGTCCAGGCTGAGCAAAACACCACCTGG |
| | GTAATTTGCATTTCTAAAATAAGTTGAGGATTCAGCCGAAACTGGAGAGGTCCTCTTTTAA |
| | CTTATTGAGTTCAACCTTTTAATTTTAGCTTGAGTAGTTCTAGTTTCCCCAAACTTAAGTT |
| | TATCGACTTCTAAAATGTATTTAGAATTCATTTTCAAAATTAGGTTATGTAAGAAATTGAA |
| | GGACTTTAGTGTCTTTAATTTCTAATATATTTAGAAAACTTCTTAAAATTACTCTATTATT |
| | CTTCCCTCTGATTATTGGTCTCCATTCAATTCTTTTCCAATACCCAAAGCATTTACAGTGA |
| | CTTTGTTCATGATCTTTTTTAGTTGTTTGTTTTGTCTTACTATTAAGACTTTGGACAGCAT |
| | TTATACAGTATCCGATGCATAGGGACAAAGAGTGGAGCGGCCGCAGCACGTGTTGACAATT |
| | AATCATCGGCATAGTATGTCGGCATAGTATAATACGACAAGGTGAGGAACTAAACCATGGG |
| | ATCGGCCATTGAACAAGATGGATTGCACGCAGGTTCTCCGGCCGCTTGGGTGGAGAGGCTA |
| | TTCGGCTATGACTGGGCACAACAGACGATCGGCTGCTCTGATGCCGCCGTGTTCCGGCTGT |
| | CAGCGCAGGGGCGCCCGGTTCTTTTTGTCAAGACCGACCTGTCCGGTGCCCTGAATGAACT |
| | GCAGGACGAGGCAGCGCGGCTATCGTGGCTGGCCACGACGGGCGTTCCTTGCGCAGCTGTG |
| | CTCGACGTTGTCACTGAAGCGGGAAGGGACTGGCTGCTATTGGGCGAAGTGCCGGGGCAGG |
| | ATCTCCTGTCATCTCACCTTGCTCCTGCCGAGAAAGTATCCATCATGGCTGATGCAATGCG |
| | GCGGCTGCATACGCTTGATCCGGCTACCTGCCCATTCGACCACCAAGCGAAACATCGCATC |
| | GAGCGAGCACGTACTCGGATGGAAGCCGGTCTTGTCGATCAGGATGATCTGGACGAAGAGC |
| | ATCAGGGGCTCGCGCCAGCCGAACTGTTCGCCAGGCTCAAGGCGCGCATGCCCGACGGCGA |
| | GGATCTCGTCGTGACCCATGGCGATGCCTGCTTGCCGAATATCATGGTGGAAAATGGCCGC |
| | TTTTCTGGATTCATCGACTGTGGCCGGCTGGGTGTGGCGGACCGCTATCAGGACATAGCGT |
| | TGGCTACCCGTGATATTGCTGAAGAGCTTGGCGGCGAATGGGCTGACCGCTTCCTCGTGCT |
| | TTACGGTATCGCCGCTCCCGATTCGCAGCGCATCGCCTTCTATCGCCTTCTTGACGAGTTC |
| | TTCTGAGCGGGACTCTGGGGTTCGAATAAAGACCGACCAAGCGACGTCTGAGAGCTCCCTG |
| | GCGAATTCGGTACCAATAAAAGAGCTTTATTTTCATGATCTGTGTGTTGGTTTTTGTGTGC |
| | GGCGCGATTTTACAGATTTTATGTTTAGATCTTTTATGCTTGCTTTTCAAAAGGCCTGCAG |
| | GCAAGTGCACAAACAATACTTAAATAAATACTACTCAGTAATAACCTATTTCTTAGCATTT |
| | TTGACGAAATTTGCTATTTTGTTAGAGTCTTTTACACCATTTGTCTCCACACCTCCGCTTA |
| | CATCAACACCAATAACGCCATTTAATCTAAGCGCATCACCAACATTTTCTGGCGTCAGTCC |
| | ACCAGCTAACATAAAATGTAAGCTTTCGGGGCTCTCTTGCCTTCCAACCCAGTCAGAAATC |
| | GAGTTCCAATCCAAAAGTTCACCTGTCCCACCGGCTTCTGAATCAAACAAGGGAATAAACG |
| | AATGAGGTTTCTGTGAAGCTGCACTGAGTAGTATGTTGCAGTCTTTTGGAAATACGAGTCT |
| | TTTAATAACTGGCAAACCGAGGAACTCTTGGTATTCTTGCCACGACTCATCTCCATGCAGT |
| | TGGACGATATCAATGCCGTAATCATTGACCAGAGCCAAAACATCCTCCTTAGGTTGATTAC |
| | GAAATACGCCAACCAAGTATTTCGGAGTGCCTGAACTGTTTTTATATGCTTTTACAAGACT |
| | TGAAATTTTCCTTGCAATAACCGGGTCAATTGTTCTCTTTCTATTGGGCACACATATAATA |
| | CCCAGCAAGTCAGCATCGGAATCTAGAGCACATTCTGCGGCCTCTGTGCTCTGCAAGCCGC |
| | AAACTTTCACCAATGGACCAGAACTACCTGTGAAATTAATAACAGACATACTCCAAGCTGC |
| | CTTTGTGTGCTTAATCACGTATACTCACGTGCTCAATAGTCACCAATGCCCTCCCTCTTGG |
| | CCCTCTCCTTTTCTTTTTTCGACCGAATTAATTCTTAATCGGCAAAAAAAGAAAAGCTCCG |
| | GATCAAGATTGTACGTAAGGTGACAAGCTATTTTTCAATAAAGAATATCTTCCACTACTGC |
| | CATCTGGCGTCATAACTGCAAAGTACACATATATTACGATGCTGTTCTATTAAATGCTTCC |
| | TATATTATATATATAGTAATGTCGTTAACCGTTGATTTTTACTGATTGG |
| 2 | TTATACAGTATCCGATGCAT (AGG) |
| 3 | ATCATGATATCCCACAAGTA (TGG) |

1. A method of preparing a chimeric immunoglobulin heavy chain locus in a mouse cell, the method comprising:
(a) deleting mouse D segments and J segments downstream of Adam6b and upstream of IgM in the endogenous mouse immunoglobulin (Ig) heavy chain locus; and
(b) introducing a human heavy chain variable region transgene into the endogenous mouse Ig heavy chain locus downstream of Adam6b and upstream of IgM, wherein the transgene comprises a plurality of unrearranged human variable segments (VHs), a plurality of human D segments (DH) and a plurality of human J segments (JH) to thereby prepare a chimeric Ig heavy chain locus in the mouse cell,
wherein a mouse comprising the chimeric Ig heavy chain locus expresses an antibody repertoire comprising mouse VH segments and human VH segments, each linked to human DH and JH segments.

2. The method of claim 1, wherein steps (a) and (b) are conducted simultaneously using CRISPR-Cas9-mediated knock-out/knock-in technology.

3. The method of claim 1, wherein the mouse D segments and mouse J segments are deleted by CRISPR-Cas9-mediated gene editing and the human heavy chain variable region transgene is introduced into the mouse Ig heavy chain locus by Cre-Lox-mediated recombination.

4. The method of claim 1, wherein the human heavy chain variable region transgene is carried on a bacterial artificial chromosome (BAC).

5. The method of claim 1, wherein a mouse comprising the chimeric Ig heavy chain locus expresses an antibody repertoire comprising at least twenty different human VH segments.

6. The method of claim 1, wherein a mouse comprising the chimeric Ig heavy chain locus expresses an antibody repertoire comprising at least thirty different human VH segments.

7. The method of claim 1, wherein a mouse comprising the chimeric Ig heavy chain locus expresses an antibody repertoire comprising at least forty different human VH segments.

8. The method of claim 1, wherein a mouse comprising the chimeric Ig heavy chain locus expresses an antibody repertoire comprising human VH segments 3-74, 3-73, 3-72, 2-70, 1-69, 3-66, 3-64, 4-61, 4-59, 1-58, 3-53, 5-51, 3-49, 3-48, 1-46, 1-45, 3-43, 4-39, 4-34, 3-33, 4-31, 3-30, 4-28, 2-26, 1-24, 3-23, 3-21, 3-20, 1-18, 3-15, 3-13, 3-11, 3-9, 1-8, 3-7, 2-5, 7-4-1, 4-4, 1-3, 1-2 and 6-1.

9. The method of claim 1, wherein a mouse comprising the chimeric Ig heavy chain locus expresses an antibody repertoire comprising at least 100 different mouse VH segments.

10. The method of claim 1, wherein a mouse comprising the chimeric Ig heavy chain locus expresses an antibody repertoire comprising at least fifteen different human DH segments.

11. The method of claim 1, wherein a mouse comprising the chimeric Ig heavy chain locus expresses an antibody repertoire comprising at least twenty-six different human DH segments.

12. The method of claim 1, wherein a mouse comprising the chimeric Ig heavy chain locus expresses an antibody repertoire comprising six different human JH segments.

13. A transgenic mouse cell comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHs), a plurality of human D segments (DH) and a plurality of human J segments (JH),
wherein the transgenic mouse cell comprising the chimeric Ig heavy chain locus expresses an antibody repertoire comprising mouse VH segments and human VH segments, each linked to human DH and JH segments.

14. A transgenic mouse comprising a chimeric immunoglobulin (Ig) heavy chain locus, wherein the chimeric Ig heavy chain locus:
(a) lacks mouse D segments and J segments downstream of endogenous Adam6b sequences and upstream of endogenous IgM constant sequences; and
(b) comprises a human heavy chain variable region transgene downstream of endogenous Adam6b sequences and upstream of endogenous IgM sequences, wherein the transgene comprises a plurality of unrearranged human variable segments (VHs), a plurality of human D segments (DH) and a plurality of human J segments (JH),
wherein the transgenic mouse comprising the chimeric Ig heavy chain locus expresses an antibody repertoire comprising mouse VH segments and human VH segments, each linked to human DH and JH segments.

15. The transgenic mouse of claim 14, which further comprises a transgene construct encoding a human immunoglobulin light chain such that the mouse expresses antibodies comprising human immunoglobulin light chains.

16. A method of generating antibodies to an antigen of interest, the method comprising administering the antigen of interest to the transgenic mouse claim 13, such that antibodies that bind to the antigen of interest are generated.

17. The method of claim 16, which further comprises isolating an antibody of interest from the mouse.

18. The method of claim 16, which further comprises isolating nucleic acid encoding an antibody of interest from the mouse and replacing mouse constant region sequences within the nucleic acid with human constant region sequences.


SEQUENCE LISTING (1 sequence)

SEQ ID NO: 1 - DNA, 149438 bp, synthetic construct
  source 1..149438: mol_type = other DNA
tctgtgccgtgcgggtgagcggcgcgtccagacgggcgggtgcacgtttctttttctgaccatttccctgtcagctcttttagatcggggctgccgtttgaaaagttttatcatcgccagacatgccataaggaatatctccaccgcctctaactgtccctaactcggtgtctttcttataactaccattactcagtgacgcgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtctactacacagacaagatgaaacaattcggcattaatacctgagagcaggatgagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttggcgtacgctgaagatcctcggcctagattagaattggcgcggtgcaggatcaccggagcaacggtaggctagcgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagatctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcattcggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcagaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaaaaccgtaaagaaaaataagcacaagttttatccggcctttattcacattcttgcccgcctgatgaatgctcatccagaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtagcgtgttacggtgaaaatctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccataacttcgtatagcatacattatacgaagttataccatcatgatatcccacaagtatggaagatacataagtagatggccagactatacaggaaagaagcaaaacttgggctaacagaagagcaaacacagagctcagacagaactacctggcaatgcgactgggcacactgaaagcactgggcatcagcactgagccccaaatatgcactcaggatcctctgcataataatgtgacataacaggaaggttagaacaggccaaaagaggaaacagaacaaatgcccccaaccaaagaagtataaacaaattgggaagagtaaagaaggattgtaaggattgagtaccacacagaacatgctcttaatggcctcaatgctgaagctaggaagaactaagttaaaagaaacatgttcaacgggattccctgtcactggacttcacaacaagcaaaattcaatctttctgttaaggagatgagaagagaatatctgaaccttgtgttgacagtgccccaccccgactgtcaggctgtgggaaatgccagagcaatcactaggaacacacaaggatgagggagacgagggttaggacacaattctcgagcgcgccgcacacaaaaaccaacacacagatcatgaaaataaagctcttttattggtaccgaattcgccagggagctctcagacgtcgcttggtcggtctttattcgaaccccagagtcccgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtttagttcctcaccttgtcgtattatactatgccgatatactatgccgatgattaattgtcaacacgtgctgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtggaaggtggcgcgaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtagcacgactcgtacgtacgtacgaagttcctattctctagaaagtataggaacttcttataaaatgtattatgaggaaacaagttgaaaattagacaatgtaggagtgacagaaagatagatatgagtatgttgaatgtcagagatacctgaaagtttatctacctgaaccctagttctctccatagtttaaggtaaacaggagagtgcaggaaaatcatccatattctgattaggcagtggcttctgcaaaccacactaggcctggccggctgtgtcctggagttggctaagggaggagtcagggccagtggtgagaagtgcaggcccagataccagaactcactcatcccagacatgagctcttagatacacagagagcccatccatgtgtggatttatcttacatctgtaagtagagaacattgactcttacagaacataatttacacacataggtaaatctgaaataaggtgatcagtgtgaagattttatcacagcacagtttcataataagcacaatttctcaaatcccattgttgtcacccatcttcctcaggacactttcatctgccctgggtcctgctctttcttcaggtgtctcaccccagagcttgatatatagtaggagacatgcaaatagggccctcactctgctgaagaaaaccagccctgcagctctgggagaggagccccagccctgggattcccagctgtttctgcttgctgatcaggactgcacacagagaactcaccatggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagacacagtgaggggaagtcaatgtgagcccagacacaaacctcgctgcaggggcatctgagaccacgagggggtgtcctgggccctgtgaactgggctgctctccgtggcagcggctggtggtgctaaaggctgattttctctcagcatctggggctgattcatcaagtttcctcagagaacctttcagatttacaattctgtacttacgtttaatgtctctgaatgtgacactttccttccctggtgtgtctttgtttttgtgacaagaggacacattctcacctccacagaagcccgagtgtcactttggggacagaaatgaccctgccctggtcaccagaatcagagtcccgaggaagcccaggaggacctgggaagtgtttttcaatcagactcagggcaggcgtctccgtgggaatctctgattggaacaggctttgggattcagattgggaccaagagggaggctcacccagggccagggtccttagaatcctgacagttttcacagtaaccccatcgtcctttaaaactgaacatctaactcagaactgacccatttggtcctttctctgtaatccattttccttttctctaggcttcattcttacacttcccttttcaccttcattctgaaaatggagggtgtgcttcctgtggtctaaaccacaggcctcagatgcattacctggaactcaggaatttcttgaaaaaaatcagtgctattttggggtcagaacaatcctcaaaaatggtccccggaaagaagcccaaatttaattgcaccagattgttgaacgatttatgctacaaaacagtgtcacatgtcagaaatgagcacagtgtaacatctacatggtttgttaagagacacaaatggtcaagtagaacaatcaggtaattaggctgtcgaaggcaacactgccaaatgacagcatttccgtggaaactgcgtgtacatccaggactgcacctgtgaacgatgacatcatacccttcacagtgtcgaggaaagagacatcactcaaacagacaagccaagggacttcagaaaatataaggggaaatacagtgtgcaaatatgtaaaaaatgcaataagatgattactcctaaatgaatatcaagacacaatcacataatatgaaattaaattttcctgaatgataggattactaccaatcaccccccaggacaccctcatctactctgtgcacagccttctcgtcaggcgtcccagcccagaccttgctatgtagcagaagacatgcaaataagacccccctttttgctgatgaaaagcagcccagccctgaccctgcagctctgggagaggagctccagccttgggattcccagctgtctccactcggtgatcggcactgaatacaggagactcaccatggagtttgggctgagctgggttttccttgttgctattttaaaaggtgattcatggggaactagagatactgagtgtgagtggacatgagtgagagaaacagtggacgtgtgtggcactttctgaccagggtgtctctgtgtttgcaggtgtccagtgtgaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagacacacagtgaggggaggtcagtgtgagcccggacacaaacctccctgcaggggcgcgcggggctaccagggggcgctcgggactcactgagggcgggacaggtcccaggaacaggtgcagcggtaggttttctttctcctcagctggagaagtcaggtttgtgttttcagaactctggagtcttacaggttgctacattttcatacagttattagtatgtatttattatcattggtatttaagttttaataattttaaaccttttatgtagtgttattttttaaaactgtttactttcatttgcagttattcttccagagtttcattaacatctattgctatgagcaactacatagctatgagagcataaatttacacctgtagacgtaggtctaaatgccacaaacctgtgcataaatgtatagtgacttatatttaacattataataagataatttttaaaatatatcctaaacgatcaaacttactgatgaactaaatataaattatcagagtaatgcataattgatttcaataattttatattgtttatattaattaatatctatttctttactgaaacataatatattggtcatttcaaaatagctacgatacatttcaaatggccttgatgctaataatgaaaagattttgaggtgattaatatgctaattagttatatttaattattccatattgggtcccacacactgttaaacaaccaaatctcatgagaactcatttactatcatagcaaggggaccgtgctaaccattattcataaatccggacccacgatacaaccaccgggccccgcctcaaacattagggaagcaaaatcacatacagttgttatgacaggtgggacatcctgaaaacctctctaggcatgtcccacacggccctggagctgtctcaggggagcagtctcctccagtgtttagacgcacaggcacggataatagggctaagtctggccagatgtgtgatattcaacacattgcacaactgctctgttctgtatgtaatttatcttctctacaaatgtaacattgatggttgcattaaatatattctgcaaatatgtaaaaataaaataagatgatgattgctaaatgattatcaaggcacaatcacataatccgaagttacaatttcctgagagataggattactgcccatgcttaccaggacactcacatctgctctgggcactgccttctcctcaggcgtcccaccccagagcttgctatatagtaggagacatgcaaatagggtcctccctctgctgatgaaaaccagcccagccctgaccctgcagctctgagagcggagccccagccccagaattcccaggtgttttcatttggtgatcagcactgaacacagaggactcaccatggagtttgggctgagctgggttttccttgttgttattttacaaggtgatttatggagaactagagatgttaagtgtgagtggacgtgagtgagagaaacagtggatttgtgtgacagtttctgaccagggtgtctctgtgtttgcaggtgtccagtgtgaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagagacacagcgaggggaggtcagtgtgagcccggacacaaacctccctgcaggggcgcgtggggccaccagggggcggtcgggaaccactgaggacgggacaggtcccaggagcaggtgcagggggcggtttcctgtcagctgcaggaggcgggtttgtttttgcaggactctggagccttatgaggttgcactattttattatggttatttatcatgtgttttttattgatatttgtgttttagtaatttttacatttatatgtaggggtattttttaaaaatacataacttcaagaaataattcttcctagtaatttacacctattcctctaaagttttattaacacctgttgatatcagcaactacatagctatgcaacattaatttacatctataaacatatgtgtgaatacatgaaactatgcatacatgtgtagtcatttatatttaacattataataaaataataaattaaataaaataattgaattaaattaaaagaattaaacttaatgatgaactaaatataaattagagtaatccataattgatttcaatccttctctatggtttacataaatcgatgtctacttgtaagcttaagtatagtgaattggtcattttaaaatagccaagaacaaatttcaaatgttcttgtcaccaaaaaatgataaggatttgaagatttgaggtgatatataaggagtcttccaattcccttgtccaaagcatccgctcattcatgggctgagaaaagggaagtcattcatgcagtctactctctccacagaactgacggggcacaaggacaacatcgatttttcatggaacatgcctctaggaatgcagctgtgtgcacacacactgctaagcacacacttctttacataattacttgtaactgtattttcttatttattctctccaatttttttacacaaattcatcacttttccccataatcaaagaggattttgatcagaatgcttgtggggagccccttgcctgccagatgcccaacatcactactcttgaagggaggaggaacggcagctctcttgatttctactctaatcctctaggactaaaaccagaaggttgcatgtccagtgcgggagcatcgaagaagatcctgtctgtagaagcaggagcgtcaagacttgactgagagccatggtgctgaaatgagatagattccctgatggagagcacacgtggacccccacacctgagggctcactgctcctcaccacagatgcactcccctactgagtcctgagacctgagtgcaccccatagagtagggctcagatgaggggatgcaaatctccaccagctccaccctcccctgggttcaaaagacgaggacagggcctcgctcagtgaatcctgctctccaccatggacatactttgttccacgctcctgctactgactgtcccgtcctgtgagtgctgtggtcaggtagtacttcagaagcaaaaaatctattctctcctttgtgggcttcatcttcttatgtcttctccacaggggtcttatcccaggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacggccgtgtattactgtgcacggataccacagagacacagcccagggcgcctcctgtacaagaacccaggctgcttctcagtggtgctccctccccacctctgcagaacaggatagtgtggctgagatgccatttcctgccagggcctgcgtttcccatccccatctgactcagagccttgttttcctccctcttctttactaataaatggcatgtcccctgttagtggttcgtgcaagcagaagctgtatcctgtttgacaaagattcagcatgaaaggttcctgttacctaaaaaaaaatagacagatgagacttaattaacctaaataatttttttcacaacaacagagtgaatacacaatttgcagaatgacagaaaacttttgcacactttgtctgtgacagggaactaatatgaagaatttgcaaggaactcaaacaactctacaacaacaacagcaacgagaaccaaataaccccattaaaatgagcaaagaacatgagtagacattttcaaaagaacacatagaaatggataataaatatataaacaatgctcaacatcactaaccatcagggaaatgcaaattaaaaccacaataagatatcatcttccaccagtcacaatgactgttactaaaaactcaaataatatcagatgttgctgaggatgggaaataaaggcaactcttagacattgttgatgaggatgtagacgagtacaaactcaaattcctatttctatttcaacccctgattcctactgtcaatgggagggaagtctcagaaccaatcacacatcagacggcaaatctgtcaaccaagagtctttccactgaaggacctgggaggtcaggaccctcaggaaagtgctggggaccctgtcttgggagtgcccagcagatctcagaactctccatgggtcctgctggacactcatgtagggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagcgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatggactggacctggaggttcctctttgtggtggcagcagctacaggtaaggggcttcctagtcctaaggctgaggaagggatcctggtttagttaaagaggattttattcacccctgtgtcctctccacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagacacagtgtgaaaacccacatcctgagagtgtcagaaaccctgagggagaaggcagctgtgccgggctgaggagatgacaggggttattaggtttaaggctgtttacaaaatgggttatatatttgagaaaaaaagaacagtagaaacaagtacatactctaattttaagataaatattccattcaagagtcgtaatataagccaaattcacagagtggaaaaggccacactctataacgttgatacaaacattccatgaaggtgctactgtgaacaagttttcaaattggatgaatacatgatttggagcaaggttatttgatcatgtggtgagactaagaatgattcttaaaaagtgccaaaagtttccttcaaatgtttctgtcactccttatcataaagtttattttacagcagttttaggattacaaagaaattgcacaggaggcgtgagaattcccatgactccctgccctacacaggcacagcctcctccactacgaccatcctgcaccgcagtcacaaatcagttacaatggaggaatctccaaggacgcttggttctttctttttctggtgatctcctaatataacaagcctaagtatctcaagattccacggttttttcagtgttttctagaactgatattagtcagagggaaagtgggtaaggctattactatttgaactctttcttccattatacttgaataaagcaataacgaagaaataaatgaatatttttgctaaaatggagcaataaaaagactgatattgacagaagaaatatgactgacttctgaaaacacacatgaaccatggttctctctgcatatttaggtgaattacagaaagttgtcataacagatggggaatcctgcagacttcactaggcatggtccacgctgccctggagttgtctcaggggagctgcctcctccggtgattagagcacaggcccagataataggattacatttttttagatgtgtaaacttagacgcactgcacagctgctgtattctctatgtaaattatcttctgtaaaatacaacattaaaggctgcattaaatatattgtgtaaatatgtaaaaataaaatcagattatgagagctaaatgttaatcaaggcacaatcacataatataaaattatattttcctgaatgatggaattactaccaatctcccccaggagacttcatctgcactgggcccggcctctcctcagatgtcccatcacagagcttgctatataatgggggacatgcaaatagggccctccctctgctgatgaaaaccagcccagccctgaccctgcagctctgggagaggagcccagcactgggattccgaggtgtttccattcagtgatctgcactgaacacagaggactcgccatggagtttgggctgagctgggttttccttgttgctattttaaaaggtgattcatggagaactagagatattgagtgtgagtgaacacgagtgagagaaacagtggatatgtgtggcagtttctaaccaatgtctctgtgtttgcaggtgtccagtgtgaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagacacagtgaggggaggccattgtgcgcccagacacaaacctccctgcaggaacgctgggggaatcagcggcagggggcgctcaggagccactgatcagagtcagccccggaggcaggtgcaggtggaggctgtttcctgtcaggatgtgggacttcatcttcttccaacagtttctctaatgaacctctctaattttagaattctgtggttcctaatgtcatctctacatattttcaaaagatcattttaacatgaggacataacctctcatgcaccaaatgcacattgatgcttacaaagatgaaaagttctcaaccattgtgaccaggatcgcagtcctgaggaagctcacgggtgtctgatgagtctcctccattcaggcccaggacagaaacctcagagggattcctggaccagaacggcagggattctgatcacagccaatagagaggctgggccagggtcagtgtcctgtagaagctcacagggttcaagtctgacccttctcctgaccctaaagccaatgagcatcagcactgatctggtgctgcttttgctcccaatacatgttctttctttggagtgtttgttctcccttttttatttgcttttctttcttcctgaaaaagaaacacatggtctctgtgatctacactccagggctcaaggcattttcttagaactcaggcaaggctcaggcttattacaaaaaaactgaaattttagacaaaaagagggtgataggaaggaacaaataaatatgttaaatgtcaaatatacctaaaaatttatttgtctgacccctagttttctccgtatttttaggtaaatgcagcaaaatcacacaggttgtcgtggcaggaagtggattctgcaaaccacactaggcccgtttatctctgtcctagagttggttaaaagagcaactgaggccagctgtgaggagcataggcccgggtactaggactcactcatgccagatataagcccttagacacatacatagcccctccatgtgtgggttcacttttacatctgtacatgaagaaaccactgattcctaaataacataatttatacacataggtaaaaataattaaaaatgtgatagttattaagtgtttatcacacaacaatttcacaataaaacagcattttcccaaatgtaatcattgtcatcgaaatccccaaggacactctcatctgccctgggccctgccctctcctcaggcatctcaccccagagcttgctatatagtaggagacatgcaaataggtccctccctctcctgatgaaaaccagcccagccctgactccgcagctctgggagaggagcccccgccctgggattcccaggtgttttcatttggtgatcagcactgaacacagaagagtcatgacggagtttgggctgagctgggttttccttgttgctatttttaaaggtgattcatgaggaaatagagatattgagtgtgagtggacatgagtgagagaaacagtggatttgtgtggcagtttctgaccttggtgtctctgtgtttgcaggtgtccagtgtgaggtgcagctggtggagtctgggggaggcttggtccagcctggggggtccctgagactctcctgtgcagcctctggattcaccttcagtagctatgctatgcactgggtccgccaggctccagggaagggactggaatatgtttcagctattagtagtaatgggggtagcacatattatgcaaactctgtgaagggcagattcaccatctccagagacaattccaagaacacgctgtatcttcaaatgggcagcctgagagctgaggacatggctgtgtattactgtgcgagagacacagtgaggagaagttaatgtgggaccatgcagaaacctccctgcgggaacgctggggaaagtcatctgcagggggcgctcaggagccactgatcagagtcagccccagcggcaggtgcagatgaaggctgatttcctgtcacgatgtgggacttcatcttcttaaagtttctctactgaacctaagttcggaattctgtgattactagggtcatttctacatatttttaaaatgattgttttaatatgaaaacctattctcctatgcagaaaatgcagattgatcctcacagaggagatgaaaagttctcaaccatggtcaccactgtcagagtcctgaggaagctcagggcttcctggtgagtcttctccagtcagactgaggacaggaactccagaaagaatcctgactagaactgaggtattgctcctcacaagagaactcaagctgcggggggtctgttcctgcagacccctgacccggtggcggatgaataaaacgtacactgacacacagatattctgttttgccagttcagctgagggtccgaggcaacttacagactccaagaagaatcctgtaaagaacggcagcagtggccctgagcagctcgcactccaggcgtgtattgagtatagaattaacaacagaagctctgacaacacacttgtggataattaacatggttaagagagtagtacatattttcaatcagaagtgtttagaggccaggacatatcttcacggtcacacattgagaaggatgtagatatgtcccactaccttctcctgagatctcagacagaatcccagatttcaaaaggacacagaaggacagctctcaggtgcttttaaaaaatgacccacttccagggacagggagcttccctataaccatggtggatgttctgaactacaataaacattggatggatccaggattgtttgaagtcactgtcattattacattcagctgctgtttcaatgtgtctgaagtagtaaatgacaatttagatgacaatttatatgaatcttcaagggtagaacaatattgaccatattccaaaatctgtccttgatccatgatcacactcatctcccagaccaggtccttcagcacgtctctttacctgaaagaagaggactctgggcttggagaggggagaccccaagaagacaactgagttctcaaagggcacagccagcatcctactcccagggcgagcccaaaagactggggcctccctcctcctttttcacctctccgtacaaaggcaccacccacatgcaaatccttacttaagcacccacaggaaaccaccacacatttccttaaattcaggttccagctcacatgggaaatactttctgagagtcctggacctcctgtgcaagaacatgaaacacctgtggttcttcctcctcctggtggcagccccgagatgtgagtgtctcagggatccagacatgggggtatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagacacagtgaggggaggtgagtgtgagcccagacacaaacctccctgcatggacgcggaggggaccggcgcaggtgctgctcaggaccagcaggtggcgcgcggggcccccagagcatgaggccgggtcaggagcaggtgcagggagggcggggcttcctcatctgctcagtggtctccgtcctcgccagcacctcgctgtcaccagggctcctctttctttattatctgtggttctgcttcctcacattcttgtgccaggaaagaaacgaggaagacaaattttcgtctatagttgaagcttcaccaattactaggaacttgcctacaagttcctgcatgacccattataacttatcgattaaaaaatatatattctaatgcttctcaccatctcttgatttgtatcatcaactgaattgtaccctctttgaaattcatatgatgaaaccttaaattcaatggatctatattggaattttaatgaaataattaaggttaaatgtggtcataattgtaagaccctaatgcaatagacgtgttgtctttataagaagaggaagagacaccagagacctctcacttttcacgtgcaggcagagaagaggccatgtggagacatagtgcactagaaggtggcccagtgcaagccaggaagaagccgcgccaagaaccagccctgccagcacactaatcttcaacatacatattttcaatcagaagtgtttagaggccaggacacatcttcaaggtcacacattgagaaggatgtagatatgtcccactaccttctcctgagatctcagacagaatcccagatttcaaaaggacacagaaggacagctctcaggtgcttttaaaaaatgacccacttccagggacagggagcttccctataaccatggtggatgttctgaactacaataaacattggatggatccaggattgtttgaagtcactgtcattattacattcagctgctgtttcaatgtgtctgaagtagtaaatgacaatttagatgacaatttatatgaatcttcaagggtagaacaatattgaccatattccaaaatctgtccttgatccatgatcacactcatctcccagaccaggtccttcagcacgtctctttacctgaaagaagaggactctgggcttggagaggggagaccccaagaagacaactgagttctcaaagggcacagccagcatcctactcccagggcgagcccaaaagactggggcctccctcctcctttttcacctctccatacaaaggcaccacccacatgcaaatcctcacttaagcacccacaggaaaccaccacacatttccttaaattcaggttccagctcacatgggaaatactttctgagagtcctggacctcctgtgcaagaacatgaaacatctgtggttcttccttctcctggtggcagctcccagatgtgagtatctcagggatccagacatggggatatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagacacagtgaggggaggtgagtgtgagcccagacaaaaacctccgtgcagggaggcggaggggaccggcgcaggtgctgctcagcgccagcagggggcgcgcggggcccacagagcaggaggcccggtcaggagcaggtgcagggagggcggggcttcctcatctgctcagtggtctccctcctcgccagcacctcagctgtccccaggggtcctctttctttattatctgtggttctgcttcctcacattcttgtgccaagaaagaaatgaggaagacaaattttcgtctgtagttgaagtttcaccaattactaggaactttcctagaagttcctgcatggcccattatagcttactgattaaaaaatatatattctaacgcttctcagcatctcttgatttgtgtcatcaactgaattgtgccctctttgaaattcatatgcagaaaccttaaattcaattgatgtatattggaattttaatgaaataattaaggttaaatgtggtcataagtgtaagactctaattcaacagacgtgtcgtctttataagaagaggaagagacaccagagacctctcacttttcacgtgcaggcagagaagaggccatgtggagacgtaatgcactagaaggtggcccagtgcaagccaggaagaagcctcaccaagaaccaaccctgccagaacattgatcttcaacgcagtgagttcatcaggaaccccaaacttctcttacgtcttctgattcctgttgtccatgagatgagaaattcagctctaattgtacatcacagggcaaatctgtaaaccaggagtgtttctattgaggatcatggtggatcaggattccaggcaggtgctggagacactgtctcaggagcgcccagatgatctcagggggacctgctggacactcacgtgggacatcagcagtcactttctcagagtaaccagtgagctgtgctggtgcctgatgggactaggatggggtcaaggcaccttctcagtgtcatggagagtgattgttccagaaatcatccaggtggtctctacgctaatcaaatatgggttcacagggaggaacatgtgctctgggtgcttggtcttcagtgaaaggacgtctggccaccaaaagtttgtaaatggagcagggcatgcatttcctcaaggaggattagggcttggagcatcagcatcccactcttgtaaggctgatgtgtcatttaccttccctttcttatcccaaatcagggtcttcagctatgaaatgctctgactcatgaatatgcaaataacctgagatgcactgaggtaaatatggatatttgtcagccctgagagcatcatccagaaaccacatccctccgctagagaagcccctgacggcacagttcctcactatggactggatttggaggatcctcttcttggtgggagcagcgacaggcaaggagatgccaagtcccagtgatgaggaggggattgagtccagtcaaggtggctttcatccactcctgtgttctctccacaggtgcccactcccaaatgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcagacacagtgtgaaaacccacatcctgagagtgtcagaaacgccaggaaggaggcagctgtactggcatggaggggatgacaaaggttattagattgaagattttcttagaaaacgacttcaagtcattaaagaagaggaacaacataaatgtgtatttgtgaaattttaattgagagatttttcatacaacatttattctgtaagctatttcagggattggaatatgaatcaaattaataaagctgatatagacatcctctgaaggcatcttcgtaaacatcaatttctgaatcagtgttgtaaatattttggaacacagacacaagatcacatttttactctacttttatctctatttttaaaaaatgccaaaaagaatcttattttgtgcatgccccattttgaattcccaccgtcaatgcatgatagttcttggttttcctcattcaagttgtcatttgtcattaagagtgttgtgtgttttaaccattctaataggtgagtaacggtatctaatttttatttaaatgcacatgtccctaaaaaattcatatttaacaatttttatataatttttggtgagatgcctctcgtgatatttggttcattttttaaatgcattttttatcagttgtaagtatgcttgcatattgattataaaagtcatttaacaaattaaaataattcatttaacaaatatgtatacttgaataaagcaataacaaagaaataaatgaatatttttgctaaaatggagcaataaaaagactgatattgacagaagaaatatgactgacttctgaaaatacacacacatgagccgtggttctctctacatatttagataaattacagaaagttgtcataactgatggggaatcctgcagacttcactaggcatagtccacactgccctggagttgtctcaggggagctgcctcctccagtggttagagcacaggcccaggtaataggactcatttttttagatgtgtaattttagacacactgcacaactgctgtgttctctgtgcaaattatctcctgtaaaatgtaacattgaaacctgccttaaatatattgtgtaaatatgtaaaaataaaatcagattgtgagagctaaatgctaatcaaggcgcaatcacgtaatatacaattatattttcctgaatgatggaattaataccaatctcccccaggacacttcatctgcacggagcccggcctctcctcagatgtcccaccccagagcttgctatatagtcggggacatgcaaatagggccctccctctgctgatgaaaaccagcccagctgaccctgcagctctgggagaggagcccagcactgggattccgaggtgtttccattcggtgatcagcactgaacacagaggactcaccatggagttttggctgagctgggttttccttgttgctattttaaaaggtgattcatggagaactagagatatcgagtgtgagtgaacacgagtgagagaaacagtggatatgtgtggcagtttctaaccaatgtctctgtgtttgcaggtgtccagtgtgaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagacacagtgaggggaggccattgtgcgcccagacacaaacctccctgcaggaacgctggggaaatcagcggcagggggcgctcaggagccactgatcagagtcagccccggaggcaggtgcagatggaggctgatttcctgtcaggatgtgggactctgtcttcttctgacggttccccagggaacctctctaagtttagcattctgtgcctatgaacgtcttctctaagtatttgaaagagattattttaatatgaagagcagttctcactcgcacaaaatgtggattgatgcttactgggatgaaaagtcctcaaacatggtcaccacgatcagagtctgagtgagctcagggcttcctgctgagtctcctcctatcagaccaaggacagggacctcagtgaggttccccgtctagaacagtctttatggatactgattgtgggcggcaagccacccaggtgccgacgcaagagaccgaggacacgagctgttccagtacaataaaatataaaacaagaatagttataccagatatagatcttagatatgattatatatgaatatcattaatcattagttggtagcaattactctttattccaatattataataatcctcactctacaatcataacctaggaaaagccaggccatacagagataggagctgaggggacatagtgagaagtgaccagaagattgggtgcagcaatgggcatcccttcaggtccatcagcaagtcagagacctaaacaaatattccctattcatgttgaattggcagagtgtaaaaatctagaagacaaaatggccaggaaaaatgtgcgattttgcatgggtgaaggtctggcaagttaatcatcttgaggactgttgaggtcctgaggtggtcctgcccttgcttgatgcccaggccgtagtccaactcacacgaaattgccagggaatagacagtagtttttttagtagttcttgttatcaggcataagtgcatttgaattttctcttcatggcctttcctggcactatttctcattttttttaacacacatagtttcaactagatttatcaccttcacagggtcacagagaagggtggaagaagggaggccctgtatgggtctcgaagaaacatggaaaagagtggagagggacaatagcagggtgtaaggaattattgagaccttactctgcccctcccaggaggctcaggccagcctttttctgcatttgaggttctgggttataaacgctgtagactcctcccttcagggcagggtgacaactatgcaaatgcaagtgggggcctccccacttaaacccagggctcccctccacagtgagtctccctcactgcccagctgggatctcagggcttcattttctgtcctccaccatcatggggtcaaccgccatcctcgccctcctcctggctgttctccaaggtcagtcctgccgagggcttgaggtcacagaggagaacgggtggaaaggagcccctgattcaaattttgtgtctcccccacaggagtctgttccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacacacagtgagagaaaccagccccgagcccgtctaaaaccctccacaccgcaggtgcagaatgagctgctagagactcactccccaggggcctctctattcatccggggaggaaacactggctgtttgtgtcctcaggagcaagaaccagagaacaatgtgggagggttcccagcccctaaggcaactgtataggggacctgaccatgggaggtggattctctgacggggctcttgtgtgttctacaaggttgttcatggtgtatattagatggttaacatcaaaaggctgcctaacaggcacctctccaatatgacagtattttaattagtgaaaattttacacagttcatcattgcttgcttgccttcctccctcctgtccactctcactcactccttcttttattttctacttaattttacaaaatcatttaacccctttttgaactattaataggttatctttgtttggtgattgttttcctttcaataatatgtactgaataattcatctttgtgccaattcataagtattctggtgtaataaagacttctttcataaaaattggataaattaaaataaagataaatttttaaaaacatacgatctatcaaaactgaaccataaagaaataaaaactctgggttgggtgtgtttgctcattcctgtaatcccagcactttgggaggccatggccggtttacaagcaaaaattgaaagttagacaagaaggagtgatagaaagagaaaatgtatattaaatttcagaaatatttaagaatgtatctgcctgaaccctagttctcaccatatctttaggtgaatgctaaaatgcagcaaaatcacgcatgttctcactacagaaagtgggttctacaaaccacactcggcacatttagctttgtcctggagttggttcagggagttattggggccagtgatgaggagcacaggccaagataccagcgatcacttatcccaaacatgagctctaacatacacacttagtcccttttccgtgtgtggtttacttccacatctgtacatggagagaccactgactgacaaaatataatttatacaaatatgtaaaattaaatagggtgatcagttcaaggtgtttatcacagcataattttacaataagacagcatatttcccaaataccatcattgtcaccaaactccttcaaggcacagtcatcttatctgggccccgtcctctcctcaggtgtcccaccccagagcttggtatatagtaggagacatgcaaataaggccctccctctgctgatgaaaatgagcccagccctgaccctgcagctctgggagaggagccccagccgtgagattcccaggagtttccacttggtgatcagcactgaacacagaccaccaaccatggagtttgggcttagctgggttttccttgttgctattttaaaaggtaattcatggtgtactagagatactgagtgtgaggggacatgagtggtagaaacagtggatatgtgtggcagtttctgaccttggtgtttctgtgtttgcaggtgtccaatgtgaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacaccgcgtctgtgaaaggcagattcaccatctcaagagatggttccaaaagcatcgcctatctgcaaatgaacagcctgaaaaccgaggacacagccgtgtattactgtactagagacacagtgaggggaggtcagtgtgagcccagacacaaacctccctgcaggggcgcacagagccaccagggggcgctagggaccgactgagtacgggacaggtcccaggagcaggtgcagggggaggtttcctttttccttggctggaaaagtcacctttatcttcccaggactcgagccttctaggctgtgatattttattacttgtatttactgttcatttattatcattagtttttaaattttggtaatttttacaactctatggatatatttttaagtgtatactttcaagaaataaacattcctaattatttgcactgattctcccagagttttattaacatttgttgacatcagcaactacatagctatagggacaaacacttttaacgatagacagttgtttaggcctgaaaccccgtttatactaaaaatttacaaaaattagcctggcgtggtgaggggcgcctgtaatcccagctactcgggaggctgagcaaggagaattgcttgaatccgggaagcggaagtcacagtgagtggagtggactgccactacactccagcctggcgacagagcgagactttgtatgaaagaaagaaaagaaaaggaagaaaggaagaaaggaaggaaggaaggaaggaaggaaggaaggaaggaaggaaggaaggaaggaaaagagagaaagaaagagagagaagcaaacaaatggaaacttagacaaggaaagagtaatagaaagacagaaaaaataagttcaatgtcagaagtacctgaaaattaatgtgcctggatcctagttctctccatattttcagaagagtgctggagggcagcaaaaccacacatgctcttattacggaatgtgggttctgataaaaacactagacacatccagctttgtcctggagttggtttagggggatgtcagagacagtgatgaagagcacagggccagataccggggttcactcatcccagacatgagctccgagacgcatacagagcccccccatgtgtgggtttacttccacttctgtaaatggagaaaatattgtctcctacagaacatagtttacatgaatatttaaaatgaaatagggtgattagtgcaaagtgtttatcacagcacaatttcataataagacagcatattttccaaatgcaatcattgccagcaaacttctacagggcaccgtcgtcttatctgggtacagcctactcctcaagggtcccaccctagagcttgctatatagtaggagatatgcaaatagggccctccctctactgatgaaaaccaacccaaccctgaccctgcagctctcagagaggtgccttagccctggattccaaggcatttccacttggtgatcagcactgaacacagaggactcaccatggagttggggctgtgctgggttttccttgttgctattttagaaggtgattcatggaaaactagagagatttagtgtgtgtggatatgagtgagagaaacagtggatatgtgtggcagtttctgaccttggtgtctctttgtttgcaggtgtccagtgtgaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacacagtgaggggaagtcagtgtgagcccagacacaaacctccctgcaggggtcccttgggaccaccagggggcgacagcacactgagcacggggctgtctccagggcaggtgcaggtgctgctgagggctggcttcctgtgatggcctggggcggcctcattgtcaaatttccccggggaacttctccagatttacaatcctgtactaatatttgatgtctctaaatgcaaccttttttttcctttttgtgtctgttttttttttttaaaacaggaggacacatcctcacctccacagaagccacagtgtcactttgggggcagaaataatcctttcgtggtcaacagggtgagagttttgaggaatcccagggaaacctggggaatgttttccaattagactcagggcagagacctccatgggaatctctgattagaacaggctttgagttctgatgggagccaagagagaggctcacccagggtcagggttcttaaaacctgatggttttcacagcaatcccccttcatcttgtgaaactgggcacatctgactcagactgattcagttgaccctctttctgctaatccattttccttcccagtagacttgattctcacagatccctttcttcttctctttcctgaaaacagaggatgtgttttctgtagtctaaattccaaggctcaggtccgcaggttattttgggaacatgatttcataaagaactccaaactcttgtttcaacttctgactcctcgtttctgtgatataagaaaaccatttccaattatgcatctcagggcaattctgtaaacccagagcatttctgctgaagatcctggggaatcaagacaccgggcaggtgatggagacactgtctcaggtgcgcccaacgaatctcagaggaacctgctggagagtcacgtggaacatctacagtcagtttctcagagtcaacagtgagctgtgttggtgcctgaggggaccatgatggggccaaggcacgtgctcagtgtcgtggacagtgatggtccagaaatgatctagatggtcttgacgctaatgaaatatgggttcagagtgaggagcataatctgtggggacttgttcttcagtgaaaggatcctgtccgcaaacagaaatggagcaggacatgcatttcttcaagcaggattagggcttggaccatcagcatcccactcctgtgtggcagatgggacatctatcttctttctcaacctcgatcaggctttgaggtatgaaataatctgtctcatgaatatgcaaataaccttagatctactgaggtaaatatggatacatctgggccctgaaagcatcatccaacaaccacatcccttctctacagaagcctctgagaggaaagttcttcaccatggactggacctggagggtcttctgcttgctggctgtagctccaggtaaagggccaactggttccagggctgaggaagggattttttccagtttagaggactgtcattctctactgtgtcctctccgcaggtgctcactcccaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacacagtgtgagaaaccacatcctcagagtgtcagaaaccctgagggaggagtcagctgtgctgagctgagaaaatgacaggggttattcagtttaagactgtttagaaaacgggttatatatttgagaacaaagaacaatagaaacacaatcgaattgtaagagaaatattccattcaagagccaccacataagccaaactgacagagtgggaaaggccacactcagtaaagttgatacaaacataccataaaggtgctactatgaacaagtttttgaattagatgaataaatcatttggagcaaggttatttggtcatatgttaagagtaagcatgattcttacaaagtgggaaaattgtctttcaaatgtttctgtcacttcttaccataaagttcattttagaggttttaggattacagtgaaattgcacagaaggtgtgagaattcccatgaatccctgccccgcacggacaccgcctcctccactacagccatcctgccccacagtcacaaataagtcacaatggatgaatctacaagaactcttggttctttctttttctggtgatcccctaatataacaagcctaaattatcttggaacacccaggtattttcaatggctttctagaagtgatattagtcagagggaaagtgagtgaggctattactatttgagcactttcttccaaaatccagtttcagtgctcactgaatggagttggaaataaaacccacaattatccataacacaaattccttggaagttattttgggataatgagttcataacctgtagaccaagagtccaagagtgtttctattgaagagcctgggggatcaagacaccaggcaggtgatgcagacactgtctaaagagtgcccagcggctctcagagggacctactggatactcacgtgggacatcagcaatcactttctcagagtcaccagtgagctgtgctggttcctgaagggtccaggatagggccaaggcacctgctctgtgtcgtggagagtgattgttccagaaatcatagaggtggtctctatgcttataaaatctatgttcacagtgagaagtctgttctgagagggcttattcttcagtgaaaggacctctgcccacaaatgttcataaatggagcagggcattcatttcctcaagcaggatcagggcttgagtcatcagcatctcactcttgcaaggctgatgtgtcgtttgtcttccctttcttatcatcgaccaggctttgagctatgaaatgccctgtctcatcaatatgcaaataacctgagatcgactgaggtaaatatggatatgtctgtgccctgagagcatcacccaacaaccacatccctcctctagagaatcccctgaaagcacagctcctcaccatggactggacctggagaatcctcttcttggtggcagcagccacaggtaaggggctcccaagtcccagtgatgaggaggggattgagtccagtcaaggtggcttttatccactcctgtgtcccctccacagatgcctactcccagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggatggatcacacctttcaatggtaacaccaactacgcacagaaattccaggacagagtcaccattactagggacaggtctatgagcacagcctacatggagctgagcagcctgagatctgaggacacagccatgtattactgtgcaagatacacagtgtgaaaacccacatcctgagaccgtcagaaaccccaaggaggaggcagcttcactgaatgaggaggttacagggcttacgatgtttaaagttgttcagaaaataggctaagcaattgaggaatatgagtaatagaaatatgtatgcactctatacaggaaatatttctaataattgtcaccctatatgcaaaattcgcagagaggtaaaagcagaaatcagtcaagctgatgcaaagttccccacgtaggctttgtgcagatgtaagttctaaaatcagatagataaataatttggagcaagattgcttgataacatggctaatgctgaatatgattcctaaaaactggccaaaatatattccaatttgtctctgccacctctcttacataaaatgtattaaaaagtagttttaagaccacagcaaaattgaacagaaggtgcagagatttcctatgtgcccctgcttcacacatgcacagccttccccactgtcaccatcctgccccagagtcatcaataagttacaatggatgaacttacatggatggattggttctttcctcttccggcggtcccttggcataccaagtctaaactatcttgaagcacaacaggttcttcgagtgggttcctgggaatgaagccagttagaggaaaagtgggtggggctattcctattaggagtcttttttaatgtttgtcaaatggtgaacatattgtgtgaagattatatgtctgtattacttcattaaagctattataaataaaagtctaatgtggtagaaaaagatgaagagagaaataaaaataatacaagaaaagtcatgaactcctgaatgaattaacccttagtttttctctattacttataaaaacaccaagatacagccaaataatatcacgatatcattataagaagagtgttttgtaaacctcactgggaatttatagctctttcctagagttaattttgggaacagttggatccaattgtgagaaatgcaggctggacactgagactggctcttatgagatgtgagctcttgtctatgtcacatggtccttccatacttgggggtttacattcacatctgtaaatgaaggaaacattgactctcaaagaacatatttcatgtgcatgtaaaagtatgaatgctagtgagaattaattacttatgaagtataatcacccacatccactcttggacacagcccactctgaggcatctgttacagaactcattatatagtaggagacatgcaaatagggtcctccctctgctgatgaaaaccagcccagccctgaccctgcagctctgggagaggagccccagccctgagattcccaggtgtttccattcggtgatcagcactgaacacagagaacgcaccatggagtttggactgagctgggttttccttgttgctattttaaaaggtgattcatggataaatagagatgttgagtgtgagtgaacatgagtgagagaaacagtggatatgtgtggcagtgtctgaccagggtgtctctgtgtttgcaggtgtccagtgtgaagtgcagctggtggagtctgggggagtcgtggtacagcctggggggtccctgagactctcctgtgcagcctctggattcacctttgatgattataccatgcactgggtccgtcaagctccggggaagggtctggagtgggtctctcttattagttgggatggtggtagcacatactatgcagactctgtgaagggccgattcaccatctccagagacaacagcaaaaactccctgtatctgcaaatgaacagtctgagaactgaggacaccgccttgtattactgtgcaaaagatacacagtgaggggaagtcagcgagagcccagacaaaaaccttgctgcaggaagacaggaggggcctgggctgcagaggccactcaagacacactgagcatagggttaactctgggacaagttgctcaggaaggttaagagctggtttcctttcagagtcttcacaatttctccatctaacagtttccccaggaaccctgtctagatctgtgatctggatctgctgaaactgcctgtgtcaccttcctcacctgtgactttgggggagctgattgtggacactccagtgtgtgggatttcttggtgacagcaattgtgtcttctgtctaggcatgtctagggctggccatcaggaagggcaggctggaatttttggaaagaggcgcacctgccatccaccaggaaattttgttgtcttttgttctgctagaattaaatcagacacaccaaggttaactagcactatcttcctagcttgagaaacttgatggcaggcttgaataacacctgtatgaagccatcagagcaacaactagattaatgtctgctagaattaaatcaggcacaccaaggttaactagcactatcttcctagctcgagaaacttgatagcaggcttgaataacacttgtatgaaaccatcagagcaacacctagaatagtgtctgatttaataattgagactatggtctagccaaggagcagaagtgtttagaggccaggacacatcttcaaggtcacacattgagaaggatggagatatgtcccactaccttctcctacgatctcagacagaatcccaaatttcaaaaggacacagaaggacagctctcaggtgcttttaaaaaatgacccacttccagggacagtgagcttccctgtaaccatggtggatgttctgaactacaataaacattggatggatccagtattgtttgaagtcactgtcattattacattcagctgttgtttcaatgtgtctgaaagggtaaatgactatttagatggcctgggtgtgtggttggttttatatgaatctttaagggttgaacagtactgaccctattccaaaatctgtccttgatccaggatcacactcatctctcagaccagctccttcagcacatctctttacctggaagaagaggactctgggcttggagaggggaggccccaagaagagaactgagttctcaaagggcacagccagcattctcctcccagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgtgagtgtttctaggatgcagacatggagatatgggaggctgcctctgatcccagggctcactgtgggtttttctgttcacaggggtcctgtcccagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgaggggaggtgagtgtgagcccagacaaaaacctccctgcagggaggctgagggggcgggcgcaggtgcagctcagggccagcagggggcgtgcggagctcacggaatacaaggccgggtcaggagcaggtgcagggtgagcggggcttgctcatcttctcagagatcatcatctccctcctcgccagcacctcagctttccgtagaggtcctctttctttattgtgtgtggttctacttcctcacatccttgtgccaggaaagaaaggagtaaggcaaattttcctgttacaattgaagtttcaccaattactaagaactttcctgcaagtacctgcacagcccattataccttatttatatatgtatatattctaatgcttctcaccatctcttgatttgtgtcatcaatttaattgtgccctttttgaaattcatatgctgaaactttaaatccaatggatctatatcggaattttaatggtataattaatgttaaatgtggtcataaatgagaccctaatgcaatagagctgttgtctttataagaagaggaagagacaccagatacctctcacttctcacatgcactcagagaagaggccacgtggagacatagtgcactagaaggtgggcctctgcaagccaggaagaagccgcaccaagaaccaaccctgccagcaccttgatcttccagaaatgtttagaggtcaggccatattctcagggttacacattgagaaggatggagatatattctactaccttctcctgagatctcacacacaatctcaaatttcaaaaggtctcagaagggcagctctcaggtactatttaaaaataacccacttcctgggacaggtagcatccttctaaccatgatggatgttctgaactacagtacacattgcatggatccaggtttgtctcaattcactgtgattattacactcagcagctgtttcaatatgtctgaaggggtaaatgacaatttaggtgacctgggtgtatggttggtgttatatgaatctttaaatgtagaacagtattaactgtattccaaaatctgtctttgatccatgatcacacttgtctcccagaccagctccttcagcacatttcctacctggaagaagaggactctgggtttggtgaggggaggccacaggaagagaactgagttctcagagggcacagccagcatacacctcccagggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaacatgaaacacctgtggttcttcctcctcctggtggcagctcccagatgtgagtgtctcaggaatgcggatatgaagatatgagatgctgcctctgatcccagggctcactgtgggtttctctgttcacaggggtcctgtcccaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcacagtgaggggaggtgagtgtgagcccagacaaaaacctccctgcaggtaggcagagggggcgggcgcaggtactgctcaagaccagcaggtggcgcgcggcgcccacagatcccgaggccgggtccggagcaggtgcaaggagggcggggcttcctcaacagctcagtggtctgtctcctcgccagcacctcagatgtccccaggactctctttctttattatctgtggttctgcttcctcacatccttgtggcagggaagaaaggaggaagacaatttttctgtttactgttgaggtttcaccaattactagaaactttcctacaagttcctgcatgactcattttgccatatatggattctcaccatctcttgatttgtttcatcaatcgaattgtgccctatttgaaattaacttactgaaaccttaaatccaatggatctatactggaattttaatgatgtaattgaggttaaatgtggtcaaagtgtgagaccctaatgcaataaaccgttgtctttataagaagaggaagagacaccagagacctctcacttttcacgtgcacacagagaagaggccatgtggagacgtagtgcactagaaggtggccctgtgcaagccaggaagaagccatactaagaaccaattttgccagctccttgatcttcgacattcagactgtagaattgtaagaattacactcgaataaagcaataaagaaactgatgagataatatttgactgaattgcatcaataaatagatcgatattaacacaaggaatataactgatttccaaaaacatacacatgaaccatggttcactctgcgtatttagataaattacagaaagttgtcataacagatggggaatcctgcagacttcactaggcatgggccatgctgccctggagttgtctcaggggagctgcctcctccagaggttagagcacaggcccaggtaataggactaaatttttagatgtgttatcttagacacactgcacaactgctgtgttctctatgtaaattatctcctgtaaaatataacattgaagcctgcattaaatatattgtgtaaatatgtaagaataaaagaaagttatgagagctaagtgttaatcaaggcacaagcatataagatataactatattttcctgaatgatggaattactaccagtctcccccaggacacttcatctgccctgagcccagcctctcctcagatgtcccacccagagcttgctatatagtgggggacatgcaaatagggccctccctctactgatgaaaaccagcccagccctgaccctgcagctctgggagaggagcccagcactagaagtcggcggtgtttccattcggtgatcagcactgaacacagaggactcaccatggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagacacagtgaggggaggtcattgtgcgcccagacacaaacctccctgcaggaacgctggggggaaatcagctgcagggggcgctcaggagccactgatcagagtcagccctggaggcaggtgcagatggaggctgtttcctgtcaggatgtgggactttgtcttcttctgacagttccccagggaacctcttaaatttagaaaactgtgcctaacaatgtcttctctatgcatatgaggaccttttctccctggcacaaaatgcagattgacgctgacacggatgaaaattcctcaaccatggtcacaaggatcagagtcctgagtaacctcagggcttcctggtgattcttctccaatcagacccaggacagggacctccgtgagattccctgactggaacagtctttatggatcctggtcacagacaatagagaggctgaaccagggtcagcgtcatgtagaacgtcacagatttcacgtctgatccttctcctgacacgaaagtatgcaaatcagtatcagcaccgatctggtgcttcttttgttcctaatccatttactttcttttttcgtcgtttttctcctttttccatttgtttttcctgctttttgcaaaaggaagatgttttccctgtgagatgcaggggatgacaattttgggagatggctggaacatccaatatcctcagggccgaccatctttcattacaggaagaaagttctgaaaatgtcactggggtgaaccacattgtgctgggcttggttcagggagcagtcaggcccagtgttgtgaccttcaaccacagaatcctgaggctgcctcaaagtccccatcagttcccggactcgctatgtttctcgatcgtatcagtgcatccggagctccctggtggctttagtgattccttgcttgccatgctgaggtctcgctgtagattatgttgggttttctaagaccgttttgctattgtaagatccactccctgggacagagcgattccctctaaacctgatggaggttctgaactacaaataacgttaagtgaatcctggtgtgtctgaactcaagtgattgttacattaagctgctgttgcaatctgtttcctcacctgggaaaagaggagccaggacatagtgagttgaggccccaggaagataactgaattctcagagggcacagccagcatcctcttcccagggagagtctaaaagactggggcctccctcatcccttttcacctgtccatacagaggcaccacccacatgcaaatctcacttaggcacccacagaaaaccaccacacatttccttaaattcagggtcctgctcacatgggaaatactttctgagagtcctggacctcctgtgcaagaacatgaaacacctgtggttcttcctcctgctggtggcagctcccagatgtgagtgtctcaaggctgcagacatggagatatgggaggtgcctctgatcccagggctcactgtgtgtctctctgttcacaggggtcctgccccaggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagacacaatgaggggaggtgagtgtgagcccagacacaaacctccctgcagggaggcggaggggaccggcgcagttgctgctcaggaccagcagggggtgcgcggggcccacagagcatgaggccgggtcaggagcaggtgcagggagggcggggcttcctcatctgctcagtgctctccctcctcgccagcacctcagctgtccccagggctcctctttctttattatctgtggttctgcttcctcacatccttgtggtaggaaaggaaagaggaaggcaaattttcctcttagagtcaaagtgtcactaattactaggaactttcctacaagttcctgaatgtcccatttttccttcttaattaaaaaaaatatatattctaatacttctcaccatctcttgatttgtgtcatcagttgaattgtgctgtctttgaaattcaaatgctgaaaccttaaatccaattgatctatattggaattttaaggatggaattaaggttaaatgtgatcataagtctgagattctaatgcaatagatctgttgtctttataagaagtggaagagtcaccagagacctctcacttttcccgtgcacgcagagaagaggccatgtggagacatagtggactagaaggtgcaagccaagaagaagccgcaccaagaaccaaccctgccagcacctggaccttggacattcaattacactcgaataaagcaataaagaaactgatgagataatatttgactgaattgcagcaataaatagatcgatattaacacaaggaatataactgacttccaaaaacatacacatgaaccgtggttcactctgcgtatttaggtaaataacagaaagttgtcataacagatggggaatcctgcagacttcactaggcatgggccatgctgccctggagttgtctcaggggagctgcctcctccagaggttagagcacaggcccaggtaataggactaaatttttagatgtgttatcttagacacactgcacaactgctgtgttctctatgtaaattatctcctgtaaaatataacattgaagcctgcattaaatatattgtgtaaatatgtaagaataaaagaaagttatgagagctaagtgttaatcaaggcacaagcatataagatataactatattttcctgaatgatggaattactaccagtctcccccaggacacttcatctgccctgagcccagcctctcctcagatgtcccacccagagcttgctatatagtgggggacatgcaaatagggccctccctctactgatgaaaaccagcccagccctgaccctgcagctctgggagaggagcccagcactagaagtcggcggtgtttccattcggtgatcagcactgaacacagaggactcaccatggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagacacagtgaggggaagtcattgtgcgcccagacacaaacctccctgcaggaacgctggcgggaaatcagcggcagggggcgctcaggagccactgatcagagtcagccctggaggcaggtgcagatggaggctgtttcctgtcaggatgtgggactttgtcttcttctgacagttccccagggaacctcttaaatttagaaaactgtgcctaacaatgtcttctctatgcatatgaggaccttttctccctggcacaaaatgcagattgacgctgacacggatgaaaattcctcaaccatggtcacaaggatcagagtcctgagtaacctcagggcttcctggtgagtcttctccaatcagacccaggacagggacctccgtgagattccctgactggaacagtctttatggatcctggtcacagacaatagagaggctgaaccagggtcagcgtcatgtagaacctcacagatttcacgtctgatccttctcctgacacgaaagtatgcaaatcagtatcagcaccgatctggtgcttcttttgttcctaatccatttactttattttttcgtcgtttttctcctttttccatttgtttttcctgctttttgcaaaaggaagatgttttccctgtgagatgcaggggatgacaattttgggagatggctggaacatccaatatcctcagggccggccatccacaccgtgctttcattacaggaagaaagatctgaaaatgtcactggggtgaaccatattgtgctgggcttggttcaggaagcagtcaggcccagtgttgtgaccttcaaccacagaatcctaaaaaataataaaagaggctcccccaaagtccccatcagttcctggactcgccatgtgtctgggccgtatcagtgcatccggagctccctggtggctttagggattccttgcttgccatgctgaggtctcgctatagattatgttgggttttctgaggccgttttgctatttaagacccactccctggcacagagcgattccctctaaacctgatagaggttctgaactacaaataacgttaagtgaatcctggtgtgtctgaactcacatgattgttacattaagctgctgttgcaatctgtttcctcacctgggaaaagaggagccaggacatagtgagttgaggccccaggaagataactgaattctcagagggcacaaccagcatcctccttccagggagagcctaaaagactggggcctccctcatcccttttcacctctccatacagaggcaccacccacatgcaaatctcacttaggcacccaagggaaaccatcacacatttccttaaattcagggtcctgctcacatgggaaatactttctgagagtcctggacctcctgtgcaagaacatgaaacacctgtggttcttcctcctgctggtggcagctcccagatgtgagtgtctcaaggctgcagacatggagatatgggaggtgcctctgagcccagggctcactgtgggtctctctgttcacaggggtcctgtcccaggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcacctactacaacccgtccctcaagagtcgagtcaccatgtcagtagacacgtccaagaaccagttctccctgaagctgagctctgtgaccgccgtggacacggccgtgtattactgtgcgagaaacacagtgaggggaggtgagtgtgagcccagacacaaacctccctgcagggaggcggaggagccggcgcaggtgctgctcaggacgagcagggggcgcgcggggcccacagagcaggaggccgggtcaggagcaggtgcagggagggcggggcttcctcatcagctcagtgctctccctcctccccagcacctcagctgtccccaggactcctctttctttattatctgtggttctacttcctcacatccttgctgcaggttttaacagtcactggcacccttttttcaacaaattcccacaaggcccattttaccttcttgacaagcaaaatattctaaggcttctcactatcccttgattggcgtcatctactgcatcgtgtctttctcttaaattcatgtgatgaaaccccaatcccagtggctctgtattggacagagggattttaagagggcaaataagttaactgtggccataaatgtgggaccctaatccagtaggactcttgtccttatgagaagtggaagacatcagaggcctctctctccacatgcacacagaggagagtccatgtggggatgcgctgcatgagaaggtggccctttgcaagcctggaagaggcctctcaaaaaacttaccctttctgcacccagatcttggacgtcaagactccagaacaataagaaaattaatatttgttgctcactcatgcgctggaagagggatgtcagagatgcagtctactcttcctaaagggctgacagggcataaagacaagactgattatccatggaacattcctataggaatgcacctatgtgcagacacacaaataagctgtgtgtcttgcagtgtgtaaatggctgagatgccatttacacttctttacacaatacatttttaaatgttttgttgatgtattctctatcattcaaaaaaatcatcactttccccctaaacaaagaggatttttatcagaacacttatgggaagccccttgctctcccagattcccaccctcatttctcctgaaggaagaaagaaaaccatctctattgatttccactctcatcctctaggactaaaaccagaaagctgcatgctccctgggtgagccttagagaagaccctgtctgtaggagcaggaatctcagagccttggctgagaggcatggtcctgaaatgagatggagtcccccatggagatcccacgtggacgcccacacctgagggctcactgctcctcaccacagatgcactcccctactgagtcctgagacctgagtgcaccccatagagtaggactcagatgagggaatgcaaatctccaccagctccaccctcctctgggttcaaaagctgagcacggggcctcgctcagtgactcctgtgccccaccatggacacactttgctacacactcctgctgctgaccaccccttcctgtgagtgctgtggtcagggacttcctcagaagtgaaacatcagttgtctcctttgtgggcttcatcttcttatgtcttctccacaggggtcttgtcccaggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggataccacagagacacagcccaggatgcctcctgtacaagaacctagctgcatctcagtggtgctccctccctacctctgcagaacaggaaagtgtgactgagatgccatttcctgccagggcttgtgtttcctataccaaccagactcagagccctgtctgttttcctattctgtactattaaatggcatgttccctgttagtgattcacgcaagcagaggctgtatcctgtttgacaaagattgagcatgaaagattcccattacctgggccacatgcatcactgatatgtggccacttatttcttgagctcatatccttccaaggggctgaatacaaatatctataacataggccatcttaaactgcaggaagtaatgttggagaagaatgtggagataaaagagcaggatgattaattaaaatccagataccatctttttttgcagagaaaaatttacactaataaagtaattttatgaaacaacaagaaagatggaatgtgtcctcatcatggagtgtctcacttgcagtgcacaaataaatttctaaaaattttatagggaaggtgtgatagatgaggctgatttccacacagggagtgattaaatacccaggtaagtataaaatccaacacttggaaaggaaaatgcctcagcttgacatcagaaacccagctcagtaagtctgaggagcaatgcggacagggagcacccagcacaggacccagccctggggcaggtgcacaggaggctggggcagggttttctctcaggaattgaatgttccttatttcaaagcaataatgacctaacatttaaataagaatttagcaagtactgatgtgcctgtaagtattttattgtatacgcagcctatacctaacccaataatttaatgcaaaccggatttaaaaggagaaatgtctaggtctttcaaatgtatttctagttaggaattgaggggtggttttattaattcaatgggtgttactgtccggagacacactcatcccagaagttagatgtgcagagggcaaggctcaggaaaagttcaggtagacagtgagccatatgaacagggaatcacattgaggaccatgtcctgtgagattctgggtttctgtgagaggagttctgtcttcatggacttctgggcatgtcagaggacaaatattaaacaaagttcagggcagggagctcagcatcccactgtggcgtggtccacatgtcacctatcttcttcctcagggtggcgtggcctgagctatgaaaaacctgcctcatgaatatgcaaatgcactgctgtctaccgaggtaaatacagatctttccttgcccagagaccatcacacaacagccacatccctcccctacagaagcccccagagcgcagcacctcaccatggactgcacctggaggatcctcttcttggtggcagcagctacaggcaagagaatccttagttccagggctgatgaggggactgggtccagttaagtggtgtctcatccactcctctgtcctctccacaggcacccacgcccaggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagacacagtgtgaaaacccacatcctgagagcgtcagaaaccctgaggaatgaggcagctgtgctgaggctgaggagatgacaggatttatgaagtttaaatctgtttagaaaatgggttgagtaattgagtaaaacagcaatggaaagatgtgtacactgtcattatgtaggaaatagtattttcaactgtcaccctataagtaacactcacagagtgggaaaggcagccatcaatcaagctgatgcaaacattcccatgggggctttgtggggacatacattttataattggatctataaatcattagaaacaggattgcttgatctcatggtaagactaaatataatttccaagaagtggccgaaatttcttccaaaatgtctttgcccttccttttacactgaatttattttaaaggagttttaggatcacaacaagtttgagtagaagaaacagagttcccatgcactcctgccccccgacgctcagccttctctgctatcaacaccctgcaccagtgtcataaatcggttacagaggatgaatctccacagacacattggttgtttccttttctggtggtccctggtataacaagcctcaactatcttgaagcaccccaggttcaagtggattcttcaagtgggttactgggaatgatgccaggtagaaggaaagtgggtgggaccattcctctttgtgcttttctttatatttgttaggtaatcaccacagtgtgtacacatcacaccatgttcccattacagagaaaaggttctgcgaacctcacgagctgtgacccctgtgtgctgggcttggttcagggagaagtcaggtccagtggtgagaagcacaggcccagatgcccaggctcactctgaccaaatgtgagcactggggacattgtaaaacccacctgtgcttttgctgataatttttcatctttaacatggaaataatattgatactatataccatggtttctctgcgtatgtaaaaataaaagatgattggtgctaactttaaaaatatgcagtttatgtagatctatggtacctcaataaaactgttttaaaataaaaattacaaaattataagatttttaggttttaaggtttaagtttatcacaaaacaaactgacaataggaaagcacaatttcccaatgctttcaatatcacagatctccccgaggacattctgacatgctctgagccccactatctccaaaggcctctcaccccagagcttactatatagtaggagatatgcaaatagagccctccgtctgctgatgaaaaccagcccagccctgaccctgcagctctgagagaggagcccagccctgggattttcaggtgttttcatttggtgatcaggactgaacagagagaactcaccatggagtttgggctgagctggctttttcttgtggctaaaataaaaggtaattcatggagaaatagaaaaattgagtgtgaatggataagagtgagagaaacagtggatacgtgtggcagtttctgaccagggtttctttttgtttgcaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagacacagtgaggggaagtcattgtgagcccagacacaaacctccctgcaggaacgatgggggggaaatcagcggcagggggcgctcaggacccgctgatcagagtcatccccagaggcaggtgcagatggaggctgtttcctgtcagggtgtgggacttcatcttcttctgacagtttctctagtgaacctctctaacctcagaattctgtgcttactaatgtcatctctacgtattttttaaaagatcattttaatatgagcacctattctcacacgcaccaaatgcagattgacgcttacagagatgaaaagttctcaaccatggtcaccaggatgagagtcctgaggaaactcaggggtgcctggtgactcttccgcaatcagtcctgggacagaaacctcagggacagcctttatgagttttgatcacatctaacagagaggatgggccagggccagagtcatgtagaacctcacaggttccacgtctgacccttctcctgacactaaagccaatcagcatcagcactgacctggtgctccttttgctcccaatccatgttctttctttggagagtttcttctcccttttttatttgcttttcctgtttcctgaaaaagaaacagatggtccccttggtccacattccagggctcaaggcattttcttggagctcaggtggggctcaggctgtggctctaaaatgtattacaagcaaatggaaatttagacaagcacggagtaatagaaagatacaaacatatgttaaatgtcagaagtatctgaaaattagtgtgtctggaccctagttctctccatattttcaggtgagtgctggtggcaacatcacacatgctcttactacagaaagtgggttctgaaaaccacaccaggcacgtccagctttgtcctggagttggtttagggggatgtcacagccagtgacgaggagcacagggccagataccagcgttcacttatcccagacatgaactcctggatgcatacagagccccctccacttgtgggtttacttccacttctgtaaaaggagaaaatattgactcctacagaacataatttacacaaatatttacagatgaaatagggtgatcagggcaaagtgtttatcacagcacaatttcataagacagcatattttccaaataccatcgtcagcaaacatctgcagggcaccgtcttattatctgggtacagcctattcctccagcgtcccaccctagagcttgttatatagtaggagatatgcaaatagggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgttgctattttagaaggtgaatcatggaaaagtagagagatttagtgtgtgtggatatgagtgagagaaacggtggatgtgtgtgacagtttctgaccaatgtctctctgtttgcaggtgtccagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagacacagtgaggggaagtcagtgtgagcccagacacaaacctccctgcaggggtccccaggaccaccagggggcgcccgggacactgtgcacggggctgtctccagggcaggtgcaggtgctgctgagggctggcttcctgtcatggcctgcggcggcctcattgtcaaatttccccagggaacttctccagatttacaattctgtactgacatttcatgtctctaaatgtaaaacttttttgtcctttttgtttctttgtttttgtaacaggaggacacaccctcacctccacagaaaccacagtgtcactttgggggcagatgatccttctgtggtcagcaggatgaaagttccgaggaatctcaggggaacccgaagagtgtttgccagttagactcagggcagcgacctccacgggaatctctgattagaacaggctttgagttctgataggagccaagagagacgctcacacagggtcagggtccttaaagcctgatggttttcacagctatcccccctggtcttgtaaaactgtgtacatatgactcagactgattcacttgaccccctttctgctaatccattttccttctctgcatacttgattctcacaattccctttcttctcttccccgaaaactgaggatgtgttttctgtagtctaaattccagggctcaggtctgcaggacctgggtacaaacggtgaacatattgtgtgaagattatttgtcaatattatctcattaaaactattacaaataaaagtgtataatttggtaaaaaaaatgaagagagaaataaaaataatacaagaaaagtcatgaactcctgactccacacgtgagttaaccctcagtttttctatgttacttagaaaaacactaagatacagccaaagaatatcatgatatctttataagaagagggttttgtaaccctcactcgaaatttctagctctgtcctagagttaatttagggagcagtcagatccagctgtgagaaacacgggccagacagtgagactggcttttaccagatgtgagctcttagacatgtcacatggcccttccgtgcttgggggtttaccttcacatctgtaaatgaaggaaactttgactcccacagaacataattcatgtgcatctataagtagaaatgctaatgagaattaattatttatgaagtataatcactcgcatccactctgggacacagcctactctgaggcatcccttccagaagtcactatatagtaggagacatgcaaatggggtcctccctctgccgatgaaaaccagcccagccctgaccctgcagctctgggagaggagccccagccctgagattcccacgtgtttccattcagtgatcagcactgaacacagaggactcgccatggagtttgggctgagctgggttttccttgttgctattttaaaaggtgattcatggatcaatagagatgttgagtgtgagtgaacacgagtgagagaaacagtggatttgtgtggcagtttctgaccagggtgtctctgtgtttgcaggtgtccagtgtgaggtgcagctggtggagtctgggggaggtgtggtacggcctggggggtccctgagactctcctttgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagacacagtgaggggaagccagtgagagcccagacacaaacgtccctgcaggaagacaggaggggcctgggctgcagagggcgctcaggacacattaagaacacagtaaactcagggaccaggtgcccagggaggttaagggctggtttcctttcagagacttagctgtttctccatctaacagtttccccagaaccctgcctagatttgtgatctgtatctgctcaaactatctgtgtcaccttcctcacctgtgactttgaggaagcttagtgtggacattccagtgggtggagagatcttggtgacagcaattgtgtgttctgtctaggcatgtctatggctggccatcaggaacgacaggctggaattcttggaaagaggcacacctgccatccatcaggaaattttgttgtcttctgttctgctagaattaaatcaggcacgcccaggttaactactactatcttcctagcttgagaaaattaatggtgggcttgaataacacctatatgaaatcatcagagcaacacctagattcgagtctcatttaataattgagactatgttctagccaaggagacacataaaactggtgattgccgtggtcagttcatatcatatatttgtcagtagaatctggctggtattatgaacagctttgccaacacatgtgttagtgttggtcttcggagaagcacatctgcagggggcgctcaggacacacagagcacaggcttccgccccagagcaggtgaaggaggctggggaggggttcctctcagggcctgggacttcctttaaaaaatctaaaataagtatttcacaaggactgccgatgtttatataaatatcctattcaattgtgagcatttatgaaactcgatgttgtaatgagaaccacttttacaatgggaatttcaaacttccctagacatcttaatagtaagcagctggaggtcaggaggagatcctttcttataaataagtgcaatttttggagaaacacactcattcccaaaatagcacattcacatattaaggtctagaaatgattcgagttgcccctgagacagtcaaatgtgggttctaagtgaggtgcgtgtcctgggggagcttgttctccagtgggggaagctctgtcaacacagagttcagggatgggtaggggatgcgtggcctctaacaggattacggcttgaaccctcagcttctacaattgtgtcgtccatgtgtcatgtatttgctctttctcatcctgggtcaggaattgggctattaaatagcatccttcatgaatatgcaaataactgaggtgaatatagatatctgtgtgccctgagagcatcacccaaaaaccacacccctccttgggagaatcccctagatcacagctcctcaccatggactggacctggagcatccttttcttggtggcagcagcaacaggtaacggactccccagtcccagggctgagagagaaaccaggccagtcatgtgagacttcacccactcctgtgtcctctccacaggtgcccactcccaggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagacacagtgtgaaaacccacatcctgagggtttcagaaaccccagggaggaggcagctgcactgaatttgaggagattacagggcttacaatgtttaaagttgtttagaaaatgagctgagcaattgaggaatgtgagtaatggaaacatggatgcactctatataggaaatgtttctttcaacagtcaccctatatgcaaaattcagaatggtaaaggcagcaatcagtgaggctgacgcaaatattcccatggaggccttgtgcagacatacgttttaaaatcagatagataaataatttggaacaagattgctggtaacgtggctaagactaaatatgattcctaaaaactggccaaaatctattccaaattgtctctgccactccttttacataaatgtattaaaaagtagttttaagaccacagcaaaattggacagaaggtgcagagagttctcatgtgcccctgcttcaccatgcacagcctttcccactgtcaccatcctgccccagagtcatcaataagttacaatggatgaacttacatgggcggattggttctttcctcttctggtggtctcttggcataccaagcccaaattatcttgaagcaccataggttctactgtaatgcctaaccttgttttttactctagtttgctactttaaattttccctttttttgtctccttaattgccaagcaaacaaatgaaaacttgcacaagaaataagtgacataaagatagaaaaaatattaaatttcagaaacacctaataatttatcttcgtgaaccctagttctcaccatatttttaggtgaatgctagaatgcagcaaaattacacatgctctcaatacagaaagtgggtttcacaaaccacactaggcatgctcagctctgtcctggagttgggttagggagtaatatagggccagtggatgaggagcacaggcctagatactggggctcactaacctcaggtatgagctcttagatacatacaaagcccctccacgcatgggtttacttccccatctgtaaattgagaaaccattgacccctaaaaatatgatttacacaaatatgtaaaaatgtaagagagtgattagtgcaaagtgtttatcacagcacaatttcataacaagacagcaagttttccaaacagcatcattgtcattagattcctgcagggcatcattaccttatctgggccctgccctctgctcaggcatcccaccccagagcttgctatatagtaggtgacatgcaaatagggccctccctctcctgatgaaaaccagcccagtcctgaccctgcagctctgggagaggagccccagccttgggattcccaagtgttttcattcagtgatcaggactgaacacagaggactcaccatggagtttgggctgagctggattttccttgctgctattttaaaaggtgatttatggagaactagagagattaagtgtgagtggacgtgagtgagagaaacagtggatatgtgtggcagtttctgatcttagtgtctctgtgtttgcaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagacacagtgaggggaggtcagtgtgagcccggacacaaacctccctgcaggggcgcgcggggccaccagggggcgctcgtgacccactgagggcgggacaggtcccaggagcaggtgccgggagaggtttcctttctcctcagctggaaaagtcaggtttatcttcgcaggactctggagtcttctaggctgtgatattttgttacttatatttattatgaattttatcattaatacttaaattttagtaattattaacattctacatattattatatttttaagtatatactttcaagaaataaacattcctaattgcataataattattgtaacataattattgtaacatataattatatcctaaatgcataataattattgtaacaatcacattctcattttgtatttgaattttcatatttattttactaagattttaattttaatataaaatatttttctacaattgtctgttttccatttttgtgagataaaattagcatatacaaaaatgcacagattctccaggtacagttagagggtttcaggcaaatgtgcacatacttgtctacagcagctaagtgagggtgaggaacaggtgaggtccatctccccacaaagtgtcctcttagtgcttccagttagttctcacataaggatttttttttatttcaactttataatttagatacagagggtatttattacaagtaaacaagtgaaattttagacaaaaaagggatgataagaaggaacaaataaatacattaaatgtcagatacaccaaaaatgtatctgcctgacgcctagttgtctctgtatttttaggtaaatgcagcaaaatcacacaggttgtcgtggcaggaagtggattctgcaaaccacactaggccgtttatctctgtcctgtagttggttcagagcaactgaggccagctgtgaggcgcataggcccaggtactaggactcactcatgccagatataagccctgagacacgtacatagcccctccatgtgtgggttcacttttacatctgtacatgaagaaaccactgactcctaaataacatcatctatacacataggtaaaaaaattaaaaatatgatagttgttaaatgtttatcgcagaacaatttcaaaataaggcagcattttcccaaatacaatcattgtcatcaaaatcccccaggacgctctcatctactctgcgccctgccttcacctcagatgtcccaccccagagcttgctatatagtaacagacatgcaaatagttgactccctctcctgatgaaaaccagcccagccctgaccctgcagctctgggagtggagccccagccttgggattcccaagtgtttgtattcagtgatcaggactgaacacacaggactcaccatggagttggggctgagctgggttttccttgttgctatattagaaggtgattcatggagaactagagatattgagtgtgaatgggcatgaatgagagaaacagtgggtatgtgtggcaatttctgacttttgtgtctctgtgtttgcaggtgtccagtgtgaggtgcagctggtggagtctgggggaggcttggtacagcctggggggtccctgagactctcctgtgcagcctctggattcaccttcagtagctacgacatgcactgggtccgccaagctacaggaaaaggtctggaatgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagagacacagtgaggggaagtcagtatgagcccagacacaaacctccctgcagaatgcctgggggaaatcagctgcagggggcgcacaggacccactgatcagagtcatccccagaggcaggtgcagatggaggctggtttcctgtcaggatgtgggacttcatctttttagagtttctctagggaacctctctaagttcagaattctgtgcttaccaatgccatccctacatatttttaaaatgattattttaatatgaaaacctattctcttatgcacgaaacacagactgatgcttacagagatgaaaagccctcaaccattgtcaccaggatcagagtattgaggaaactcaggggtacctggtgggtcttctccactcagactcaggacagaaacctcagtgagtttccctgactaggacggtctttaggaattgtgatcgcagccaatagagagtctgggcaaggatcagtgtcatgtagaacttcacaggtttcacttctgacccttctcctgacactaaagtatacaacttagtgtcagcactgatctggggccccttttgctcttagcccattctatttctttttatttgttgttgttgttcttgcttttccttgtagtgttcctgctccctgtaaagtggggatgtggctcttgctgccaaagctccaggtctcaagcccattccctgcagctcaaagcagtaacaaaaatgagtatattggctgaggaagagcaagaaagagatgaatattgacacttgaataatcacggactcctgaaaatacacacatgtgaacactgattgcatatttcaggtaaatactagaaaaagcaaagtcacataaagtcgttatgacaggtgggataccctgcaaatcacactaggcatgtcccacactgccctggagctgtctcaggggagcagtctcctccagtgtttagaggcacaggcacggataatagggctgacgctgtccagatgtgtgatattggacacattgcacaactgctctgttatgtatgtaattcatcttctctaaaaatgtaacattgacacttgcactgaatatattctgcaaatatgtaaacattaaataagatgatgactgctaattgatcatcaagtcacaatcacataatctgaagttatattttcctgagagataggattacctccagtgttttccgggaccctctcatctgctctgggcactgccctctcctccagcgtcccactagagcttgctatatagtaggagacatgcaaatagggccctccctctgctgataaaaaccagccgagcccagaccctgcagctctgggagaagagccccagccccagaattcccaggagtttccattcggtgatcagcactgaacacagaggactcaccatggagtttgggctgagctgggttttccttgttgctataataaaaggtgatttatggagaactagagacattgagtggacgtgagtgagataagcagtgaatatatgtggcagtttctgaccaggttgtctctgtgtttgcaggtgtccagtgtcaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagacacagtgaggggaggtcagtgtgagcccagacacaaacctccctgcaggggtccccaggaccaccagggggcgcccgggacactgtgcacggggctgtctccagggcaggtgcaggtgctgctgagggctggcttcctgtcatggcctggggctgcctcattgtcaaatttccccaggaacttctccagatttacaattctgtactaacatttgatgtctctaaatgcaatactttttttgtcctttttgtttctttgtttttttgcaacaggagtacatatcctcagctccacagaagccagggtgtcactttgggggcagaaataatcctttcatggttaccaggataagagtcctgaggaatcccagggaaacctggagagtgttttccagttagactcagggcagagacctccatgggaatctctgattagaacaggccttgagctctgacgggagccaagagagaggctcgcccagggtcagagtccttaaaacctgatggttttcacagctatcccccctcgtcttgtaaactcagactgattcagttgaccctctttctgctaatccatttccttctctgtaggtttgattctcacagttcgctttcttcttctcttccctgaaaacagacgatgtgttttctgtagtcaaaatcccagggctcaggtctgcaggacctgggtaggctacgggtcaaattttgaatgtattgtttgaagattgttatttgtcaatattacctcattaaaactattaaaaatggaagtgtataatttggtagaaaaggattaaaagaaagataaaaataatacaagaaaagtcacagactcctgaatccacatgtgaattagcactcagtttttctatattatttataaaaacactaagatacagccaaataatatcatgatatccttataagaagagtgtttcgtaaacctcactgggaatttctagctctgtcctagagttaatttagggagcagtcggatccagtcctgagcaacacaggccagacactgagacttaccacatcttaccagatgtgagctcttagacacgtcacatggcctctccatgcttgggggtttaccttcacatctgtaaatgaaggaaacattgactgccacagaacatacttcatgtgcatctatacagtagaaatgctaataagaattaattgtttatgaagtgtaatcactctgggacacagcccactcagaggcatcccttccagaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgattcatggagaaatagagagattgagtgtgagtggacatgagtggatttgtgtggcagtttctgaccttggtgtctctgtgtttgcaggtgtccagtgtgaagtgcagctggtggagtctgggggaggcttggtacagcctggcaggtccctgagactctcctgtgcagcctctggattcacctctgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatacacagtgaggggaagtcagcgagagcccagacaaaaacctcctgcaggaagacaggaggggcctgggctgcagagggcactcaagacacactgaaaacacggttaacactgggacaagttgctcaggaaggttaagggctggtttcctttcagagtctttgctgtttctgtatctaacagtttccccaggaaccctgtctagatttgcgatcttgatctgcttaaactgtctctgtgtcatttttcttacctgtgactttaggggagctgatcgtggacactccagtgtgtggagatttcctggtgacagcaattgtgtgttctgtctaagcatgtctagggccggtcatcaggaaggacaggaaggacaggctggaattcttggaaacagctgcacctgccatccaccagggaactttattgtcttctgttctgctaaaattaaatcaggcacaccaagtttaactagcactatcttcctagcttaagaaaattgatggcaggtttcaataacacctatattaaaccttcagagcaacacctacattagtgtctgatttgataattgagactatgttctagccaagcagacacataaaactggtgattgccatgcttagttcatatttcatatttatcaatagaatctggctggtgttataaacagatttgccaacacatgtgtgttagtgtttgtcttcaaattcctatttctatttaaactcccgattcctactctcagtgggagggaaaactcacagccaatcacacatcacaggacaaatctgtaaacgaagagtcattcctctgaaggtcctgggtgttcaggactctcaggcaggtgctgaggaccctgtcttgggagtgcccagcagatctcagaaccctacatggggcctgctggacactcatgtgggataactagtcgccacttattcagagttaccagtgagctttgactgttccgaatgggaccagcatggagtcaaggtgcctgctcaatgtcagagacagcgatggtctcagaaacaatccaggtaatctctaggccaataaaatgtggattcacagtgagaagtacatcctggaggtggagcttgttcttcagtgggaagagtgctgtgcacagaaagcttagaaatggggaagggggtgcgtttcctcaggcaggattagggcttcgtccctcagcgtcccactcttgtatggctgatgtggcatctgtgttttctttctcatactagataaggctttgagctgtgaaataccctgcctcatgaatatgcaaataacctgagctcttctgaggtaaatataggtatattggtgccctgagagcatcactcaacaaccacatctgtcctctagagaaaaccctgtgagcacagctcctcaccatggactggacctggaggatcctcttcttggtggcagcagctacaagtaaggggcttcctagtctcaaagctgaggaacggatcctggttcagtcaaagaggattttattctctcctgtgttctctccacaggtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcacagtgtgaaaaaccacatcctcagagagtcagaaaccctaggggagaaggcagctgtgctgggctgaggagatgacaggggttatcaggtttaaggcttttttgaaaatgggttatatatttgagaaaaaaataacaatagaaacaagtacacactctaattttaagagatatattcaattcaagaattgtagaagccgaattcacagtgggaaaggccacactcaataaagttgataaaaacattccaggaaggtgctactgtgaacaagttttcaaattagatgaataaataatttggagcaatgttatttgatcatgaggtgagacagtatgattcttaaaaagtggaaaaaattctttcaaatgtttctgccactctttatcataaaattaattttagagcagttttaggattacaatgaaattgcacagaaggcatgagaattcccatgactccctgccctacacaggcagagcctcctccactatgaccatccagcaccacagtcacaaatcagttacaatggaggaatctccaaggacatttgattctttcttttttggtgatgccctaatataacaagcctaatttatctcggaataccacaagttttgttcagtggatttctaggaatcatattagttagagggaaagttggtgaggctcttactatttgaactcattcttccaaaatccacaaaggttttttggtaagacccagagtcacagagaattcaaatattgttaagctgcttaatagaaaaacaaattatggtaaatgtgttcactggaatactacccatgatttataataaataaatgcctgacacacagaacagcagcaaaaccacacatgctcttattacagaaagtggcttctgaaaaccacaccgggcatgtacagctttgtcctggagttggtttagggggatgtcagagccagtgacgagaagcacagggccagatggcagcgttcactcatcccagacatgagctcctgggtgcatacagagcccccccatgtgtgggtttacttccacttctgtaaaaggagaaaatactgactcctacagagcataatttacacattttttaaaaaatgtaatagggtgatcagggcaaagtgtttatcacagcacaatttcataagacagcatattttccaaataccatcattgtcagcaaacttctgcagagcaccgtcttcttatatgggtacagcctattcctccagcatcccactagagcttcttatatagtaggagacatgcaaatagggccctccctctactgatgaaaaccaacccaaccctgaccctgcaggtctcagagaggagccttagccctggactccaaggcctttccacttggtgatcagcactgagcacagaggactcaccatggagttggggctgagctgggttttccttgttgctattttagaaggtgattcatggaaaactaggaagattgagtgtgtgtggatatgagtgtgagaaacagtggatttgtgtggcagtttctgaccttggtgtctctttgtttgcaggtgtccagtgtgaggtgcagctggtggagtctgggggaggcttggtccagcctggggggtccctgagactctcctgtgcagcctctggattcacctttagtagctattggatgagctgggtccgccaggctccagggaaagggctggagtgggtggccaacataaagcaagatggaagtgagaaatactatgtggactctgtgaagggccgattcaccatctccagagacaacgccaagaactcactgtatctgcaaatgaacagcctgagagccgaggacacggccgtgtattactgtgcgagagacacagtgaggggaagtcagtgtgagcccagacacaaacctccctgcaggggtcccttgggaccaccagggggcgacagggcattgagcacggggctgtctccagggcaggtgcaggtgctgctgagggctggcttcctgtcgcggtctggggctgcctcgttgtcaaatttccccaggaacttctccagatttacaattctgtactgacatttcatgtctctaaatgcaatactttttttgtcctttttgtttctttgtttttttgcaacaggagtacatatcctcagctccacagaagccagggtgtcactttgggggcagaaataatcctttcgtggtcaacaggatgagagtcctgaggaatcccagggaaacctggagagtgttttccaattagactcagggcagagacctccatgggaatttctgattagaacgggctttgagctctgatgggagccaagagacaggctcacccagggtcggggtccttaaagcctgatggttttcacagctatcccccctcgtcttgtaaactcagactgattcagttgaccctttttctgctaatccattttgcttctctgtaggtttgattctcacagttcgctttcttcttctcttccctgagaacaaacgatgtgttttctgttttcagggaaaatcccagggctcaggtctgcaggacctgggtaggcttggaaaaggtaaatgtggtctccgtgacctcaattcaagggctgaagccctttccctgtagctcagctggggctcaggctgtggctactgcagccatgtggaagaggctgaagggactttcttcactctccttgctcaggaccatccactgtattgtgtataggcttctctggaaatgcaagtggccatttgtagtgaaagaaatatgtttgtctggttaaaatgggaggtggatgtagagttaattggctgctacataaactgtccttctccaccagtgcttttaggatgagattgtgaaatttgtaagaatcaaaatggagtcacatatgttaaaaccctgacaaatggattcaggaagtgtagggagaattcttacacacatatccctgacaacaagaactatcataaaatagttcttgcaaaaagaccaacatgacctcataatcatgacttctgcaaagacttctactcagaatctacttgcccagccttagattaatgccatctgaattacactgatcatgttactatcactgctcctcaccacagatgcaacaccctcctgagtcctgaaacctgactccatcccatagagtagggcacagatgaggggaatgcaaatctccaccagctccaccctcctctgggttgaaaaagccgagcacaggtcccagctcagtgactcctgtgccccaccatggacacactttgctccacgctcctgctgctgaccatcccttcatgtgagtgctgtggtcagggactccttcacgggtgaaacatcagttttcttgtttgtgggcttcatcttcttatgctttctccacaggggtcttgtcccagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagaccacaaagacacagcccagggcacctcctgtacaaaaacccaggctgcttctcattggtgctccctccccacctctgcagaacaggaaagtgcagctgagatacgttttcctgccagggcctgcatttcccatccccattagactcagagccctgtcttcctccttcttctttaataataaatggcatgactcctgttaatagttcatagaagcagaagctgagtcctgtttgtcaaacattcagcatgaaatgttcatgttacctgggccagatgcatcactggtatgtggccgccagtttaatgggctcatactactccaagcggccgaatacaaaacagcagaacatatgcctttattaaactgcaggaaatgacactggagaaaaatgtggggataagggagcaggaggaattgtgaaaattcagataatagcttttctcgtagagagaaaggtgcgttaataaaataattttataaaacaataaaacagatggaatgtgtccccatcacggagtgtctcacagtaacagtgcagaaataaatttctaaaaatgtcgtagggtaggtgtgatagacaaggcttatttccacacagggagcccataaatacccatgtaattaaaaatccagctcttagaaagggagtgcctcaacttgacatcagaaacccacgtcagtgagtctgaggagcaatgtgctctgcagtgcaagggctgggctgcaggggacgctcaaagcccacccagtacaagtttcagccccagagcaggtgcacaggaggctggggagggattcctctcgagatctgtgtcattctttaaaaaatctaaaatacatatttgacaactgaacattctataaatatcctattcaattgcgagcatttatcaaacttgatgttgtaatgagaaccacttttacactggagatttctaacccttctagatatcttaatagtatgcagctggaggttaaggaaactacttttcttttctataaataagtgaaacttttggagaaacacactcatcccccaaataatacattcatgtattaaagcctagaaatgcttatattacctctgagccatttgagtgtgggttcccagtgaagtcctgttctagggaaaattgttagccaatgggagaagctctgtcaacatagagcttagggatatggcagggctcacatggcctctaaggggattacagcttgaaccttgagcatcctccgtgtgtcatctctctgctctttctcatgcaatatcaggtatgaaataggatcactcatgaatatgcaaataactgaggtaaatatagctatctttgggccctgagagcatcacccaacaaccacacccctcctaagaagaagcccctagaccacagctccacaccatggactggacctggaggatcctcttcttggtggcagcagcaacaggtaaggggctccccagtcactgggctgagggagaaaccagcacagtcaagtgagacttcatgcactcccatctcctctccacaggtgcccactcccaggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagacacagtgtggaaacccacatcctgagagtgtcaaaaaccctgtgggaggaggcagctgtgctgagctgaggcagtgacagggacaacgtggctgcaccctttgtagggttataaacatacatatatatgcacccaacagtggagtacccagatatatcaagcaaacattattagagctaaacagagagatagacccaatacagtaaaagctggagactttaacaccccaccctcagcattggacagatcttccagacagaaaattaacaatgaaatcttagactaaatgtacatgacagaccaaatggacataatggatattgacagaacacttcatccaatggctgtagagtgcacagtcttttcctaaacatatggatcagtctcaaggatagaccatatagtagaccagaaaattagtcattttttaattgagaagatatcaagtaccttctctgatcataatggaagaaaactaaattaataagaggaatcttggaaaactatgcaaacacatggaaattaaacaatatgctccctaattaaacaatgtgctcccaaattgaaccacatgctccaaaatgggttaataaagacataaaaaattttaaaatttattgaaagaaatgataaagaagacaaagcatatgaaaacctatgaaatacagtgaaagcagtattaagaggaaagtttatagctcaggcctgtaatcccagcattttgggaggctaaggcaagtggatcacctgaggtcgggacattgagactagcttggccaacatggtgaaaccttatctctactaaaaatataaaaattagacgggggtggttgtgcgtgcctgtactcccagctactcgggaggctgaggcaggagaatcacttgaacccaggaggcggaggttgcggtgagccgagatcgggacactgcactctagcctgggcaaaggagcaaaagttcatctaaaaaatttattttaatttaaaaattttaaaaaaatggcccactccctagaacagagagattccctctaaacatgatggaggtcccgaactatacattaagtgaatcctggtgtgtctgaactcacatgattattacgttaagctgctgttccaatctacttcctcacctgggaaaagaggagccagggcatggctagttgaggccccaggaagagaactgagttctcaaagggcaaagcaagcatcctcatcccagggcgagcctaaaagactggggcctccctcatcccttttcacctctttatacaaaggcaccacctacatgcaaatcctcacttaggcacccacaggaaaccaccacacatttccttaaattcagggtccagctcacatgggaaatactttctgagagtcatggacctcctgcacaagaacatgaaacacctgtggttcttcctcctcctggtggcagccccgagatgtgagtgtctcaaggctgcagacatgggggtatgggaggtgcctctgatcccagggctcactgtgggtctctctgttcacaggggtcctgtctcaggtgcagctgcaggagttgggcccaggactggtgaagcctccggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagacacagtgaggggaggtgagtgtgagcccagacacaaacctccctgcagggaggcggaggggaccggcgcaggtgctgctcagagccagcagggggcgcgcggggcccacagagcaagaggccgggtctggagcaggtgcagggagggcggggcttcctcatcagctcagtgctctccctcctcgccaggacctcagctgtccccaggcctcctctttctttattatctgtggttctgcttcctcacatcctcgctgcaggcaagaaagaagggagaaatttctctgtttaccattaaggttttaacagtcactggcacctttttttcaacaaattcccacaaggcccattttaccttcttgaaaaaatatttcaaggcttcccaccatctcttgattggcgtcatcggctgcatcgtgtcctgctcttaaattcatgtgatgaaaccccaatcccagtggctctgtatttggagagagggattttaagaaggcaaataagttaactgtggtcataaatgtgggaccctaatccagtaggactcttgtccttatgggaagtggaagacatcagaggcctctctctccacatgcacacagaggagaggccatgtggggacgcgctgcatgagaaggtggccctttgcaagcctggaagagggctctcaaaaaactaaccctttctgcacccagatcttggaccaggagcctctctgcagtgcaagggctgggctgcagggggcgctcaaggcccacccagcacaggctccagccccagagcaggtgcacaggaggctgcggatggagtccccttaagatctgtgtcattcttctaaaaaaatctaaaataagtatttgacaaaaactgctgaagagtccataaatatcctattcaattgcaagaatttatcaatttacactggagatttctaaccctgctacatatcttaatagtaagcatctggaggtcaattaagcttttattttatataaataagtgcaacttttggagaaacacactcatcccccaaataacacattcatgtattaaagcctagaaatactttaaattacctctgagctattcaagtgtgggttcccagtgaagtcctgttctagggaaaattgttctccagtgggagaagctctgtcaacatacagcttagggatatggcagggcacacatggcctctaaggggattatagcttgaacccttagcatcctcctgttgggtaatccatgtgtcatctctccactctttctcatgctgtgttaggtatgaaatagcatcgctcatgaatatgcaaataactgagatgactatagatatctttgtgccctgagagcatcacccaacaaccacatccctcctcagaagcccccagagcacaacgcctcaccatggactggacctggaggatcctctttttggtggcagcagccacaggtaaggggctgccaaatcccagtgaggaggaagggatcgaggccagtcaaggggaattccatccactcctgtgtcttctctacaggtgtccactcccaggttcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggagcaacgctggcaatggtaacacaaaatattcacaggagttccagggcagagtcaccattaccagggacacatccgcgagcacagcctacatggagctgagcagcctgagatctgaggacatggctgtgtattactgtgcgagagacacagtgtgaaaacccacatcctgagagtgtcagaaaccccaggggggaagcagctgtgctggcatggaggaaatgacaaagattattagattgaagactttctcagaaaatgatattaagtcattaaggaaaaggaacaatataaacatgtatttgagaaattttaattatttgagagatttttcatacaatatttattctgcaagcaaatttcagggattgaattaataaaactgatacagaacttcctctgtaggtatctgtgtaaacatcaatttctgaatcagtgttgtaaatattttggaacacacacacaaatcacattttatctctacttttatctctattattaaaaatgccaaaaaaactcattttgtgcatgtagcattttgaattcccaccatcaatgcatgatagttcttggttttccacattcatattgccatttatcattatgagaattgtgtgttttaaccattctaataggtgagtaatggtatctaatttttagttaaatgcacatttccgtaataaaaattcacatttaacaattttcatataatttttgccgagatgcctcttctcatatttggttcatttttaactgcattgttttcttttgattagttgtaagtttacttgcatattgattataaaatcatttaacaaattaaaagaattcatttaacaaatatgtgacttggaagtattttctccaagtctgcggctgtcttttactcccttatcagcacaggctgcagccctggagcaggtgcaagggaggctggggaggggttcctcccagggtctgatgtcttccttttctcggacaaacatgctttaataagttaaacaagactttagtaaagactattgatgtgtctttgtgtctttcagtatacagttctatttgtaggatttatctaacctaacaagtcaatgagaatcacatgtaaaaggagaaatttctaggattttcagatatcttaataggtaggagatggagaaaagggatggttttattaattcagtgcttgccaatcttaacagagacagtagtaagacatgcagaaagcaaagcccagaaaagtatgaaggtgtcaaagtgccatttaagtatgggttcacttggaggaccatgttctgcgggaacttgttttcagcagacaatctattttagcagagttctgggcatacaaggggacacacatcattaaacaaggattgggacagggacttcagcgtcccactgttgcatggcccataaattatgtgtgttctctttctcatcttggatcaagtctagagctatgaaatagtatccctcatgaatatgcaaataacctgagatttactgaagtaaatacagatctgtcctgtgccctgagagcatcacccagcaaccacatctgtcctctagagaatcccctgagagctccgttcctcaccatggactggacctggaggatcctcttcttggtggcagcagccacaggtaagaggctccctagtcccagtgatgagaaagagattgagtccagtccagggagatctcatccacttctgtgttctctccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccagtaccagggacacgtccatcagcacagcctacatggagctgagcaggctgagatctgacgacacggtcgtgtattactgtgcgagagacacagtgtgaaaacccacatcctgagggtgtcagaaaccccagggaggaggcagctgtgctggggctgagaaatgaaagggattactatttttaatgttgtttacagtatgtcattaataaattgaaaaaaagtaacaatagaagtatatactctaattatatgggaactttgttttttcagttttttcatttttttttttttttttggtttgtttgtgacagagtctcactctgccacccaggctggagtgtaacggcacaatctcagctcactacaacctccacctcccaggttcaagcaattctcctgcctcggcctccagagtagttgggattacaggcacccgccaccatgcccggtgaatttttgtatttttagtagagacggggtttcaccatgttagctaggctggtctcaaactgctgatctcaggtgatctaccctcctcagcctcccaaagtcctgggattacaggcgtgagccactgcgcctggcccaattatatgggaattgtttatataattatcaccctataagcaaaattcatggaggaggaaaagctctactgaagaaagctgataccggcattcccatgaaagtatctgtgtagaagtaagtattaaaatcagttgaataggcaaggcatggtggctcacgcctataatcccagcactttgggagaccgaggcaggtggatccccttggggtttgggatgagctgcccttgcccactgtgctctgtggacctccctttagaagctcacagctccctgcactcggctccatcctgccccaccacacagaagcaaaacccctctcctttccactgcaggcttttcctggaccagaatgctgacctgctgcccttcactcccgaagtggtgggactgcctggggtggtgtgggtgttgagccttcttactctagggacctggcacctggccccaggggcacagggatggtgcatctgcctagggatgcctcctcatgccagggggtgggggttagtaccatcggccctcaggatttgttgcatgaatgagtgaatgggtgaataaatgaaggggatctgatctatgaataagggtatatagactttggttgatgtaggacgccaaatgctggaatttcagagtcatcacacccaggggccctgcctctgagctcctctttgcatccaatctgctgaagaacatggctctagggaaacccagttgtagacctgagggccccggctcttcaatgagccatctccgtcccggggccttatatcagcaagtgacgcacacatgcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcacaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagacacagtgaggggaagtcagtgtgagcccagacacaaacctccctgcagggatgctcaggaccccagaaggcacccagcactaccagcgcagggcccagaccaggagcaggtgtggagttaagcaaaaatggaacttcttgctgtgtcttaaactgttgttgttttttttttttttttggctcagcaacagagatcatagaaaaccctttttcatatttttgaaatctgttcttagtctaatggagattctctaatatgtgacaatgtttttctcttgctgtttttggaattctttgtctttgacttttgacaacttgacttttgacagtgtgcctcaaagaagttctattttgggttctgtgaacctcctggatctgggaagttttcagctatgatttcattaaacgtgttttctacaccatttccctactcttttggaatacccataatgcaaatatttgttcacttaattgtgtcccataaatgctggggattttcttcattcctttttactctttttttctttttattcatctgcctgaattatttcaaaagatctgtcttcaacttcagaaactcttttgcttggcctagtctaatcttgaaggtctcaattgtacttttaatttcattcattgaattcttcaactctggaatttctgttggttcttttttatgatacttatctctttgttgaattcgaacacaaaatagtgacagcagagatgacggcaagttggcatttttcttttctagcaataaaacttaaagctgactcaaggagaaatggaaatcataattggaacagtaatcctcaagaaagcattaagattattaaataattgccctcacagatgacttcaggccaagatggctttatgggtgaagtttagactttcacaaaactaatcagttcccataagaactgctccaggatttggaggaacatgggaaagtctattaaagggatcacaattcacagtccccagagtaaaacatgggctaacttgcattttggcaaagagccaaatgttataaatgacatcctagaaggccaaattctgtccatctcgttgaacaaggacttacaccaggaatttagaactatttatagctcatcccaccactcaggccaatgatgacccatgatcatctcaccagaaatggaaagactcagatgattaatagagtctcaatttctctgagacatctaagagcccagcccaagcccagacccaggagggcacccaggcctggacagagaacactgatatcacaccagccctccagagggaagcagagactccttcaagctctggaaacacaggcccagacagctgcctaaagttgggcaggcttcactgcaaacccaaatcatgaagctaggtaacacctttacagattctttacatttaaaaatcatcaaaacaagagtaaataataaactcaaataatattaatctaatatgtaaaggtcttgtaccattattatgcaaacaacatacataagctaataagaaaaagaacaaatcccttaagaaatcggcaaaaaggatataacacaatttctaaaagaaaacaaatggctagcacacataaggaaaacactttgtgaacagacattcttcagaacattatttataattataaaatagttgaaagcaagatagtgcctgaagaaattatggtgcatacattagtgggactattctgcaaacattcccaattatacttgtcacatatctgtgataacgtgacagccagcattcatggggtgacctcatttggtaaaagggtgcaaagctcaacacgcattgtgagatgactgtggtgtaaaattagtgggattattccgcaaacattcccaattatacttaccgcatatctgtgataacatgacagcattcatggggtgacctcatttggtaaaagggtgcaaagctcaacacgcattgtgagatgactggtgtaaatacaaagaccaaactgtgaaaaggagtccatcaattaatcgatgcttaccttcagttttgggctaatttttaaagtatgctataagcatatgctcctgttataacagaatggagggattatgagagatgatgcaggtgtgtcctgggcctcccctggcccactgggccctagagatgccttcccaggcatcgctgtcagggcttccctcagagggagtcctgtattgacctcaccaccaaggtctggagcaggggatccttagatattggttggggttatctcaccttaggtctgaatttggggttgtcttagactgttttgtgctgttagaatagaatacccaagactgggaaatttatactgaacggaaatttatttctcacagttctagaggctgtgaagtccaagagcacaggtgccagagcaagtccaagagcaagggaaagtccaaagcaagtccaggagcatctggcgaggaccttcttgctgtgtcatcacatggcggaaggcaagaaagagagcaagagggggccgaactcacccttttataacagcaccaatcccacccatgaggtggggaccttatgacctaatcactcttcatactgttacaatggcaatgaaatttcaacatgagttttggaggagagaagcattcaaaccacagcaagggtgctcctacctcctctctcagggcatctgcagaaagagctgcaactgcacgtccttcctccgtccatcctccatcccttcccaatgtccgtgcatatcctgtgacccaggaggtctggcatagggggtgctcctgccttaggtctgaggccctgtctgaagaggggtaggtgaggaggccatctgatggtctgggccaagacagtcacaggacgcatcatttatcatcaaggaggctgagggttgagtctccaggtccagggaactccccacaaagtgggaaccctgcccagctccacacagcctctgctgggggaccctgctctggtgcagagcctggggacaggtcttgagctcagccagagtctgcctccctgtcatttaggaactaaaccaagcggcaggatgctggagcccagcccccatctgaccttacagggccaaggctggggccctgggttcccctcaaggcgcagcaggactggagccccaggcagtgcaggagtggccaaagctggggcttcctccagagcccccaagcatcacggcaccaagaagggtaggaccctggcctgaggaattggcaccaaagccccagaaactaccctggacaccatggagagaggcctggaggggaagcaccaggcactgcctccccttctgatcccacctgaggtggctgccaagcccagagagccgctctgatgtcccccagccctgcagcccagggatacctgtactgtgcccctgggggacccctggccagtctgtgcaaagaagtcaccaccctacactcagagacagtgggggtcctcgtcccacatcctcagagcatggcccggctgctgcagggatggtctcctggtcctcagagcatggcccggctgctgcagggatggtctcctggtcctcagagcatggcccagctgctgcagggatggtctcctggaggccccccagtgctctattgtcagggctccctccaccccccgcaccaagagagagccagaccccagcaaggcttccagtggcttcaggtcacacccctaggctgaccccagccccattaacacctgcctgagaaagctccacgcaccagaactgaccgtctgctccaactcttgacctcccgttctcagggcgtctgctgaaaaggctgcaactgcacatccttcctccgtccgttcccgatgtccgtgtgtctcctgtggccaggaaggtctttctcgggacctgagagccgctccctgaagtgtccccattgggaaggatggggcctgtgtctccaggctctgggaggacagaatcctgacctcaacagtggccggcacggacacaactggccccatcccggggacgctgaccagcgctgggcaacttttcccttccccgacgactgagccccgagcaccctccctgctcccctaccacctccctttacaaggctgtggcctctgcacagatgataatggagcttggctcattcccctagagtcggtagggagttaaggacaaaactcagtttcctccacctgaactcaagtctgcctatgtttacctaatcacacctggtggacagtttggacaaacttgcacactcagagacacagacacttctagaaatcattatctccctgccccggggaccccactccagcagaagtctgctaggcactggcctgggccctcctgctgtcctaggaggctgctgacctcctgcctggctcctgtccccaggtccagagtcagagcagactccagggacgctgcaggctaggaagccgccccctccaggcgagggtctagtgcaggtgcccaggacaagaaagattgtgaatgcaggaatgactgggccacacccctcccgtgcacgccccctcctgccctgcaccccacagcccagccccccgtgctggatgcccccccacagcagaggtgctgttctgtgatcccctgggaaagacgccctcaacctccaccctgtcccacggcccaaggaagacaagacacaggccctctcctcacagtctccccacctggctcctgctgggaccctcaaggtgtgaacagggaggatggttgtctgggtggcccctaggagcccagatcttcactccacagaccccaacccaagcacccccttctgcagggcccagctcatccccctcctcctccctctgctctcctctcgtcgcctctacgggaaatccgggactcagcagtaaccctcaggaagcagggcccaggcgccgtttaataggaggcttcctcacaatgaaacttttagaaagccttgactacaatgatgaccttggtgtggctgtgaacactgtcagctcccacagctgctgcagcaaaaaatgtccatagacagggtgggggcccggggtcgtctgctgtcctgctcagcccacagcacgcatggaggatctgaggtgccacacctgacgcccaggccagaacatgcctccctccagggtgacctgccatgtcctgcattgctggagggacaggggcagcctatgaggatctggggccaggagatgaatcctattaacccagaggaaaactaacaggacccaagcaccctccccgttgaagctgacctgcccagaggggcctgggcccaccccacacaccggggcggaatgtgtacaggccccggtctctgtgggtgttccgctaactggggctcccagtgctcaccccacaactaaagcgagccccagcctccagagcccccgaaggagatgccgcccacaagcccagcccccatccaggaggccccagagctcagggcgccggggcagattctgaacagccccgagtcacggtgggtacaactggaacgaccaccgtgagaaaaactgtgtccaaaactctctcctggcccctgctggaggccgcgccagagaggggagcagccgccccgaacctaggtcctgctcagctcacacgacccccagcagccagagcacagtggagtccccactgaaccccactgaatggtgaggacggggaccagggctccagggggtcatggaaggggctggaccccatcctactgctatggtcccagtgctcctggccagaactgaccctaccaccgacaagagtccctcagggaaacgggggtcactggcacctcccagcatcaaccccaggcagcacaggcataaaccccacatccagagccgactccaggagcagagacaccccagtaccctgggggacaccgaccctgatgactccccactggaatccaccccagagtccaccaggaccaaagaccccgccccggtctctgtccctcactcaggacctgctgcggggcgggccatgagaccagactcgggcttagggaacaccactgtggccccaacctcgaccaggccacaggcccttccttcctgccctgcggcagcacagactttggggtctgtgcagagaggaatcacagaggccccaggctgaggtggtgggggtggaagacccccaggaggtggcccacttcccttcctcccagctggaacccaccatgaccttcttaagataggggtgtcatccgaggcaggtcctccatggagctcccttcaggctcctccctggtcctcactaggcctcagtcccggctgtgggaatgcagccaccacaggcacaccaggcagcccagacccagccagcctgcagtgcccaagcccacattctggagcagagcaggctgtgtctgggagagtctgggctccccaccgccccccgcacaccccacccacccctgtccaggccctatgcaggagggtcagagccccccatggggtatggacttagggtctcactcacgcggctcccctcctgggtgaaggggtctcatgcccagatccccacagcagagctggtcaaaggtggaggcagtggccccagggccaccctgacctggaccctcaggctcctctagccctggctgccctgctgtccctgggaggcctggactccaccagaccacaggtccagggcaccgcccataggtgctgcccacactcagttcacaggaagaagataagctccagacccccaagactgggacctgccttcctgccaccgcttgtagctccagacctccgtgcctcccccgaccacttacacacgggccagggagctgttccacaaagatcaaccccaaaccgggaccgcctggcactcgggccgctgccacttccctctccatttgctcccagcacctctgtgctccctccctcctccctccttcaggggaacagcctgtgcagcccctccctgcaccccacaccctggggaggcccaaccctgcctccagccctttctcccccgctgctcttcctgcccatccagacaaccctggggtcccatccctgcagcctacaccctggtctccacccagacccctgtctctccctccagacacccctcccaggccaaccctgcacatgcaggccctccccttttctgctgccagagcctcagtttctaccctctgtgcctaccccctgcctcctcctgcccacaactcgagctcttcctctcctggggcccctgagccatggcactgaccgtgcactcccacccccacactgcccatgccctcaccttcctcctggacactctgaccctgctcccctcttggacccagccctggtatttccaggacaaaggctcacccaagtcttccccatgcaggcccttgccctcactgcccggttacacggcagcctcctgtgcacagaagcagggagctcagcccttccacaggcagaaggcactgaaagaaatcggcctccagcaccctgatgcacgtccgcctgtgtctctcactgcccgcacctgcagggaggctcggcactccctgtaaagacgagggatccaggcagcaacatcatgggagaatgcagggctcccagacagcccagccctctcgcaggcctctcctgggaagagacctgcagccaccactgaacagccacggagcccgctggatagtaactgagtcagtgaccgacctggagggcaggggagcagtgaaccggagcccagaccatagggacagagaccagccgctgacatcccgagcccctcactggcggccccagaacaccgcgtggaaacagaacagacccacattcccacctggaacagggcagacactgctgagcccccagcaccagccctgagaaacaccaggcaacggcatcagagggggctcctgagaaagaaaggaggggaggtctccttcaccagcaagtacttcccttgaccaaaaacagggtccacgcaactcccccaggacaaaggaggagccccctgtacagcactgggctcagagtcctctccaacacaccctgagtttcagacaaaaaccccctggaaatcatagtatcagcaggagaactagccagagacagcaagaggggactcagtgactcccgcggggacaggaggattttgtgggggctcgtgtcactgtgaggatattgtagtagtaccagctgctatgcccacagtgacacagccccattcccaaagccctgctgtaaacgcttccacttctggagctgaggggctggggggagcgtctgggaagtagggcctaggggtggccatcaatgcccaaaacgcaccagactcccccccagacatcaccccactggccagtgagcagagtaaacagaaaatgagaagcagctgggaagcttgcacaggccccaaggaaagagctttggcgggtgtgcaagaggggatgcgggcagagcctgagcagggccttttgctgtttctgctttcctgtgcagatagttccataaactggtgttcaagatcgatggctgggagtgagcccaggaggacagtgtgggaagggcacagggaaggagaagcagccgctatcctacactgtcatctttcaagagtttgccctgtgcccacaatgctgcatcatgggatgcttaacagctgatgtagacacagctaaagagagaatcagtgaaatggatttgcagcacagatctgaataaattctccagaatgtggagccacacagaagcaagcacaaggaaagtgcctgatgcaagggcaaagtacagtgtgtaccttcaggctgggcacagacactctgaaaagccttggcaggaactccctgcaacaaagcagagccctgcaggcaatgccagctccagagccctccctgagagcctcatgggcaaagatgtgcagaacatatgtttgtcatagccccaaactgagaatgaagcaaacagccatctgaaggaaaacaggcaaataaacgatggcaggttcatgaaatgcaaacccagacagccagaaggacaacagtgagggttacaggtgactctgtggttgagttcatgacaatgctgagtaattggagtaacaaaggaaagtccaaaaaatactttcaatgtgatttcttctaaataaaatttacagccggcaaaatgaactatcttcttaagggataaactttccactaggaaaactataaggaaaatcaagaaaaggatgatcacataaacacagtggtcgttacttctactggggaaggaagagggtatgaactgagacacacagggttggcaagtctcctaacaagaagagaacaaatacattacagtaccttgaaaacagcagttaaaattctaaattgcaagaagaggaaaatgcacacagctgtgtttagaaaattctcagtccagcactgttcataatagcaaagacattaacccaggttggataaataaacgatgacacaggcaattgcacaatgatacagacatacattcagtatatgagacattgatgatgtatccccaaagaaatgactttaaagagaaaaggcctgatatgtggtggcactcacctccctgggcatccccggacaggctgcaggcacactgtgtggcagggcaggctggtacctgctggcagctcctggggcctgatgtggagcaggcacagagccgtatccccccgaggacatatacccccaaggacggcacagttggtacattccggagacaagcaactcagccacactcccaggccagagcccgagagggacgcccatgcacagggaggcagagcccagctcctccacagccagcagcacctgtgcaggggccgccatctggcaggcacagagcatgggctgggaggaggggcagggacaccaggcagggttggcaccaactgaaaattacagaagtctcatacatctacctcagccttgcctgacctgggcctcacctgacctggacctcacctggcctggacctcacctggcctagacctcacctctgggcttcacctgagctcggcctcacctgacttggaccttgcctgtcctgagctcacatgatctgggcctcacctgacctgggtttcacctgacctgggcttcacctgacctgggcctcatctgacctgggcctcactggcctggacctcacctggcctgggcttcacctggcctcaggcctcatctgcacctgctccaggtcttgctggaacctcagtagcactgaggctgcaggggctcatccagggttgcagaatgactctagaacctcccacatctcagctttctgggtggaggcacctggtggcccagggaatataaaaagcctgaatgatgcctgcgtgatttgggggcaatttataaacccaaaaggacatggccatgcagcgggtagggacaatacagacagatatcagcctgaaatggagcctcagggcacaggtgggcacggacactgtccacctaagccaggggcagacccgagtgtccccgcagtagacctgagagcgctgggcccacagcctcccctcggtgccctgctacctcctcaggtcagccctggacatcccgggtttccccaggcctggcggtaggtttggggtgaggtctgtgtcactgtggtattacgatttttggagtggttattatacccacagtgtcacagagtccatcaaaaacccatccctgggaaccttctgccacagccctccctgtggggcaccgctgcgtgccatgttaggattttgactgaggacacagcaccatgggtatggtggctaccgcagcagtgcagcctgtgacccaaacacacagggcagcaggcacaacagacaagcccacaagtgaccaccctgagctcctgcctgccagccctggagaccatgaaacagatggccaggattatcccataggtcagccagacctcagtccaacaggtctgcatcgctgctgccctccaataccagtccggatggggacagggccggcccacattaccatttgctgccatccggccaacagtcccagaagcccctccctcaaggctgggccacatgtgtggaccctgagagccccccatgtctgagtaggggcaccaggaaggtggggctggccctgtgcactgtcactgcccctgtggtccctggcctgcctggccctgacacctgggcctctcctgggtcatttccaagacagaagacattcccaggacagctggagctgggagtccatcatcctgcctggccatcctgagtcctgcgcctttccaaacctcacccgggaagccaacagaggaatcacctcccacaggcagagacaaagaccttccagaaatctctgtctctctccccagtgggcaccctcttccagggcagtcctcagtgatatcacagtgggaacccacatctggatcgggactgcccccagaacacaagatggcccacagggacagccccacagcccagcccttcccagacccctaaaaggcgtcccaccccctgcatctgccccagggctcaaactccaggaggactgactcctgcacaccctcctgccagacatcacctcagcccctcctggaagggacaggagcgcgcaagggtgagtcagaccctcctgccctcgatggcaggcggagaagattcagaaaggtctgagatccccaggacgcagcaccactgtcaatgggggccccagacgcctggaccagggcctgcgtgggaaaggcctctgggcacactcaggggctttttgtgaagggtcctcctactgtgtgactacagtaactaccacagtgatgaacccagcagcaaaaactgaccggactcccaaggtttatgcacacttctccgctcagagctctccaggatcagaagagccgggcccaagggtttctgcccagaccctcggcctctagggacatcttggccatgacagcccatgggctggtgccccacacatcgtctgccttcaaacaagggcttcagagggctctgaggtgacctcactgatgaccacaggtgccctggccccttccccgccagctgcaccagaccccgtcctgacagatgccccgattccaacagccaattcctggggccaggaatcgctgtagacaccagcctccttccaacacctcttgccaattgcctggattcccatcccggttggaatcaagaggacagcatcccccaggctcccaacaggcaggactcccacaccctcctctgagaggccgctgtgttccgtagggccaggctgcagacagtccccctcacctgccactagacaaatgcctgctgtagatgtccccacctggaaaagaccactcatggagcccccagccccaggtacagccatagagagagtctctgaggcccctaagaagtagccatgcccagttctgccgggaccctcggccaggctgacaggagtggacgctggagctgggcccacactgggccacataggagctcaccagtgagggcaggagagcacatgccggggagcacccagcctcctgctgaccagaggcccgtcccagagcccaggaggctgcagaggcctctccagggggacactgtgcatgtctggtccctgagcagccccccatgtccccagtcctgggggcccctggcacagctgtctggaccctctctattccctgggaagctcctcctgacagccccgcctccagttccaggtgtggttattgtcagggggtgtcagactgtggtggatacagctatggttaccacagtggtgctgcccatagcagcaaccaggccaagtagacaggcccctgctgtgcagccccaggcctccagctcacctgcttctcctggggctctcaaggctgctgttttctgcactctcccctctgtggggagggttccctcagtgggagatctgttctcaacatcccagggcctcattcctgcaaggaaggccaatggatgggcaacctcacatgccgcggctaagatagggtgggcagcctggcggggacaggacatcctgctggggtatctgtcactgtgcctagtggggcactggctcccaaacaacgcagtcctcgccaaaatccccacggcctcccccgctaggggctggcctgatctcctgcagtcctaggaggctgctgacctccagaatggctccgtccccagttccagggcgagagcagatcccaggccggctgcagactgggaggccaccccctccttcccagggttcactgcaggtgaccagggcaggaaatggcctgaacacagggataaccgggccatcccccaacagagtccaccccctcctgctctgtaccccgcacccccaaggccagcccatgacatccgacaaccccacaccagagtcactgcccggtgctgccctagggaggacccctcagcccccaccctgtctagaggactggggaggacaggacacgccctctccttatggttcccccacctggctctggctgggacccttggggtgtggacagaaaggacgcttgcctgattggcccccaggagcccagaacttctctccagggaccccagcccgagcacccccttacccaggacccagccctgcccctcctcccatctgctctcctctcatcaccccatgggaatccagaatccccaggaagccatcaggaagggctgagggaggaagtggggccactgcaccaccaggcaggaggctccgtctttgtgaacccagggaggtgccagcctcctagagggtatggtccaccctgcctatggctcccacagtggcaggctgcagggaaggaccagggacggtgtgggggagggctcagggccccgcgggtgctccatcttggatgagcccatctctctcacccacggactcacccacctcctctccaccctggccacacgtcgtccacaccatcctaagtcccacctacaccagagccggcacagccagtgcagacagaggctggggtgcaggggggccgccagggcagctttggggagggaaggatggaggaaggggagttcagtgaagaggcccccctcccctgggtccaggatcctcctctgggacccccggatcccatcccctccaggctctgggaggagaagcaggatgggagaatctgtgcgggaccctctcacagtggaatacctccacagcggctcaggcaagacccaaaagcccctcagtgagccctccactgcagtcctgggcctgggtagcagcccctcccacagaggatgaacccagcaccccgaggatgtcctgccagggggagctcagagccatgaaggagcaggatatgggacccccgatacaggcacagacctcagctccattcaggactgccacgtcctgccctgggaggaacccctttctctagtccctgcaggccaggaggcagctgactcctgacttggacgcctattccagacaccagacagaggggcaggccccccagaaccagggatgaggacgccccgtcaaggccagaaaagaccaagttgtgctgagcccagcaagggaaggtccccaaacaaaccaggaagtttctgaaggtgtctgtgtcacagtggagtatagcagctcgtcccacagtgacactcgccaggccagaaaccccatcccaagtcagcggaatgcagagagagcagggaggacatgtttaggatctgaggccgcacctgacacccaggccagcagacgtctcctgtccatggcaccctgccatgtcctgcatttctggaagaacaagggcaggctgaagggggtccaggaccaggagatgggtcccctctacccagagaaggagccaggcaggacacaagccccctccccattgaggctgacctgcccagagggtcctgggcccaccccacacaccggggcggaatgtgtgcaggcctcggtctctgtgggtgttccgctagctggggctcacagtgctcaccccacacctaaaacgagccacagcctcagagcccctgaaggagaccccgcccacaagcccagcccccacccaggaggccccagagcacagggcgccccgtcggattctgaacagccccgagtcacagtgggtataactggaactaccactgtgagaaaagcttcgtccaaaacggtctcctggccacagtcggaggccccgccagagaggggagcagccaccccaaacccatgttctgccggctcccatgaccccgtgcacctggagccccacagtgtccccactggatgggaggacaagggccgggggctccggcgggtcggggcaggggcttgatggcttccttctgccgtggctccagtgcccctggctggagttgacccttctgacaagtgtcctcagagagtcagggatcagtggcacctcccaacatcaaccccacgcagcccaggcacaaaccccacatccagggccaactccaggaacagagacaccccaataccctgggggaccccaaccctgatgactcccgtcccatctctgtccctcacttggggcctgctgcggggcgagcacttgggagcaaactcaggcttaggggacaccactgtgggcctgacctcgagcaggccacagacccttccctcctgccctggtgcagcacagactttggggtctgggcagggaggaacttctggcaggtcaccaagcacagagcccccaggctgaggtggccccagggggaaccccagcaggtggcccactacccttcctcccagctggaccccatgtcttccccaagataggggtgccatccaaggcaggtcctccatggagcccccttcaggctcctctccagaccccactgggcctcagtccccactctaggaatgcagccaccacgggcacaccaggcagcccaggcccagccaccctgcagtgcccaagcccacaccctggaggagagcagggtgcgtctgggaggggctgggctccccacccccacccccacctgcacaccccacccacccttgcccgggccccctgcaggagggtcagagcccccatgggatatggacttagggtctcactcacgcacctcccctcctgggagaaggggtctcatgcccagatccccccagcagcgctggtcacaggtagaggcagtggccccagggccaccctgacctggcccctcaggctcctctagccctggctgccctgctgtccctgggaggcctgggctccaccagaccacaggtctagggcaccgcccacactggggccgcccacacacagctcacaggaagaagataagctccagacccccaggcccgggacctgccttgctgctacgacttcctgccccagacctcgttgccctcccccgtccacttacacacaggccaggaagctgttcccacacagaccaaccccagacggggaccacctggcactcaggtcactgccatttccttctccattcacttccaatgcctctgtgcttcctccctcctccttccttcgggggagcaccctgtgcagctcctccctgcagtccacaccctggggagacccgaccctgcagcccacaccctggggagacctgaccctcctccagccctttctcccccgctgctcttgccacccaccaagacagccctggggtcctgtccctacagcccccacccagttctctacctagacccgtcttcctccctctaaacacctctcccaggccaaccctacacctgcaggccctcccctccactgccaaagaccctcagtttctcctgcctgtgcccacccccgtgctcctcctgcccacagctcgagctcttcctctcctagggcccctgagggatggcattgaccgtgccctcgcacccacacactgcccatgccctcacattcctcctggccactccagccccactcccctctcaggcctggctctggtatttctgggacaaagccttacccaagtctttcccatgcaggcctgggcccttaccctcactgcccggttacagggcagcctcctgtgcacagaagcagggagctcagcccttccacaggcagaaggcactgaaagaaatcggcctccagcgccttgacacacgtctgcctgtgtctctcactgcccgcacctgcagggaggctcggcactccctctaaagacgagggatccaggcagcagcatcacaggagaatgcagggctaccagacatcccagtcctctcacaggcctctcctgggaagagacctgaagacgcccagtcaacggagtctaacaccaaacctccctggaggccgatgggtagtaacggagtcattgccagacctggaggcaggggagcagtgagcccgagcccacaccatagggccagaggacagccactgacatcccaagccactcactggtggtcccacaacaccccatggaaagaggacagacccacagtcccacctggaccagggcagagactgctgagacccagcaccagaaccaaccaagaaacaccaggcaacagcatcagagggggctctggcagaacagaggaggggaggtctccttcaccagcaggcgcttcccttgaccgaagacaggatccatgcaactcccccaggacaaaggaggagccccttgttcagcactgggctcagagtcctctccaagacacccagagtttcagacaaaaaccccctggaatgcacagtctcagcaggagagccagccagagccagcaagatggggctcagtgacacccgcagggacaggaggattttgtgggggctcgtgtcactgtgaggatattgtactaatggtgtatgctatacccacagtgacacagccccattcccaaagccctactgcaaacgcattccacttctggggctgaggggctgggggagcgtctgggaaatagggctcaggggtgtccatcaatgcccaaaacgcaccagactcccctccatacatcacacccaccagccagcgagcagagtaaacagaaaatgagaagcaagctggggaagcttgcacaggccccaaggaaagagctttggcgggtgtgtaagaggggatgcgggcagagcctgagcagggccttttgctgtttctgctttcctgtgcagagagttccataaactggtgttcgagatcaatggctgggagtgagcccaggaggacagcgtgggaagagcacagggaaggaggagcagccgctatcctacactgtcatctttcgaaagtttgccttgtgcccacactgctgcatcatgggatgcttaacagctgatgtagacacagctaaagagagaatcagtgagatggatttgcagcacagatctgaataaattctccagaatgtggagcagcacagaagcaagcacacagaaagtgcctgatgcaaggacaaagttcagtgggcaccttcaggcattgctgctgggcacagacactctgaaaagccctggcaggaactccctgtgacaaagcagaaccctcaggcaatgccagccccagagccctccctgagagcctcatgggcaaagatgtgcacaacaggtgtttctcatagccccaaactgagagcaaagcaaacgtccatctgaaggagaacaggcaaataaacgatggcaggttcatgaaatgcaaacccagacagccacaagcacaaaagtacagggttataagcgactctggttgagttcatgacaatgctgagtaattggagtaacaaagtaaactccaaaaaatactttcaatgtgatttcttctaaataaaatttacaccctgcaaaatgaactgtcttcttaagggatacatttcccagttagaaaaccataaagaaaaccaagaaaaggatgatcacataaacacagtggtggttacttctgctggggaaggaagagggtatgaactgagatacacagggtgggcaagtctcctaacaagaacagaacgaatacattacagtaccttgaaaacagcagttaaacttctaaattgcaagaagaggaaaatggacacagttgtgtttagaaaattctcagtccagcactgttcataatagcaaagacattaacccaggtcggataaataagcgatgacacaggcaattgcacaatgatacagacatatatttagtatatgagacatcgatgatgtatccccaaataaacgactttaaagagataaagggctgatgtgtggtggcattcacctccctgggatccccggacaggttgcaggctcactgtgcagcagggcaggcgggtacctgctggcagttcctggggcctgatgtggagcaagcgcagggccatatatcccggaggacggcacagtcagtgaattccagagagaagcaactcagccacactccccaggcagagcccgagagggacgcccacgcacagggaggcagagcccagcacctccgcagccagcaccacctgtgcacgggccaccaccttgcaggcacagagtgggtgctgagaggaggggcagggacaccaggcagggtgagcacccagagaaaactgcagacgcctcacacatccacctcagcctcccctgacctggacctcactggcctgggcctcacttaacctgggcttcacctgaccttggcctcacctgacttggacctcgcctgtcccaagctttacctgacctgggcctcaactcacctgaacgtctcctgacctgggtttaacctgtcctggaactcacctggccttggcttcccctgacctggacctcatctggcctgggcttcacctggcctgggcctcacctgacctggacctcatctggcctggacctcacctggcctggacttcacctggcctgggcttcacctgacctggacctcacctggcctcgggcctcacctgcacctgctccaggtcttgctggagcctgagtagcactgagggtgcagaagctcatccagggttggggaatgactctagaagtctcccacatctgacctttctgggtggaggcagctggtggccctgggaatataaaaatctccagaatgatgactctgtgatttgtgggcaacttatgaacccgaaaggacatggccatggggtgggtagggacatagggacagatgccagcctgaggtggagcctcaggacacaggtgggcacggacactatccacataagcgagggatagacccgagtgtccccacagcagacctgagagcgctgggcccacagcctcccctcagagccctgctgcctcctccggtcagccctggacatcccaggtttccccaggcctgccggtaggtttagaatgaggtctgtgtcactgtggtattacgatattttgactggttattataaccacagtgtcacagagtccatcaaaaacccatgcctggaagcttcccgccacagccctccccatggggccctgctgcctcctcaggtcagccccggacatcccgggtttccccaggctgggcggtaggtttggggtgaggtctgtgtcactgtggtattactatggttcggggagttattataaccacagtgtcacagagtccatcaaaaacccatccctgggagcctcccgccacagccctccctgcaggggaccggtacgtgccatgttaggattttgatcgaggagacagcaccatgggtatggtggctaccacagcagtgcagcctgtgacccaaacccgcagggcagcaggcacgatggacaggcccgtgactgaccacgctgggctccagcctgccagccctggagatcatgaaacagatggccaaggtcaccctacaggtcatccagatctggctccgaggggtctgcatcgctgctgccctcccaacgccagtccaaatgggacagggacggcctcacagcaccatctgctgccatcaggccagcgatcccagaagcccctccctcaaggctgggccacatgtgtggacactgagagccctcatgtctgagtaggggcaccaggagggaggggctggccctgtgcactgtccctgcccctgtggtccctggcctgcctggccctgacacctgagcctctcctgggtcatttccaagacagaagacattcctggggacagccggagctgggcgtcgctcatcctgcccggccgtcctgagtcctgctcatttccagacctcaccggggaagccaacagaggactcgcctcccacattcagagacaaagaaccttccagaaatccctgcctctctccccagtggacaccctcttccaggacagtcctcagtggcatcacagcggcctgagatccccaggacgcagcaccgctgtcaataggggccccaaatgcctggaccagggcctgcgtgggaaaggtctctggccacactcgggctttttgtgaagggccctcctgctgtgtgactacagtaactaccatagtgatgaacccagtggcaaaaactggctggaaacccaggggctgtgtgcacgcctcagcttggagctctccaggagcacaagagccgggcccaaggatttgtgcccagaccctcagcctctagggacacctgggccatctcagcctgggctggtgccctgcacaccatcttcctccaaataggggcttcagagggctctgaggtgacctcactcatgaccacaggtgacctggcccttccctgccagctataccagaccctgtcttgacagatgccccgattccaacagccaattcctgggaccctgaatagctgtagacaccagcctcattccagtacctcctgccaattgcctggattcccatcctggctggaatcaagaaggcagcatccgccaggctcccaacaggcaggactcccgcacaccctcctctgagaggccgctgtgttccgcagggccaggccctggacagttcccctcacctgccactagagaaacacctgccattgtcgtccccacctggaaaagaccactcgtggagcccccagccccaggtacagctgtagagagagtcctcgaggcccctaagaaggagccatgcccagttctgccgggaccctcggccaggccgacaggagtggacgctggagctgggcccacactgggccacataggagctcaccagtgagggcaggagagcacatgccggggagcacccagcctcctgctgaccagaggcctgccccagagcccaggaggctgcagaggcctctccagggagacactgtgcatgtctggtacctaagcagccccccacgtccccagtcctgggggcccctggctcagctgtctggaccctccctgttccctgggaagctcctcctgacagccccgcctccagttccaggtgtggttattgtcaggcgatgtcagactgtggtggatatagtggctacgattaccacagtggtgccgcccatagcagcaaccaggccaagtagacaggcccctgctgcgcagccccaggcatccacttcacctgcttctcctggggctctcaaggctgctgtctgtcctctggccctctgtggggagggttccctcagtgggaggtctgtgctccagggcagggatgattgagatagaaatcaaaggctggcagggaaaggcagcttcccgccctgagaggtgcaggcagcaccacggagccacggagtcacagagccacggagcccccattgtgggcatttgagagtgctgtgcccccggcaggcccagccctgatggggaagcctgtcccatcccacagcccgggtcccacgggcagcgggcacagaagctgccaggttgtcctctatgatcctcatccctccagcagcatcccctccacagtggggaaactgaggcttggagcaccacccggccccctggaaatgaggctgtgagcccagacagtgggcccagagcactgtgagtaccccggcagtacctggctgcagggatcagccagagatgccaaaccctgagtgaccagcctacaggaggatccggccccacccaggccactcgattaatgctcaaccccctgccctggagacctcttccagtaccaccagcagctcagcttctcagggcctcatccctgcaaggaaggtcaagggctgggcctgccagaaacacagcaccctccctagccctggctaagacagggtgggcagacggctgtggacgggacatattgctggggcatttctcactgtcacttctgggtggtagctctgacaaaaacgcagaccctgccaaaatccccactgcctcccgctaggggctggcctggaatcctgctgtcctaggaggctgctgacctccaggatggctccgtccccagttccagggcgagagcagatcccaggcaggctgtaggctgggaggccacccctgcccttgccggggttgaatgcaggtgcccaaggcaggaaatggcatgagcacagggatgaccgggacatgccccaccagagtgcgccccttcctgctctgcaccctgcaccccccaggccagcccacgacgtccaacaactgggcctgggtggcagccccacccagacaggacagacccagcaccctgaggaggtcctgccagggggagctaagagccatgaaggagcaagatatggggcccccgatacaggcacagatgtcagctccatccaggaccacccagcccacaccctgagaggaacgtctgtctccagcctctgcaggtcgggaggcagctgacccctgacttggacccctattccagacaccagacagaggcgcaggccccccagaaccagggttgagggacgccccgtcaaagccagacaaaaccaaggggtgttgagcccagcaagggaaggcccccaaacagaccaggaggtttctgaaggtgtctgtgtcacagtggggtatagcagcagctggtaccacagtgacactcacccagccagaaaccccattccaagtcagcggaagcagagagagcagggaggacacgtttaggatctgagactgcacctgacacccaggccagcagacgtctcccctccagggcaccccaccctgtcctgcatttctgcaagatcaggggcggcctgagggggggtctagggtgaggagatgggtcccctgtacaccaaggaggagttaggcaggtcccgagcactctccccattgaggctgacctgcccagagagtcctgggcccaccccacacaccggggcggaatgtgtgcaggcctcggtctctgtgggtgttccgctagctggggctcacagtgctcaccccacacctaaaatgagccacagcctccggagcccccgcaggagaccccgcccacaagcccagcccccacccaggaggccccagagctcagggcgccccgtcggattccgaacagccccgagtcacagcgggtataaccggaaccaccactgtcagaatagctacgtcaaaaactgtccagtggccactgccggaggccccgccagagagggcagcagccactctgatcccatgtcctgccggctcccatgacccccagcacgcggagccccacagtgtccccactggatgggaggacaagagctggggattccggcgggtcggggcaggggcttgatcgcatccttctgccgtggctccagtgcccctggctggagttgacccttctgacaagtgtcctcagagagacaggcatcaccggcgcctcccaacatcaaccccaggcagcacaggcacaaaccccacatccagagccaactccaggagcagagacaccccaataccctgggggaccccgaccctgatgacttcccactggaattcgccgtagagtccaccaggaccaaagaccctgcctctgcctctgtccctcactcaggacctgctgccgggcgaggccttgggagcagacttgggcttaggggacaccagtgtgaccccgaccttgaccaggacgcagacctttccttcctttcctggggcagcacagactttggggtctgggccaggaggaacttctggcaggtcgccaagcacagaggccacaggctgaggtggccctggaaagacctccaggaggtggccactccccttcctcccagctggaccccatgtcctccccaagataagggtgccatccaaggcaggtgctccttggagccccattcagactcctccctggaccccactgggcctcagtcccagctctggggatgaagccaccacaagcacaccaggcagcccaggcccagccaccctgcagtgcccaagcacacactctggagcagagcagggtgcctctgggaggggctgagctccccaccccacccccacctgcacaccccacccacccctgcccagcggctctgcaggagggtcagagccccacatggggtatggacttagggtctcactcacgtggctcccatcatgagtgaaggggcctcaagcccaggttcccacagcagcgcctgtcgcaagtggaggcagaggcccgagggccaccctgacctggtccctgaggttcctgcagcccaggctgccctgctgtccctgggaggcctgggctccaccagaccacaggtccagggcaccgggtgcaggagccacccacacacagctcacaggaagaagataagctccagacccccagggccagaacctgccttcctgctactgcttcctgccccagacctgggcgccctcccccgtccacttacacacaggccaggaagctgttcccacacagaacaaccccaaaccaggaccgcctggcactcaggtggctgccatttccttctccatttgctcccagcgcctctgtcctccctggttcctccttcgggggaacagcctgtgcagccagtccctgcagcccacaccctggggagacccaaccctgcctggggcccttccaaccctgctgctcttactgcccacccagaaaactctggggtcctgtccctgcagtccctaccctggtctccacccagacccctgtgtatcactccagacacccctcccaggccaaccctgcacctgcaggccctgtcctcttctgtcgctagagcctcagtttctcccccctgtgcccacaccctacctcctcctgcccacaactctaactcttcttctcctggagcccctgagccatggcattgaccctgccctcccaccacccacagcccatgccctcaccttcctcctggccactccgaccccgccccctctcaggccaagccctggtatttccaggacaaaggctcacccaagtctttcccaggcaggcctgggctcttgccctcacttcccggttacacgggagcctcctgtgcacagaagcagggagctcagcccttccacaggcagaaggcactgaaagaaatcggcctccagcaccttgacacacgtccccccgtgtctctcactgcccgcacctgcagggaggctccgcactccctctaaagacaagggatccaggcagcagcatcacgggagaatgcagggctcccagacatcccagtcctctcacaggcctctcctgggaagagacctgcagccaccaccaaacagccacagaggctgctggatagtaactgagtcaatgaccgacctggagggcaggggagcagtgagccggagcccataccatagggacagagaccagccgctgacatcccgagctcctcaatggtggccccataacacacctaggaaacataacacacccacagccccacctggaacagggcagagactgctgagcccccagcaccagccccaagaaacaccaggcaacagtatcagagggggctcccgagaaagagaggaggggagatctccttcaccatcaaatgcttcccttgaccaaaaacagggtccacgcaactcccccaggacaaaggaggagccccctatacagcactgggctcagagtcctctctgagacaccctgagtttcagacaacaacccgctggaatgcacagtctcagcaggagaacagaccaaagccagcaaaagggacctcggtgacaccagtagggacaggaggattttgtgggggctcgtgtcactgtgaggatattgtagtggtggtagctgctactcccacagtgacacagacccattcccaaagccctactgcaaacacacccactcctggggctgaggggctgggggagcgtctgggaagtagggtccaggggtgtctatcaatgtccaaaatgcaccagactgcccgccaaacaccaccccaccagccagcgagcagggtaaacagaaaatgagaggctctgggaagcttgcacaggccccaaggaaagagctttggcgggtgtgcaagaggggatgcaggcagagcctgagcagggccttttgctgtttctgctttcctgtgcagagagttccataaactggtgttcaagatcagtggctgggaatgagcccaggagggcagtctgtgggaagagcacagggaaggaggagcagccgctatcctacactgtcatctttcaaaagtttgccttgtgaccacactattgcatcatgggatgcttaagagctgatgtagacacagctaaagagagaatcagtgagatgaatttgcagcatagatctgaataaactctccagaatgtggagcagtacagaagcaaacacacagaaagtgcctgatgcaaggacaaagttcagtgggcaccttcaggcattgctgctgggcacagacactctgaaaagccctggcaggatctccctgcgacaaagcagaaccctcaggcaatgccagccccagagccctccctgagagcgtcatggggaaagatgtgcagaacagctgattatcatagactcaaactgagaacagagcaaacgtccatctgaagaacagtcaaataagcaatggtaggttcatgcaatgcaaacccagacagccaggggacaacagtagagggctacaggcggctttgcggttgagttcatgacaatgctgagtaattggagtaacagaggaaagcccaaaaaatacttttaatgtgatttcttctaaataaaatttacaccaggcaaaatgaactgtcttcttaagggataaactttcccctggaaaaactacaaggaaaattaagaaaacgatgatcacataaacacagttgtggttacttctactggggaaggaagagggtatgagctgagacacacagagtcggcaagtctccaagcaagcacagaacgaatacattacagtaccttgaatacagcagttaaacttctaaatcgcaagaagaggaaaatgcacacagctgtgtttagaaaattctcagtccagcactattcataatagcaaagacattaacccaggttggataaataaatgatgacacaggcaattgcacaatgatacagacatacatttagtacatgagacatcgatgatgtatccccaaagaaatgactttaaagagaaaaggcctgatgtgtggtggcactcacctccctgggatccccggacaggttgcaggcacactgtgtggcagggcaggctggtacatgctggcagctcctggggcctgatgtggagcaagcgcagggctgtatacccccaaggatggcacagtcagtgaattccagagagaagcagctcagccacactgcccaggcagagcccgagagggacgcccacgcacagggaggcagagcccagctcctccacagccaccaccacctgtgcacgggccaccaccttgcaggcacagagtgggtgctgagaggaggggcagggacaccaggcagggtgagcacccagagaaaactgcagaagcctcacacatccacctcagcctcccctgacctggacctcacctggtctggacctcacctggcctgggcctcacctgacctggacctcacctggcctgggcttcacctgacctggacctcacctggcctccggcctcacctgcacctgctccaggtcttgctggaacctgagtagcactgaggctgcagaagctcatccagggttggggaatgactctggaactctcccacatctgacctttctgggtggaggcatctggtggccctgggaatataaaaagccccagaatggtgcctgcgtgatttgggggcaatttatgaacccgaaaggacatggccatggggtgggtagggacatagggacagatgccagcctgaggtggagcctcaggacacagttggacgcggacactatccacataagcgagggacagacccgagtgttcctgcagtagacctgagagcgctgggcccacagcctcccctcggtgccctgctgcctcctcaggtcagccctggacatcccgggtttccccaggccagatggtaggtttgaagtgaggtctgtgtcactgtggtattatgattacgtttgggggagttatcgttatacccacagcatcacacggtccatcagaaacccatgccacagccctccccgcaggggaccgccgcgtgccatgttacgattttgatcgaggacacagcgccatgggtatggtggctaccacagcagtgcagcccatgacccaaacacacagggcagcaggcacaatggacaggcctgtgagtgaccatgctgggctccagcccgccagccccggagaccatgaaacagatggccaaggtcaccccacagttcagccagacatggctccgtggggtctgcatcgctgctgccctctaacaccagcccagatggggacaaggccaaccccacattaccatctcctgctgtccacccagtggtcccagaagcccctccctcatggctgagccacatgtgtgaaccctgagagcaccccatgtcagagtaggggcagcagaagggcggggctggccctgtgcactgtccctgcacccatggtccctcgcctgcctggccctgacacctgagcctcttctgagtcatttctaagatagaagacattcccggggacagccggagctgggcgtcgctcatcccgcccggccgtcctgagtcctgcttgtttccagacctcaccagggaagccaacagaggactcacctcacacagtcagagacaaagaaccttccagaaatccctgtctcactccccagtgggcaccttcttccaggacattcctcggtcgcatcacagcaggcacccacatctggatcaggacggcccccagaacacaagatggcccatggggacagccccacaacccaggccttcccagacccctaaaaggcgtcccaccccctgcacctgccccagggctaaaaatccaggaggcttgactcccgcataccctccagccagacatcacctcagccccctcctggaggggacaggagcccgggagggtgagtcagacccacctgccctcgatggcaggcggggaagattcagaaaggcctgagatccccaggacgcagcaccactgtcaatgggggccccagacgcctggaccagggcctgcgtgggaaaggccgctgggcacactcaggggctttttgtgaaggcccctcctactgtgtgactacggtgactaccacagtgatgaaactagcagcaaaaactggccggacacccagggaccatgcacacttctcagcttggagctctccaggaccagaagagtcaggtctgagggtttgtagccagaccctcggcctctagggacaccctggccatcacagcggatgggctggtgccccacatgccatctgctccaaacaggggcttcagagggctctgaggtgacttcactcatgaccacaggtgccctggccccttccccgccagctacaccgaaccctgtcccaacagctgccccagttccaacagccaattcctggggcccagaattgctgtagacaccagcctcgttccagcacctcctgccaattgcctggattcacatcctggctggaatcaagagggcagcatccgccaggctcccaacaggcaggactcccgcacaccctcctctgagaggccgctgtgttccgcagggccaggccctggacagttcccctcacctgccactagagaaacacctgccattgtcgtccccacctggaaaagaccactcgtggagcccccagccccaggtacagctgtagagagactccccgagggatctaagaaggagccatgcgcagttctgccgggaccctcggccaggccgacaggagtggacactggagctgggcccacactgggccacataggagctcaccagtgagggcaggagagcacatgccggggagcacccagcctcctgctgaccagaggcccgtcccagagcccaggaggctgcagaggcctctccagggggacactgtgcatgtctggtccctgagcagccccccacgtccccagtcctgggggcccctggcacagctgtctggaccctccctcttccctgggaagctcctcctgacagccccgcctccagttccaggtgtggttattgtcagggggtgtcagactgtggtggatacagctatggttaccacagtggtgctgcccatagcagcaaccaggccaagtagacaggcccctgctgtgcagccccaggcctccagctcacctgcttctcctggggctctcaaggtcactgttgtctgtactctgccctctgtggggagggttccctcagtgggaggtctgttctcaacatcccagggcctcatgtctgcacggaaggccaatggatgggcaacctcacatgccgcggctaagatagggtgggcagcctggcgggggacagtacatactgctggggtgtctgtcactgtgcctagtggggcactggctcccaaacaacgcagtcctcgccaaaatccccacagcctcccctgctaggggctggcctgatctcctgcagtcctaggaggctgctgacctccagaatgtctccgtccccagttccagggcgagagcagatcccaggccggctgcagactgggaggccaccccctccttcccagggttcactggaggtgaccaaggtaggaaatggccttaacacagggatgactgcgccatcccccaacagagtcagccccctcctgctctgtaccccgcaccccccaggccagtccacgaaaaccagggccccacatcagagtcactgcctggcccggccctggggcggacccctcagcccccaccctgtctagaggacttggggggacaggacacaggccctctccttatggttcccccacctgcctccggccgggacccttggggtgtggacagaaaggacacctgcctaattggcccccaggaacccagaacttctctccagggaccccagcccgagcacccccttacccaggacccagccctgcccctcctcccctctgctctcctctcatcaccccatgggaatccggtatccccaggaagccatcaggaagggctgaaggaggaagcggggccgtgcaccaccgggcaggaggctccgtcttcgtgaacccagggaagtgccagcctcctagagggtatggtccaccctgcctggggctcccaccgtggcaggctgcggggaaggaccagggacggtgtgggggagggctcagggccctgcgggtgctcctccatcttcggtgagcctcccccttcacccaccgtcccgcccacctcctctccaccctggctgcacgtcttccacaccatcctgagtcctacctacaccagagccagcaaagccagtgcagacaaaggctggggtgcaggggggctgccagggcagcttcggggagggaaggatggagggaggggaggtcagtgaagaggcccccttcccctgggtccaggatcctcctctgggacccccggatcccatcccctcctggctctgggaggagaagcaggatgggagaatctgtgcgggaccctctcacagtggaatatccccacagcggctcaggccagacccaaaagcccctcagtgagccctccactgcagtcctgggcctgggtagcagcccctcccacagaggacagacccagcaccccgaagaagtcctgccagggggagctcagagccatgaaagagcaggatatggggtccccgatacaggcacagacctcagctccatccaggcccaccgggacccaccatgggaggaacacctgtctccgggttgtgaggtagctggcctctgtctcggaccccactccagacaccagacagaggggcaggccccccaaaaccagggttgagggatgatccgtcaaggcagacaagaccaaggggcactgaccccagcaagggaaggctcccaaacagacgaggaggtttctgaagctgtctgtatcacagtggggtatagcagtggctggtaccacagtgacactcgccaggccagaaaccccgtcccaagtcagcggaagcagagagagcagggaggacacgtttaggatctgaggccgcacctgacacccagggcagcagacgtctcccctccagggcaccctccaccgtcctgcgtttcttcaagaataggggcggcctgagggggtccagggccaggcgataggtcccctctaccccaaggaggagccaggcaggacccgagcaccgtccccattgaggctgacctgcccagacgggcctgggcccaccccacacaccggggcggaatgtgtgcaggccccagtctctgtgggtgttccgctagctggggcccccagtgctcaccccacacctaaagcgagccccagcctccagagccccctaagcattccccgcccagcagcccagcccctgcccccacccaggaggccccagagctcagggcgcctggtcggattctgaacagccccgagtcacagtgggtataactggaacgaccaccgtgagaaaaactgtgtccaaaactgactcctggcagcagtcggaggccccgccagagaggggagcagccggcctgaacccatgtcctgccggttcccatgacccccagcacccagagccccacggtgtccccgttggataatgaggacaagggctgggggctccggtggtttgcggcagggacttgatcacatccttctgctgtggccccattgcctctggctggagttgacccttctgacaagtgtcctcagaaagacagggatcaccggcacctcccaatatcaaccccaggcagcacagacacaaaccccacatccagagccaactccaggagcagagacaccccaacactctgggggaccccaaccgtgataactccccactggaatccgccccagagtctaccaggaccaaaggccctgccctgtctctgtccctcactcagggcctcctgcagggcgagcgcttgggagcagactcggtcttaggggacaccactgtgggccccaactttgatgaggccactgacccttccttcctttcctggggcagcacagactttggggtctgggcagggaagaactactggctggtggccaatcacagagcccccaggccgaggtggccccaagaaggccctcaggaggtggccactccacttcctcccagctggaccccaggtcctccccaagataggggtgccatccaaggcaggtcctccatggagcccccttcagactcctcccgggaccccactggacctcagtccctgctctgggaatgcagccaccacaagcacaccaggaagcccaggcccagccaccctgcagtgggcaagcccacactctggagcagagcagggtgcgtctgggaggggctaacctccccaccccccaccccccatctgcacacagccacctaccactgcccagaccctctgcaggagggccaagccaccatggggtatggacttagggtctcactcacgtgcctcccctcctgggagaaggggcctcatgcccagatccctgcagcactagacacagctggaggcagtggccccagggccaccctgacctggcatctaaggctgctccagcccagacagcactgccgttcctgggaagcctgggctccaccagaccacaggtccagggcacagcccacaggagccacccacacacagctcacaggaagaagataagctccagaccccagggcgggacctgccttcctgccaccacttacacacaggccagggagctgttcccacacagatcaaccccaaaccgggactgcctggcactagggtcactgccatttccctctccattccctcccagtgcctctgtgctccctccttctggggaacaccctgtgcagcccctccctgcagcccacacgctggggagaccccaccctgcctcgggccttttctacctgctgcacttgccgcccacccaaacaaccctgggtacgtgaccctgcagtcctcaccctgatctgcaaccagacccctgtccctccctctaaacacccctcccaggccaactctgcacctgcaggccctccgctcttctgccacaagagcctcaggttttcctacctgtgcccaccccctaacccctcctgcccacaacttgagttcttcctctcctggagcccttgagccatggcactgaccctacactcccacccacacactgcccatgccatcaccttcctcctggacactctgaccccgctcccctccctctcagacccggccctggtatttccaggacaaaggctcacccaagtcttccccatgcaggcccttgccctcactgcctggttacacgggagcctcctgtgcgcagaagcagggagctcagctcttccacaggcagaaggcactgaaagaaatcggcctccagtgccttgacacacgtccgcctgtgtctctcactgcctgcacctgcagggaggctccgcactccctctaaagatgagggatccaggcagcaacatcacgggagaatgcagggctcccagacagcccagccctctcgcaggcctctcctgggaagagacctgcagccaccactgaacagccacggaggtcgctggatagtaaccgagtcagtgaccgacctggagggcaggggagcagtgaaccggagcccataccatagggacagagaccagccgctaacatcccgagcccctcactggcggccccagaacaccccgtggaaagagaacagacccacagtcccacctggaacagggcagacactgctgagcccccagcaccagccccaagaaacactaggcaacagcatcagagggggctcctgagaaagagaggaggggaggtctccttcaccatcaaatgcttcccttgaccaaaaacagggtccacgcaactcccccaggacaaaggaggagccccctgtacagcactgggctcagagtcctctctgagacaggctcagtttcagacaacaacccgctggaatgcacagtctcagcaggagagccaggccagagccagcaagaggagactcggtgacaccagtctcctgtagggacaggaggattttgtgggggttcgtgtcactgtgagcatattgtggtggtgactgctattcccacagtgacacaaccccattcctaaagccctactgcaaacgcacccactcctgggactgaggggctgggggagcatctgggaagtatggcctaggggtgtccatcaatgcccaaaatgcaccagactctccccaagacatcaccccaccagccagtgagcagagtaaacagaaaatgagaagcagctgggaagcttgcacaggccccaaggaaagagctttggcaggtgtgcaagaggggatgtgggcagagcctcagcagggccttttgctgtttctgctttcctgtgcagagagttccataaactggtattcaagatcaatggctgggagtgagcccaggaggacagtgtgggaagagcacagggaaggaggagcagccgctatcctacactgtcatcttttgaaagtttgccctgtgcccacaatgctgcatcatgggatgcttaacagctgatgtagacacagctaaagagagaatcagtgaaatgcatttgcagcacagatctgaataaatcctccagaatgtggagcagcacagaagcaagcacacagaaagtgcctgatgccaaggcaaagttcagtgggcaccttcaggcattgctgctgggcacagacactctgaaaagcactggcaggaactgcctgtgacaaagcagaaccctcaggcaatgccagccctagagcccttcctgagaacctcatgggcaaagatgtgcagaacagctgtttgtcatagccccaaactatggggctggacaaagcaaacgtccatctgaaggagaacagacaaataaacgatggcaggttcatgaaatacaaactaggacagccagaggacaacagtagagagctacaggcggctttgcggttgagttcatgacaatgctgagtaattggagtaacagaggaaagcccaaaaaatacttttaatgtgatttcttctaaataaaatttacacccggcaaaatgaactatcttcttaagggataaactttcccctggaaaaactataaggaaaatcaagaaaacgatgatcacataaacacagtggtggttacttctactggggaaggaagagggtatgagctgagacacacagagtcggcaagtctcctaacaagaacagaacaaatacattacagtaccttgaaaacagcagttaaacttctaaatcgcaagaagaggaaaatgcacacacctgtgtttagaaaattctcagtccagcactgttcataatagcaaagacattaacccaggttggataaataagcgatgacacaggcaattgcacaatgatacagacatacattcagtatatgagacatcgatgatgtatccccaaagaaatgactttaaagagaaaaggcctgatgtgtggtggcaatcacctccctgggcatccccggacaggctgcaggctcactgtgtggcagggcaggcaggcacctgctggcagctcctggggcctgatgtggagcaggcacagagctgtatatccccaaggaaggtacagtcagtgcattccagagagaagcaactcagccacactccctggccagaacccaagatgcacacccatgcacagggaggcagagcccagcacctccgcagccaccaccacctgcgcacgggccaccaccttgcaggcacagagtgggtgctgagaggaggggcagggacaccaggcagggtgagcacccagagaaaactgcagaagcctcacacatccacctcagcctcccctgacctggacctcacctggcctgggcctcacctgacctggacctcacctggcctgggcttcacctggcctgggcttcacctgacctggacctcacctggcctcgggcctcacctggcctgggcttcacctggcctgggcttcacctgacctggacctcacctggcctgggcctcacctgacctggacctcacctggcctgggcttcacctggcctgggcttcacctggcctgggcttcacctgacctggacctcacctggcctgggcttcacctgacctggacctcacctggcctcgggcctcacctgcacctgctccaggtcttgctggagcctgagtagcactgaggctgtagggactcatccagggttggggaatgactctgcaactctcccacatctgacctttctgggtggaggcacctggtggcccagggaatataaaaagccccagaatgatgcctgtgtgatttgggggcaatttatgaacccgaaaggacatggccatggggtgggtagggacagtagggacagatgtcagcctgaggtgaagcctcaggacacaggtgggcatggacagtgtccacctaagcgagggacagacccgagtgtccctgcagtagacctgagagcgctgggcccacagcctcccctcggggccctgctgcctcctcaggtcagccctggacatcccgggtttccccaggcctggcggtaggtttgaagtgaggtctgtgtcactgtggtattactatgatagtagtggttattactaccacagtgtcacagagtccatcaaaaactcatgcctgggagcctcccaccacagccctccctgcgggggaccgctgcatgccgtgttaggattttgatcgaggacacggcgccatgggtatggtggctaccacagcagtgcagcccatgacccaaacacacggggcagcagaaacaatggacaggcccacaagtgaccatgatgggctccagcccaccagccccagagaccatgaaacagatggccaaggtcaccctacaggtcatccagatctggctccaaggggtctgcatcgctgctgccctcccaacgccaaaccagatggagacagggccggccccatagcaccatctgctgccgtccacccagcagtcccggaagcccctccctgaacgctgggccacgtgtgtgaaccctgcgagccccccatgtcagagtaggggcagcaggagggcggggctggccctgtgcactgtcactgcccctgtggtccctggcctgcctggccctgacacctgagcctctcctgggtcatttccaagacattcccagggacagccggagctgggagtcgctcatcctgcctggctgtcctgagtcctgctcatttccagacctcaccagggaagccaacagaggactcacctcacacagtcagagacaatgaaccttccagaaatccctgtttctctccccagtgagagaaaccctcttccagggtttctcttctctcccaccctcttccaggacagtcctcagcagcatcacagcgggaacgcacatctggatcaggacggcccccagaacacgcgatggcccatggggacagcccagcccttcccagacccctaaaaggtatccccaccttgcacctgccccagggctcaaactccaggaggcctgactcctgcacaccctcctgccagatatcacctcagccccctcctggaggggacaggagcccgggagggtgagtcagacccacctgccctcaatggcaggcggggaagattcagaaaggcctgagatccccaggacgcagcaccactgtcaatgggggccccagacgcctggaccagggcctgtgtgggaaaggcctctggccacactcaggggctttttgtgaagggccctcctgctgtgtgactacggtggtaactcccacagtgatgaaaccagcagcaaaaactgaccggactcgcagggtttatgcacacttctcggctcggagctctccaggagcacaagagccaggcccgagggtttgtgcccagaccctcggcctctagggacacccgggccatcttagccgatgggctgatgccctgcacaccgtgtgctgccaaacaggggcttcagagggctctgaggtgacttcactcatgaccacaggtgccctggtcccttcactgccagctgcaccagaccctgttccgagagatgccccagttccaaaagccaattcctggggccgggaattactgtagacaccagcctcattccagtacctcctgccaattgcctggattcccatcctggctggaatcaagagggcagcatccgccaggctcccaacaggcaggactcccacacaccctcttctgagaggccgctgtgttccgcagggccaggccgcagacagttcccctcacctgcccatgtagaaacacctgccattgtcgtccccacctggcaaagaccacttgtggagcccccagccccaggtacagctgtagagagagtcctcgaggcccctaagaaggagccatgcccagttctgctgggaccctcggccaggccgacaggagtggacgctggagctgggcccacactgggccacataggagctcaccagtgagggcaggagagcacatgccggggagcacccagcctcctgctgaccagagacccgtcccagagcccaggaggctgcagaggcctctccagggggacacagggcatgtctggtccctgagcagcccccaggctctctagcactgggggcccctggcacagctgtctggaccctccctgttccctgggaagctcctcctgacagccccgcctccagttccaggtgtggttattgtcagggggtgccaggccgtggtagagatggctacaattaccacagtggtgccgcccatagcagcaaccaggccaagtagacagacccctgccacgcagccccaggcctccagctcacctgcttctcctggggctctcaaggctgctgtctgccctctggccctctgtggggagggttccctcagtgggaggtctgtgctccagggcagggatgactgagatagaaatcaaaggctggcagggaaaggcagcttcccgccctgagaggtgcaggcagcaccacagagccatggagtcacagagccacggagcccccagtgtgggcgtgtgagggtgctgggctcccggcaggcccagccctgatggggaagcctgccccgtcccacagcccaggtccccaggggcagcaggcacagaagctgccaagctgtgctctacgatcctcatccctccagcagcatccactccacagtggggaaactgagccttggagaaccacccagccccctggaaacaaggcggggagcccagacagtgggcccagagcactgtgtgtatcctggcactaggtgcagggaccacccggagatccccatcactgagtggccagcctgcagaaggacccaaccccaaccaggccgcttgattaagctccatccccctgtcctgggaacctcttcccagcgccaccaacagctcggcttcccaggccctcatccctccaaggaaggccaaaggctgggcctgccaggggcacagtaccctcccttgccctggctaagacagggtgggcagacggctgcagataggacatattgctggggcatcttgctctgtgactactgggtactggctctcaacgcagaccctaccaaaatccccactgcctcccctgctaggggctggcctggtctcctcctgctgtcctaggaggctgctgacctccaggatggcttctgtccccagttctagggccagagcagatcccaggcaggctgtaggctgggaggccacccctgtccttgccgaggttcagtgcaggcacccaggacaggaaatggcctgaacacagggatgactgtgccatgccctacctaagtccgcccctttctactctgcaacccccactccccaggtcagcccatgacgaccaacaacccaacaccagagtcactgcctggccctgccctggggaggacccctcagcccccaccctgtctagaggacttggggggacaggacacaggccctctccttatggttcccccacctggctcctgccgggacccttggggtgtggacagaaaggacgcctgcctaattggcccccaggaacccagaacttctctccagggaccccagcccgagcacccccttacccaggacccagccctgcccctcctcccctctgctctcctctcatcactccatgggaatccagaatccccaggaagccatcaggaagggctgaaggaggaagcggggccgctgcaccaccgggcaggaggctccgtcttcgtgaacccagggaagtgccagcctcctagagggtatggtccaccctgcctggggctcccaccgtggcaggctgcggggaaggaccagggacggtgtgggggagggctcaggtccctgcaggtgctccatcttggatgagcccatccctctcacccaccgacccgcccacctcctctccaccctggccacacgtcgtccacaccatcctgagtcccacctacaccagagccggcagagccagtgcagacagaggctggggtgcaggggggccgccagggcagctttggggagggaggaatggaggaaggggaggtcagtgaagaggcccccctcccctgggtctaggatccacctttgggacccccggatcccatcccctccaggctctgggaggagaagcaggatgggagaatctgtgcgggaccctctcacagtggaatacctccacagcggctcaggccagatacaaaagcccctcagtgagccctccactgcagtgctgggcctgggggcagcccctcccacagaggacagacccagcaccccgaagaagtcctgccagggggagctcagagccatgaaggagcaagatatggggaccccaatactggcacagacctcagctccatccaggcccaccaggacccaccatgggtggaacacctgtctccggcccctgctggctgtgaggcagctggcctctgtctcggacccccattccagacaccagacagagggacaggccccccagaaccagtgttgagggacacccctgtccagggcagccaagtccaagaggcgcgctgagcccagcaagggaaggcccccaaacaaaccaggaggtttctgaagctgtctgtgtcacagtcgggtatagcagcggctaccacaatgacactgggcaggacagaaaccccatcccaagtcagccgaaggcagagagagcaggcaggacacatttaggatctgaggccacacctgacactcaagccaacagatgtctcccctccagggcgccctgccctgttcagtgttcctgagaaaacaggggcagcctgaggggatccagggccaggagatgggtcccctctaccccgaggaggagccaggcgggaatcccagccccctccccattgaggccatcctgcccagaggggcccggacccaccccacacacccaggcagaatgtgtgcaggcctcaggctctgtgggtgccgctagctggggctgccagtcctcaccccacacctaaggtgagccacagccgccagagcctccacaggagaccccacgcagcagcccagcccctacccaggaggccccagagctcagggcgcctgggtggattctgaacagccccgagtcacggtgggtatagtgggagctactaccactgtgagaaaagctatgtccaaaactgtctcccggccactgctggaggcccagccagagaagggaccagccgcccgaacatacgaccttcccagccctcatgacccccagcacttggagctccacagtgtccccattggatggtgaggacgggggccggggccatctgcacctcccaacatcacccccaggcagcacaggcacaaaccccaaatccagagccgacaccaggaacacagacaccccaataccctgggggaccctggccctggtgacttcccactgggatccacccccgtgtccacctggatcaaagaccccaccgctgtctctgtccctcactcagggcctgctgaggggcgggtgctttggagcagactcaggtttaggggccaccattgtggggcccaacctcgaccaggacacagatttttctttcctgccctggggcaacacagactttggggtctgtgcagggaggaccttctggaaagtcaccaagcacagagccctgactgaggtggtctcaggaagacccccaggagggggtttgtgccccttcctctcatgtggaccccatgccccccaagataggggcatcatgcagggcaggtcctccatgcagccaccactaggcaactccctggcgccggtccccactgcgcctccatcccggctctggggatgcagccaccatggccacaccaggcagcccgggtccagcaaccctgcagtgcccaagcccttggcaggattcccagaggctggagcccacccctcctcatccccccacacctgcacacacacacctaccccctgcccagtccccctccaggagggttggagccgcccatagggtgggcgctccaggtctcactcactcgcttcccttcctgggcaaaggagcctcgtgccccggtcccccctgacggcgctgggcacaggtgtgggtactgggccccagggctcctccagccccagctgccctgctctccctgggaggcctgggcaccaccagaccaccagtccagggcacagccccagggagccgcccactgccagctcacaggaagaagataagcttcagaccctcagggccgggagctgccttcctgccaccccttcctgccccagacctccatgccctcccccaaccacttacacacaagccagggagctgtttccacacagttcaaccccaaaccaggacggcctggcactcgggtcactgccatttctgtctgcattcgctcccagcgcccctgtgttccctccctcctccctccttcctttcttcctgcattgggttcatgccgcagagtgccaggtgcaggtcagccctgagcttggggtcacctcctcactgaaggcagcctcagggtgcccaggggcaggcagggtgggggtgaggcttccagctccaaccgctccactagccgagactaaggaagtgagaggcagccagaaatccagaccattccatagcaaatggatttcattaaagttaccagacttcagtgtaagtaacatgagccccatgcacaacaatcccttatgaaggggaagtcagtgtcgcctcggatttcttgaaaaacacaaaaacttatcaatgcctgtaaaagtctgttggaaagaaaatatgattcaagaatgttatgcccaacaaagctggcatattttctacccggacacactcagggaatgtggtcccttgagtgcttctctcactgcgtaaatcctacgtggtgtttaagcatattcataaatgtgtatgtctatttttatgtgtaagatggttcatttttattttatttattcaatatgtacaataaagaatattgacaaataggctggacatggtggctcccacctgtaatcccagccctttgggaggccgaggcgggcagatcacctgaggtctggagttcgagaccagcctggccaacatgatgaaaacccatctctactaaaaatacaaagattagccaggcatggtggtgcatgcctgtaatcccagccactcaggaggctgagacaggagaaatgcgtgaacccggaaggcggaggttgcagtgagccgagatcacaccactgcactccagcctggcgacagagcaagattccatctcaaaaaaaaaaagacaaagaaatttgtttttttgaataaagacaaatttcatcacacgaagataaagatgcaaagctccagacaggaaggcacggacagcacagtgaagcccggagcgggcgctggggggccaggggcatggcgggggtgccagcgtctctcggtgcctaccatggccactccagcctgtgttctcacgaggatggctgtgcaatgctaggagcgtgttcgaagctctagggcaaccactggaagtgaggctgaggagcagagcccagaggcccgtggagctgatgaaaagaaagctggagaaagtgtttgctgcctcccaacatggtaagaaaagatagaaagagagagcacacggcaaagggagcttgctgagggactctttacaatggcttgcacagagctcagggggtctgggaggctagggccctgcgcagggcagtcaccccagcctgctgaccaaggtttgctgcaggcagctctgggggtggttgaggcgcggtccctggagccacccctcaagggaacgaggcagcagagtgggccaaggcccaggtcggctgcaaggctgcccaggacttggggtccttacatcagcagccactgatgcagctggcccagagagaggcgccgagcaggttgcctccaggggacaaaccaggtcggagagggtgaggcagtggatggagccacaacaaccccgggcacgggtgacacgcacgttcatgcacatctgacccttcctccctcaccaaacaggtccccctgccttccccatggttgcgaaaaagcaaaatgtagacgttttttcttttttaattcatgttttaattgacaaatgaagccgtatatatttattgtgtacaacatgatgctttaaaatatgtatacatcgtggaacagcaacgttgagctaatttaacacgcattacttcacatacttgtcatcttttgtggcgagaatgcttaaaatccactctcttagtattttttaagaatgcaatacattgttgtcaactgtggtcaccgtcatgcatagccaagctcccgacctcaccctcctgccagctcaggctgtgcatcctttcaccagcatcccccaccccggcccctggccctggtaactaccactctatactctacgtatgagttcagctttttaagattccacagatgaatgagatcatacagtatttgctttctatgcctggcttattttagttaacacactgtcctccagatccatccgttgttgcaaatgacagggtttcattctttttaaagtctaaagagtattccattgtgtcaatggacctcatttgctttatccatgcatcaactatggacatttaggttgattccatttcttagctgttgtggatggtgctgcagtaaacatggggctgcagatgtctcttcaacatactgacatcatgtcctttggataaatacccagtagtgggatcgctggatcacaatgtacagttttttttttaatggaaactttcattttttggtgaaattaggaaaacagataaaacccacagaatccaaaatatatgtgaagatgccaaaaacagttgacattgggcagaggtcacatggaaggaagtgaatacatgacggggtgtgagggcccagaggcagctgaaatacgctttctaaacacaaggacctcttctgagagggcagaagttttatcctgcacatgcaatgaccagcacagctaaaatacactttctaaacatgaggacctcttctgagagggcagctttatcctgcaaatgcaatgaccagcacaggacccagaataaagagagttgccagcggacgcctggtgtccatgtgtccaggtgagttcgagatgcggacggcgctggccagccagtcacaccctaagtcaatctgctgcatgcatttgtccttgccacagcagaaaacgagaaagcctttgggctgcaaagcttcacaggctcctcttctcccgactccatggaaacagctacaaagagcaggcccagtagagcttaattcatgaaaatgagtaataaacttgaactggaacagtatcgactttttagaaacggcagcaaagtgtataaaaaatattcaccagaacaatatttccaaacgatgagatgagaatttcagccaagtaatcctccatggatagaaaataatgaagggattggatttatgaaggaaaatcatggagctcaaatacaagagaagagaatcaaaaatgaacaggaggagataaaatatggtttggccaaagttacaaaataaattttttaaaaacccttcatcatggcaagtagaaagagcgagaggaaaaacagatcccgtggaagacacaaataggacatggggagaaaaatgaatgagatgaaacagagcagaaataaaattttacggaactaaagacaagtgatctgaacctgcctggggcctgggggacctcgccaccctgaagggaaagaacatgcctggctggctttgccacctgctcattgcagagccccacagcttgcaacaaacataggcggtagccagggagtggttacagcaggccttgagcaagacccagtgttgtgctgacttcaggtctgacccagcactgtcatagtggtggtgtccatagtggtagtgggggtgcttgtgtcactccacccccatctccaggaggctcagaacagacagagagagactccatttgtttgggagaaagtaagggatgagaacaagagtctctgcctggtaatccagagaattattctagatcttggccaagattatcaaagcagtacctctatgagtcttttgggcttggagtccccctaaagcagatatagctaagatcacaacacccaagtccttttgaatatgtgggaagacttcccaaggacaggagcaaacaaacaagcccagactgcaaaaaaacaagcccagactgcaataaacacctcactcttcaatgcccaggcactgaagaacatctcctagcagcaacaccatccaggaaaacatggcctcaaccagtgaactaaataaggcaccagggaccagtctcggagaaatagaggtatgttatctttcagagaattcaaagtagctttgttgaggaaactcaaagaaattcaagataacacagtgaaggaattcagaatcctatccgataaatttaacagagattgaagcaattaaaaagaattaagcagaaattatggagctgaaaaatgcaattggcatactgaaaaatgcatcagagtattttcatagcctcttatatcaagtagaagaaagaattagtgagcttgaaaacaggctatttggaaaagcacgataaaaggagacaaaagagaaaagaataaataacaatgaagcatatctacaggatctagaaaatagcctcaaaaggccaaatctaagaattattagccttaaagaggaggtagagaaagagggatggagagtttattcaaagggataataacagaaaacttcccaaacctagagaaagatatcaatatccaaatgcaagaaggatgtagtacaccaaggagatttaatgcaaagaagactacctcaaggcattcaatactcaaactcccatatgacaaggactttaaaaagatcctaaaagcagcaaaagaaaagaaatgaataaaatactatggagctccaatatgtctggcagcagacttttcagtgaagactttatatgccaggagagagtgtcataatggatttaaagtgctgaaggaaaaaacttttaccctcgaacagtatagctggtgaaattatccttcaaacatgaaggagaaataatttgtttccagacaaatgttgagggatttcatgaacaccagacctgtcttttaagaaatgctaaagggagtacttcaatcagaaagaaacacgttagtgaacaataagaaatcatctgaaggcacaaaactcaccggtaatagtaagtacacagaaaaacacagaatattataacactgtaactgtggtgtgtaaactccttttgtttgtttgtttgtttgtttgtttgtttgtttgtttttgtttttagacggagttttgctccagcccaggctggagtgcaatggcacaatctcagctcactgcaacttccacctcccgggttcaagcaattctcctgcctcagcctcccaagtagctgggattacaggcatgtgctaccatgtccagctaattttgtattttagtagagacggtgtttcaccatgttggtcaggctagccttatcttgagtagaaaaactaaatgatgaagcaatgaaaaataataactacaacttttcaagacatagtacaataagatataaatcataacaaaaagttaaaaggtggagggatgaagttaaggcaaagagtctttattagttttctttttacttgtctgtttatgcaaacagtgttaagttgtcatcagtttaaaataatgggtcataagatactatttgcaagcctcatggtaacgtcaaaccaaaagcaatacaacagatacacaaaaaacaaaaagcaagaagctaaattacgtcatcagagaaaatcaccttcactaaaaggaagacggagaaaagaatgaagagagagaagaccaaaagcaaatagcaatatggcaggagtaagtccttacttatcaataataccattgaatgtaaatggactaaactctccaatcaaaagacatagagtggctgaatcaattaaagaaaaaacaagacccattgatctgttgtccacaagaaacacactttatctataaagacacacatagactgaaaacaaagggatggaaaaagatactccacgccaatggaaaccaaagaaagagcaggagtagctacacttatatcaggcaaaatagatttcaagacaaaaactataagaagagacaaggtcactaatgataaacaggtcaattcagcaagaggatataacaattgtaaatatatatgcacccaatgctggagcacccagatatataaagcaagtatttactagagctaaagagagaaatagactccaatgcaataatagctggagatttcaacatcccactttcaacattgaacagatcctccagatagaaaatcaacaaagaaatattggacttaatctgcactatcgaccaaatggatctaacagatatttacagaacatttcatccaacagctgcagaacacacattcttttcctcagcacatagatcattctcaaggatagaccatatgttgggtcacaaaacaagttttaaaatattcaaatacattgaaataatatcaagcatcttctgtgaccacaatggactaaaactagaaatcaataacaagaggaattttggaaactatataaatatatggaaattaatgaatgctgagtgggtcaatgaagcaattaagaaggaaactgaaatttttcttggaacgaatgatcatggaaacagaaaataccaaaacctatgggatacagcaaaagcagtactaagagggaagtttacagctacaaatgcttacattaaaaaagaagaaaaacttcaataaaaaaacctaacaatgcatcttaaagaactagaaaagcaagaggaaatcaaatccaaaattagtagaagaaaacagtaaaggtcagagcagaaataagtaaaattgaaatgaagaaaacaatacaaaagatcaataaaacaacaggttgttttcttgaaaagttaaacaaaattgacaaacctttagccagactaagaaaaaaagacagaagatccaaataaataaaatcagagatgaaaaaggtgacattacaacttacaccacagaaattcaaaggatcattagtggctactataagcaactatatgccaataaattggaaaatctagaagaaatgcagaaattcctagacacatacaacctcccaagattaaaccaagaagaaattcaaaacctgaacagactgataacaagtaatgagatcaaagccgtaataaaaagcctcccagtaaagagaagcccaggacccgacggcttcactgctgaattctaccaaacatttaaagtagaactaataccaatcctactcaaactattccaaaaaatagaggtggaaggaatacttcaaaactcattatacgaggccagtattaacctgacaccaaaactagacaaagacacatgaaaaaaagaaaactacaggccaatatgtctgatgaatattgacacaaaaatcctcaacaaaatactagcaaaccaaattcaactacacattagaaagttcactcatcatgaccaagtggaatttatctaacttgggatgcaaagatggttcaacatatgcaaatcaatcaatgtgatacatcatatcaacagaatgaacaacaaaaaccatttgatcatttaattgatactgaaaaagcatttgataaaattcaacattccttcataataaaaattctcttctatactaggtacaaaagaaacttacctcaacataataaagccatatatgacagtcccacagtatgatactaaatgaggaaaaactgagagcctttcctctacgatctggaacatgacaaagatgcccactttcatcactgttattcaacatagtactggaagtcctagctggagcgatcagacaagagaaagatataaaagacatccaaattggaaaggaataagtcaaattatcctcatttgcatatggtatgatcttctatttagagctaactaaagactccaccaaaaaaagttattagaactgacgaacaaattcagtaaagctgcaggatacaaaatcaacatacaaaaatcagtagcatttctatatgccaacaatgaccaatgtgaaaaagaaattaaaaagtaaccctatttacaataaccacaaataaacacctaggaattaaccaaagaggtaaaagatttctgtaatgaaaactataaaaactgatgaaagaaattgaagagtacaccaaaaaatggaaagcaattgcatgttcatggattagaagaatcagtgttgttataatgtccatactatccaaagcaatctacagattcaatgcaatccttatcaaaataccaatgacatcattcacagaaatagaaaaaaaaaatcctaaaatttacgtggaaccacaaagacccagaatagccaaagctctcctaagcaaaaagaacgaaactgtaggaatgacattgcctgtcttcaaattctactacagagctatagatagtaaccaaaacagcgtggtactagcataaaaacagacacagagacaaacagaacaaaatttaaaaacccagaaataaatccacacacctacagcaaattcatttttgacaaagttgccaagaacatactctggggaatagataatgatatctcttcaataaataatgtggggaaaactggatatccatatacataacagtgaaactagacccctctctctctcactatatacaaaaatcaaatcaaaattgtttaaggacttaaatctaagacctcatactatgaaaccactgcaagacaaccttggcggaaactctccaagacatcagtccaggcaaagatttcttgagtaatatcccacaagcacagacaaccaaagcaaaaatggacaaatgggatcacatcaagttaaaaagcttctgcacagtaagggaaacaaccaacaaaatgaagagacaacccacagaatgggagaaaatatttgaaaaatacccatctggcaagggattaaaaaccagaatatatgcagaatatataaggagctcaaacagtgctatagaaaaaaaaatctaataatctgatttaaaaatgggaaaaatgttagaatagacatttcttaaaataagacatacagatggcaaaccgacatggaacggtgctcaacatcatggattatcacagaaacacaatcaatcaaaactaaaactaaaatgtgctatcatctcaccccagttaaaatggctgatatccagaagacaggcaataacaaatgctggcaaggatgtggggaaaagggagcccccatacactgttgctgggattgtaaattagtacaaccactgtggagagcagcatgaaagttcctcaaaaaactgaaagaaagctaccataggatccagcaatcccactgctgtgtatatactacaaaagaaaggaagtcagtatatgaagaggtatctgcactcccatgtttgttgcagccctgttcacaacagccaagatttggaagcaacctaagtgtccatcagcagttgaatgtataaagaaaatgtggtgcatatacacaatggagtattattcaataataaaaaggaatgagattgagtcatttgcaacaacatggatggaactggagatcattatgtgaagtgaaataagccaggcacagaaagacaaacattacaatgttcttacttattaatgagatctaaaaatcaaaacaattgcacccatgttcataaagagtaaaaggatggttaccagatgctgagaacggtggtggggggatagggaaaggtggcagtggttaacgggtacaaaaaaatagaaagaatgaataagacttactacttgatagcacagcaaggtggctatagtcagtaatttagttgtatatttttaataatgaaaggtgtataattggattgtttctaacacaaaggataatgcttaagaggatggataccccattttccatgatgtgattatttcacattgcacgcctagatcaaaacatccaatgtaccccataaatatatacatcttctatgtacccataaaaattctgtaaaataaaatatataaaaagaggtgacagatatggaagacaggcaaagaagagacgacatccacataatccgagtacctaagaaagaatggagtccagtgcatctcaggagccaccattctaagccaattttctctggttctctcagtcaccctactaatacgtgggcaatcttgttttatttcaggatagagtttttgaaattatagatttaagtatgctttctgttctattacttttggtaattaattttagaaagaactaatttgggcacaaatttgaaaaaattctaaatccaaaaaaaaaaagaaaaaaacacacacacaatcatctataagggggatgatgaccagtcctagatttctcaccagccacattcaagatcagtaaatggtaggacaaaacctgtagggtccttaagggggaaagaagtagtggatagtccagagtctatatacagccaactgttcttgaagaaaaaaggctgctgaaaaggagttccaaacattctataatccataatctcatgatgaaactactagaggaagaccaccagccatcaaaaggtgcttggagaacccagggccaagaaccaaaagtaaatattaagtgtccttaactgcgagactaagatagaaatgactgtgggggaccatgtggcctcaacagaggtgaaatggtgtctgcctgacaaagtggacattttacaatgatcaaaacacagaatatgagatagagagcacttctgaattactgcctcactccaaataactctcagccaaaggacttcagtaaaaccaaattgggcatattagacagtacaaacaaattctaagaaaataatattactgattacaatcacatgatgctagagatggaggggaaaaggaagaggaaaccaggtaatttcatactcgtatatagtaaagaactaaagtacattgtccaaagaagaacaaagaatattttggaaagttataaaggtagccactacacatagaagatagcaaagaacaagaaaacttaagatggaaaactttttggaagcataaaaatagaaaatataaactactaagataagattgaagccaaacagatctatgaaaacaacaaacatcaatggccttaacttgcctattaaaaggaagagactttcaaattggaccacaagataaaacccaactctatatagcatatgagtattacacacaaaatgggaaaagctgaaaaaacttgggcaaaattcaccccaagcaaattccactgtttcctttgggacaaaatgccaagctccatgccagggaagatgattctcctcagaccttctcctcactctcccagtcctcttagggaaggaattgggtgttagaggagggagactctgtcgattatcagctgaagcagtggtgtgctcctgcgttgcttctgacctgggaaatgaagcagcaagactctttctgctgtgtctttgcccagaagggccatccccccagagcagagtacccaggccggcaggagcagtggtggaagcgtggaaaccacgtctcctacagcagagaccatcagaagcggagcctcgggtataagggaaacaacgcgttctccctaacctgggagtgacagacagcgtcattcctcacagtgataccctgtgttctagccatctggcccatgacagagccagcccagagccagcccagagccagcccctcaccaacctggagcctggccagctcgccaagctgcaccataggcctggaaggcgtggagacctgcggcagtgccctgtcctcccgtgaggcctgccatccctgccaggggtcgcctctggcttctcctccaggaccgcacggtccagaggctcagtgcctggagtaggtgttgcccccctgcttctaggcccagaccctcccttgttcctgaccccgggcctttccctctggcttggacatccagggccctgtctcagctggggagctgctcctgctcaaggactgtcttccgcgggatcgaaaggccgcgtcctgaacaatgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgggagcggagcagactctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgggagcggagcagactctaaaggccgcgtcctaaacagtgtgtgggccacgtgagcggagcaggctctaaaggccgcgtcctaaacagtgcgtgggccacgggagcggagcagactctaaaggccgcgtcctaaacagtgtgtgggccacgtgagcggagcgccctctccactgccctcggggccgcagctcccagctcagctcccagccctgctcagggcagccaggccaggaggtaccatccaggctaagtgaccctcaggggggacaggtgccccaggagatgccagctgttgggagaggctgggggaccaactcgacctggcctgtgggccctgccctggccacccattgtaggatccagccgccacgcctgtgacactcgtgtgctttccctggtgtgtgcttgtggcaggtgggggcagagggtcctcaggccagagagccactcccccagcgccagaccaccctcttcctcactcccccacctcaccccctcacaggtgcctcccaggccatcagggcccaaccacccctaaacaaatgggttctcggcccctcgtggctggaggtgggttctctcaccattcccagcctaaggctccatccccatgctggcagctgttcaaccatgtctagagagatccactgtcccagacagcacctcagggtcccccgtcctgcctggaaccctgtaggaaactccacaaaccgccgccattctgtccacacccctacaggagccccaaccctctccccacatccaggcttccctcccagacccctcatccctgcccgcacggtgcctgagggggccttcttgggcagcgcctaagcaagcccccagcacccttcggccccttcaaggcacacaggccccctttccacccagcctcaggaaaccacctgtgtcctccaacgacaggtcccagcctcccagcctttgccttgcctgttcctctccctggaactctgccccgacacagaccctccccagcaagcccgcaggggcacctcccctgcccccagacaccctgtgcccgtcagttcatccccagcagaggccctcaccaggcacacccccatgctcacacctggccgcaggcctcagcctccctgagggccccacccagcccgcgtctggccagtggtgcgtgcaaagcccctcacccagactcggcggaaggcagccagtgcaggcctggggaggggctctccttagaccaccttgcaccttccctggcacccaccatgggaagagctgagactcactgaggaccagctgaggctcagagaagggacccagcactggtggacacgcagggagcccacgccagggcgccgtggtgagtgaggcccagtgccacccactgaggcctcccgttcagtgggacgacggtgaacaggtggaaccaaccaggcaacccccgccgggccccacagacgggatcagagcaggaaaggcttcctgcccctgcaggccagcgaggagccctggcgggggccatggccctccaggcgaggaggctcccctggccaccgccacccgggcctctctgctgctgggaaaacaagtcagaaagcaagtggatgagaggtggcgtgacagacccagcttcagatctgctctaatttacaaaagaaaaggaaaaacacacttggcagccttcagcactctaatgattcttaacagcagcaaattattggcacaagactccagagtgactggcagggttgagggctggggtctcccgcgtgttttggggctaacagcggaagggagagcactggcaaaggtgctgggggcccctggacccgacccgccctggagaccgcagccacatcagcccccagccccacaggccccctaccagccgcagggttttggctgagctgagaaccactgtgctaactggggacacagtgattggcagctctacaaaaaccatgctcccccgggaccccgggctgtgggtttctgtagcccctggctcagggctgactcaccgtggctgaatacttccagcactggggccagggcaccctggtcaccgtctcctcaggtgagtctgctgtctggggatagcggggagccaggtgtactgggccaggcaagggctttggcttcagacttggggacaggtgctcagcaaaggaggtcggcaggagggcggagggtgtgtttttgtatgggagaagcaggagggcagaggctgtgctactggtacttcgatctctggggccgtggcaccctggtcactgtctcctcaggtgagtcccactgcagccccctcccagtcttctctgtccaggcaccaggccaggtatctggggtctgcagccggcctgggtctggcctgaggccacaccagctgccatccctggggtctccgccatgggctgcatgccagagccctgctgtcacttagccctggggccagctggagcccccaaggacaggcagggaccccgctgggcttcagccccgtcagggaccctccacaggtagcaagcaggccgagggcagggacgggaaggagaagttgtgggcagagcctgggctggggctgggcgctggctgttcatgtgccggggaccaggcctgcgctttagtgtggctacaagtgcttggagcactggggccagggcagcccggccaccgtctccctgggaacgtcacccctccctgcctgggtctcagcccgggggtctgtgtggctggggacagggacgccggctgcctctgctctgtgcttgggccatgtgacccattcgagtgtcctgcacgggcacaggtttatgtctgggcaggaacagggactgtgtccctgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggccatgtggggccctccggggctccttctccggctgtttgggaccacgttcagcagaaggcctttctttgggaactgggactctgctgctggggcaaagggtgggcagagtcatgcttgtgctggggacaaaatgaccttgggacacggggctggctgccacggccggcccgggacagtcggagagtcaggtttttgtgcaccccttaatggggcctcccacaatgtgactactttgactactggggccagggaaccctggtcaccgtctcctcaggtgagtcctcacaacctctctcctgctttaactctgaagggttttgctgcatttttggggggaaataagcgtgctgggtctcctgccaagagagccccggagcagcctggggggctcaggaggatgccctgaggcaacagcggccacacagacgaggggcaagggctccagatgctccttcctcctgagcccagcagcacgggtctctctgtggccagggccaccctgggcctctggggtccaatgtccaacaacccccgggccctccccgggctcagtctgagagggtcccagggacttagcggggtgccagttcttgcctggggtcctggcattgttgtcacaatgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaagctggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgatagggacaaagagtggagtggggcactttctttagatttgtgaggaatgttccgcactagattgtttaaaacttcatttgttggaaggagagctgtcttagtgattgagtcaaggaagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactatgactttaaaatgtgagagggttttcaagtactcatttttttaaatgtccaaaatttttgtcaatcaatttgagtcatgtttgtgtagaactgatattactgaaagtttaaccaaggaataggaatgatgctctttcataccctattcagaactgacttttaacaataataaattaagtttaaatatttttaaatgaattgagcaatgttgagttggagtcaagatggccgatcagaaccagaacacctgcagcagctgtcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccaaagcatttacagtgactttgttcatgatcttttttagttgtttgttttgtcttactattaagactttggacagcatttatacagtatccgatgcatagggacaaagagtggagcggccgcagcacgtgttgacaattaatcatcggcatagtatgtcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgattttacagattttatgtttagatcttttatgcttgcttttcaaaaggcctgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccaccggcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaatacgccaaccaagtatttcggagtgcctgaactgtttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgttaaccgttgatttttactgattgg